(19) United States
(12) Patent Application Publication
(10) Pub. No.: US 2015/0352189 A1
(43) Pub. Date: Dec. 10, 2015

(54) ACTIVATION OF THE ENDOGENOUS ILEAL BRAKE HORMONE PATHWAY FOR ORGAN REGENERATION AND RELATED COMPOSITIONS, METHODS OF TREATMENT, DIAGNOSTICS, AND REGULATORY SYSTEMS

(71) Applicants: Jerome SCHENTAG, (US); Joseph FAYAD, (US)

(72) Inventors: Jerome Schentag, Amherst, NY (US); Joseph Fayad, Las Vegas, NV (US)

(21) Appl. No.: 14/759,283

(22) PCT Filed: Jan. 8, 2014
(86) PCT No.: PCT/US14/10617

§ 371 Date: Jul. 6, 2015

Related U.S. Application Data
(60) Provisional application No. 61/750,042, filed on Jan. 8, 2013.

(51) Int. Cl. A61K 38/26; A61K 31/4985; A61K 38/17; A61K 45/06; A61K 31/138; A61K 31/40; A61K 31/25; A61K 38/05; A61K 31/4178; A61K 31/155; A61K 9/28

(52) U.S. Cl. CPC A61K 38/26; A61K 31/155; A61K 31/4985; A61K 38/1709; A61K 45/06; A61K 9/2846; A61K 9/288; A61K 31/40; A61K 31/25; A61K 38/05; A61K 31/4178; A61K 31/138

(57) ABSTRACT

In one embodiment, the invention provides a method of regenerating organs and tissues in a subject suffering from one or more organ or tissue manifestations of glucose supply side associated metabolic syndrome, the method comprising: (a) confirming that the subject suffers from or is at risk for suffering from organ and/or tissue damage associated with a glucose supply side associated metabolic syndrome; and (b) co-administering to the subject an effective amount of a pharmaceutical composition comprising a first and optionally a second active composition, said first active composition comprising an ileal brake hormone releasing substance encapsulated within an enteric coating which releases said substance within said subject's ileum and ascending colon causing release of at least one ileal brake hormone from L-cells of said subject, said optional second active composition being formulated in immediate and/or early release form in an over coating onto said enteric coating, wherein said second composition is beneficial to at least one aspect of said subject's metabolic syndrome manifestations. Coadministration methods with a second pharmaceutical composition are also disclosed.

### RELATED APPLICATIONS

This application claims the benefit of priority of U.S. provisional application No 61/750,042, entitled “Activation of the Endogenous Ileal Brake Pathway for Organ Regeneration and Related Compositions, Methods of Treatment, Diagnostics, and Systems, filed Jan. 8, 2013, the entire contents of which is incorporated by reference herein.

### FIELD OF THE INVENTION

The inventors disclose herein a new pathway and system controllers for organ and tissue regeneration, and pharmaceutical compositions to regulate and control said processes. Accordingly, the invention provides pharmaceutical compositions, methods for the treatment, diagnostics and computer-implementable systems that relate to regeneration of organs damaged by a variety of metabolic syndromes, including hyperlipidemia, insulin resistance, hypertension, atherosclerosis, fatty liver diseases and certain chronic inflammatory states, among others.

In one embodiment, the invention provides a method of regenerating pancreatic beta cells of a subject suffering from Type II diabetes (T2D). The method comprises administering to the subject in need a pharmaceutical dosage form comprising an ileal brake hormone releasing substance comprising at least one enteric coated or delayed release microencapsulated sugar, lipid, or amino acid, in an effective amount, whereby release of said substance from the pharmaceutical dosing form activates the subject's ileal brake in a manner similar to RYGB surgery. In preferred embodiments, metformin in a daily dosage of 500 mg to about 1000 mg (low dose metformin) is ideally coated onto the outer surface of the enteric coated pharmaceutical dosage form. In an alternative embodiment, microcapsules of metformin are mixed with microcapsules of the ileal brake hormone releasing substance in a dosing form ideally given to T2D patients once daily. The release of ileal brake hormones increases pancreatic beta cell mass in the T2D patient and typically and uniquely normalize the patient's insulin secretion, insulin resistance and HBA1c. As further demonstration of heretofore unexpected pancreatic regeneration, the effects of daily use of the dosage form for 6 months persist for prolonged periods even if the medication is not taken.

In another embodiment, the invention provides a method of regenerating hepatic cells of a subject in need suffering from Hepatic Steatosis or Non Alcoholic Fatty Liver Disease (NAFLD). The regeneration method comprises administering to the subject in need a pharmaceutical dosage form comprising an effective amount of said ileal brake hormone releasing substance comprising at least one enteric coated or microencapsulated sugar, lipid, or amino acid, whereby release of said substance from the pharmaceutical dosing form activates the subject's ileal brake in manner similar to RYGB surgery. Atorvastatin in a daily dosage of 5.0 mg to about 20 mg is ideally coated onto the enteric coated pharmaceutical dosage form or microcapsules of atorvastatin are combined with microcapsules of the ileal brake hormone releasing substance in a dosing form ideally given to Hepatic Steatosis or NAFLD patients once daily. In a further combination of the ileal brake hormone releasing substance, the atorvastatin may be replaced in the dosage form by any other available statin in a low dosage equivalent in potency to the chosen dosage of atorvastatin. In a further practice of the invention, berberine may be substituted for a statin in the formulation. Release of ileal brake hormones increases hepatic cell mass and decreases the number of inflamed hepatic cells in the Hepatic Steatosis patient and typically and uniquely normalizes triglycerides, hepatic enzymes, alpha-fetoprotein and cholesterol. As further demonstration of heretofore unexpected hepatocellular regeneration, the effects of daily use of the dosage form for 6 months persist for prolonged periods even if the medication is not taken.

In another embodiment, the invention provides a method of decreasing cellular inflammation and regenerating neural cells, including neural cells of a subject in need suffering from neuropathy, neurodegenerative diseases or Alzheimer's disease as associated with T2D or Metabolic Syndrome. The regeneration method comprises administering to the subject a pharmaceutical dosage form comprising an effective amount of said ileal brake hormone releasing substance comprising at least one enteric coated or microencapsulated sugar, lipid, or amino acid, whereby release of said substance from the pharmaceutical dosing form activates the subject's ileal brake in manner similar to RYGB surgery. Memantine in a daily dosage of 5.0 mg to about 20 mg is ideally coated onto the enteric coated pharmaceutical dosage form or microcapsules of atorvastatin are combined with microcapsules of the ileal brake hormone releasing substance in a dosing form ideally given to Alzheimer's disease afflicted patients once daily. In a further combination of the ileal brake hormone releasing substance, donepezil or any medicament known to be active for improvement in brain function may be substituted in the formulation in an effective dose. The inventors have shown that release of ileal brake hormones improves neuronal function and decreases the number of inflamed neuronal cells in the patient and typically and uniquely normalizes brain biomarkers of Alzheimer's such as APP, tau and beta amyloid precursor proteins, among others(1). As further demonstration of heretofore unexpected neuro protective effects and neural regeneration, the effects of daily use of the dosage form for 6 months persist for prolonged periods even if the medication is not taken.

In another embodiment, the invention provides a method of decreasing cellular inflammation and regenerating vascular endothelial and cardiac myocyte cells, including vascular endothelial cells of a subject in need suffering from Atherosclerosis, Atherosclerotic Cardiovascular disease (ASCVD), hypertensive cardiovascular diseases, in particular but not limited to ASCVD as associated with T2D or Metabolic Syndrome. The regeneration method comprises administering to said subject a pharmaceutical dosage form comprising an effective amount of said ileal brake hormone releasing substance comprising at least one enteric coated or microencapsulated sugar, lipid, or amino acid, whereby release of said substance from the pharmaceutical dosing form activates the subject's ileal brake in manner similar to RYGB surgery. Lisinopril in a daily dosage of 5.0 mg to about 20 mg is ideally coated onto the enteric coated pharmaceutical dosage form or microcapsules of lisinopril are combined with microcapsules of the ileal brake hormone releasing substance in a dosing form ideally given to ASCVD afflicted patients once daily. In a further combination of the ileal brake hormone releasing substance, any available ACE inhibitor or AII inhibitor or any medicament known to be active for improvement in cardiovascular function may be substituted in the formulation in an effective dose. Release of ileal brake hormones lowers systemic inflammation and improves cardiovascular function and decreases the number of inflamed endovascular cells in the patient and typically and uniquely normalizes cardiovascular biomarkers such as hsCRP, insulin resistance, triglycerides, cholesterol, HBA1c, among others. As further demonstration of heretofore unexpected cardio protective effects and endovascular regeneration, the effects of daily use of the dosage form for 6 months persist for prolonged periods even if the medication is not taken.

In another embodiment of the invention, any medicament employed for treatment of one or more components of metabolic syndrome or its associated diseases, or certain probiotic organisms, may be combined with the enteric coated or microencapsulated ileal brake hormone releasing substance, said compositions and methods acting by treatment of the component of metabolic syndrome in combination with substances that activate the ileal brake, which acts in the pancreas, gastrointestinal tract and the liver of a mammal to control metabolic syndrome manifestations and thereby reverse or ameliorate damage (pancreatic beta cell death or apoptosis, atherosclerosis, hepatic steatosis, hypertension, lipid accumulation, and the like) resulting from progression of metabolic syndrome and associated inflammation. It is noted that when a second bioactive agent is combined with the ileal brake hormone releasing substance for treatment of a subject, the amount of such agent which is used therapeutically is often in low dose, i.e., the amount of the second agent which can be used effectively in pharmaceutical compositions according to the present invention is generally substantially less than the dosage used when the agent is administered alone (i.e., often as little as about 5% to 80% or 10% to about 50%, or about 20% to about 35% of the normal dosage administered to patients in the absence of the ileal hormone releasing substance). It is also noted that in alternative embodiments, the second bioactive agent (an additional bioactive agent) may be used and administered in a separate pharmaceutical formulation/composition in a coadministration embodiment which relies on more than one pharmaceutical composition to effect the intended result on organ/tissue regeneration and treatment, including inhibition of damage to organs and tissue.

### BACKGROUND AND DESCRIPTION OF THE INVENTION

This application incorporates by reference the complete disclosure of U.S. provisional application No. 61/750,042, entitled “Activation of the Endogenous Beal Brake Pathway for Organ Regeneration and Related Compositions, Methods of Treatment, Diagnostics, and Systems, filed Jan. 8, 2013 and references incorporated therein.

Background information regarding the nature and interrelationship of Roux-en-Y gastric bypass (RYGB)) and the ileal brake is provided in the related applications identified above and U.S. patent application Ser. No. 12/911,497, described above.

A significant but poorly recognized problem with metabolic syndrome and certain end organ manifestations like T2D is the progressive loss of hormone mediated pancreatic, Liver, kidney, GI, cardiovascular, brain and other organ repair and regeneration capabilities. The pace of metabolic syndrome damage increases as endogenous repair and regeneration pathways and processes shut down. Meanwhile, a continual supply of immediately available carbohydrates drives the excessive output of the pancreatic beta cells. Glucose supply driven pancreatic stress in absence of ileal hormone signaled pancreatic repair leads to pancreatic exhaustion, acceleration of insulin resistance, T2D, and non-alcoholic fatty liver disease (NAFLD), all of which are core end-organ manifestations of Glucose Supply Side driven metabolic syndrome.

The bacterial metabolism of nutrients in the gut is able to drive the release of bioactive compounds (including short-chain fatty acids or lipid metabolites), which interact with host cellular targets (enterocytes called L-cells for example) to control energy metabolism and immunity. Both animal and human data demonstrate that phylogenic changes occur in the microbiota composition in obese versus lean individuals; they suggest that the count of specific bacteria is inversely related to fat mass development, T2D, and/or the low levels of inflammation associated with cardiovascular risk. In particular, certain microbial species that disappear during acceleration of metabolic syndrome include Faecalibacterium prausnitzii, Bacteroides thetaiotaomicron, and Lactobacillus johnsonii, among others. In specific examples of this invention, ileal brake hormone releasing substances are beneficially combined with these probiotic bacterial species to lower the intensity of metabolic syndrome and its manifestations in the human patient. To the extent that replacing the dysbiosis strains with these beneficial strains occurs, the systemic inflammation associated with metabolic syndrome declines.

Pancreatic beta-cell deficiencies of insulin production are a pathophysiologic component of diabetes mellitus and a primary result of islet dysfunction. Islet cell dysfunction is a prerequisite for the development of T2D since individuals with insulin resistance do not develop hyperglycemia unless beta-cell compensatory production of insulin also fails. Current therapeutic approaches to T2D involve the administration of exogenous insulin or stimulating the weakened pancreas to produce more. Current approaches do not address the excess glucose supply. Thus, there is no reversal or regeneration effect in current therapy. In T2D, the primary defect is increased beta-cell apoptosis. Since replicating beta-cells are more vulnerable to apoptosis, the pro-apoptotic diabetic milieu limits the regenerative capacity of the islet cell mass and directly causes accelerated islet cell loss. Neither insulin, DPP-IV inhibitors, nor TZDs address this problem. Pancreatic decline continues in a progressive manner.

Clearly, therapeutic approaches for T2D caused by metabolic syndrome need to lower the glucose supply, lower insulin resistance in tissues and thus decrease the demands on the pancreas. Secondly, therapeutic approaches need to address the dynamics of islet turnover (regeneration and cell loss) in order to be successful. It may be anticipated that such an intervention is also most effective early in the course of diabetes or in pre-diabetic conditions. The present invention of an orally active Roux-en-Y (RYGB) mimetic demonstrates, for the first time, a pharmaceutical which simultaneously decreases glucose supply, causes a decline in insulin resistance, and increases beta cell output of insulin by regenerating pancreatic beta cells, an unexpected result. The disclosed pharmaceutical combination of a first controlled release active agent with a second immediate (release in the stomach) or early (such as in the duodenum or jejunum) release active agent create a normal pattern of homeostasis and a favorable improvement on regeneration pathways, accompanied by a reduction in apoptotic loss of cell mass. Even more novel in the environment of treatments that palliate rather than cure, the present invention also demonstrates regeneration properties for other organs and tissues, such as liver, GI tract, neuronal tissue and others.

FIGS. 9-14 herein depict various nutritional and hormonally mediated metabolic relationships implicated in the regeneration of organs damaged by a variety of metabolic syndromes, as explained and generally described hereinafter, including, more specifically, in the brief description of the figures.

FIG. 9 shows the system that includes the master controller, called the ileal brake, a metabolic regulatory process based in the distal intestine (jejunum, ileum, right colon). The system includes Drivers, a Metasensor, Effectors and Beneficiary organs and tissues that are regenerated including pancreas, liver, GI, CV and CNS. The hormones regulating this axis of nutritional and metabolic control are released under control of both probiotic organisms and intestinal enterocytes, which together form a Metasensor (multiple components interacting to provide regulatory balance). The Metasensor effects changes in metabolism via release of both stop signals (appetite suppression, satiety) and repair/regenerate signals (immunomodulatory, anti-apoptotic, mitotic). The system efficiency is optimized so that excess nutrient is stored as adipose and released as needed to aid repair or provide energy supply.

FIG. 10 shows the normal Nutritional and Metabolic System in Homeostasis, with all components of the Metasensory System in balance. Dietary intake is normal and some excess nutrition reaches the distal intestine because it is not absorbed proximally in the duodenum and early jejunum. However, when the patient ingests only IR (immediate release)-CHOs (carbohydrates), the bacteria in the ileum are not achieving nutrition (nutrients are all absorbed proximally leaving no distal nutrition). They react by signaling a suppression of ileum L-cell output and hunger ensues. If, on the other hand, the patient is having a balanced diet with portions reaching the bacteria, they have no reason to suppress the L-cell output and normal eating produces satiety.

FIG. 11 demonstrates the impact of “Supply Side” mediated excessive intake of CHOs with immediate release characteristics: What ensues is a Metasensor mediated hunger from a DIETARY IMBALANCE(2, 3); there is rapid duodenal absorption of IR-CHO in with closely linked pancreatic stimulation; CHO Storage short term as visceral fat; Insulin Resistance; minimal to no regeneration occurs in absence of ileal brake signaling. The result of excessive IR carbohydrate loading is a Metasensor system out of balance; Nutrient imbalance develops and creates a distal flora imbalance; e.g. a plentiful supply of IR (immediate release) CHO (carbohydrates), for example sugar sweetened beverages. Bacteria are Hungry so the mammalian host is hungry, Excess insulin production drives central adiposity (favors storage at these sites) and insulin resistance accelerates in response to a progressive flood of IR nutrition as the host becomes more and more hungry to feed this dysbiosis pattern.

In FIG. 12, we demonstrate the mechanism of action of nutrients ingested in a patient who is post RYGB surgery. RYGB mechanically diverts ingested contents past the absorptive (but non-signaling) area, and bombards the signaling areas further downstream in late jejunum and ileum. Specifically, there is a diversion of the sugar to the distal ileum, where the L-cells are stimulated and the distal intestinal flora are now receiving excessive nutrition. Both combine to extinguish the hunger signals. Since caloric intake is dramatically lowered, in this setting fat is mobilized from both liver and adipose storage, and the pancreatic stress is lowered considerably. Insulin resistance is resolved by RYGB surgery. The arrival of massive nutrients at the ileum in such a large quantity creates a “malabsorptive emergency” and initiates the satiety signal by shutting down the hormonal release from the L-cells to regenerate signaling to a certain extent with the same or less amount of food needed, therefore restoring maintenance and regeneration. And because it is not individualized, RYGB surgery will trigger more regeneration than signaling, to the point where 2-4 years following the procedure, the jejunum segment will have evolved to restore proximal absorption to a baseline levels

Notwithstanding the advances that have been made in understanding and treating metabolic syndromes, the need continues to exist for a comprehensive treatment strategy that not only addresses end organ manifestations such as T2D, but also ameliorates concomitant disorders such as NAFLD, hypertension, neuronal damage and fundamental gastrointestinal changes including intestinal flora disruption. Ideally, as in the present invention disclosed herein, the primary treatment benefit offered to patients with T2D and other metabolic syndrome manifestations is the regeneration of the important organs of nutrition, and the lowering of systemic inflammation. The primary benefit of the disclosed oral mimetic of RYGB surgery is an equivalent regeneration signal to RYGB surgery itself, which is a very novel observation, considering that when following the teachings of the instant invention, pancreatic regeneration is produced by approximately 10 grams of a refined sugar, typically dextrose but not limited to that molecule, applied by formulation to the ileum and right colon, the site of the ileal Brake. We call the effective formulation Brake™.

There are no currently effective regeneration strategies for the pancreas, which is why end stage Type I diabetes (T1D) is treated with pancreas beta cell transplants. The problem is that once cells are transplanted, there is an accelerated loss to inflammation and apoptosis and soon there is a need for additional transplanted cells. Current approaches to drug therapy replace missing components, such as insulin. This is widely known as effective, but it does not repair the underlying problem of diabetes. RYGB surgery on the other hand is widely known to resolve diabetes, and the best consensus of the effect is a regeneration of pancreas, liver and GI tract, as well as nearly complete reversal of cardiovascular injury and indeed, metabolic syndrome itself. Overall, however, this highly effective treatment is restricted to use in patients with morbid obesity, and it has not been well understood why metabolic syndrome is also improved in these patients who undergo surgery. The inventors none the less calibrated the hormonal effects of RYGB against the disclosed oral mimetic formulation called Brake™, for purposes of inventing an organ and tissue regenerative approach to metabolic syndrome and T2D.

As shown in FIG. 13, the formulation called Brake™ and disclosed herein acts distally in the jejunum and ileum in the same manner as RYGB surgery. There is the same sensation of a “malabsorptive emergency”, the same activation of L-cells, the output of which promotes regeneration in GI, Liver and Pancreas: The same subsequent response is noted, as hunger disappears into a strong signal of satiety. We calibrated the dosage of Brake™ to produce the same hormonal output as RYGB surgery. The ileal hormone signal from Brake™ occurs later than that of RYGB, and the peak of GLP-1 output is not as high as produced by RYGB. However the GLP-1 signal can be more prolonged because of the delayed release formulation. Thus with Brake™, the intensity of the stimulation will be more moderate and closer to physiological and therefore regeneration proceeds in liver, pancreas, GI enterocytes in a much more natural and physiological way compared to surgery. The stress on the pancreas recedes, the distal ileum receives the nutrients, quieting the bacteria and increasing the output of the L-cells. Fat is mobilized from both liver and adipose tissue. As expected, weight loss is more rapid with RYGB than Brake™ treatment, since RYGB surgery also physically decreases the size of stomach, limiting ingestion in a second, profound manner over the ileal brake pathway alone.

### SUMMARY OF THE INVENTION

The present invention provides pharmaceutical compositions comprised of a controlled release core of an ileal brake hormone releasing substance and an over-coated outer immediate (stomach) or early (duodenum or jejunum) release layer of a second active agent. These medicaments beneficially affect glucose supply, insulin resistance and when used in patients afflicted are effective methods of regenerating organs and tissues in a patient afflicted with one or more organ or tissue manifestations of glucose supply side associated metabolic syndrome, when the syndrome is accompanied by suppressed regenerating processes and progressively failing organs. A pharmaceutical composition in an effective dosage is provided to said metabolic syndrome patient, which activates the dormant ileal brake sensor and initiates renewed hormonal signals to regenerate candidate organs and tissues including but not limited to the pancreas, the liver, the gastrointestinal tract, including enterocytes of the GI tract, kidneys, lungs, cardiovascular system, central nervous system (brain) and the associated signal transmitting neurons.

By way of example, directly regenerating pancreas, liver and gastrointestinal tract functions are specifically described herein and attributed to treatment with a specific pharmaceutical composition having its primary action on the L-cells of the ileum, said action being release of hormones and signaling molecules. These actions are assured in the practice of the instant invention by measured biomarkers of both the ileal hormone process and the resolution of metabolic syndrome and organ repair. In particular, the present invention generally proceeds when the steps in practice of the invention include the testing for abnormal biomarker patterns; the administration of a pharmaceutical composition targeted to a specific receptor cell in the distal intestine; measurement of biomarkers demonstrating the precise sequence of ordered hormonally produced events beginning with cessation of hunger; a wake up stimulation of distal intestinal L-cells that have been quieted by actions of altered intestinal bacteria or consequences of metabolic disease; release of hormones and signals from said L-cells; said released hormones traveling in portal blood to pancreas, liver and GI tract, said organs regenerated from available growth factors with actions choreographed by pharmaceutical dosage form-controlled actions of said ileal brake hormones and hormone signals, measured biomarkers of the FS index demonstrating the successful regeneration and said regenerated organs then signaling the patient, preferably a human, to resume adequate nutrition seeking behavior as directed by normalized signals of hunger. Specific actions on organ regeneration are confirmed by measured biomarkers and analysis of the results. In certain cases where biomarkers are measured, the results may be used to change the dosage or dosing frequency or dosing time to optimize the regeneration of said patients organs and tissues.

Dependent on reserve capabilities of the organs of the patient at hand, and depending on composition and administered dosage of the pharmaceutical composition, the present invention relates to dramatic improvement or potential cure of metabolic syndrome manifestations including but not limited to T2D, hyperlipidemia, atherosclerosis, insulin resistance, hypertension, and Hepatic Steatosis. pancreas and/or pancreatic beta cell damage, hepatic steatosis, NAFLD, hyperlipidemia, elevated triglycerides, abdominal adiposity, atherosclerosis, cardiovascular diseases such as myocardial infarction, stroke, angina, congestive heart failure, hypertension, ASCVD, reduced lung capacity (COPD), Rheumatoid arthritis, diabetic nephropathy leading to kidney failure, gastrointestinal tract damage, gastrointestinal dysbiosis, inflammatory bowel disease, brain damage, neurodegenerative disorders, diabetic neuropathy, cognitive impairment associated with obesity and early Alzheimer's disease, among others.

Surprisingly, we have discovered a novel method of treating metabolic syndromes including T2D by administering to a subject in need thereof a relatively small amount (e.g. about 10-20 grams) of refined sugar formulations. Said formulations are specifically encoated in order to ensure the release of these refined sugars at the enteral target of the distal intestinal tract L-cells which regulate the glucose supply via appetite, and which regulate cellular regeneration in pancreas, liver and the GI tract itself. While not wishing to be bound by any theory, we postulate that our oral mimetics of RYGB surgery work by three interlocking mechanisms. First, by appetite suppressive signaling from the ileal brake, they decrease ingestion of sweets and fats and thus decrease the glucose supply of refined sugars. Second, the lowering of supply of immediate release glucose rapidly and permanently decreases insulin resistance. Third, the small amount of distal delivered refined sugar acts by ileal brake hormone release to enhance the beta cell response to the demands for insulin, thereby directly causing a moderate level of pancreatic beta cell regeneration, resolution of hepatic steatosis, and regeneration of GI tract enterocytes.

More specifically, we have discovered that the target pH for release of formulations of the invention must be optimized to a range of between about 7.2 and 7.5. While not wishing to be bound by any theory, we observed that this pH range is the same as that of the “sleeping ileal brake” in a T2D patient. We observed that the major defect of the ileal brake in T2D patients is not atrophy of the L-cells, but rather a lack of signaling attributable to three causes. First, the dietary ingestion of refined sugars leads to a huge bolus of glucose absorbed from the duodenum, and NONE of this sugar load reaches the ileum to trigger satiety or any of the other beneficial responses of the ileal brake, such as repair and regeneration of pancreas, liver and GI tract cells and functions. The absence of ileal brake regulatory signaling is a primary reason for pancreatic exhaustion in the collapse of compensatory pancreatic beta cell response. Secondly, the absence of an ileal brake signal to regenerate beta cell mass is a consequence of the rapidly absorbed high duodenal load of sugar. This refined sugar-fast forward pathway to obesity and T2D may be termed the glucose supply pathway to T2D(2, 3) which now appears to progresses unopposed by the ileal brake. Third, the ileal brake is quiescent if there is no glucose reaching the ileum to signal L-cells and quickly apply the ileal brake. The consequences of a quiescent ileal brake pathway are rapid weight gain and pancreatic exhaustion, as well as other organ damage. These pathways are further described earlier in FIGS. 9-14.

Methods of treatment and pharmaceutical compositions of the invention also lower the risk of cardiovascular complications of metabolic syndrome by acting in the distal intestine and liver to remove fat and lower the insulin demand. Insulin resistance declines immediately (within the first 24 hr to the first 7 days after application), even before there is any substantial weight loss from the formulation or the surgery.

The methods of treatment and pharmaceutical compositions of the invention stand in a marked contrast to the prevailing viewpoint that there is a deficiency of, or resistance to, insulin and an exhausted pancreas in T2D. Our novel metabolic syndrome treatment approach affects positively other organs affected by metabolic syndrome; the small amount of formulated sugar administered improves the liver, the kidney, the gastrointestinal tract and reduces lipid abnormalities leading to atherosclerosis. Moreover, the novel aspect beyond this first observation is long lasting regeneration of these organs of nutrition and metabolism.

Benefits of the novel methods of treatment and pharmaceutical compositions of the invention include, but are not limited to, ileal brake directed pancreatic beta cell regeneration, ileal brake directed hepatocyte regeneration and removal of excess fatty liver (NAFLD or Hepatic steatosis), and ileal brake directed promotion of maturation and replacement of gastrointestinal epithelial lining cells. Another benefit can be weight loss, although weight loss follows the other benefits and the other benefits occur even if the patient does not lose weight.

Accordingly, in one embodiment, the invention provides a method of regenerating organs and tissues in a subject suffering from one or more organ or tissue manifestations of glucose supply side associated metabolic syndrome, the method comprising:

(a) confirming that the subject suffers from organ and/or tissue damage associated with a glucose supply side associated metabolic syndrome by calculating the subject's FSindex, and optionally determining whether the subject's ileum has a pH of around 7.2 to around 7.5; and
(b) administering to the subject a pharmaceutical composition comprising between about 5 grams to about 20 (also, about 10 to about 20) grams of a refined sugar which is microencapsulated within an enteric coating which dissolves in vivo at a pH of around 7.2 to around 7.5, and optionally an effective amount of an additional bioactive agent as described herein.

Confirming that the subject suffers from organ and/or tissue damage associated with a glucose supply side associated metabolic syndrome by determining whether the subject's ileum has a pH of around 7.2 to around 7.5 may be accomplished by the Smart Pill® GI Monitoring System (Given Imaging; Yoqneam, Israel) or through use of other diagnostic techniques which are well-known to those of ordinary skill in the art. A pH-sensitive, radio-transmitting capsule whose location can be determined by X-ray is a preferred means of determining whether the subject's ileum has a pH of around 7.2 to around 7.5.

The enteric coating of pharmaceutical compositions used in the methods described herein may comprise one or more compositions selected from the group consisting of cellulose acetate trimellitiate (CAT), hydroxypropylmethyl cellulose phthalate (HPMCP), hydroxypropylmethyl cellulose, ethyl cellulose and mixtures of hydroxypropylmethyl cellulose and ethyl cellulose each of which contains a subcoating, polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, and mixtures thereof. Preferably, the enteric coating comprises one or more compositions selected from the group consisting of shellac, Eudragit® L, Eudragit® S, Eudragit® RL, Eudragit® RS and mixtures thereof.

Preferably, in the methods described herein, subsequent to administration of the pharmaceutical composition, the subject's level of GLP-1 expression is increased by a minimum of about two fold compared to pre-treatment levels.

The methods described herein may be used to treat a subject who suffers from Type 1 diabetes or Type 2 diabetes. Such a subject may express organ or tissue manifestations of glucose supply side associated metabolic syndrome such as pancreatic beta cell damage or death.

In certain aspects, the pharmaceutical composition administered in the methods described herein also comprises berberine, or a flavonoid such as coluteolin, apigenin, tricin and their pharmaceutically acceptable analogues and derivatives, or a flavonoid derived from the flavonoid rich fraction (FRF) of Oreocnide integrifolia leaves.

The pharmaceutical composition administered in the methods described herein may ideally also comprise any of the usual medicaments used for the treatment of any of the individual manifestations of metabolic syndrome. In each case immediate or early release forms of these medicaments may either be over-coated onto the enteric release dosage form of the ileal brake hormone releasing substance, or the microgranule formulation of ileal brake hormone releasing substance may be blended with immediate release micro granules of the medicament.

Metformin is an example of an optimal medicament to use in combination with Brake™. Metformin, which decreases hepatic gluconeogenesis, acts on the glucose supply side of the nutritional pathways of the ileal brake. Metformin is ideally given in combination with Brake™ in a dosage lower than metformin alone. In the combination product, Brake™ acts the distally in the same way as RYGB surgery. There is the same sensation of a “malabsorptive emergency” the same activation of L-cells, the output of which produce regeneration and make hunger disappear into satiety. In this case the additional benefit of metformin is some additional activation of the L-cell pathway and a decrease in the amount of glucose synthesized by the liver. Otherwise the coordinates of the response model are the same as RYGB surgery or Brake™ alone.

By way of specific example and preferred embodiments, when apportioning the daily dose of metformin onto the daily dose of ileal brake hormone releasing substance in the enteric coated tablet form, the 1.0 gram tablets are over-coated with the immediate release metformin in a weight ratio of approximately 0.025 to 0.10 parts metformin to each 1.0 part refined sugar; and/or the enteric coated core of the pharmaceutical composition may also comprise approximately 60-90% dextrose and 20-40% of a plant-derived lipid; and/or the pharmaceutical composition may also comprise one or more statins in a weight ratio of approximately 0.001 parts atorvastatin or its equivalent potency to each 1.0 part refined sugar or approximately 0.005 part statin:1.0 part refined sugar (e.g. statins selected from the group consisting of atorvastatin, simvastatin, pravastatin, rosuvastatin, lovastatin, fluvastatin and pitavastatin); and/or the enteric coated core of the pharmaceutical composition may also comprise approximately 60-80% refined sugar, 0-40% of a plant-derived lipid and 0-40% of a plant-derived lipid; and/or when apportioning the daily dose of lisinopril onto the daily dose of ileal brake hormone releasing substance in the enteric coated tablet form, the 1.0 gram tablets are over-coated with the immediate release lisinopril in a weight ratio of approximately 0.0005 to 0.002 parts lisinopril to each 1.0 part refined sugar (e.g. ACE inhibitors selected from the group consisting of lisinopril, enalapril, ramipril, perindopril, quinapril, and e.g., any of the AII inhibitors selected from the group consisting of losartan, olmesartan, valsartan, all at dosage equivalents to lisinopril); and/or the enteric coated core of the pharmaceutical composition may also comprise approximately 60-80% dextrose and 20-40% of a plant-derived lipid; and/or the enteric coated core of the pharmaceutical composition may also comprise approximately 60-80% refined sugar, 0-40% of a plant-derived lipid, and 0-40% of a probiotic organism known to be deficient in the intestinal tract of patients with metabolic syndrome, including for example, F. prausnitzii, B. thetaiotaomicron, L. johnsonii and others; and/or the pharmaceutical composition may comprise approximately 60-80% refined sugar, 0-40% of a plant-derived lipid, and optionally from about 0-40% of probiotic organisms including F. prausnitzii, B. thetaiotaomicron, L. johnsonii and others and 0-40% of a flavoring agent, preferably a natural flavoring agent.

In certain embodiments, the pharmaceutical composition further comprises approximately 0-40% of one or more pharmaceutically active ingredients selected from the group consisting of a proton pump inhibitor, an anti-inflammatory corticosteroid, an anti-diarrhea agent, Teduglutide, a phosphodiesterase-IV inhibitor, methotrexate or another anti-TNF agent, a beta blocker and an anti-inflammatory agent.

Methods of the invention can be used to treat a subject who suffers from one or more glucose supply side associated metabolic syndromes selected from the group consisting of Type 1 diabetes, Type 2 diabetes, a cardiovascular disease, ASCVD, Congestive heart failure (CHF), rheumatoid arthritis, Crohn's disease, ulcerative colitis, coeliac disease, esophagitis, an immune mediated or genetically linked malabsorption syndrome associated with inflammation, COPD, Alzheimer's disease and NAFLD.

In other embodiments, the invention provides a method of treatment comprising increasing pancreatic beta cell mass in a subject suffering from a glucose supply side associated metabolic syndrome by co-administering to the subject in need thereof the controlled release core of ileal brake hormone releasing substance, and over-coating comprised of pharmaceutically effective amounts of a dipeptidyl peptidase-4 inhibitor (DPP-IV) and a proton pump inhibitor (PPI). Preferably, in these methods of treatment:

(a) the DPP-IV is selected from the group consisting of alogliptin, carmegliptin, denagliptin, dutogliptin, linagliptin, melogliptin, saxagliptin, sitagliptin, and vildagliptin; and
(b) the proton pump inhibitor is selected from the group consisting of omeprazole, lansoprazole, rabeprazole, pantoprazole and esomeprazole.

In other embodiments, the invention provides a method of regenerating pancreatic beta cells in a subject suffering from Type 1 diabetes, the method comprising:

(a) confirming that the subject suffers from pancreatic beta cell damage associated with Type 1 diabetes, determining that the subject suffers from metabolic syndrome manifestations by calculating the subject's FS index, and/or using a SmartPill to determine that the subject's ileum has a pH of around 7.2 to around 7.5;
(b) administering to the subject a pharmaceutical composition comprising between about 10 grams to about 20 grams of a refined sugar which is microencapsulated within an enteric coating which dissolves in vivo at a pH of around 7.2 to around 7.5; and.
(c) thereafter, confirming pancreatic beta cell regeneration by determining an increase in insulin, proinsulin secretion and optionally, expression levels of one or more markers selected from the group consisting of Ki67, MCM-7 and PCNA.

A pH-sensitive, radio transmitting capsule whose location can be determined by X-ray may be used to determine that the subject's ileum has a pH of around 7.2 to around 7.5.

In other embodiments, the invention provides a method of regenerating pancreatic beta cells in a subject suffering from Type 1 diabetes, the method comprising:

(a) confirming that the subject suffers from pancreatic beta cell damage associated with Type 1 diabetes by measurement of insulin and/or proinsulin, calculation of FS index and/or determining that the subject's ileum has a pH of around 7.2 to around 7.5;
(b) administering to the subject a pharmaceutical composition comprising between about 10 grams to about 20 grams of a refined sugar which is microencapsulated within an enteric coating which dissolves in vivo at a pH of around 7.2 to around 7.5; and
(c) thereafter, confirming pancreatic beta cell regeneration by determining an increase over time in levels of pancreatic beta cells in pancreatic tissue samples obtained from the subject by surgical biopsy.

In other embodiments, the invention provides a method of regenerating pancreatic beta cells and increasing pancreatic beta cell mass in a subject suffering from Type 1 diabetes, the method comprising:

(a) confirming that the subject suffers from pancreatic beta cell damage associated with Type 1 diabetes by determining through use of a pH-sensitive, radio-transmitting capsule whose location can be determined by X-ray that the subject's ileum has a pH of around 7.2 to around 7.5;
(b) administering to the subject (1) a pharmaceutical composition comprising between about 10 grams to about 20 grams of a refined sugar which is microencapsulated within an enteric coating which dissolves in vivo at a pH of around 7.2 to around 7.5, and (2) pharmaceutically effective amounts of a dipeptidyl peptidase-4 inhibitor (DPP-IV) and a proton pump inhibitor (PPI); and
(c) thereafter, confirming pancreatic beta cell regeneration by determining an increase in expression levels of one or more markers selected from the group consisting of Ki67, MCM-7 and PCNA and/or confirming pancreatic beta cell regeneration by determining an increase over time in levels of pancreatic beta cells in pancreatic tissue samples obtained from the subject by surgical biopsy.

Pharmaceutical compositions as described above are also within the scope of the invention.

Thus, the invention provides a comprehensive treatment strategy that not only addresses maladies such as T2D, but also ameliorates effectively concomitant disorders such as Hepatic Steatosis, ASCVD, secondary organ damage and fundamental gastrointestinal changes including intestinal flora disruption.

In an alternative embodiment, the present invention is directed to a method of regenerating or inhibiting damage to organs and tissues in a subject suffering from one or more organ or tissue manifestations caused by glucose supply side associated metabolic syndrome, the method comprising:

(a) confirming that the subject suffers from or is at risk for suffering from organ and/or tissue damage associated with a glucose supply side associated metabolic syndrome SD; and
(b) co-administering to the subject an effective amount of a pharmaceutical composition comprising a first and optionally a second active composition, the first active composition comprising an ileal brake hormone releasing substance encapsulated within an enteric coating which releases said substance within said subject's ileum and ascending colon causing release of at least one ileal brake hormone from L-cells of said subject, said optional second active composition being formulated in immediate and/or early release form in an over-coating onto said enteric coating, wherein said second composition is beneficial to at least one aspect of said subject's metabolic syndrome manifestations. Thus the present method contemplates coadministration of at least one ileal brake hormone releasing substance alone, or in combination with at least one additional active agent, which may be formulated in the same composition with the ileal brake hormone releasing substance or coadministered in a second pharmaceutical composition to the subject to be treated.

A method wherein said pharmaceutical composition comprises a first active composition in the presence or absence of said second active composition and said pharmaceutical composition is coadministered with at least one additional active agent beneficial to at least one aspect of said subject's metabolic syndrome manifestations, wherein said additional active agent is administered to said subject in a second pharmaceutical composition at the same or a different time as the first active composition.

A method wherein said confirming step occurs by determining or calculating the subject's FS index.

A method wherein said confirming step evidences a FS index of at least 60 in said patient.

A method wherein the confirming step occurs by determining that the subject's ileum has a pH of around 7.2 to around 7.5.

A method wherein the confirming step evidences a FS index of at least about 60 in said patient, a GLP-1 concentration below 20 and a pH of around 7.2 to around 7.5 in the ileum of said subject.

A method wherein the confirming step occurs by determining the subject's food stimulated GLP-1 plasma concentration.

A method wherein the confirming step evidences a food stimulated GLP-1 concentration below 20 or the 10 hour area under the curve plasma concentration of GLP-1 is less than 50.

A method wherein the confirming step is evidenced in said subject by metabolic syndrome and insulin resistance as determined by an elevated HOMA-IR measurement and optionally, a diagnosis of prediabetes, type 1 diabetes or type 2 diabetes.

A method wherein the enteric coating comprises one or more compositions selected from the group consisting of cellulose acetate trimellitiate (CAT), hydroxypropylmethyl cellulose phthalate (HPMCP), hydroxypropylmethyl cellulose, ethyl cellulose and mixtures of hydroxypropylmethyl cellulose and ethyl cellulose each of which contains a subcoating, polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, and mixtures thereof.

A method wherein the enteric coating comprises one or more compositions selected from the group consisting of shellac, Eudragit® L, Eudragit® S, Eudragit® RL, Eudragit® RS and mixtures thereof.

A method wherein subsequent to administration of the pharmaceutical composition to the subject results in the subject's FS index to fall to below 50 and/or the subject's level of GLP-1 expression is increased by between 50% and 90% compared to pre-treatment levels.

A method wherein said ileal brake hormone is at least one hormone selected from the group consisting of GLP-1, glicentin, C-terminally glycine-extended GLP-1 (7 37) intervening peptide-2, GLP-2, GRPP, oxyntomodulin or a peptide fragment thereof, PYY 1-36, PYY 3-36, enteroglucagon and neurotensin.

A method wherein the subject suffers from Type 1 or type 2 diabetes, myocardial infarction, stroke, angina, congestive heart failure (CHF), ASCVD, rheumatoid arthritis, Crohn's disease, ulcerative colitis, coeliac disease, esophagitis, an immune mediated or genetically linked malabsorption syndrome associated with inflammation, COPD, Alzheimer's disease or NAFLD.

A method wherein the organ or tissue manifestations of glucose supply side associated metabolic syndrome measured by elevated FS index of said patient is pancreas and/or pancreatic beta cell damage, myocardial infarction, stroke, angina, congestive heart failure, hypertension, kidney failure, Alzheimer's disease or atherosclerosis.

A method wherein the organ or tissue manifestations of glucose supply side associated metabolic syndrome is one or more of pancreas and/or pancreatic beta cell damage, hepatic steatosis, NAFLD, hyperlipidemia, elevated triglycerides, abdominal adiposity, atherosclerosis, cardiovascular diseases such as myocardial infarction, stroke, angina, congestive heart failure, hypertension, ASCVD, reduced lung capacity (COPD), Rheumatoid arthritis, diabetic nephropathy leading to kidney failure, gastrointestinal tract damage, gastrointestinal dysbiosis, inflammatory bowel disease, brain damage, neurodegenerative disorders, diabetic neuropathy, cognitive impairment associated with obesity and early Alzheimer's disease, which may lead to death of the patient.

A method wherein the second active composition or said additional active agent comprises metformin in an effective amount.

A method wherein the second active composition or said additional active agent comprises an effective amount of at least one agent selected from the group consisting of metformin, a DPP-IV inhibitor, a proton pump inhibitor, an insulin sensitizer, a thiazolidinedione, a PPAR modulator, a PPAR-sparing medicament, an alpha glucosidase inhibitor, a colesevelam mimetic agent, a HMG-CoA reductase inhibitor, an angiotensin II inhibitor, a PDE-5 inhibitor, a reversible acetylcholinesterase inhibitor, a NMDA receptor antagonist, an inhibitor of beta amyloid protein formation, an ACE inhibitor, an antiviral agent, a GLP-1 pathway mimetic, a short acting corticosteroid and mixtures thereof.

A method wherein the second active composition or said additional active agent comprises metformin, sitagliptin, saxagliptin, methotrexate, olanzapine, donepezil, memantine, risperidone, ziprasidone, colesevelam or a mixture thereof.

A method wherein the second active composition or said additional active agent comprises methotrexate, lorcaserin, topiramate, olanzapine, risperidone, ziprasidone or a mixture thereof.

A method wherein the second active composition comprises about 70 to about 150 mg. metformin.

A method wherein the first active composition comprises dextrose in an effective amount and optionally, a plant-derived lipid.

A method wherein the second active composition further comprises one or more statins in an effective amount.

A method wherein the one or more statins are selected from the group consisting of atorvastatin, simvastatin, pravastatin, rosuvastatin, lovastatin, fluvastatin and pitavastatin.

A method wherein the first active composition comprises approximately 60-90% by weight refined sugar and 0-40% by weight of a plant-derived lipid by weight.

A method wherein the first active composition comprises approximately 60-90% by weight refined sugar; 0-40% by weight of a plant-derived lipid; and 0-40% by weight of one or more species of a probiotic bacterial organism.

A method wherein the first active composition comprises approximately 60-90% by weight refined sugar; 0-40% by weight of a plant-derived lipid; 0-40% by weight of a probiotic bacterial organism; and 0-40% by weight of a flavoring agent.

A method wherein the second active is selected from the group consisting of metformin, a DPP-IV inhibitor, a proton pump inhibitor, an anti-inflammatory corticosteroid, an anti-diarrhea agent, Teduglutide, a phosphodiesterase-IV inhibitor, an ACE inhibitor, an Angiotensin II inhibitor, a beta blocker, an anti-inflammatory agent or a mixture thereof

A method wherein the organ or tissue to be regenerated in said subject is any one or more of pancreas, gastrointestinal tract, heart, lungs, brain, liver or kidney.

A method wherein the confirming step evidences a FS index of at least about 100.

A method wherein the second active composition or said additional active agent works in concert with said first active composition to promote regeneration of damaged organs and tissues or inhibition of damage to organs and tissues of said subject.

A method wherein the daily dose of said pharmaceutical composition comprises a first active composition comprising about 5 grams to about 10 grams of glucose and said second active composition or said additional active agent comprises an effective amount of a DPP-IV inhibitor and optionally, an effective amount of a proton pump inhibitor.

A method wherein the DPP-IV inhibitor is included in said composition at a daily dose of about 50-200 mg and said proton pump inhibitor is included in said composition at a daily dose of about 10-50 mg.

A method wherein the organ or tissue manifestations of glucose supply side associated metabolic syndrome in said subject is pancreas and/or pancreatic beta cell damage.

A method wherein the confirming step is evidenced in said subject by metabolic syndrome and insulin resistance as determined by an elevated HOMA-IR measurement and optionally, a diagnosis of prediabetes, type 1 diabetes or type 2 diabetes.

A method wherein the first active composition comprises about 80 to 96% by weight D-glucose, about 0.1 to 1% by weight chlorella, about 0.1 to 1% alfalfa leaf, about 0.1 to 1% by weight barley grass juice concentrate, about 0.1 to 1% by weight chlorophyllin and optionally, an effective amount of at least one further component selected from the group consisting of lubricants, disintegrating agents and excipients, said first active composition being enteric coated with about 6% to about 8% by weight shellac.

A method wherein the first active composition comprises D-glucose in a daily dose of about 5 to about 20 grams and the second active composition or said additional active agent is included in said pharmaceutical composition and comprises an effective amount of a biguanide compound, said method further resolving metabolic syndrome in said patient.

A method wherein the biguanide is metformin included in said pharmaceutical composition at a daily dose of about 250-500 mg.

A method wherein the first active composition comprises D-glucose in a daily dose of about 5 to about 20 grams and said second active composition or said additional active agent comprises an effective amount of a DPP-IV inhibitor, and optionally an effective amount of a proton pump inhibitor, said method further resolving metabolic syndrome in said patient.

A method wherein the DPP-IV inhibitor is sitagliptin included in said pharmaceutical composition at a daily dose of about 100-200 mg and said optional proton pump inhibitor is omeprazole included in said pharmaceutical composition at a daily dose of about 10 mg to about 50 mg.

A method wherein resolution of the subject's metabolic syndrome and regeneration of said subject's pancreas and/or pancreatic islet cells is confirmed by a fall in the subject's FS index to below 50, a rise in plasma GLP-1 concentration at 3.5 post administration to a level above 60 and/or HBA1c level falls below 6.5 after 6 months of treatment.

A method wherein the organ or tissue manifestations of glucose supply side associated metabolic syndrome in said subject is hepatic steatosis.

A method wherein the first active composition comprises D-glucose in a daily dose of about 5 to about 20 grams and the second active composition or said additional active agent comprises an effective amount of a statin or berberine.

A method wherein the wherein the organ or tissue manifestations of glucose supply side associated metabolic syndrome in said subject is hepatic steatosis and NALFD with hepatitis C.

A method wherein the first active composition comprises D-glucose in a daily dose of about 5 to about 20 grams and the second active composition or said additional active agent comprises an effective amount of a statin or berberine in combination with an anti-hepatitis C agent.

A method wherein the subject is also at risk for hepatocellular cancer.

A method wherein the anti-hepatitis C agent is ribavirin included in said pharmaceutical composition at a daily dose of about 600-1200 mg.

A method wherein confirmation of the organ or tissue manifestation of glucose supply side associated metabolic syndrome is confirmed by elevated HOMA-IR measurement for metabolic syndrome, by elevated AST and optionally AlfaFetoProtein for inflammation and a medical diagnosis of hepatic steatosis, optionally hepatic fibrosis or cirrhosis and optionally a hepatic viral infection.

A method wherein the organ or tissue manifestations of glucose supply side associated metabolic syndrome in said subject is atherosclerosis (endovascular damage).

A method wherein the first active composition comprises D-glucose in a daily dose of about 5 to about 20 grams and the second active composition or said additional active agent comprises an effective amount of a beta blocker.

A method wherein the wherein the beta blocker is propranolol.

A method wherein the organ or tissue manifestations of glucose supply side associated metabolic syndrome in said subject is hypertension.

A method wherein the first active composition comprises D-glucose in a daily dose of about 5 to about 20 grams and the second active composition or said additional active agent comprises an effective amount of an ACE inhibitor, preferably lisinopril.

A method wherein the organ or tissue manifestations of glucose supply side associated metabolic syndrome in said subject is diabetic nephropathy.

A method wherein the first active composition comprises D-glucose in a daily dose of about 5 to about 20 grams and the second active composition or said additional active agent comprises an effective amount of an angiotensin II inhibitor.

A method wherein the wherein the organ or tissue manifestations of glucose supply side associated metabolic syndrome in said subject is diabetic neuropathy, Alzheimer's disease or early cognitive impairment.

A method wherein the first active composition comprises D-glucose in a daily dose of about 5 to about 20 grams and the second active composition or said additional active agent comprises an effective amount of an NMDA receptor antagonist (e.g. memantine) or an acetyl cholinesterase inhibitor (e.g. donepezil).

A method wherein the organ or tissue manifestations of glucose supply side associated metabolic syndrome in said subject is liver damage, pancreas and/or pancreatic islet cell damage and GI tract damage.

A method wherein the first active composition comprises D-glucose in a daily dose of about 5 to about 20 grams and the second active composition or said additional active agent comprises an effective amount of berberine.

A method wherein the berberine is included in said pharmaceutical composition at a daily dosage of about 1000 mg.

A method wherein the regeneration or treatment of liver damage, pancreas and/or pancreatic islet cell damage and GI tract damage results in regeneration of hepatocellular architecture, increased pancreatic islet cell mass and improved function of GI enterocytes.

A method wherein the subject's metabolic syndrome is also resolved.

A method wherein resolution of the subject's metabolic syndrome and regeneration of hepatocellular architecture, increased pancreatic islet cell mass and improved function of GI enterocytes is confirmed by a fall in the subject's FS index to less than 50, a rise in GLP-1 plasma concentration at 3.5 hours post administration to above 60, and an AST decline to 40 or below and an alpha-fetoprotein decline to 4.0 or below six months after said subject first started treatment.

A method wherein the organ or tissue manifestations of glucose supply side associated metabolic syndrome in said subject is inflammation, atherosclerosis, ASCVD, hyperlipidemia, hypertension and optionally, congestive heart failure and/or COPD with an increased risk for stroke, myocardial infarction or death from cardiovascular cause.

A method wherein the first active composition comprises D-glucose in a daily dose of about 5 to about 20 grams and the second active composition or said additional active agent comprises an effective amount of statin.

A method wherein the improvement or favorable treatment of said subject's vascular endothelial architecture, cardiac cells and lipid transport are confirmed by a fall in FS index to below 50, a rise in GLP-1 plasma concentration at 3.5 hours post administration above 60, a hsCRP decline to 2.0 or below, triglyceride decline to 150 or below and diastolic pressure decline to below 90 after 6 months of treatment.

A method wherein the organ or tissue manifestations of glucose supply side associated metabolic syndrome in said subject is vascular damage, cardiac cell damage, or lipid transport damage.

A method wherein the first active composition comprises D-glucose in a daily dose of about 5 to about 20 grams and the second active composition or said additional active agent comprises an effective amount of an ACE inhibitor.

A method wherein the ACE inhibitor is lisinopril included in said pharmaceutical composition at a daily dose of about 10 mg.

A method wherein the first active composition comprises D-glucose in a daily dose of about 10 to about 20 grams and the second active composition or said additional active agent comprises an effective amount of a statin and optionally, an ACE inhibitor.

A method wherein the statin is atorvastatin included in said pharmaceutical composition at a daily dose of about 10 mg and said optional ACE inhibitor is lisinopril included in said pharmaceutical composition at a daily dose of about 10 mg.

A method wherein the organ or tissue manifestations of glucose supply side associated metabolic syndrome in said subject is inflammation, confirmed by elevated hsCRP, cognitive impairment, diabetes associated with Alzheimer's disease, diabetic neuropathy, optional transient ischemic attacks and an increased risk for stroke, or death from cardiovascular causes.

A method wherein the first active composition comprises D-glucose in a daily dose of about 5 to about 20 grams and the second active composition or said additional active agent is an NMDA receptor antagonist and/or an acetyl cholinesterase inhibitor.

A method wherein the NMDA receptor antagonist is memantine included in said pharmaceutical composition at a daily dose of 10 mg. and said acetyl cholinesterase inhibitor is donepezil included in said pharmaceutical composition at a daily dose of between 5 and 10 mg.

A method wherein the second active composition is a combination of an NMDA receptor antagonist and an acetyl cholinesterase inhibitor.

A method wherein the improvement or favorable treatment of the subject is confirmed by a fall in FS index to below 50, a rise in GLP-1 plasma concentration at 3.5 hours post administration above 60, a hsCRP decline to 2.0 or below, triglyceride decline to 50 or below and diastolic pressure decline to below 90 after 6 months of treatment.

A method wherein the organ or tissue manifestations of glucose supply side associated metabolic syndrome in said subject is inflammation associated with rheumatoid arthritis, atherosclerosis, central adiposity, ASCVD with an increased risk for stroke, myocardial infarction or death from cardiovascular cause.

A method wherein the first active composition comprises D-glucose in a daily dose of about 5 to about 20 grams and the second active composition or said additional active agent comprises an effective amount of methotrexate.

A method wherein the methotrexate is included in the pharmaceutical composition at a daily dose of about 0.5 mg.

A method wherein the improvement or favorable treatment of said subject's inflamed joints, vascular endothelial architecture, synovial cells and associated immunomodulatory processes is confirmed by a fall in FS index to below 50, a rise in GLP-1 plasma concentration at 3.5 hours post administration above 60, a hsCRP decline to 2.0 or below, normal AST levels and resolution of join inflammation after three months of treatment.

A method wherein the organ or tissue manifestations of glucose supply side associated metabolic syndrome in said subject is inflammation confirmed by elevated hsCRP and a medical diagnosis of diabetic neuropathy, hypertension and optionally central adiposity, ASCVD with an increased risk for stroke, myocardial infarction or death from cardiovascular causes and renal failure.

A method wherein the first active composition comprises D-glucose in a daily dose of about 5 to about 20 grams and the second active composition or said additional active agent comprises an effective amount of an angiotensin II inhibitor.

A method wherein the angiotensin II inhibitor is selected from the group consisting of losartan, candesartan, irbesartan, valsartan, olmesartan, telmisartan and mixtures thereof.

A method wherein the improvement or favorable treatment of said subject's renal nephron mass is confirmed by a fall in FS index to below 50, a rise in GLP-1 plasma concentration at 3.5 hours post administration above 60, a hsCRP decline to 2.0 or below, fall in diastolic pressure to below 90 and a decline in serum creatinine of 0.5 mg/dl from a pre-treatment baseline after 3 months of treatment.

A method wherein the wherein the organ or tissue manifestations of glucose supply side associated metabolic syndrome in said subject is inflammation confirmed by elevated hsCRP and a medical diagnosis of inflammatory bowel disease and/or gastrointestinal microbiome dysbiosis and optionally central adiposity.

A method wherein the first active composition comprises D-glucose in a daily dose of about 5 to about 20 grams and the first or second active composition or said additional active agent comprises an effective amount of a short acting corticosteroid.

A method wherein the corticosteroid is budesonide at a daily dose of about 3 mg.

A method wherein the second active composition or said additional active agent comprises at least one probiotic organism.

A method wherein the probiotic organism is Faecalibacterium prausnitzii at a dose ranging from about 10⁶ to 10⁸ colony forming units.

A method wherein the probiotic organism is released from said second active composition at a pH of at least about 7.0.

A method wherein regeneration of the subject's gastrointestinal enterocytes and rebalancing of associated immunomodulatory processes is confirmed by a fall in FS index to below 50, a rise in GLP-1 plasma concentration at 3.5 hours post administration above 60, a hsCRP decline to 2.0 or below, a fall in Crohn's disease activity score below 60; and a decline in the number or frequency of gastrointestinal exacerbations from a pre-treatment baseline after 3 months of treatment.

In other alternative embodiments the present invention is directed to a pharmaceutical composition in unit dosage form comprising a first composition and a second composition, said first composition comprising a daily dose of between about 5 grams to about 20 grams of an ileal brake hormone releasing agent which is encapsulated within an enteric coating which dissolves in vivo at a pH of around 7.2 to around 7.5 and releases said substance within said subject's ileum and ascending colon causing release of at least one ileal brake hormone from L-cells of said subject, said second active composition being formulated in immediate and/or early release form in an over-coating onto said enteric coating, wherein said second composition works in concert with said first composition to treat a subject's metabolic syndrome manifestations.

A pharmaceutical composition wherein the second active composition comprises an effective amount of at least one agent selected from the group consisting of metformin, a DPPIV inhibitor, a proton pump inhibitor, an insulin sensitizer, a thiazolidinedione, a PPAR modulator, a PPAR-sparing medicament, an alpha glucosidase inhibitor, a colesevelam mimetic agent, a HMG-CoA reductase inhibitor, an angiotensin II inhibitor, a PDE-5 inhibitor, a reversible acetylcholinesterase inhibitor, a NMDA receptor antagonist, an inhibitor of beta amyloid protein formation, an ACE inhibitor, an antiviral agent, a GLP-1 pathway mimetic, a short acting steroid and mixtures thereof.

A pharmaceutical composition wherein the second active composition comprises metformin, sitagliptin, saxagliptin, methotrexate, olanzapine, donepezil, memantine, risperidone, ziprasidone, colesevelam or a mixture thereof.

A pharmaceutical composition wherein the second active composition comprises methotrexate, lorcaserin, topiramate, olanzapine, risperidone, ziprasidone or a mixture thereof.

A pharmaceutical composition wherein the enteric coating comprises one or more compositions selected from the group consisting of cellulose acetate trimellitiate (CAT), hydroxypropylmethyl cellulose phthalate (HPMCP), hydroxypropylmethyl cellulose, ethyl cellulose and mixtures of hydroxypropylmethyl cellulose and ethyl cellulose each of which contains a subcoating, polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, and mixtures thereof.

A pharmaceutical composition wherein the enteric coating comprises one or more compositions selected from the group consisting of shellac, Eudragit® L, Eudragit® S, Eudragit® RL, Eudragit® RS and mixtures thereof.

A pharmaceutical composition wherein the composition comprises a first composition comprising a refined sugar as the ileal brake hormone releasing substance and a second composition comprising metformin, said metformin and said sugar being included in said pharmaceutical composition in a weight ratio of approximately 0.025 to 0.05 parts metformin:1.0 part refined sugar.

A pharmaceutical composition wherein the first active composition comprises approximately 60-90% dextrose and about 20-40% of a plant-derived lipid.

A pharmaceutical composition wherein the composition comprises a first composition comprising a refined sugar as the ileal brake hormone releasing substance and a second composition comprising a statin, said statin and said sugar being included in said pharmaceutical composition in a weight ratio of approximately 0.001 to 0.005 parts statin:1.0 part refined sugar.

A pharmaceutical composition wherein the one or more statins are selected from the group consisting of atorvastatin, simvastatin, pravastatin, rosuvastatin, lovastatin, fluvastatin and pitavastatin.

A pharmaceutical composition wherein the first active composition comprises approximately 60-90% refined sugar, 0-40% of a plant-derived lipid and 0-40% of a plant-derived lipid.

A pharmaceutical composition wherein the first active composition comprises approximately 60-90% refined sugar; 0-40% of a plant-derived lipid; 0-40% of a plant-derived lipid; and 0-40% of a probiotic bacterial organism.

A pharmaceutical composition wherein the first active composition comprises approximately 60-90% refined sugar; 0-40% of a plant-derived lipid; 0-40% of a plant-derived lipid; 0-40% of a probiotic bacterial organism; and optionally, an effective amount of a flavoring agent.

A pharmaceutical composition wherein the second active composition comprises from 0-40% by weight of said pharmaceutical composition and is selected from the group consisting of Metformin, a DPP-IV inhibitor, a proton pump inhibitor, an anti-inflammatory corticosteroid, an anti-diarrhea agent, Teduglutide, a phosphodiesterase-IV inhibitor, an ACE inhibitor, a beta blocker and an anti-inflammatory agent.

A pharmaceutical composition wherein the second active composition comprises from 0% to 40% by weight of said pharmaceutical composition and is selected from the group consisting of metformin, a DPP-IV inhibitor, a proton pump inhibitor, an insulin sensitizer, a thiazolidinedione, a PPAR modulator, a PPAR-sparing medicament, an alpha glucosidase inhibitor, a colesevelam mimetic agent, a HMG-CoA reductase inhibitor, an angiotensin II inhibitor, a PDE-5 inhibitor, a reversible acetylcholinesterase inhibitor, a NMDA receptor antagonist, an inhibitor of beta amyloid protein formation, an ACE inhibitor, an antiviral agent, a GLP-1 pathway mimetic, a short acting corticosteroid and mixtures thereof.

A pharmaceutical composition wherein the second active composition or the additional active agent comprises metformin, sitagliptin, saxagliptin, methotrexate, olanzapine, donepezil, memantine, risperidone, ziprasidone, colesevelam or a mixture thereof.

A pharmaceutical composition wherein the second active composition or the additional active agent comprises methotrexate, lorcaserin, topiramate, olanzapine, risperidone, ziprasidone or a mixture thereof.

A pharmaceutical composition wherein the second active composition comprises about 70 to about 150 mg. metformin.

In an another alternative embodiment, the present invention is directed to a method of treatment comprising increasing pancreatic beta cell mass in a subject suffering from a glucose supply side associated metabolic syndrome by co-administering to the subject in need of regeneration of pancreatic beta cells pharmaceutically effective amounts of a dipeptidyl peptidase-4 inhibitor (DPP-4i) and a proton pump inhibitor (PPI) in combination with an effective amount of enteric coated glucose which releases in said subject's ileum at a pH ranging from 7.2-7.5.

In another alternative method:

(a) the dipeptidyl peptidase-4 inhibitor is selected from the group consisting of alogliptin, carmegliptin, denagliptin, dutogliptin, linagliptin, melogliptin, saxagliptin, sitagliptin, and vildagliptin; and
(b) the proton pump inhibitor is selected from the group consisting of omeprazole, lansoprazole, rabeprazole, pantoprazole and esomeprazole.

In an alternative embodiment, a method is directed to regenerating pancreatic beta cells in a subject suffering from Type 1 diabetes, the method comprising:

(a) confirming that the subject suffers from pancreatic beta cell damage associated with Type 1 diabetes by determining the FS index of said subject, and/or measuring to determine that the subject's ileum has a pH of around 7.2 to around 7.5;
(b) administering to the subject an effective amount of a pharmaceutical composition comprising between about 10 grams to about 20 grams of a refined sugar which is microencapsulated within an enteric coating which dissolves in vivo at a pH of around 7.2 to around 7.5, and optionally an effective amount of a proton pump inhibitor and/or a DPP-IV inhibitor; and.
(c) thereafter, confirming pancreatic beta cell regeneration by determining an increase in expression levels of one or more markers selected from the group consisting of insulin, proinsulin, c-peptide and Ki67, MCM-7 and PCNA.

A method wherein a pH-sensitive, radio transmitting capsule whose location can be determined by analysis of data output is used to determine that the subject's ileum has a pH of around 7.2 to around 7.5.

In an alternative embodiment, a method is directed to regenerating pancreatic beta cells in a subject suffering from Type 1 diabetes, the method comprising:

(a) confirming that the subject suffers from pancreatic beta cell damage associated with Type 1 diabetes by determining that the subject has elevated FS index, decreased concentrations of insulin, pro-insulin, and C-peptide;
(b) administering to the subject an effective amount of a pharmaceutical composition comprising between about 10 grams to about 20 grams of a refined sugar which is microencapsulated within an enteric coating which dissolves in vivo at a pH of around 7.2 to around 7.5; and.
(c) thereafter, confirming pancreatic beta cell regeneration by determining that FS index values have decreased over time, and that there is an elevation in C-peptide concentrations, an increase in insulin output and a reduction in required dose of insulin needed to control hyperglycemia.

In an alternative embodiment, a method is directed to regenerating pancreatic beta cells and increasing pancreatic beta cell mass in a subject suffering from Type 1 diabetes, the method comprising:

(a) confirming that the subject suffers from pancreatic beta cell damage associated with Type 1 diabetes by determining lab tests of c-peptide, insulin, proinsulin and FS index in the subject;
(b) administering to the subject (1) an effective amount of a pharmaceutical composition comprising between about 10 grams to about 20 grams of a refined sugar which is microencapsulated within an enteric coating which dissolves in vivo at a pH of around 7.2 to around 7.5, and (2) pharmaceutically effective amounts of a dipeptidyl peptidase-4 inhibitor (DPP-4i) and a proton pump inhibitor (PPI); and
(c) thereafter, confirming pancreatic beta cell regeneration by determining an increase in expression levels of one or more markers selected from the group consisting of insulin, proinsulin, c-peptide, Ki67, MCM-7 and PCNA and/or confirming pancreatic beta cell regeneration by determining an increase over time in these levels and subjects FS index.

In an alternative embodiment, a method is directed to regenerating organs and tissues in a subject suffering from one or more organ or tissue manifestations of glucose supply side associated metabolic syndrome, the method comprising:

(a) confirming that the subject suffers from or is at risk for suffering from organ and/or tissue damage associated with metabolic syndrome SD; and
(b) administering to the subject an effective amount of a pharmaceutical composition comprising between about 10 grams to about 20 grams of a refined sugar which is microencapsulated within an enteric coating which dissolves in vivo in the ileum of said subject at a pH of around 7.2 to around 7.5, wherein said organ to be regenerated is the subject's liver, GI tract, cardiovascular system, kidney, lungs and brain.

A method wherein the organ to be regenerated is the subject's brain and said regeneration improves the patient's cognition.

A method where the subject suffers from Alzheimer's disease.

A method wherein the confirming step occurs by determining or calculating the subject's FS index.

A method wherein the confirming step evidences a FS index of at least 60 in said patient.

A method wherein the confirming step occurs by determining that the subject's ileum has a pH of around 7.2 to around 7.5.

A method wherein the confirming step evidences a FS index of at least about 60 in said patient and a pH of around 7.2 to around 7.5 in the ileum of said subject.

In another embodiment, the invention is directed to a medicament for use in the regeneration of organs and tissues in a subject suffering from one or more organ or tissue manifestations of glucose supply side associated metabolic syndrome, said medicament comprising a pharmaceutical dosage form comprising an inner controlled release component comprising an ileal brake hormone releasing substance comprising about 10 grams to about 20 grams of a refined sugar which is encapsulated within an enteric coating which releases at least about 50% by weight of said ileal brake hormone releasing substance in the ileum and ascending colon of said subject, and an optional outer release component over-coating said inner controlled release component, said outer release component over-coating comprising an immediate or early release layer of a second active medicament, said second active medicament acting synergistically with the inner core ileal brake hormone releasing substance upon one or more manifestations of said patient's metabolic syndrome.

In still another embodiment, the invention is directed to a method of regenerating or inhibiting damage to organs and tissues in a subject suffering from one or more organ or tissue manifestations caused by glucose supply side associated metabolic syndrome, the method comprising:

(a) confirming that the subject suffers from or is at risk for suffering from organ and/or tissue damage associated with a glucose supply side associated metabolic syndrome; and
(b) administering to the subject an effective amount of a pharmaceutical composition comprising between about 10 grams to about 20 grams of a refined sugar which is encapsulated within an enteric coating which dissolves in vivo at a pH of around 7.2 to around 7.5 and releases at least about 50% by weight of said sugar in the ileum of said subject, said composition optionally comprising an additional bioactive agent formulated in an over-coating of said enteric coating in immediate or early release form.

The various embodiments of the present invention and other aspects are described further in the Detailed Description of the Invention.

### BRIEF DESCRIPTION OF THE FIGURES

FIG. 1. GLP-1 concentrations under different conditions in humans, including after a 400-500 kcal meal challenge. Note the early peak of GLP-1 after RYGB surgery, caused by rapid arrival of nutrition in the distal intestines from the surgical removal of stomach and shortening of intestine. In contrast, the instant invention Brake™ formulation arrives at this same site after about 3 hrs, and its dosage is calibrated to the same GLP-1 AUC as seen in RYGB surgery. Meal challenge shows this location is not responding to food in lean, obese, or obese T2D patients. Hence, there is less appetite suppression signal in obese individuals and in particular obese T2D individuals, even compared to lean individuals. Compared to lean individuals, the ileal brake is quieted in obese and obese T2D by bacterial dysbiosis, by rapid absorption of refined IR carbohydrates, or both. This problem is not solved by DPP-IV drugs because GLP-1 must be stimulated for them to work properly. Exogenous GLP-1 drugs such as exenatide (Byetta) produce about the same peak AUC of GLP-1 as Brake™ or RYGB. Overall, the graph shows the importance of this new means of organ and tissue regeneration, the stimulation of ileal brake hormones. RYGB and Brake™ have similar potency in hormone release characteristics.

FIG. 2. Impact of T2D, obesity or both on intestinal pH. Data were collected in SmartPill experiments over several years. Note the distinctly lower pH values in the ileum of obese T2D patients, which is thought to reflect the local dysbiosis and overgrowth of gram negative organisms. These data are useful in targeting the ileum for release of ileal brake hormones using the disclosed formulations

FIG. 3. Impact of 7 different coating formulations on release of GLP-1 from human subjects, each tested for optimal coating to reach the ileal brake and release GLP-1. Under the stated calibration conditions, the selected formulation would have the same 0-10 hr AUC of GLP-1 as observed in a patient having RYGB surgery. In this manner the purpose of the ileal brake hormone releasing formulation is to mimic the action of the RYGB surgery procedure on the distal intestinal Metasensor, including resolution of metabolic syndrome and regeneration of GI, pancreas and liver. From these testing procedures, formulation #2 was chosen for treatment of metabolic syndrome in patients.

FIG. 4. Preservation of beta cell mass. This figure shows the impact of different points of intervention on patients with T2D. It also shows the HBA1c patterns of conventionally treated T2D patients where there is a slow loss of effect of either metformin and/or sulfonylureas (Gibenclamide in this example). HBA1c rises steadily, forcing a change in therapy in most patients over 1-3 years. The conventional T2D regimens slowly lose their effects because they fail to preserve or augment pancreatic beta cell functions in the presence of unrelenting IR carbohydrate loading. Conventional data are plotted from those in the UK Prospective Diabetes Study. On the other hand, RYGB surgery causes pancreatic regeneration and lowers HBA1c to normal as a result. Thus far, Brake™, when added to metformin or when used alone as a mimetic of RYGB surgery has also returned HBA1c to normal, indicating a similar effect on pancreatic regeneration as RYGB surgery.

FIG. 5. The average pattern of loss of metformin effect over 5-10 years in a composite group of 61 patients treated with metformin. In this group, the FS index is calculated at 3-6 months intervals from the component parameters of metabolic syndrome. Shown vs. time (descending order) are the glucose SD ratio, the HBA1c, diastolic blood pressure, BMI, the impact of vasodilator drugs, the drug metformin, the calculated FS index, the Triglyceride concentrations, the hepatic enzymes AST and ALT, and the combined CV risk score for MACE events. All laboratory parameters that are components of the FS index, as well as the risk for MACE events rise over time, indicating progression of T2D to increasing CV risk. We describe this as a slow loss of diabetic and metabolic syndrome control.

FIG. 6. The rapid resolution of T2D and all of metabolic syndrome in a composite group of 36 patients with RYGB surgery, some of whom also received metformin. In this group, the FS index is calculated at 3-6 months intervals from the component parameters of metabolic syndrome, and it can be seen just how rapidly the metabolic syndrome parameters normalize. Shown vs. time (descending order) are the glucose SD ratio, the HBA1c, diastolic blood pressure, BMI, the impact of vasodilator drugs, the drug metformin, the calculated FS index, the Triglyceride concentrations, the hepatic enzymes AST and ALT, and the combined CV risk score for MACE events. All laboratory parameters that are components of the FS index, as well as the risk for MACE events, fell rapidly to normal, even before any weight loss, indicating the rapid resolution of metabolic syndrome after RYGB mediated hormonal actions from the food stimulated ileal brake. We present this as evident pancreatic, GI and hepatic regeneration following RYGB surgery and newly conferred ileal brake mediated metabolic syndrome control.

FIG. 7. The rapid resolution of T2D and all of metabolic syndrome in a composite group of 18 patients treated with Formulation 2 of Brake™, some of whom also received metformin. In this group, the FS index is calculated at 3-6 months intervals from the component parameters of metabolic syndrome, and it can be seen just how rapidly the metabolic syndrome parameters normalize, in fact at about the same rate as RYGB patients even though they lose less weight. Shown vs. time (descending order) are the glucose SD ratio, the HBA1c, diastolic blood pressure, BMI, the impact of vasodilator drugs, the drug metformin, the calculated FS index, the Triglyceride concentrations, the hepatic enzymes AST and ALT, and the combined CV risk score for MACE events. All laboratory parameters that are components of the FS index, as well as the risk for MACE events, fell rapidly to normal, even before any weight loss, indicating the rapid resolution of metabolic syndrome after Brake™ actions releasing the hormones of the ileal brake. We present this as evident pancreatic, GI and hepatic regeneration following Brake™ therapy and newly conferred ileal brake mediated metabolic syndrome control.

FIG. 8. Weight change in a 55 year old female subject over 80 days, illustrating the typical pattern of loss in a subject in normal metabolic and nutritional balance, where the primary change is lowered dietary intake. The data illustrate daily monitoring of sustained weight reduction at a steady weight of approximately 1-2 lbs per week. This pattern was associated with a subject in Metasensor balance with a dietary reduction of approximately 150 calories per day, resulting in steady utilization of stored fat. Exercise patterns did not change over this period, and weight loss was even across storage sites including abdominal and visceral, buttocks, neck and breasts.

FIG. 9. The ileal brake, a metabolic regulatory process based in the distal intestine (jejunum, ileum, Right colon). The system includes Drivers, a Metasensor, Effectors and Beneficiary organs and tissues that are regenerated including pancreas, liver, GI, CV and CNS. The hormones regulating this axis of nutritional and metabolic control are released under control of both probiotic organisms and intestinal enterocytes, which together form a Metasensor (multiple components interacting to provide regulatory balance). The Metasensor effects changes in metabolism via release of both stop signals (appetite suppression, satiety) and repair/regenerate signals (immunomodulatory, anti-apoptotic, mitotic). The system efficiency is optimized so that excess nutrient is stored as adipose and released as needed to aid repair or provide energy supply.

FIG. 10. Normal Nutritional and Metabolic System in Homeostasis, with all components of the Metasensory System in balance. Dietary intake is normal and some excess reaches the distal intestine. However, when the patient ingests only IR (immediate release)-CHOs (carbohydrates), the bacteria in the ileum are not achieving nutrition (nutrients are all absorbed proximally leaving no distal nutrition). The distal intestinal organisms react by Suppression of L-cell output and hunger ensues. If on the other hand the patient is having a balanced diet with portions reaching the bacteria, they have no reason to suppress the L-cell output and normal eating produces satiety.

FIG. 11. Supply Side mediated excessive intake of CHOs with immediate release characteristics: Metasensor mediated Hunger from a DIETARY IMBALANCE; absorption of IR-CHO in Overdrive with pancreatic stimulation, in this example sugary soft drinks; CHO Storage short term as visceral fat; Insulin Resistance; minimal regeneration. Metasensor system out of balance; Nutrient imbalance develops and creates a distal flora imbalance; e.g. a plentiful supply of IR (immediate release) CHO (carbohydrates), for example sugar sweetened beverages. Distal intestinal bacteria are hungry so via their effects on hormone signaling pathways, the mammalian host is hungry. Excess inulin production drives central adiposity (favors storage at these sites) and insulin resistance accelerates in response to a progressive flood of IR nutrition as the host becomes more and more hungry to feed this dysbiosis pattern.

FIG. 12. RYGB surgery mechanically diverts ingested contents past the absorptive (but non-signaling) area, and bombards the signaling areas further downstream in late jejunum and ileum. Specifically, there is a diversion of the sugar to the distal ileum, where the L-cells are stimulated and the distal intestinal flora are receiving nutrition. Both combine to extinguish the hunger signals. In this setting fat is mobilized from both liver and adipose storage, and the pancreatic stress is lowered considerable. Insulin resistance is resolved by RYGB surgery. The arrival of massive nutrients at the ileum in such a large quantity creates a “malabsorptive emergency” and initiates the satiety signal by shutting down the hormonal release from the L-cells to regenerate signaling to a certain extent with the same or less amount of food needed, therefore restoring maintenance and regeneration. And because it is not individualized, RYGB surgery will trigger more regeneration than signaling, to the point where 2-4 years following the procedure, the jejunum segment will have evolved to restore proximal absorption to a baseline levels

FIG. 13. Brake™ acts the distally in the jejunum and ileum in the same way as RYGB surgery. There is the same sensation of a “malabsorptive emergency” the same activation of L-cells, the output of which promotes regeneration in GI, Liver and Pancreas: hunger disappears into a strong signal of satiety. We calibrate the dosage of Brake™ to produce the same hormonal output as RYGB surgery. The strength of the ileal signal is not as potent as RYGB, but it can be more prolonged because of the delayed release formulation. Thus with Brake™, the intensity of the stimulation will be more moderate and closer to physiological and therefore regeneration proceeds in Liver, pancreas, GI enterocytes in a much more natural and physiological way compared to surgery. The stress on the pancreas recedes, the distal ileum receives the nutrients, quieting the bacteria and increasing the output of the L-cells. Fat is mobilized from both liver and adipose tissue. Of no great surprise, weight loss is more rapid with RYGB, since RYGB surgery also physically decreases the size of stomach, limiting ingestion in a second, profound manner over the ileal brake pathway alone.

FIG. 14. Metformin, which decreases hepatic gluconeogenesis, acts on the glucose supply side of the nutritional pathways of the ileal brake. Metformin is ideally given in combination with Brake™ in a dosage lower than metformin alone. In the combination product, Brake™ acts the distally in the same way as RYGB surgery. There is the same sensation of a “malabsorptive emergency” the same activation of L-cells, the output of which produce regeneration and make hunger disappear into satiety. In this case the additional benefit of metformin is a decrease in the amount of glucose synthesized by the liver. Otherwise the coordinates of the response model are the same as RYGB surgery or Brake™ alone. The strength of the ileal signal is not as potent as RYGB, but it can be more prolonged because of the delayed release formulation.

FIG. 15 sets forth the equation for determining a subject's FS index pursuant to the present invention.

FIG. 16. Table of GLP-1 response to 7 formulations, each given to 7 volunteers (demographics shown as group means). Formulation 2 was chosen for clinical development on the basis of these data.

FIG. 17. Table of comparisons between RYGB patients (N=16) and Brake treated patients (N=16). Table 5A, shows that a notable reversal of CV disease risk following RYGB surgery and Brake™ therapy according to the present invention has been associated with resolution of elevated triglycerides, elevation of HDL, lowering of LDL, and lowering of hepatic inflammation, as was seen using the FS index to monitor the course of these parameters in treated patients. In the last column, the Brake response is provided as a ratio to the RYGB response.

FIG. 18. Weight change in patients with RYGB in comparison with treatment with Brake alone, Brake with Metformin and Brake with Atorvastatin. Also shown are control patients given Atorvastatin alone and Metformin alone. In these latter cases the patients did not receive Brake or RYGB surgery. Only patients with initial abnormal values are displayed here, since the question is how long to normalize the parameter. RYGB patients lost more weight than Brake patients, and in general metformin patients either stayed the same or lost a few pounds, in most cases less than Brake or RYGB patients. Additional medications in the control patients are shown at the table on the bottom

FIG. 19. HBA1c change in patients with RYGB in comparison with treatment with Brake alone, Brake with Metformin and Brake with Atorvastatin. Also shown are control patients given Atorvastatin alone and Metformin alone. In these latter cases the patients did not receive Brake or RYGB surgery. Only patients with initial abnormal values are displayed here, since the question is how long to normalize the parameter. RYGB patients normalized their HBA1c values at the fastest rate, but there was little difference between Brake and RYGB. In general metformin patients either stayed the same or had a minor drop in HBA1c, in most cases less than Brake or RYGB patients. Additional medications in the control patients are shown at the table on the bottom

FIG. 20. HDL change in patients with RYGB in comparison with treatment with Brake alone, Brake with Atorvastatin. Also shown are control patients given Atorvastatin or other statins alone. Notably all except one of the control patients were taking 10 mg doses of Atorvastatin, and it is clear why there was essentially no change in HDL as a result of the low dosing. In the control cases the patients did not receive Brake or have RYGB surgery. Only patients with initially abnormal values are displayed here, since the question is how long to normalize the parameter. RYGB patients normalized their HDL values at the fastest rate, and there was little difference between Brake and RYGB. In general atorvastatin patients at 10 mg doses either stayed the same or had a minor drop in HDL, in most cases less than Brake or RYGB patients. Additional medications in the control patients are shown at the table on the bottom, note that some were taking fish oil products.

FIG. 21. Triglyceride (TG) changes in patients with RYGB in comparison with treatment with Brake alone, Brake with Atorvastatin. Also shown are control patients given Atorvastatin (usually 10 mg doses) or other statins. In these latter cases the patients did not receive Brake or RYGB surgery. Only patients with initial abnormal values are displayed here, since the question is how long to normalize the parameter. RYGB patients normalized their TG values at the fastest rate, although there was little difference between Brake and RYGB. In general atorvastatin patients either stayed the same or had a minor drop in TG, in most cases less than Brake or RYGB patients unless they were also taking fish oil products, in which case the control patients were similar to the Brake and RYGB patients. Additional medications in the control patients are shown at the table on the bottom.

FIG. 22. Aspartate Transaminase enzyme concentrations (AST, formerly the SGOT) and rate of change in patients with RYGB in comparison with treatment with Brake alone, Brake with atorvastatin. Also shown are control patients given Atorvastatin or other statins alone. In these latter cases the patients did not receive Brake or RYGB surgery. Only patients with initial abnormal values are displayed here, since the question is how long to normalize the parameter. RYGB patients normalized their TG values at the fastest rate, although there was little difference between Brake and RYGB. In general atorvastatin patients either stayed the same or had a minor drop in TG, in most cases less than Brake or RYGB patients unless they were also taking fish oil products, in which case the control patients were similar to the Brake and RYGB patients. Additional medications in the control patients are shown at the table on the bottom.

FIG. 23. Change in HBA1c over time in patient MF, who was taking both Brake and Januvia (sitagliptin).

FIG. 24. Change in alpha-fetoprotein over time in Patient E1, who had Hepatitis C and was taking Interferon (IFN), Ribavirin and Brake™ for concomitant hepatic steatosis and fibrosis. A normal value of alpha-fetoprotein is 2.0.

### DETAILED DESCRIPTION OF THE INVENTION

The term “patient” or “subject” is used throughout the specification within context to describe an animal, generally a mammal and preferably a human, to whom treatment, including prophylactic treatment, with the compositions and/or methods according to the present invention is provided. For treatment of a particular condition or disease state which is specific for a specific animal such as a human patient, the term patient refers to that specific animal. Preferred subjects include humans and domesticated animals, including dogs, cats, horses, cows, pigs, among others.

The term “effective” is used herein, unless otherwise indicated, to describe an amount of a compound, composition or component and for an appropriate period of time which, in context, is used to produce or effect an intended result, whether that result relates to the treatment of a disorder or condition associated with the present invention or alternatively, is used to produce another compound, agent or composition. This term subsumes all other effective amount or effective concentration terms which are otherwise described in the present application. In many instances, with the administration of D-glucose (dextrose) as a ileal brake hormone releasing substance in compositions and methods according to the present invention, an effective amount of D-glucose ranges from about 500 mg. to about 12.5 grams or more up to about 20 grams, preferably at least about 5 grams to about 10 grams up to about 20 grams used on a daily basis.

The term “nutritional substance” is used synonymously with “pharmaceutical composition” and “ileal brake hormone releasing substance” in certain contexts herein and refers to the substance which produces the intended effect in the ileum of a patient or subject pursuant to the present invention. A “nutritional substance” includes, but is not limited to, proteins and associated amino acids, fats including saturated fats, monosaturated fats, polyunsaturated fats, essential fatty acids, Omega-3 and Omega-6 fatty acids, trans fatty acids, cholesterol, fat substitutes, carbohydrates such as dietary fiber (both soluble and insoluble fiber), starch, sugars (including monosaccharides, fructose, galactose, glucose, disaccharides, lactose, maltose, sucrose, and alcohol), polymeric glucoses including inulin and polydextrose, natural sugar substitutes (including brazzein, Curculin, erythritol, fructose, glycyrrhizin, glycyrrhizin, glycerol, hydrogenated starch hydrosylates, isomalt, lactitol, mabinlin, maltitol, mannitol, miraculin, monellin, pentadin, sorbitol, stevia, tagatose, thaumatin, and xylitol), sahlep, berberine in its available forms and halwa root extract. D-glucose (dextrose) is a preferred ileal brake hormone releasing substance. Ileal brake hormone releasing substances include all compositions that yield the aforementioned nutrients upon digestion or that contain such nutrients, including polymeric forms of these nutrients.

Additional ileal brake hormone releasing components which may be included in compositions according to the present invention include, barley grass, known to be a rich source of highly metabolizable vitamins and minerals such as vitamins A, B 1, B2, B6, B 12 and C, potassium, magnesium, and zinc. In addition, barley grass also has a high concentration of the enzyme superoxide dismutase (SOD), which has been shown to have high levels of antioxidant activity. Barley grass is believed to be an important nutrient in the regulation of the digestive process because the micronutrients, enzymes (e.g., SOD), and fiber contained in barley grass are believed to improve intestinal function.

Alfalfa fresh or dried leaf tea is also usable in the invention, to promote appetite, and as a good source of chlorophyll and fiber. Alfalfa contains biotin, calcium, choline, inositol, iron, magnesium, PABA, phosphorus, potassium, protein, sodium, sulfur, tryptophan (amino acid), and vitamins A, B complex, C, D, E, K, P, and U. Alfalfa supplements are recommended for treating poor digestion, and were shown to lower cholesterol levels in animal studies. Alfalfa is categorized as Generally Regarded as Safe (GRAS) by the FDA. Dosages can range from 25-1500 mg, preferably 500-1000 mg dried leaf per day.

Chlorella is yet another substance usable in the invention in combination with the ileal brake hormone releasing substance (preferably D-glucose or dextrose), being a genus of unicellular green algae, grown and harvested in tanks, purified, processed and dried to form a powder. Chlorella is rich in chlorophyll, carotenes, and contains the full vitamin B complex, vitamins E and C, and has a wide range of minerals, including magnesium, potassium, iron and calcium. Chlorella also provides dietary fiber, nucleic acids, amino acids, enzymes, CGF (Chlorella Growth Factor) and other substances. Dosages can range from 300-1500 mg/day.

Chlorophyllin is yet another ileal brake hormone releasing substance, being a known food additive and has been used as an alternative medicine. Chlorophyllin is a water-soluble, semi-synthetic sodium/copper derivative of chlorophyll, and the active ingredient in a number of internally-taken preparations intended to reduce odors associated with incontinence, colostomies and similar procedures, as well as body odor in general. It is also available as a topical preparation, purportedly useful for treatment and odor control of wounds, injuries, and other skin conditions, such as for radiation burns.

Sodium alginate may also be used as a nutritional substance, preferably in combination with D-glucose or dextrose.

The term “ileum” is used to describe the third (of three) portion of the small intestine just before the small intestine becomes the large intestine in the gastrointestinal tract. The ileum is the final section of the small intestine in higher vertebrates, including mammals. The ileum follows the duodenum and jejunum in the small intestine, and is separated from the “Cecum” or “Colon” by the ileocecal valve (ICV). In humans, the ileum is about 2-4 meters long, and the pH usually ranges between about 7 and 8 (neutral or slightly alkaline). The function of the ileum is mainly sensory and regulatory, and in that regard facilitates detection of malabsorption upstream. Additional functions of the ileum, include the absorption of certain vitamins, bile salts and whatever products of digestion were not absorbed by the jejunum. The wall itself is made up of folds, each of which has many tiny finger-like projections known as “villi” on its surface. In turn, the epithelial cells which line these villi possess even larger numbers of microvilli. Therefore, the ileum has an extremely large surface area both for the adsorption of enzyme molecules and for the absorption of products of digestion. The DNES (diffuse neuroendocrine system) cells that line the ileum contain lesser amounts of the protease and carbohydrase enzymes (gastrin, secretin, and cholecystokinin) responsible for the final stages of protein and carbohydrate digestion. These enzymes are present in the cytoplasm of the epithelial cells.

The term “delays the release in vivo of the majority of the ileal brake hormone releasing substance until the dosage form reaches the subject's ileum” means: (1) that not less than around 50% by weight, not less than around 70% by weight, more preferably not less than around 80% by weight, and more preferably not less than around 90% and in certain instances substantially all of the ileal brake hormone releasing substance remains unreleased in vivo prior to the dosage form's arrival at a subject's ileum; and (2) that not less than around 50%, not less than around 70% by weight, more preferably not less than around 80% by weight, and more preferably not less than around 90%, of the ileal brake hormone releasing substance is remains unreleased in vivo by the time when the dosage form enters the subject's ileum. In preferred aspects of the invention this amount is at least about 1 gram, at least about 2.5 grams, at least about 3 grams, often at least about 5 grams, at least about 7.5 grams, preferably about 10 grams to about 12-12.5 grams or more (about 12.5 to about 20 grams, especially of polymeric materials such as polydextrose or those compounds of higher molecular weight) of the ileal brake hormone releasing substance and in particular, glucose, is released within the small intestine in the ileum in order to stimulate ileum hormones and related hormones and effect the intended result associated with lowering the manifestations of metabolic syndrome and/or influencing one or more of insulin resistance (decrease resistance), blood glucose (decrease in/stabilize glucose levels), glucagon secretion (decrease), insulin release (decrease and/or stabilize release and/or levels), ileum hormone release (increase) or other hormone release, in particular, one or more of GLP-1, glicentin, C-terminally glycine-extended GLP-1 (7 37), (PG (78 108)); C-peptide, intervening peptide-2 (PG (111 122) amide); GLP-2 (PG (126 158), GRPP (PG (1 30)), oxyntomodulin (PG (33 69), and other peptide fractions to be isolated, PYY (1-36), PYY (3-36), enteroglucagon, neurotensin, as well as leptin, IGF-1 and IGF-2, and preferably, one or more, two or more, three or more, four or more, five or more, six or more, seven or more, or all of GLP-1, GLP-2, C-peptide, PYY (1-36 and/or 3-36), glucagon, leptin, IGF-1 and IGF-2.

The term “ileum hormones” includes all hormones that are associated with intraluminal food substances stimulating the release of said hormones, could be associated with action of the ileal brake and associated feedback from the ileum or ileum-related stimulation of insulin secretion or inhibition of glucagon secretion. “Ileum hormones” therefore include, but are not limited to, GLP-1, glicentin, C-terminally glycine-extended GLP-1 (7 37), (PG (78 108)); intervening peptide-2 (PG (111 122) amide); GLP-2 (PG (126 158), GRPP (PG (1 30)), oxyntomodulin (PG (33 69), and other peptide fractions to be isolated, PYY (PYY 1-36) and (PYY 3-36), enteroglucagon and neurotensin.

The term “ileum hormone-stimulating amount of a nutritional substance” means any amount of a nutritional substance that is effective to induce measurable hormone release in the ileum, and induce feedback from the ileum or ileum-related stimulation of insulin secretion or inhibition of glucagon secretion, or other effect such as shutting down or decreasing insulin resistance and increasing glucose tolerance. Consequently, an “ileum hormone-stimulating amount of a nutritional substance” can vary widely in dosage depending upon factors such as the specific nutrient at issue, the desired effect of administration, the desired goal of minimizing caloric intake, and the characteristics of the subject to whom the ileal brake hormone releasing substance is administered. For example, at least about 500 mg of D-glucose is used, and a particularly preferred ileum hormonal-stimulating amount of D-glucose includes between about 5 to 20 grams, often about 7.5-8 g to about 12-12.5 g (preferably around 10 g).

The following terms and/or concepts also help define the present invention.

SD Ratio Derivation, CV Risk Definition in T2D:

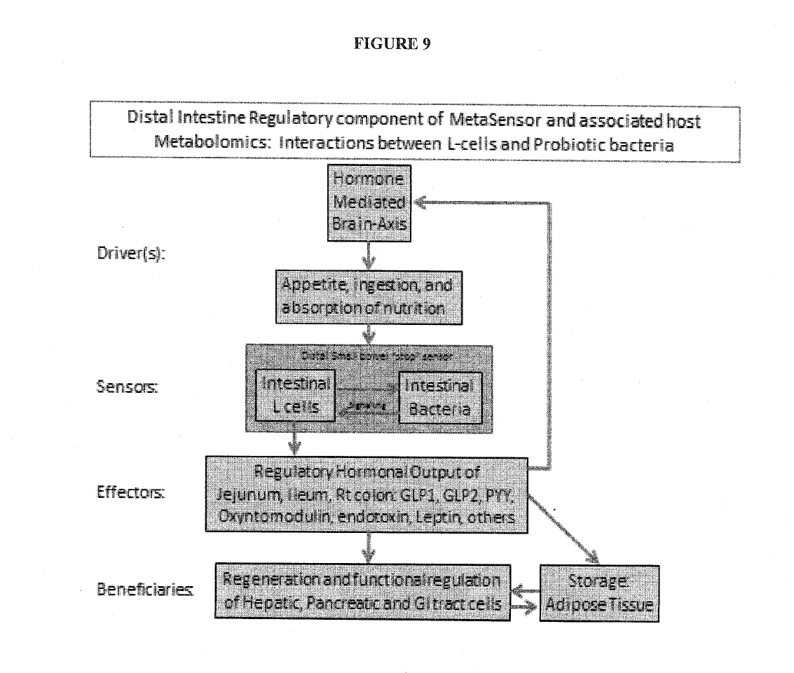

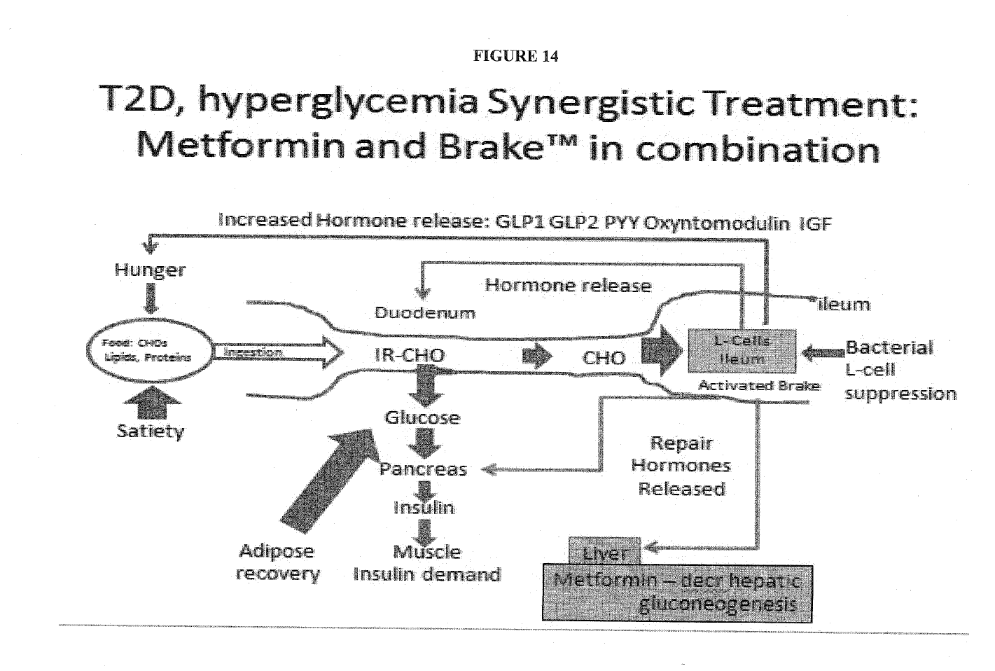

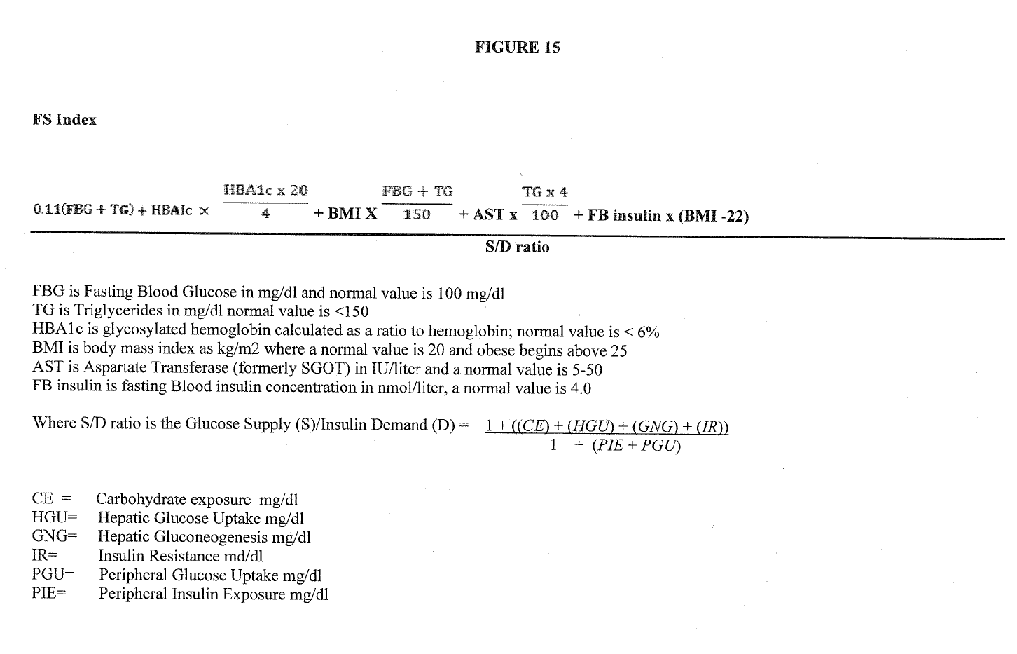

SD (Supply/Demand) index was developed by the inventors to quantify the impact of dietary glucose load on T2D, and to develop a means of rank ordering the impact of effective treatments that change T2D responsiveness by interrupting glucose supply. (see Monte U.S. Pat. No. 8,367,418, incorporated by reference in its entirety herein). By identifying quantitative differences between antidiabetic agents on carbohydrate exposure (CE), hepatic glucose uptake (HGU), hepatic gluconeogenesis (GNG), insulin resistance (IR), peripheral glucose uptake (PGU), and peripheral insulin exposure (PIE), the inventors created a pharmacokinetic/pharmacodynamic model to characterize the effect of the agents on the glucose supply and insulin demand dynamic. Glucose supply was defined as the cumulative percentage decrease in CE, increase in HGU, decrease in GNG, and decrease in IR, while insulin demand was defined as the cumulative percentage increase in PIE and PGU (See FIG. 15 for the recitation of SD ratio). According to the teachings of Supply Side and by reference to treatments with a high SD ratio number (a value above 2.0), the inventors show that a drug used for T2D has beneficial interaction with a lowering of glucose supply, considered now an essential component to lowering of insulin resistance and insulin demand from immediate release glucose load (see FIGS. 9-14).(3) The new observation on the glucose supply side is that the compositions of the instant invention, having a high SD ratio (Metformin, Brake™ and the combination thereof) act in a synergistic manner to improving the regeneration of the pancreatic beta cells.

Because T2D cardiovascular outcome trials have not demonstrated macrovascular benefit with more aggressive blood glucose reduction when using conventional algorithms that predominantly focus on insulin demand, it would appear logical to consider a model that incorporates both the extent of blood glucose lowering as HBA1c and the means by which the blood glucose was reduced (SD ratio) when considering macrovascular outcomes.

It was our objective to test the hypothesis that, in conjunction with HBA1c, patients managed on the glucose supply side of the model would have fewer CV events versus those managed on the insulin demand side. In a study of matched cases, we found that patients managed at higher glucose values and on the insulin demand side of the model have increased cardiovascular risk(2).

Glucose, Hunger and Metabolic Syndrome:

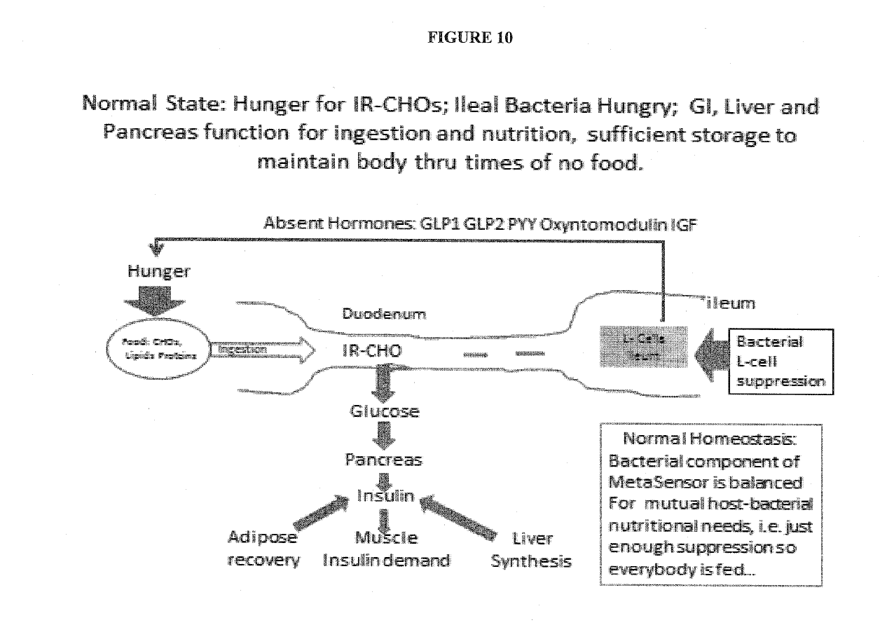

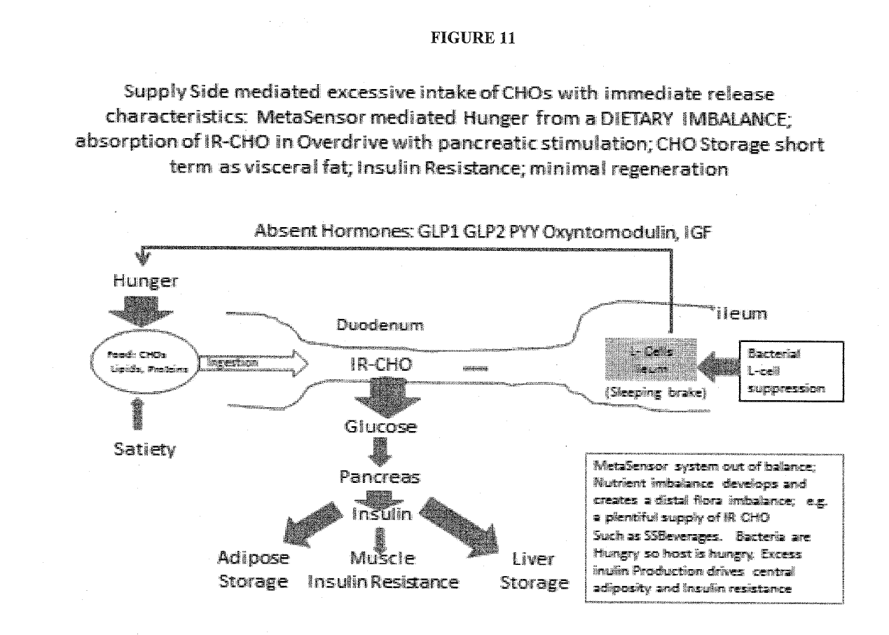

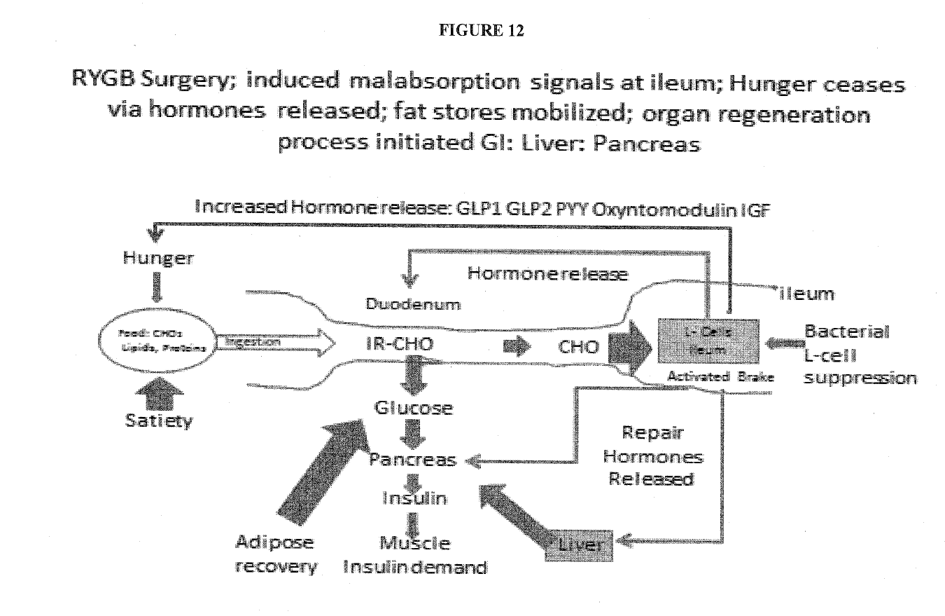

Continued hunger is the driver of glucose supply side driven metabolic syndrome, and there are accelerating hunger signals emitted from the L-cells of the ileum in absence of sufficient supply of distally available carbohydrate to satisfy the needs of the bacterial flora in the ileum, and in fact the L-cells themselves (see FIGS. 10-11). Alterations of intestinal micro flora numbers and species, and their need for nutrition continues to signal hunger to the host by use of L-cell signaling procedures to turn off satiety signals, with the core driver of hunger being the demand for nutrition by the organisms. In the absence of carbohydrates at the level of the ileum, the host and host bacterial signal is for continued hunger; the organisms suppress the ileal hormone output from the L-cells. Eventually the resulting host over-nutrition spills over to the ileum, removing the bacterial suppression of the ileal hormones and allowing a satiety signal until absence of nutrients at the ileal sensor begins the cycle again. In certain conditions such as malabsorption or RYGB surgery, excessive carbohydrates arrive at the ileum and in this case, the signals completely over-ride hunger. Ileal brake associated hormone outputs not only produce satiety over the longer term but also begin to rapidly (i.e., within a day to several days) trigger the endogenous repair of pancreas, liver and GI tract cells. Together, these are the “stop and repair” processes that are programmed into our bodies to optimize the balance between ingestion and nutritional needs. As these systems are mainly programmed to satisfy basic needs for nutrition, they are most efficient in a relative lack of glucose as nutrition. Our current excessive nutrient ingestion patterns, especially a growing preference for rapidly absorbed immediate release and duodenally absorbed sugars that deny nutrition to distal intestinal bacteria, create an overdrive of the hunger pathways directly to organ exhaustion and obesity without the benefit of the triggered repair. A simple fix for over-nutrition with rapidly absorbed sugars is to perform RYGB surgery (see FIG. 12). An often preferred and less invasive approach to provide oral formulations of carbohydrates pursuant to the present invention that are directly released at the L-cells in a dose sufficient to trigger the stop and repair processes that protect us from accelerations in metabolic syndrome.

The present invention provides pharmaceutical compositions and methods of regenerating organs and tissues in a patient afflicted with one or more organ or tissue manifestations of glucose supply side associated metabolic syndrome, when the syndrome is accompanied by suppressed regenerating processes and progressively failing organs. A pharmaceutical composition in an effective dosage is provided to said metabolic syndrome patient, which wakes up the dormant ileal brake sensor and initiates renewed hormonal signals to regenerate candidate organs and tissues including but not limited to the pancreas, the liver, the enterocytes of the GI tract and the associated signal transmitting neurons, as well as the cardiovascular system, the lungs, the kidneys and brain (in some cases resolving or limiting the debilitation of Alzheimer's disease and other neurodegenerative disease states and/or enhancing cognition). These actions are assured by measured biomarkers of both the ileal hormone process and the resolution of metabolic syndrome and organ repair.

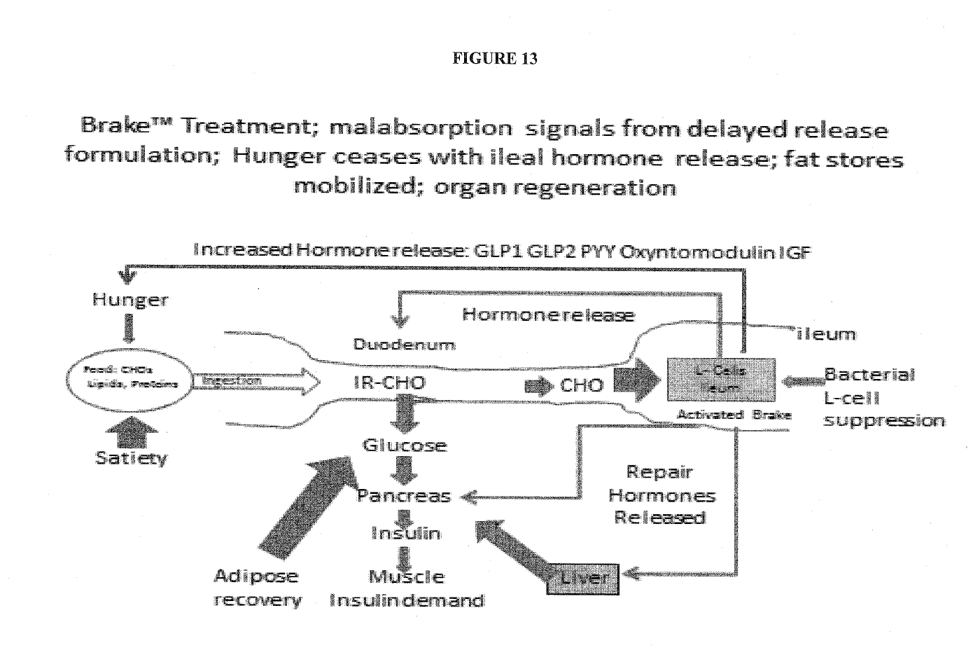

A pharmaceutical composition in an effective dosage is disclosed herein. When provided to said metabolic syndrome patient, the beneficial effect is activation of the dormant ileal brake Metasensor (see FIG. 9) and the newly activated Metasensor initiates renewed hormonal signals to regenerate candidate organs and tissues including but not limited to the pancreas, the liver, the enterocytes of the GI tract and the associated signal transmitting neurons (see FIG. 13), among others. By way of example, directly regenerating pancreas, liver and gastrointestinal tract functions are specifically described herein and attributed to treatment with a specific pharmaceutical composition. These actions are assured by measured biomarkers of both the ileal hormone process and the resolution of metabolic syndrome and organ repair.

Serially measured biomarkers of metabolic syndrome progression or regression, in this case the components of the FS index in patients given the compositions according to the present invention (Brake™) or taken to RYGB surgery, demonstrate the successful regeneration. Once regeneration is accomplished by Brake™ or RYGB surgery, the regenerated organs then signal the patient, to resume adequate nutrition seeking behavior as directed by restored signals of hunger. Specific actions on organ regeneration are confirmed by measured biomarkers and analysis of the results (including a lowered value for the FS index, as disclosed fully below). Dependent on reserve capabilities of the patient at hand, and depending on composition and administered dosage of the pharmaceutical composition, the present invention relates to dramatic improvement or potential cure of metabolic syndrome manifestations including but not limited to T2D, hyperlipidemia, atherosclerosis, insulin resistance, hypertension, and ASCVD.

Specific actions on organ regeneration are confirmed at each stage of treatment by measured biomarkers and FS index calculation, and analysis of the results and use of the index to adjust dosage and duration of treatment with the pharmaceutical composition. Dependent on reserve capabilities of the patient at hand, and depending on composition and administered dosage of the pharmaceutical composition, the present invention relates to dramatic improvement or potential cure of metabolic syndrome manifestations including but not limited to T2D, hyperlipidemia, atherosclerosis, insulin resistance, hypertension, and hepatic steatosis, as well as reversing organ damage for pancreas, liver, kidneys, heart and cardiovascular system (atherosclerosis and related manifestations of heart disease), GI tract and brain (including reversing, resolving and inhibiting Alzheimer's and other cognitive disorders by improving brain function. The FS index demonstrates these effects of said composition to the treating physician and thereby provides a roadmap to the regeneration of organs and tissues and associated lowering of cardiovascular risk.

The SD model certainly represents a new perspective on T2D causes and treatments. As the problem of glucose driven cardiovascular injury was further examined, it became clear to the inventors that a perspective broader than T2D alone needed to be considered as treatments were discovered based on RYGB mechanisms. This is the basis of the creation of the FS index, which is used to indicate damage to organs as a consequence of Metabolic Syndrome and other disease manifestations and also to indicate the extent of organ regeneration.

Metabolic Syndrome, Beyond T2D with FS Index

Compositions disclosed herein are effective for the treatment of Metabolic Syndrome in patients. There are 5 key components defining Metabolic Syndrome: Abdominal adiposity (Male >40 in waist, Female >35 inch waist), Elevated Triglycerides (>150), Low HDL Cholesterol (<40 male, <50 female), High blood pressure (>135/85), and Hyperglycemia (FBS>120 or HBA1c>7) (4-17) Note that the consensus definition of metabolic syndrome contains hyperglycemia, which is covered by the SD ratio. The other elements of metabolic syndrome were not covered until the inventors developed the FS index. There are several other variants within the consensus definitions as might be anticipated by a research community that does not consider this all to have a common cause or a common treatment methodology.

In the art of medicine, treating physicians consider each of the various aspects of metabolic syndrome to be a single disease, and they use a single lab test to diagnose or monitor treatment progress. An example would be the use of BMI to diagnose or monitor weight gain, HBA1c or glucose to monitor diabetes, or cholesterol to diagnose or monitor hyperlipidemia. None of these approaches consider direct effects of pharmaceutical treatments, which themselves change the organ damage risk within each disease, as well as overall.

The FS index (Fayad/Schentag) of MS (the relevant equation for which appears in attached FIG. 15) considers the following: Fasting Blood Glucose, Fasting Insulin, HBA1c, BMI, AST, Triglycerides, Glucose Supply-Demand (SD) index, and Proinsulin. Each parameter is mathematically arranged to increase as MS worsens, and weighted approximately equally in the prediction of MS progression and risk for organ damage.

The FS index of metabolic Syndrome provides or describes a predictive measurement of damage to a patient's organs pursuant to metabolic syndrome and/or related conditions of a patient and the necessity of administering the therapy of the present invention in order to regenerate those organs which have been damaged pursuant to the present invention. FS index was therefore invented to quantify the Metabolic Syndrome and the degree of organ and/or tissue damage to patients. As discovered, patients with Metabolic Syndrome have many different manifestations, while each individual may have an index made up of varying severity of T2D, hyperlipidemia, hypertension or NAFLD. Before FS index was invented, there was no means of tracking progression of metabolic syndrome in patient populations that may have any or all of these conditions to varying degree.

The goal of the inventors of FS index was to provide a means of identifying the likelihood that a patient will manifest organ and/or tissue damage from metabolic syndrome. Once risk can be quantified from each component of metabolic syndrome, compositions and effective treatments can be disclosed to mitigate the risk, and thereby show benefit when administered to patients. Thus, the FS index can be used to score metabolic syndrome manifestations in a patient such that the FS index is predictive of organ damage and corrective therapeutic steps may be taken to repair/regenerate those damaged organs using methods and compositions according to the present invention.

FS Index Methods

$$\frac{\begin{matrix}0.11 ( F B G + T G ) + HBAIc \times \frac{HBA 1 c \times 20}{4} + B M I \times \\ \frac{F B G + T G}{150} + A S T \times \frac{T G \times 4}{100} + F B insulin \times ( B M I - 22 )\end{matrix}}{S / D ratio}$$

$$F B G is Fasting Blood Glucose in mg \text{/} dl and normal value$$

$$is 100 mg \text{/} dl$$

$$T G is Triglycerides in mg \text{/} dl normal value is < 150$$

$$H B A 1 c is glycosylated hemoglobin calculated as a ratio to$$

$$hemoglobin ; normal value is < 6 \%$$

$$B M I is body mass index as kg / m 2 where a normal value$$

$$is 20 and obese begins above 25$$

$$A S T is Aspartate Transferase ( formerly S G O T ) in$$

$$IU \text{/} liter and a normal value is 5 \text{-} 50$$

$$F B insulin is fasting Blood insulin concentration in$$

$$\underline{nmol} \text{/} liter , a normal value is 4.0$$

$$Where S / D ratio is the Glucose Supply ( S ) / Insulin Demand ( D ) = \frac{1 + ( ( C E ) + ( H G U ) + ( G N G ) + ( I R ) )}{1 + ( P I E + P G U )}$$

$$\underline{C E =} Carbohydrate exposure mg \text{/} dl$$

$$H G U = Hepatic Glucose Uptake mg \text{/} dl$$

$$G N G = Hepatic Gluconeogenesis mg \text{/} dl$$

$$I R = Insulin Resistance md \text{/} dl$$

$$P G U = Peripheral Glucose Uptake mg \text{/} dl$$

$$P I E = Peripheral Insulin Exposure mg \text{/} dl$$

The FS index was then applied to well-studied patient populations already in databases, using a neural net model and the equation set forth in FIG. 15. The database included previously published 50 patients with T2D having CV events principally myocardial infarctions, and controls of a precisely matched group of T2D patients without these events (2, 3). The database included previously published 45 patients with T2D having AMIs, 45 precisely matched T2D controls without AMIs, 41 patients with RYGB surgery and reversal of MS, 300 patients with COPD and T2D, and 18 patients given Brake™ therapy for Hepatitis C, NAFLD, or prediabetes. For each study patient, we had complete access to all raw data, measured vital signs, culture results, and clinician assessments. Many of these measures were incorporated as inputs into the neural net models, and are also illustrated on the Y axis as standard deviations above the defined normal mean of the parameter. The primary aim of the neural net modeling effort was to model CV events and CV mortality in relation to time course of Input parameters, with a second primary effort to model time course of organ failure as a metric in relation to Input factors such as those in the laboratory biomarkers listed above.

FS index values were calculated from serial laboratory and clinical data over timeframes ranging between 2 and 10 years. In these patient populations, a normal FS index value was 20-50. Patients with two or more manifestations of MS and increased organic damage risk profiles have FS index values above about 60, often above about 100, and quite often above 200. Maximum FS index values are above 500, typical when nearly every MS component is highly abnormal.

It was notable that many of the highest FS index patients the inventors observed are from morbidly obese patients who subsequently undergo RYGB surgery. Surprisingly, the surgical procedure cures every aspect of their metabolic syndrome. This discovery and studies of the mechanism of this cure via ileal brake hormone release and associated organ regeneration, directly led the inventors to the instant invention Brake™, the first oral mimetic of RYGB to cure metabolic syndrome.

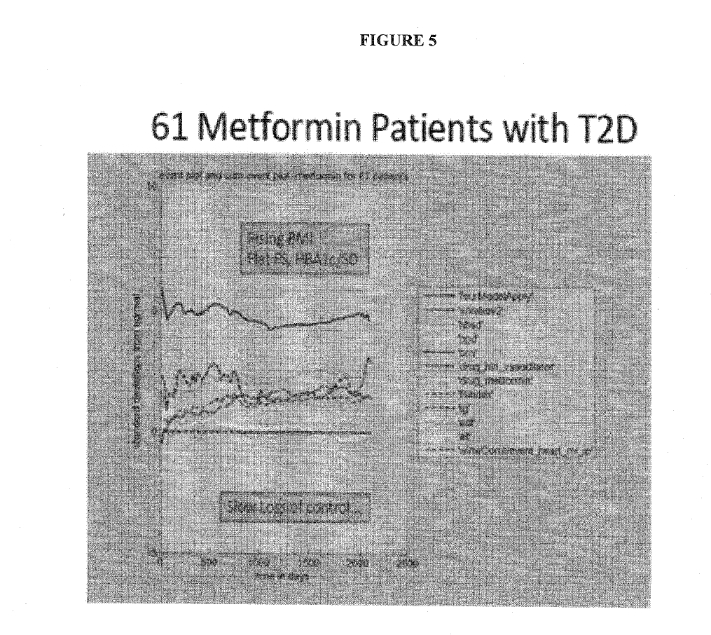

Using the Neural Net model in MatLab, the initial association of biomarker-mortality response surface was confirmed and extended by performing subset analyses to identify the most informative input biomarkers. Throughout this description of methods developed and applied, raw data from each patient are displayed vs. time via hyperlinks, and cumulative graphics are displayed. Unless otherwise stated, standard deviations (z-score) vs. time are presented in the individual and mean population graphics (FIGS. 5-7 are examples of such output).

Except where specifically noted, standard deviations for each parameter are displayed on the Y axis, as this factor normalizes the different range of parameters for visual illustration of behavior patterns in groups on a common Y axis. Unless otherwise noted, the X-axis displays time throughout this report.

For purposes of analysis, clinical and laboratory parameters were converted into modified z-scores as follows:

A mean normal value (described as “mean” in the following) was selected based on review of literature and various published laboratory compendia. The Standard Deviation (SD) of each parameter was set to one half of the normal range. The modified z-score is calculated as follows:

z=(Patient value−“mean”)/SD

On the graphs, the z-score is reported as the number of SD.

Outputs of the many runs of the database thru the Neural Net models can be presented both in graphical format and tables. In general, we use graphical displays for individual patients and groups of similar patients, and we use tables to present the results of runs of aggregate analyses performed on the individual patients. The general theme of presenting some highlights of the results is outlined as follows:
Input/output relationships for Groups of patients
Subsets of metabolic syndrome patients with common characteristics
Individual patients with top 10 informative parameters displayed over time (examples in FIGS. 5-7)

In each Input/Output graphic, the x axis is time and the y axis is multiples of SD over the normal value which is set at zero. This allows all parameters approximately equal weight in the display, recognizing that parameters behaving in a non-linear fashion will always appear more important in terms of large changes and that display bias cannot be completely removed from the display.

Ranked correlation lists for:
Metabolic syndrome components and related events
CV events
Pharmacoeconomic Analyses
Drug impacts on metabolic syndrome endpoints

Tables of rank ordered correlation parameters provided herein are all based on the somewhat time independent link between Inputs (usually the baseline parameter value at time of metabolic syndrome diagnosis) and Outputs calculated as cumulative or AUC variables; the multiples here stated are used to rank order the input in connection to the magnitude of the output, connecting inputs and outputs regardless of timing. Output Error is the Root Mean Squared (RMS) error between the enrichment model based on the input parameter (in this case the baseline biomarker) and the desired output of (for example, cumulative CV risk score), based on each input parameter for all the patients. A lower output error means that the parameter on its own is a better predictor, and the model seeks to find the best single parameter in all cases of RMS rank ordering.

These displays generally use the top two parameters for a ranked correlation and display them in 3D against a Z axis parameter of defined importance, such as cumulative CV, other organ damaging events, such as cumulative organ failures, etc. In some settings we use a parameter of interest even if it does not achieve “top 2 status” in ranked correlation, simply because it allows the study of the parameter more specifically across the entire population

These two dimensional graphical displays order the x-axis to start with the patient of lowest risk at zero, and the patient of highest risk at the last value. The y-axis is the risk score itself. Then we use color to define which patients have the event in question. For example, one can show increasing risk for CV events on these graphs, and the mark the patients with the actual events vs. their risk in an easy to identify display. Calculations of risk over zero (point separating half above and half below) allows an overall estimate of increasing or decreasing probability that roughly follows the more widely applied odds ratio. The advantage of doing the analysis with a neural net is that non-linear behavior is not excessively weighted over linear behavior

Final tables aggregating patterns of population behavior are derived from analysis of each individual, once again rank ordering inputs to outputs. In this run of the neural net, the question asked is what are the top 2-4 inputs for their particular behavior pattern. The tabulation of these data are used to define subsets that might be a focus for enrichment studies

FS Index Study Results

By way of summary, high FS index values generally precede and therefore predict organ damage events in metabolic syndrome patients, regardless of the specific components of Metabolic Syndrome that were abnormal. Abnormal and rising FS index values predict organ damage, although it does not predict the time of the event. A rapid rise in the FS index over 3-6 months is a good predictor of impending organ damage events. When Metabolic Syndrome is studied as the equal weight of its components using the FS index, it is apparent why clinical strategies treating only one component of Metabolic Syndrome do not predict or remove all risk of organ damage events.

Abnormal FS index values, when subsequently normalized, indicate resolution of each component of Metabolic Syndrome, raising the possibility that specific treatments of Metabolic Syndrome might halt progression or reverse Metabolic Syndrome and resulting organ damage entirely. A cure is often effected.

High FS index values (at least about 60, often 100, or 200, at least about 300, at least about 400, at least about 500 or more) predicts organ damage and a necessity to regenerate organs in such patients, regardless of the specific components of metabolic syndrome that were abnormal. Abnormal and rising FS index values predicted a greater likelihood of organ damage and identify a more urgent need for organ regeneration. When MS is studied as the equal weight of its components using the FS index, it is apparent why drug treatments effective for only one component of MS do not remove all risk of subsequent CV events. The index also explains why drug therapies that improve one aspect of MS but worsen others may not mitigate organ damage or provide organ regeneration.

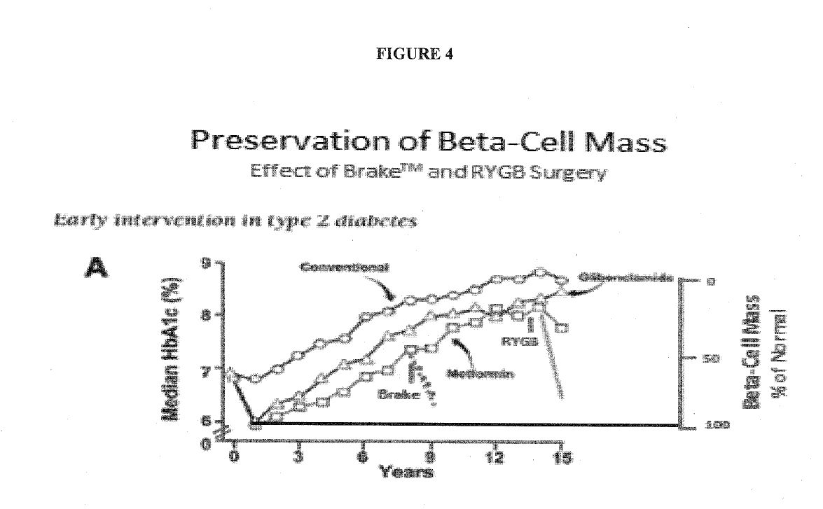

An example might be Metformin when used alone (see FIG. 5) where it is clear that improvement in T2D alone is still associated with increasing risk over time even though the diabetic control is improved. By way of further example, showing the relationship between treating only T2D in patients with metabolic syndrome, consider the example in FIG. 4, wherein the inventors disclose a slow loss of pancreatic islet cell function while taking metformin alone. Note that application of either RYGB surgery or Brake™ therapy preferably in conjunction with metformin can completely normalize HBA1c in these cases, but it accomplishes this because it repairs the REMAINING components of the patients Metabolic Syndrome (of which T2D is only one). Thus the teachings and use of the FS index in patients and the impact of the present invention on therapeutic intervention are profound.

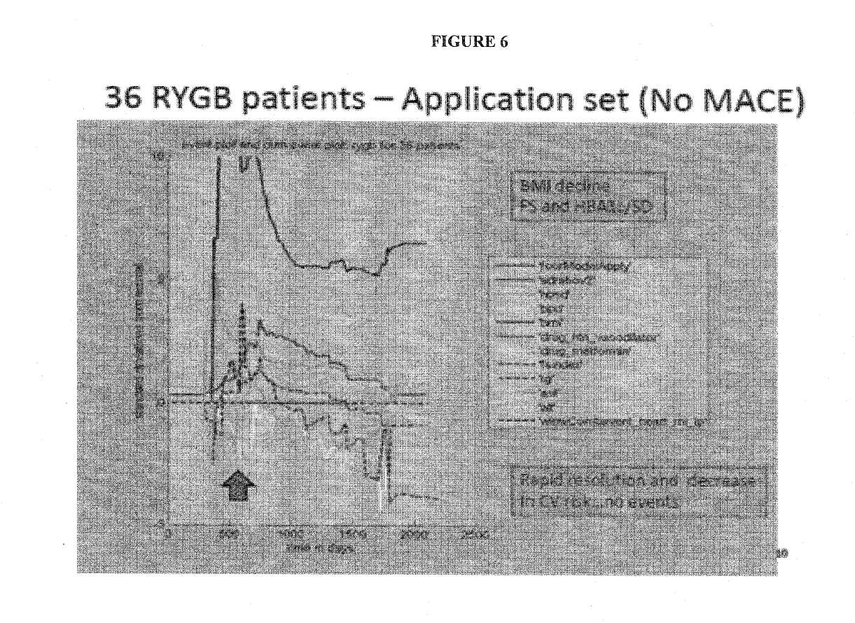

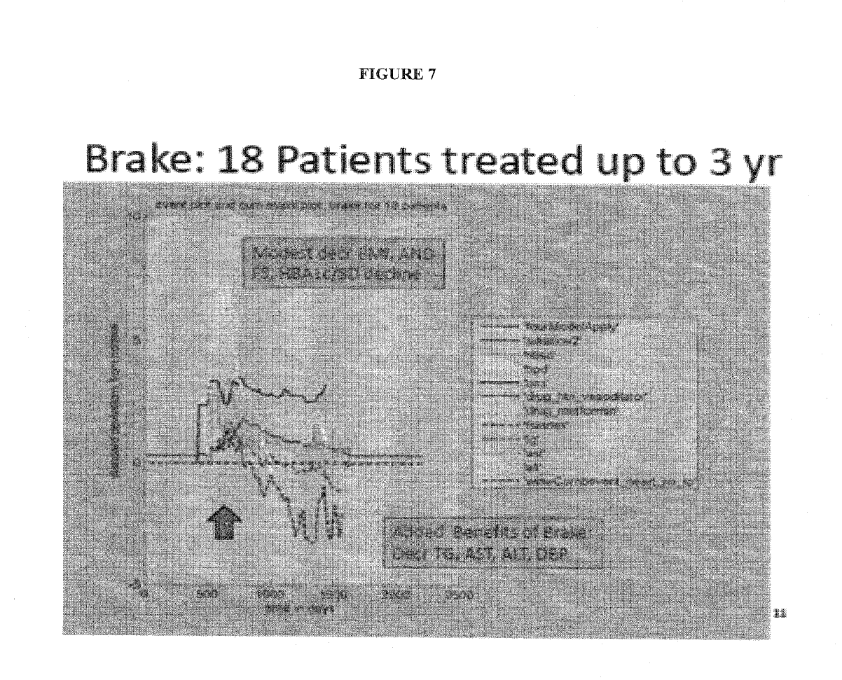

Abnormal FS index values which are subsequently normalized through administration of a composition according to the present invention (i.e. Brake™), indicated resolution of each component of MS syndrome, raising the possibility that specific treatments of MS might halt progression or reverse MS entirely, all the while working synergistically with the usual drugs applied and considered effective for each separate component of the metabolic syndrome. For example, changes in FS index in patients with RYGB surgery (FIG. 6) or after patients were administered a composition according to the present invention (FIG. 7) were dramatic, taking scores of some of these patients from above 250 to values below 20 in many cases. Reversal of organ damage is also a resulting effect, provided that the lowering of FS index occurs into the normal range and is kept there was a period sufficient to reverse the organ damage. Such is the importance of treating the entire metabolic syndrome, and the key to that is the disclosed FS index which is a measure of the risk to the patient from the entire metabolic syndrome.

The index also at least partially explains why drug therapies that improve one aspect of Metabolic Syndrome, but worsen others, appear not to mitigate organ damage risk or remove organ damaging events in complex Metabolic Syndrome patients. The index does also show that combination therapies consisting of individual drugs, each used for one component of metabolic syndrome may lower FS index by altering each component. One advantage of using the FS index is its perspective on the importance of combination therapy and in these specific examples to follow the FS index shows the importance of certain combination therapy beneficial on the glucose supply side, such as the composition therapy according to the present invention (Brake™ therapy).

Metabolic Syndrome-a Composite Condition vs. a Series of Loosely Related Parts

There are many isolated laboratory predictors for the presence of individual diseases such as T2D predicted by HBA1c or fasting blood glucose. Such parameters predict the disease and can be used to monitor the control of the parameter, such as when insulin lowers the blood glucose. Laboratory predictors of diseases are designed to be applied to disease detection over very broad populations of heterogeneous patients. These patients may have complex mixtures of diseases and treatments, and thus a broadly applicable but single parameter index such as “high LDL cholesterol” might separate some of the more obvious high risk atherosclerosis patients who have underlying lipid abnormalities, but not perform well on individuals who might deviate from the core metabolic syndrome model used to derive the index. There is currently no global index which predicts the risk of metabolic syndrome organ damage, and in fact other than the inventors, the idea that all of these diseases are phenotypic manifestations of an underlying metabolic syndrome pattern is not found in the literature. Since many of these patients have more than one metabolic syndrome parameter abnormal, we have since then expanded the concept of SD into patients who have other metabolic syndrome associated diseases concomitant with diabetes, such as Congestive Heart Failure, fatty Liver disease, Hepatitis C, COPD, Alzheimer's disease, sepsis and others.

Expanding the concept, the newly developed FS index (the method defining effective treatment with the disclosed pharmaceutical compositions in this application) handles all the common manifestations of Metabolic Syndrome, each of which was previously thought to be variably related to CV endpoints. So, the now highly novel FS index is designed to broadly model all the important aspects of metabolic syndrome (weight, triglycerides, liver inflammation, insulin production and SD ratio) and derive organ damage (generally) risk therefrom. Important to note that SD ratio is built into FS index as one of its 6 main components.

The term “metabolic syndrome manifestation” refers to a physiological effect, including a secondary effect which occurs in a subject who suffers from metabolic syndrome. Specific metabolic syndrome manifestations include but are not limited to T2D, hyperlipidemia, atherosclerosis, insulin resistance, hypertension, and Hepatic Steatosis. pancreas and/or pancreatic beta cell damage, hepatic steatosis, NAFLD, hyperlipidemia, elevated triglycerides, abdominal adiposity, atherosclerosis, cardiovascular diseases such as myocardial infarction, stroke, angina, congestive heart failure, hypertension, ASCVD, reduced lung capacity (COPD), Rheumatoid arthritis, diabetic nephropathy leading to kidney failure, gastrointestinal tract damage, gastrointestinal dysbiosis, inflammatory bowel disease, brain damage, neurodegenerative disorders, diabetic neuropathy, cognitive impairment associated with obesity and early Alzheimer's disease, among others as otherwise described herein.

The term “gastrointestinal disorder” includes diarrheal states, malabsorption in the upper gut (i.e., chronic pancreatitis, celiac disease), fatty liver, atrophic gastritis, short bowel syndrome, radiation enteritis, irritable bowel disease, Crohn's disease, post infectious syndrome, mild reflux, certain gut dysmotility, post chemotherapy disorder, malnutrition, malabsorption, and voluntary or involuntary long term starvation. The present invention may be used to treat each of these conditions, alone or secondary to the treatment or resolution of symptoms associated with T2D, pre-diabetic symptoms, metabolic syndrome and insulin resistance. To the extent that patients with Type 1 diabetes (T1D) manifest these metabolic syndrome properties, the present invention may also beneficially impact their outcome and slow progression of cardiovascular injury.

Formulations, Dosage Forms and Combinations

In particular, the present invention generally proceeds when the steps in practice of the invention include the testing of the patient for laboratory biomarker patterns, use of the results of testing to calculate the FS index, determining the risk of organ damaging events from the FS index calculation (when the FS index measures at least about 60, 100, 150, 200, 300, 400 or 500 and above), then the application of personalized treatment to lower the FS index, most preferably by the administration of a pharmaceutical composition targeted to a specific receptor (on the L-cells) in the distal intestine, in a dosage and duration of treatment to lower the FS index of the patient upon repeat measurements.

The effect of the medicament on the measured biomarkers demonstrates beneficial properties of the ileal brake hormone releasing substance on the laboratory tests that comprise the FS index. In the ordinary assessment of the precise sequence of hormonally produced events, the patient experiences cessation of hunger. The patient benefits from the ileal brake hormone release with regeneration of organs and tissues, typically pancreas, liver and gastrointestinal tract.

With respect to the sequence of signaling molecules from the ileum, a response to the medicament entails a wake up stimulation of distal intestinal L-cells that have been quieted by actions of intestinal bacteria or metabolic disease; there is a release of hormones and signals from said L-cells; said released hormones traveling in portal blood to pancreas, liver and GI tract, said organs regenerated from available growth factors and hormone signals, measured biomarkers of the FS index demonstrating the successful regeneration and said regenerated organs then signaling the patient, preferably a human, to resume adequate nutrition seeking behavior as directed by restored signals of hunger.

Dosage forms used in methods of the invention can be in a form suitable for oral use, for example, as tablets, troches, lozenges, suspensions, micro suspensions, dispersible powders or granules, emulsions, micro emulsions, hard or soft capsules. Useful dosage forms include osmotic delivery systems as described in U.S. Pat. Nos. 4,256,108; 5,650,170 and 5,681,584, multiparticulate systems as disclosed in U.S. Pat. No. 4,193,985; systems in which the nutritional substance is coated with a mixed film of a hydrophobic organic compound-enteric polymer as disclosed in U.S. Pat. No. 6,638,534; systems such as those described in U.S. Pat. Nos. 7,081,239; 5,900,252; 5,603,953; and 5,573,779; enteric-coated dry emulsion formulations (e.g., Journal of Controlled Release, vol. 107, issue 1 20 Sep. 2005, Pages 91-96), and emulsions such as the emulsion system of Olibra® and those disclosed in U.S. Pat. No. 5,885,590. Those of ordinary skill in the prior art know how to formulate these various dosage forms such that they release the majority of their nutritional substance in a subject's ileum (preferably within a pH range of about 7.0 to about 8.0, often about 7.2 to about 7.5) as otherwise described herein.

Exemplary dosage forms that will release the majority of the ileal brake hormone releasing substance (i.e., at least about 50% of the substance administered) in vivo upon reaching the ileum include oral dosage forms such as tablets, troches, lozenges, dispersible powders or granules, or a hard or soft capsules which are formed by coating the ileal brake hormone releasing substance with an enteric coating (e.g., an enteric cellulose derivative, an enteric acrylic copolymer, an enteric maleic copolymer, an enteric polyvinyl derivative, or shellac). Preferred enteric coatings have a pH dissolution profile that delays the release in vivo of the majority of the ileal brake hormone releasing substance until the dosage form reaches the ileum. Enteric coatings can consist of a single composition, or can comprise two or more compositions, e.g., two or more polymers or hydrophobic organic compound-enteric polymer compositions as described in U.S. Pat. No. 6,638,534).

A “material having a pH dissolution profile that delays release in vivo of the majority of the ileal brake hormone releasing substance until the dosage form reaches the ileum” includes but is not limited to cellulose acetate trimellitiate (CAT), hydroxypropylmethyl cellulose phthalate (HPMCP), polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, a mixture of amylose-butan-1-ol complex (glassy amylose) with Ethocel® aqueous dispersion (Milojevic et al., Proc. Int. Symp. Contr. Rel. Bioact. Mater. 20, 288, 1993), a coating formulation comprising an inner coating of glassy amylose and an outer coating of cellulose or acrylic polymer material (Allwood et al. GB 9025373.3), calcium pectinate (Rubenstein et al., Pharm. Res., 10, 258, 1993) pectin, chondroitin sulfate (Rubenstein et al. Pharm. Res. 9, 276, 1992), resistant starches (PCT WO 89/11269), dextran hydrogels (Hovgaard, et al., 3rd Eur. Symp. Control. Drug Del., Abstract Book, 1994, 87) modified guar gum such as borax modified guar gum, (Rubenstein and Gliko-Kabir, S. T. P. Pharma Sciences 5, 41-46, 1995), beta-cyclodextrin (Sidke et al., Eu. J. Pharm. Biopharm. 40 (suppl), 335, 1994), saccharide containing polymers, e.g., a polymeric construct comprising a synthetic oligosaccharide-containing biopolymer including methacrylic polymers covalently coupled to oligosaccharides such as cellobiose, lactulose, raffinose and stachyose, or saccharide-containing, natural polymers including modified mucopolysaccharides such as cross-linked pectate (Sintov and Rubenstein PCT/US 91/03014); methacrylate-galactomannan (Lehmann and Dreher, Proc. Int. Symp. Control. Rel. Bioact. Mater. 18, 331, 1991) and pH-sensitive hydrogels (Kopecek et al., J. Control. Rel. 19, 121, 1992), and resistant starches, e.g., glassy amylase, among others known in the art.

Methylmethacrylates or copolymers of methacrylic acid and methylmethacrylate are preferred materials having a pH dissolution profile that delays release in vivo of the majority of the ileal brake hormone releasing substance until the dosage form reaches the ileum. Such materials are available as Eudragit® polymers (Rohm Pharma, Darmstadt, Germany). For example, Eudragit® L100 and Eudragit® S100 can be used, either alone or in combination. Eudragit® L100 dissolves at pH 6 and upwards and comprises 48.3% methacrylic acid units per g dry substance; Eudragit® S100 dissolves at pH 7 and upwards and comprises 29.2% methacrylic acid units per g dry substance. Generally, the encapsulating polymer has a polymeric backbone and acid or other solubilizing functional groups. Polymers which have been found suitable for purposes of the present invention include polyacrylates, cyclic acrylate polymer, polyacrylic acids and polyacrylamides. Another preferred group of encapsulating polymers are the polyacrylic acids Eudragit® L and Eudragit® S which optionally may be combined with Eudragit® RL or RS. These modified acrylic acids are useful since they can be made soluble at a pH of 6 or 7.5, depending on the particular Eudragit chosen, and on the proportion of Eudragit® S to Eudragit® L, RS, and RL used in the formulation. By combining one or both of Eudragit® L and Eudragit® S with Eudragit® RL and RS (5-25%), it is possible to obtain a stronger capsule wall and still retain the capsule's pH-dependent solubility. In additional preferred aspects of the invention, a coating of shellac (which also includes one or more emulsifiers such as hypromellose and/or triacetin) which is chosen to have a suitable pH-dependent dissolution profile for release the contents of a dosage form such as a tablet within the ileum of a patient or subject may be used. This type of coating provides a nutrateric approach to delayed and/or controlled release using naturally occurring, non-synthetic components.

A delayed and/or controlled release oral dosage form used in the invention can comprise a core containing an ileum L-cell-stimulating amount of an ileal brake hormone releasing substance that is coated by an enteric coating. In some embodiments, the coating comprises Eudragit® L100 and shellac, or food glaze Eudragit® S100 in the range of 100 parts L100:0 parts S100 to 20 parts L100:80 parts S100, more preferably 70 parts L100:30 parts S100 to 80 parts L100:20 parts S100. As the pH at which the coating begins to dissolve increases, the thickness necessary to achieve ileum-specific delivery decreases. For formulations where the ratio of Eudragit® L100:S100 is high, a coat thickness of the order 150-200 μm can be used. For coatings where the ratio Eudragit® L100:S100 is low, a coat thickness of the order 80-120 μm can be used. Dosage forms used in methods of the invention can include one or more pharmaceutically acceptable carriers, additives, or excipients. The term “pharmaceutically acceptable” refers to a carrier, additive or excipient which is not unacceptably toxic to the subject to which it is administered. Pharmaceutically acceptable excipients are described at length by E. W. Martin, in “Remington's Pharmaceutical Sciences”, among others well-known in the art. pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk glucoses, as well as high molecular weight polyethylene glycols and the like.

Generally, the membrane or sustained release coating around the core, when formed with shellac, will comprise from about 5% to about 10% and preferably about 6% to about 8% based upon the total weight of the core and coating.

Independent of the core ileal brake hormone releasing substance is a second active drug, in a preferred embodiment a metformin derivative (e.g. a biguanide).

Additional preferred embodiment second active drugs of the invention include 10 mg of atorvastatin (Lipitor) or any statin in an equivalent amount, chosen from the alternative listing: Fluvastatin (Lescol), Lovastatin (Mevacor), Pitavastatin (Livalo), Pravastatin (Pravachol), Rosuvastatin (Crestor), Simvastatin (Zocor), among other possible statins.

Angiotensin Converting Enzyme (ACE) inhibitors with preferred example a 10 mg daily dose of Lisinopril (Prinivil, Zestril) or a suitable alternative in an equivalent amount chosen from those marketed ACE inhibitors: benazepril (Lotensin), captopril (Capoten), enalapril (Vasotec), fosinopril (Monopril), moexipril (Univasc), perindopril (Aceon), quinapril (Accupril), ramipril (Altace), trandolapril (Mavik). among other possible alternative ACE inhibitors.

Angiotensin II inhibitors with preferred example an 80 mg dose of Losartan or an equivalent amount of alternative Angiotensin II inhibitor including but not limited to candesartan, irbesartan, valsartan, olmesartan, Telmisartan, among other possible Angiotensin II inhibitors.

Beta Blockers with preferred example propranolol (Inderal) in a dose of 20 mg or a suitable alternative in an equivalent amount chosen from the list of beta blockers: acebutolol (Sectral); atenolol (Tenormin); betaxolol (Kerlone); bisoprolol (Zebeta); carteolol (Cartrol); esmolol (Brevibloc); metoprolol (Lopressor); penbutolol (Levatol); nadolol (Corgard); nebivolol (Bystolic); pindolol (Visken); timolol (Blocadren); sotalol (Betapace); carvedilol (Coreg); labetalol (Trandate), among other possible beta blockers.

Ribavirin or any antiviral agent; methotrexate or any anti-inflammatory agent; memantine or any anti-alzheimer's agent; sitagliptin or any DPP-IV anti-hyperglycemic agent; phentermine or any anti-obesity agent; berberine; vitamin B12; omeprazole or any proton pump inhibitor; sildenafil or any PDE-5 inhibitor; olanzapine, risperidone or any of the major tranquilizers.

In preferred embodiments by way of example, for each of the second active drugs listed above, when applied as an over-coating, the daily dose will be apportioned over 7 tablets of core ileal brake hormone releasing substance, such that each controlled release tablet is over-coated with 1/7^{th} of the total effective daily dose of second active agent. The range of effective daily doses for each of the over-coated second active drugs are as follows: atorvastatin (10 mg) or any statin (5-25 mg); lisinopril (10 mg) or any ACE inhibitor (5-100 mg); olmesartan (5-20 mg) or any Angiotensin II inhibitor (10-100 mg); propranolol (10-40 mg) or any beta blocker (5-100 mg); ribavirin (600-1200 mg) or any antiviral agent; methotrexate (1-5 mg) or any anti-inflammatory agent; memantine (5-20 mg) or any anti-alzheimer's agent; sitagliptin (50-100 mg) or any DPP-IV anti-hyperglycemic agent (5-100 mg); phentermine (18-37 mg) or any anti-obesity agent; berberine (500-1500 mg) in its available forms; vitamin B12 (5-25 mcg); omeprazole (10-20 mg) or any proton pump inhibitor (5-100 mg); sildenafil (10-50 mg) or any PDE-5 inhibitor (5-50 mg); olanzapine (5-20 mg), risperidone (1-5 mg) or any of the major tranquilizers.

The second active drug may be formulated often as an over-coating on the tablets of the ileal brake hormone releasing substance, in order to provide an immediate or early release of the second drug. Optionally, the second drug layer may be over-coated with a seal coat after the layer is applied. In one embodiment of the present invention, the metformin derivative is applied in the form of a layer to a controlled release core comprising the ileal brake hormone releasing substance as a layer using a binder and other conventional pharmaceutical excipients such as absorption enhancers, surfactants, plasticizers, antifoaming agents and combinations of the foregoing. An absorption enhancer may be present in the metformin derivative layer in an amount up to about 30% w/w in comparison to the weight of the metformin derivative. A binding agent may be present in an amount up to about 150% w/w of the metformin derivative. A second active drug immediate release formulation may be incorporated into a single dosage form by coating onto the membrane or sustained release coating of the dosage form by conventional methods. The incorporation of the second active drug may be performed by, but would not be limited to, the processes selected from the group consisting of drug layering, lamination, dry compression, deposition, printing and the like.

When the metformin derivative is coated onto a membrane or controlled release coating of an osmotic tablet core, the metformin coating should be applied from a coating solution or suspension that employs an aqueous solvent, an organic solvent or a mixture of an aqueous and an organic solvent. Typical organic solvents include acetone, isopropyl alcohol, methanol and ethanol. If a mixture of aqueous and organic solvents is employed, the ratio of water to organic solvent should range from about 98:2 to 2:98, preferably about 50:50 to 2:98, most preferably about 30:70 to 20:80 and ideally about 25:75 to 20:80. If a mixed solvent system is employed, the amount of binder required for coating the metformin derivative onto the membrane or sustained release coating may be reduced. For example, successful coatings have been obtained from a mixed solvent system where the ratio of binder to metformin derivative is 1:9 to 1:11. Although acceptable coatings can be obtained when the metformin coat is applied directly to the membrane or sustained release coating, a preferred approach is to first coat the membrane or sustained release coating with a seal coat prior to the application of the metformin coating. As used herein a seal coat is a coating that does not contain an active pharmaceutical ingredient and that rapidly disperses or dissolves in water.

The metformin coating solution or suspension may also contain a surfactant and a pore forming agent. A pore forming is preferably a water-soluble material such as sodium chloride, potassium chloride, sucrose, sorbitol, mannitol, polyethylene glycols (PEG), propylene glycol, hydroxypropyl cellulose, hydroxypropyl methylcellulose, hydroxypropyl methylcellulose phthalate, cellulose acetate phthalate, polyvinyl alcohols, methacrylic acid copolymers, poloxamers (such as LUTROL F68, LUTROL F127, LUTROL F108 which are commercially available from BASF) and mixtures thereof.

In addition, various diluents, excipients, lubricants, dyes, pigments, dispersants, etc., which are disclosed in Remington's Pharmaceutical Sciences (1995), may be used to optimize the above listed formulations of the subject invention.

Biguanides, such as metformin are commonly administered in dosage forms containing 500 mg, 750 mg, 850 mg, and 1000 mg. Ileal Brake hormone releasing substances, such as Brake™, are commonly administered as individual controlled release tablets, and multiple tablets are combined in a single daily dose of between about 5.0 and 20.0, often about 7.5 and 15, often about 10 to about 12.5 grams of active ileal brake hormone releasing substances. The preferred embodiment of a combination of metformin and the ileal brake hormone releasing substance would overcoat 500 mg of metformin onto 7 tablets of the ileal brake hormone releasing core. Ideally, there would be about 70 mg of metformin overcoat on each of the 7 Brake™ tablets. The present invention is intended to encompass the above listed therapeutic combinations, without providing a specific example of each possible combination of compounds and their respective dosage amounts. It is noted that when agents other than or in addition to biguanides are utilized in combination with Brake™, similar formulations may be provided with a controlled release ileal brake hormone releasing core and an overcoat of the additional bioactive agent(s) formulated for immediate or quick release.

Emulsions and microemulsions may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the ileal brake hormone releasing substance, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

Techniques for formulating the aforementioned useful dosage forms are either disclosed in the references cited above or are well-known to those of ordinary skill in the art.

“Stabilizing a subject's blood glucose and insulin levels” means lowering the subject's blood glucose and insulin levels to healthy levels within normal or close to normal ranges.

The terms “obesity” and “overweight” are generally defined by body mass index (BMI), which is correlated with total body fat and estimates the relative risk of disease. BMI is calculated by weight in kilograms divided by height in meters squared (kg/m2). Normal BMI is defined as a BMI of about 18.5 to 24.9 kg/m2. Overweight is typically defined as a BMI of 25-29.9 kg/m2, and obesity is typically defined as a BMI of at least 30 kg/m2. See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998). Obesity and its associated disorders are common and very serious public health problems in the United States and throughout the world. Central or Abdominal (18) adiposity is the strongest risk factor known for T2D and is a strong risk factor for cardiovascular disease. Central adiposity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. Obesity reduces life-span and carries a serious risk of the co-morbidities listed above, as well as disorders such as infections, varicose veins, acanthosis nigricans, eczema, exercise intolerance, insulin resistance, hypertension hypercholesterolemia, cholelithiasis, orthopedic injury, and thromboembolic disease (18).

Because they specifically mimic the effects of RYGB surgery and thereby effectively treat, remediate or often cure metabolic syndrome, the present compositions are also useful for treating central adiposity, and favorably impact the conditions which often occur secondary to metabolic syndrome.

“Once-daily administration to the subject of a delayed and/or controlled release dosage form” includes self-administration of the dosage form by the subject.

The term “coadministration” is used to describe the administration of two or more active compounds, in a number of embodiments according to the present invention, in a pharmaceutical composition comprising at least one ileal brake hormone releasing substance in a first active composition and optionally, at least one second active composition formulated in the same pharmaceutical composition, often with different release characteristics as otherwise described herein. In a number of embodiments according to the present invention a first pharmaceutical composition (which may or may not contain an active other than the at least one ileal brake hormone releasing substance) may be administered with a second distinct pharmaceutical composition comprising an additional active agent to treat subjects according to the present invention. Although the term coadministration preferably includes the administration of two or more active compounds to the patient at the same time and often in one pharmaceutical composition comprising different release characteristics, it is not necessary that the compounds actually be administered at the exact same time, only that amounts of the different active compound will be administered to a patient or subject such that effective concentrations of the active compounds are found in the blood, serum or plasma, ileum or treated tissue at the same time.

“Dietary components” in the phrase “wherein the nutritional substance comprises an enteric coated tablet or micro-encapsulation of glucose, lipids and dietary components” means any natural substance which either itself evidences impact on the ileal brake, or alternatively, enhances the impact that glucose and/or lipids have on the ileal brake, such components including other complex carbohydrates and nutritional components as otherwise described herein including, for example, alfalfa leaf, chlorella algae, chlorophyllin and barley grass juice concentrate, among a number of other agents.

Combination therapy is ordinarily given as a treatment for the disclosed components of metabolic syndrome, and most patients are treated with statins and fish oils for their hyperlipidemia, metformin or DPP-IV inhibitors for their diabetes, ACE or AII inhibitors for their hypertension, phentermine containing or phentermine-topiramate products for weight loss, and mixtures of dietary supplements, vitamins, and the like. The present invention, by acting to release the metabolic regulatory hormones of the ileal brake, is designed to offer combination treatment to patients with an overall more successful control of the underlying metabolic syndrome and its various manifestations. In this manner, aspects of the present invention can include combinations of the enteric coated tablet or micro granules with any of these medicaments. Medicaments may be over-coated onto the finished core invention, such as, for a non-limiting example, atorvastatin 10 mg coated onto 10 gram of tablets, wherein each enteric coated tablet is coated with 2.0 mg of atorvastatin in an immediate or early release form. By way of alternative example, formulation of the identical components, the 10 mg of atorvastatin may be formulated into immediate or early release micro granules and these may be mixed with 10 gm of microgranule formulation of the enteric coated instant invention. This combination form of the medicament may be given to the patient one or more times a day.

### DESCRIPTION OF PREFERRED EMBODIMENTS AND METHODS

As summarized above, the system, diagnostics and pharmaceutical inventions disclosed provide treatment methods and organ regeneration methods for patients with metabolic syndromes including hyperlipidemia, weight gain, insulin resistance, hypertension, atherosclerosis, fatty liver diseases and certain chronic inflammatory states. These treatment methods can entail the calculation of indexes used to assess the severity of metabolic syndrome, such as FS index. Methods can further entail testing of biomarkers; testing of breath, blood or body fluid biomarkers and selection of pharmaceutical compositions to resolve one or more of the metabolic syndrome conditions including but not limited to hyperlipidemia, weight gain, hepatic steatosis, insulin resistance, hypertension, and atherosclerosis, fatty liver and chronic inflammatory states.

Thus, the invention provides a method of treatment of metabolic syndromes, wherein personalized treatments and pharmaceutical compositions are selected using the results of biomarker testing including but not limited to testing such as HBA1c, glucose, GLP-1, PYY, GLP-2, insulin, Proinsulin, CRP, hsCRP, endotoxin, IL-6 and the like. Personalized treatment and pharmaceutical compositions can be selected using a Glucose Supply Side computerized algorithm and system, wherein said Glucose Supply Side treatment method for diabetes consists of an algorithm (incorporated herein in its entirety) ranking favorable attributes of pharmaceutical compositions acting by minimizing excess glucose inside cells, and minimizing the amount of glucose that reaches target cells of the metabolic syndrome afflicted patient.

The invention also provides a method of treatment of metabolic syndromes, wherein personalized treatment and pharmaceutical compositions are selected by comparison of biomarker behavior patterns between patients having responded to Roux-en-Y bariatric surgery and their own response to oral dosing with pharmaceutical formulations comprised of carbohydrates, lipids or amino acids which activate the ileal brake response of the ileum in a manner similar to RYGB surgery. The method specifically entails orally administered pharmaceutical compositions that mimic the action of RYGB surgery on the ileal brake. Even more specifically, the formulation for treatment of metabolic syndrome comprises the micro-encapsulation of glucose, lipids and components of diet formulated to release these active compositions at pH values preferably between about 7.2 and 7.5, which targets the action of said medicaments at the ileal brake in the distal intestine. The encapsulated compositions disclosed are a preferred medicament to decrease appetite for glucose, and thereby lower inflammation and benefit to the treatment of patients with metabolic syndrome, according to the results of testing of targeted biomarkers.

In a preferred embodiment of a method of treatment of metabolic syndromes according to the invention, oral dosing with about 2,000 to 12,500 up to about 20,000, about 2500-3,000 to 10,000, about 7,500-10,000 milligrams of a pharmaceutical formulation of microencapsulated sugars, lipids, and/or amino acids activates the ileal brake in a dose increasing magnitude and treats one or more of the following components of metabolic syndrome: hyperlipidemia, weight gain, hepatic steatosis, insulin resistance, hypertension, atherosclerosis, fatty liver diseases and chronic inflammatory states. The name of this medicament is Brake™.

In another embodiment, the invention provides a pharmaceutical formulation for treatment of metabolic syndrome, wherein the microencapsulated activation of the ileal brake is produced at a pH of about 6.5 to about 7.5 and involves the release of about 2,000 to about 12,500 up to about 20,000, about 2,500-3,000 to 10,000, about 7,500 to 10,000 milligrams of glucose, fructose, dextrose, sucrose or other glucose compositions active on the ileal brake in mammals at dosages between about 2,000 and about 10,000-12,500 milligrams, and as presented above.

In another embodiment, the invention provides a pharmaceutical formulation wherein the microencapsulated activation of the ileal brake is produced by approximately pH 6.5 to 7.5 release of about 2,000 to about 6,000, about 2,500-3,000 to about 10,000-12,500 milligrams of dextrose and about 2,000-4,000 milligrams of a lipid such as olive oil, corn oil, palm oil, omega3 fatty acid or other suitable lipid substances active on the ileal brake of mammals.

In one embodiment, a pharmaceutical formulation for treatment of metabolic syndrome of the invention can achieve the microencapsulated activation of the ileal brake at about pH 6.5 to 7.5 by release of about 2,000 to about 10,000-12,500 up to about 20,000, about 2,500-3,000 to about 10,000, about 7,500-10,000 milligrams given once, twice or three times daily.

In another embodiment, a method of treatment of metabolic syndromes according to the invention involves oral treatment and includes use of pharmaceutical formulations as described above that activate the ileal brake and which act in the gastrointestinal tract and the liver of a mammal to control metabolic syndrome manifestations, regenerate organs and tissues and thereby reverse or ameliorate the cardiovascular damage (atherosclerosis, hypertension, lipid accumulation, and the like) resulting from progression of metabolic syndrome.

In another preferred embodiment, a composition or a method of treatment of metabolic syndromes according to the invention involves an oral formulation mimetic of RYGB and includes use of said oral formulation over-coated with a second active agent, chosen from medicaments ordinarily used for treatments of separate manifestations of metabolic syndrome including but not necessarily limited to T2D, hyperlipidemia, atherosclerosis, hypertension, hepatic steatosis, insulin resistance, or chronic inflammation. The second active pharmaceutical agent can be, by way of specific example, metformin, sitagliptin, saxagliptin, methotrexate, olanzapine, donepezil, memantine, atorvastatin, simvastatin, lovastatin, olmesartan, Enalapril, lisinopril, candesartan, irbesartan, roflumilast, among others. Such compositions are the first to combine treatment of all of the primary metabolic syndrome manifestations into one product given once or twice daily to patients with all or many of the manifestations of metabolic syndrome, and the newly discovered organ regeneration capability is responsible for the long lasting efficacy of these medicament combination pharmaceuticals and in certain cases, an actual cure of the patient.

In a preferred example, a disclosed composition of the invention can act to limit hepatic gluconeogenesis in the same manner as metformin, as well as add pancreatic regeneration and many other actions beneficial to the treatment of metabolic syndrome. The class of compounds related to and including metformin is called biguanide antihyperglycemic agents. While metformin is illustrative, and the combination product therefrom is called MetaBrake™, the list of biguanides is not exclusive beyond metformin, and additional metformin mimetic or biguanide medicaments can be added to the formulations of the invention without departing from the practice of treatments for metabolic syndrome that combine oral mimetics of RYGB surgery effects on the ileal brake in conjunction with conventional anti-diabetes medicaments of the class represented by metformin. When used together with biguanide medicaments with particular emphasis on metformin, the dosage required to lower glucose, lipids, hepatic steatosis and inflammation may be reduced. When combined into an oral dosage form of Brake™ and a biguanide such as metformin, each of 7 tablets would contain about 1000 mg of ileal hormone releasing substances and 75 mg of metformin. In this manner the total dose of metformin per day would be about 500 mg and the ileal hormone releasing substance would be less than about 10,000 mg, yet the combined product would control glucose, lower body weight, control triglycerides, lower systemic inflammation, and effect regeneration of organs and tissues, beneficial actions that are substantially beyond those of metformin alone.

In one aspect of a composition or a method of treatment of metabolic syndromes according to the invention, the second active pharmaceutical agent is from the class of DPP-IV inhibitors, including but not limited to formulations whereby the composition acts in the same way as DPP-IV inhibitors and the like. Examples of similar orally administered agents, thought to act by inhibition of DPP-IV, include Alogliptin, Vildagliptin, Sitagliptin, Dutogliptin, Linagliptin and Saxagliptin. While illustrative, this list is not meant to be exhaustive and it is readily apparent to persons skilled in the art of T2D care that additional DPP-IV inhibitors can be added to the formulations of the invention without departing from the practice of preparing oral treatments for metabolic syndrome that combine oral mimetics of RYGB surgery effects on the ileal brake in conjunction with conventional anti-diabetes medicaments of the class represented by DPP-IV inhibitors. When used together with so called DPP-IV inhibitors, the dosage required to lower glucose, lipids, triglycerides and inflammation may be reduced to the benefit of reduction of the side effects of DPP-IV inhibitors, in particular the pancreatitis, which is presumed to be related to dosage of DPP-IV inhibitor chosen for treatment. When combined into an oral dosage form of Brake™ and a DPP-IV inhibitor such as sitagliptin, by way of example, each tablet would contain about 1000 mg of ileal brake hormone releasing substances and 10 mg of sitagliptin. In this manner the total dose of sitagliptin per day would be less than 100 mg, yet the combined product would, in a completely novel way, control glucose, lower body weight, control triglycerides, lower systemic inflammation and regenerate organs and tissues in a similar manner as RYGB surgery. This combination product of Brake™ and sitagliptin, called JanuBrake™ would be given once or twice daily and be suitable for consumer use of sitagliptin with an increased safety profile over that of sitagliptin alone. Similar gains in potency at lower doses, broad array of treatment responses in metabolic syndrome, and safety advantages over the statin alone would be seen with each of the DPP-IV inhibitors reduced to practice, and the disclosure of invention of a synergistic combination encompasses all DPP-IV inhibitor combinations with Brake™ prepared in this manner for these purposes.

In another aspect of a composition or a method of treatment of metabolic syndromes according to the invention, the second active pharmaceutical agent is from the class of insulin sensitizers, also known as TZDs or Thiazolidinediones which are also known to be active on PPAR. Examples of similar agents, thought to act on the defined insulin sensitizer pathway, include pioglitazone, rosiglitazone, rivoglitazone, aleglitazar and the PPAR-sparing agents MSDC-0160, MSDC-0602. While illustrative, this list is not meant to be exhaustive and it is readily apparent to persons skilled in the art that additional insulin sensitizers, thiazolidinediones or PPARs or PPAR-sparing medicaments can be added to the formulations of the invention without departing from the practice of oral treatments for metabolic syndrome that combine oral mimetics of RYGB surgery effects on the ileal brake in conjunction with conventional anti-diabetes medicaments of the class represented by insulin sensitizers.

In another aspect of a composition or a method of treatment of metabolic syndromes according to the invention, the second active pharmaceutical agent is an alpha glucosidase inhibitor including but not limited to acarbose. The pharmaceutical thereby acts in the gastrointestinal tract, combining the effects on the ileal brake hormone release with the interruption of glucose absorption in the same way as acarbose, with fewer adverse effects, and to specifically include delayed release preparations of Acarbose, Miglitol, Voglibose and the like.

A composition or a method of treatment of metabolic syndromes according to the invention can also include the additional use of colesevelam, or can involve the use of a composition that acts in the gastrointestinal tract and on the ileal brake to limit glucose supply and to lower the lipid content of the blood in the same manner as colesevelam. While illustrative, the selection of a combination including colesevelam is not meant to be exhaustive and it is readily apparent that additional colesevelam mimetic medicaments can be added to the pharmaceutical composition of the invention without departing from the practice of oral treatments for metabolic syndrome that combine oral mimetics of RYGB surgery effects on the ileal brake in conjunction with conventional anti-diabetes medicaments of the class represented by colesevelam.

In another aspect of a composition or a method of combination treatment of metabolic syndromes according to the invention, the second active pharmaceutical agent is from the class of statins, also known as cholesterol synthesis inhibitors or HMG-CoA reductase inhibitors. Examples of similar agents, thought to act on the defined statin pathway or by HMG-CoA reductase inhibition, include atorvastatin, simvastatin, lovastatin, ceruvastatin, pravastatin. While illustrative, this list of available statin drugs is not meant to be exhaustive and it is readily apparent to persons skilled in the art that additional statins can be added to the formulations of the invention without departing from the practice of oral treatments for metabolic syndrome that combine oral mimetics of RYGB surgery effects on the ileal brake in conjunction with conventional anti-hyperlipidemic medicaments of the class represented by statins. When used together with so called statins, the dosage required to lower lipids and triglycerides may be reduced to the benefit of reduction of the side effects of statins, in particular the myopathy, which is known in the art to be related to higher dosages such as 80 mg of simvastatin. When combined into an oral dosage form of Brake™ and a statin such as atorvastatin, by way of example, each tablet would contain 1000 mg of ileal hormone releasing substances coated to release said ileal brake hormone releasing substances in the ileum and be over-coated with 2 mg of atorvastatin or a related agent in an effective amount with conventional release characteristics for targeted release in the duodenum. In this manner the total dose of atorvastatin per day would be less than 20 mg, yet the combined product would control glucose, lower body weight, control triglycerides, lower systemic inflammation, and regenerate organs and tissues. This product, called LipidoBrake™ would be given once or twice daily and be suitable for consumer use of atorvastatin with an improved safety profile over that of atorvastatin alone. Similar gains in potency at lower doses, a broad array of treatment responses in metabolic syndrome, and safety advantages over the statin alone would be seen with each of the statins reduced to practice, and the disclosure encompasses all statin combinations with Brake™ prepared in this manner for these purposes.

In another aspect of a composition or a method of combination treatment of metabolic syndromes according to the invention, the second active pharmaceutical agent is from the class of angiotensin II inhibitors, also known as AII inhibitors. Examples of similar AII inhibitor agents, thought to act on the defined hypertension pathway, include Valsartan, Olmesartan, Candesartan, Irbesartan, Losartan, Telmisartan and the like. While illustrative, this list is not meant to be exhaustive and it is readily apparent to persons skilled in the art that additional AII inhibitors can be added to the formulations without departing from the practice of oral treatments for metabolic syndrome that combine oral mimetics of RYGB surgery effects on the ileal brake in conjunction with conventional anti-hypertensive medicaments of the class represented by AII inhibitors, the effect of both being mediated by organ and tissue regeneration.

A composition or a method of combination treatment of metabolic syndromes according to the invention can use a second active pharmaceutical agent that includes a PDE-5 inhibitor such as sildenafil (Viagra), vardenafil (Levitra) and Tadalafil (Cialis) phosphodiesterase type 5 inhibitor, often shortened to PDE-5 inhibitor, is a drug used to block the degradative action of phosphodiesterase type 5 on cyclic GMP in the smooth muscle cells lining the blood vessels supplying the corpus cavernosum of the penis. These drugs are used in the treatment of erectile dysfunction. While illustrative, this list is not meant to be exhaustive and it is readily apparent to persons skilled in the art that additional medicaments active in the treatment of erectile dysfunction can be added to the formulations of the invention without departing from the practice of oral treatments for metabolic syndrome that combine oral mimetic of the RYGB surgery effect on the ileal brake in conjunction with conventional PDE-5 inhibitors used in the treatment of erectile dysfunction.

A composition or a method of combination treatment of metabolic syndromes according to the invention can also use a second active pharmaceutical agent such as methotrexate, Lorcaserin, topiramate, olanzapine (Zyprexa), risperidone or Ziprasidone, a second active pharmaceutical agent that is active in the treatment of secondary weight gain and metabolic syndrome that leads to onset of Alzheimer's disease, including but not limited to Donepezil, (Aricept) a centrally acting reversible acetylcholinesterase inhibitor, memantine (Namenda), an NMDA receptor antagonist involved with the action of glutamate or known inhibitors of beta amyloid protein formation.

A composition or a method of combination treatment of metabolic syndromes according to the invention can also use a second active pharmaceutical agent such as an ACE inhibitor including but not limited to members of this class illustrated by captopril, lisinopril, enalapril, quinapril, perindopril, trandolapril, a GPR119 agonist, including but not limited to the following candidates in early phase human trials: Array Biopharma 0981; Arena/Ortho McNeil APD597; Metabolex MBX-2982; Prosidion/OSI PSN821 and the like, one or more of the active compositions used to treat HIV associated diseases, one or more of the active compositions used to treat Hepatitis B, C or other forms of chronic Hepatitis most preferably sofosbuvir or ribavirin, or the method or composition my also include the use of an intestinal probiotic mixture of bacteria formulated to release at pH between about 6.5 and about 7.5, which replaces the bacterial flora of the intestine at the location of the ileum.

In one embodiment of a composition or a method of treatment of metabolic syndromes according to the invention, the second active pharmaceutical agent acts as a mimetic of the incretin pathway to lower glucose in the same or similar way as exenatide, including orally administered and parenterally administered sustained release preparations of exenatide and the like. Examples of similar agents, thought to act specifically on the defined GLP-1 pathway, include liraglutide, Lixisenatide, and taspoglutide. While illustrative, this list is not meant to be exhaustive and it is readily apparent to persons skilled in the art of T2D care that additional GLP-1 pathway mimetics that are not DPP-IV inhibitors can be added to this list without departing from the practice of oral treatments for metabolic syndrome that combine oral mimetics of RYGB surgery effects on the ileal brake in conjunction with conventional anti-diabetes medicaments of the class represented by incretin pathway mimetics.

In another embodiment of a composition or a method of treatment of metabolic syndromes according to the invention, the orally active ileal brake hormone releasing substances may be combined with insulin formulated for oral administration, including orally administered sustained release preparations of insulin and the like. Micro-spheres or nano-spheres formed of polymers or proteins such as insulin are known to those skilled in the art, and can be tailored for passage through the gastrointestinal tract directly into the bloodstream. Alternatively, the insulin or therapeutic peptide or protein compound can be incorporated into cholestosomes (see US 2007/0225264A1), bio-erodible polymers, and/or micro-spheres/nano-spheres, or composites of these delivery vehicles. See, for example, U.S. Pat. Nos. 4,906,474, 4,925,673 and 3,625,214, and Jein, TIPS 19:155-157 (1998), the contents of which are hereby incorporated by reference. Examples of these oral formulations of insulin include HDV-1 insulin and oral insulin formulations by Emisphere, Biocon and Oramed. While illustrative, this list is not meant to be exhaustive and it is readily apparent to persons skilled in the art of diabetes care that additional formulations of oral insulin can be added to this list without departing from the practice of oral treatments for metabolic syndrome that combine oral mimetics of RYGB surgery effects on the ileal brake in conjunction with conventional anti-diabetes medicaments of the class represented by the oral insulin pathway mimetics.

In still another embodiment of a composition or a method of treatment of metabolic syndromes according to the invention, personalized treatment and pharmaceutical compositions can be selected for treatment of metabolic syndrome manifestations including, but not limited to T2D, hepatic steatosis, insulin resistance, hypertension, hyperlipidemia, fatty liver disease, and chronic inflammation.

In still another embodiment of a composition or a method of treatment of metabolic syndromes according to the invention, the combination pharmaceutical formulation of an anti-diabetic drug and sugars, lipids and amino acids of Brake™ activates the ileal brake and thereby reduces insulin resistance, lowers blood glucose, lowers body weight in central adiposity, lowers systemic inflammation, lowers fatty liver disease, lowers triglycerides and other lipids and regenerates organs and tissues in a patient with any or all of the components of metabolic syndromes.

In still another embodiment of a composition or a method of treatment of metabolic syndromes according to the invention, the combination pharmaceutical formulation of a lipid lowering drug and sugars, lipids and amino acids of Brake™ activate the ileal brake and thereby reduces insulin resistance, lowers blood glucose, lowers body weight in central adiposity, lowers systemic inflammation, lowers fatty liver disease, lowers triglycerides and other lipids and regenerates organs and tissues in a patient with any or all of the components of metabolic syndromes.

In still another embodiment of a composition or a method of treatment of metabolic syndromes according to the invention, the combination pharmaceutical formulation of an anti-obesity drug and disclosed sugars and/or lipids activates the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndrome.

In still another embodiment of a composition or a method of treatment of metabolic syndromes according to the invention, the combination pharmaceutical formulation of an anti-inflammatory drug such as methotrexate with sugars and/or lipids activate the ileal brake to produce beneficial immunoregulatory actions and thereby reduces insulin resistance, lowers blood glucose, lowers body weight in obesity, lowers systemic inflammation, lowers fatty liver disease, lowers triglycerides and other lipids and regenerates organs and tissues in a patient with any or all of the components of metabolic syndromes.

In still another embodiment of a composition or a method of treatment of metabolic syndromes according to the invention, the combination pharmaceutical formulation of an anti-hypertensive drug with sugars and/or lipids activates the ileal brake and thereby reduces blood pressure, lowers insulin resistance, lowers blood glucose, lowers body weight in obesity, lowers systemic inflammation, lowers fatty liver disease, lowers triglycerides and other lipids and regenerates organs and tissues in a patient with any or all of the components of metabolic syndromes.

In still another embodiment of a composition or a method of combination treatment of metabolic syndromes according to the invention, the combination pharmaceutical formulation of an anti-atherosclerosis drug, with sugars and/or lipids activates the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes.

In still another embodiment of a composition or a method of treatment of metabolic syndromes according to the invention, personalized treatment and pharmaceutical compositions are selected for treatment of metabolic syndrome manifestations of Erectile Dysfunction that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of chronic obstructive pulmonary disease, or COPD, that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes.

In still another embodiment of a composition or a method of treatment of metabolic syndromes according to the invention, personalized treatment and pharmaceutical compositions are selected for treatment of metabolic syndrome manifestations of Rheumatoid Arthritis, or RA, that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes. For treatment of RA the preferred medicament for overcoat formulation is methotrexate

In still another embodiment of a composition or a method of combination treatment of metabolic syndromes according to the invention, personalized treatment and pharmaceutical compositions are selected for treatment of metabolic syndrome manifestations of Alzheimer's disease, preferably the variant of Alzheimer's disease associated with T2D that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes. For treatment of Alzheimer's disease the preferred medicaments for overcoat formulation are memantine or donepezil.

In still another embodiment of a composition or a method of combination treatment of metabolic syndromes according to the invention, personalized treatment and pharmaceutical compositions are selected for treatment of metabolic syndrome manifestations of Multiple Sclerosis that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of Crohn's Disease that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of Non-Alcoholic Fatty Liver Disease (NAFLD) that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes. For treatment of NAFLD the preferred medicament of over-coating of the ileal brake hormone releasing formulation is berberine as available forms in a daily amount of about 500-1000 mg.

In still another embodiment of a composition or a method of treatment of metabolic syndromes according to the invention, personalized treatment and pharmaceutical compositions are selected for treatment of metabolic syndrome manifestations of Hepatitis that act on the ileal brake and thereby reduces insulin resistance, lowers blood glucose, lowers body weight, lowers systemic inflammation, lowers liver disease, lowers triglycerides and other lipids and regenerates organs and tissues in a patient with any or all of the components of metabolic syndromes.

In still another embodiment of a composition or a method of combination treatment of metabolic syndromes according to the invention, personalized treatment and pharmaceutical compositions are selected for treatment of metabolic syndrome manifestations of HIV diseases that act on the ileal brake and thereby reduces insulin resistance, lowers blood glucose, lowers body weight, lowers systemic inflammation, lowers fatty liver disease, lowers triglycerides and other lipids and regenerates organs and tissues in a patient with any or all of the components of metabolic syndromes.

The invention also provides a process for the combination oral treatment of metabolic syndromes including but not limited to T2D and conditions associated with diabetes mellitus, wherein said process comprises diagnosing said disease states and/or conditions through calculation of FS index and SD ratio for the patient, testing of ileum pH values using the SmartPill device, testing of breath biomarkers which include oxygen, glucose, acetoacetate, betahydroxybutyrate, and other suitable free fatty acids and ketone bodies well known in the art; testing isoprostane and other metabolites of prostaglandins or any other analytes that are considered markers of oxidative stress; Nitrous oxides, methyl nitrous oxide metabolites; cytokines, proteins, GLP-1. GLP-2, PYY, proinsulin, insulin, incretins, peptides, adiponectin, C-Reactive Protein, hsCRP, endotoxin, procalcitonin, troponin, alpha-fetoprotein, electrolytes, and other markers of the inflammatory pathways or those of cardiovascular injury. The processes specifically incorporate the testing of these and other biomarkers and use of the results to select pharmaceutical compositions that act on the ileal brake and incorporate other currently available pathway specific biomarkers for metabolic syndrome manifestations. While illustrative, this list of medicaments for combination oral treatment is not meant to be exhaustive and it is readily apparent to persons skilled in the art of diabetes care that additional biomarkers and combinations of medicaments can be added to this list without departing from the practice of testing of biomarkers and using these results to select personalized treatments for patients with metabolic syndromes.

For example, in such practices of the invention of combination treatments for metabolic syndrome manifestations that include an active medicament and the disclosed formulations that act as ileal brake hormone releasing agents, the condition to be treated is T2D, T1D, Rheumatoid Arthritis, Alzheimer's disease, Crohn's disease, Multiple Sclerosis, Irritable Bowel syndrome (IBS), COPD, Psoriasis, HIV or AIDS, Non-Alcoholic Fatty Liver Disease, Hepatitis C, Congestive Heart Failure, Myocardial infarction, Stroke, angina, Atherosclerosis, Chronic Inflammation, Hypertension, Hyperlipidemia and Erectile Dysfunction.

In certain embodiments of a pharmaceutical composition of the invention used in the treatment of metabolic syndrome according to the practices of the invention disclosed herein, the ileal brake hormone releasing composition is over-coated with a necessary amount of Vitamins A, D, E or B12, or a necessary daily amount of Aspirin, ranging between about 81 to about 325 mg, or a necessary amount of omega-3, as derived from fish oils, or a necessary amount of micro-encapsulated food grade chocolate, either as dark chocolate, milk chocolate or white chocolate, each alone or as mixed components. In other embodiments, a pharmaceutical composition of the invention includes the substances disclosed herein and the remainder of the dosage form comprises mixtures of food components of sugars, lipids and amino acids and acts in the same way as pH encapsulated glucose, releasing at a pH of about 6.8 to about 7.5 to lower appetite, selectively modify taste and thereby change taste preferences for foods and nutrients, regulate the immune system and lower systemic inflammation, restore normal compositions of bacteria, regenerate organs and tissues in metabolic syndromes and associated conditions. Examples of active compositions include combinations of pH encapsulated microparticulates of different pH release for glucose, over-coated with immediate or early release DPP-IV inhibitors, TZD compounds, ACE inhibitors, AII inhibitors, Incretin pathway mimetics, PDE5 inhibitors, pH encapsulated probiotic organisms, Statins, antibiotics, and GLP-1 mimetics. While illustrative, this list of combinations and pH release encapsulated compounds is not meant to be exhaustive and it is readily apparent to persons skilled in the art of metabolic syndrome treatment that additional pH encapsulated compounds and additional classes of supply side beneficial substances can be added to this list without departing from the practice of testing of biomarkers and using these results to select personalized treatments for patients with metabolic syndrome.

In another aspect, the invention provides a Glucose Supply Side method for the treatment of T2D and an FS index calculation method for the treatment of metabolic syndrome component conditions beyond T2D. The Glucose Supply Side method comprises the administration to a human or non-human mammal in need thereof of any of the pharmaceutical compositions described above in any combination and each in any dosage according to the results of testing of biomarkers. While illustrative, this list of combinations is not meant to be exhaustive and it is readily apparent to persons skilled in the treatment of metabolic syndromes, that additional combinations and medicaments can be added to this list without departing from the practice of testing of biomarkers and using these results to select personalized treatments for patients with metabolic syndrome.

In one embodiment of a method for the treatment of T2D and conditions associated with diabetes mellitus, using a system Glucose Supply Side algorithm and method according to the invention, the method comprises testing of each patient for genomic markers of response to Glucose Supply Side selected pharmaceutical compositions, and then using the results of genomic testing and/or epigenetic testing and/or metabolomics testing to individualize the dosage of said compound using genomic markers of the Glucose Supply Side and of the patients individual metabolism of said composition alone or in combination with the results of the Glucose Supply Side test biomarkers

In another embodiment of a method for treatment of diabetes mellitus and conditions associated with diabetes mellitus in a human patient according to the invention and using the Glucose Supply Side and FS index algorithms incorporated by reference, the practice of said method comprises identifying said patient by inspection of medical records of care and results of tests. Glucose SD values and FS index values are calculated from serial laboratory and clinical data over timeframes. In these patient populations, a normal FS index value is about 20-50. Patients with two or more manifestations of Metabolic Syndrome that are above 200 are abnormal and are treated with the present invention.

In another aspect, the Glucose Supply Side method and associated FS index computational process uses an input/output (I/O) device coupled to a processor; a communication system coupled to the processor; and a medical computer program and system coupled to the processor, the medical system configured to process medical data of a user and generate processed medical information, wherein the medical data includes one or more of anatomical data, diabetes associated biomarkers, test specimen data, biological parameters, health information of the user, wherein the processor is configured to dynamically control operations between the communication system and the medical system.

The operations of the communication system can include one or more of a mobile device, wireless communication device, cellular telephone, Internet Protocol (IP) telephone, Wi-Fi telephone, server, personal digital assistant (PDA), and portable computer (PC). Also, the biological parameters can include one or more of current and historical biological information of the user comprising one or more of weight, height, age, temperature, body mass index, medical analyses results, body fluid analyses, blood analyses results, breath testing results, electrical activity of a body of the user, heart activity, heart rate, and blood pressure. Health information used in the processes can include one or more of current and historical health information of the user, wherein the health information includes one or more of dietary data, types of food consumed, amounts of food consumed, medications consumed, times of food consumption, physical activity exercise regimen, work schedule, activity schedule, and sleep schedule.

Additionally, the communication system can be configured to communicate one or more of the medical data and the processed medical information to a remote device located one or more of on the user, in a home, in an office, and at a medical treatment facility, the remote device including one or more of a processor-based device, mobile device, wireless device, server, personal digital assistant (PDA), cellular telephone, wearable device, and portable computer (PC). Also, the processed medical information can be used for one or more of observation, research study, real time monitoring, periodic monitoring, correlation, diagnosis, treatment, database archival, communication, command, and control.

The communication process may be configured to communicate alert information in response to the processed medical information, wherein the alert information includes one or more of a message, a visual alert, an audible alert, and a vibratory alert communicated to the user, wherein the alert information includes one or more of voice data, text, graphics data, and multimedia information. Further, the communication process may be configured to process medical data comprises correlating one or more of the medical data and processed medical information with categorical data of the user, wherein the categorical data includes one or more of data of an age category of the user, data of a body type of the user, and parametric data of the user. The processor can be configured to convert one or more of the medical data and the processed medical information from a first form to a second form.

A system of the invention useful in the implementation of the processes described above can comprise a memory device coupled to the processor, wherein the memory device is configured for storing one or more of the medical data and the processed medical information. The system can comprise a positioning device coupled to the processor, the positioning device automatically determining a location of the user and outputting information of the location, wherein the positioning device is a Global Positioning System (GPS) receiver, wherein the location includes one or more of a latitude, a longitude, an altitude, a geographical position relative to a land-based reference. The r/o device may be configured to provide communication via a network comprising a wired network and a wireless network. The system may include a port configured to receive one or more of a specimen from a body of the user and a substrate including the specimen. Further, the system may also comprise an analyzer coupled to xerogel-based substrates for concentration-dependent analyte detection, the analyzer including a xerogel-based sensor coupled to a processor configured to analyze the specimen and generate the processed medical information, wherein analysis of the specimen includes correlating parameters of the specimen with the medical data.

The specimen used in processes and systems of the invention can be a biological sample, which could include breath, saliva or any fluid or tissue from a patient, wherein the processed medical information includes one or more of a chemical analysis of the specimen.

A device of the invention comprises the components of the invention's system as described above and can comprise at least one auxiliary port for coupling to at least one other device. The device may include a medicament delivery system coupled to the processor, the delivery system including at least one reservoir that contains at least one composition, the delivery system configured to administer at least one composition for use in treating the user, wherein the composition is administered under control of the processor and the processed medical information. The delivery system is configured to automatically administer the composition or medicament. Also, the delivery system may be configured to administer the composition under manual control of the user.

Processed medical information employed in the processes, systems, and devices of the invention may include a mathematical expression for choice of medicament among a plurality of dosages, wherein the composition is administered under at least one of the plurality of dosages when personalized for the care of the patient with one or more manifestations of metabolic syndrome. The processed medical information includes information of the at least one composition, wherein the information of the at least one composition includes one or more of composition identification information, an amount released, and a time of release. The processor may configure to generate and receive control signals.

In certain embodiments of the invention, personalizing one or more metabolic syndrome treatment profiles associated with a monitored analyte concentration in a specimen includes retrieving a current analyte pharmacokinetic rate of change information, calculation of a modified analyte rate of change information based on the received analyte data associated with monitored analyte concentration, and generating one or more modifications to the medicament composition from the pharmacokinetic calculations performed thereon.

In certain embodiments of a device of the invention, the processor generates the control signals one or more of automatically and in response to an input from the user. Control signals may be configured to control one or more of devices coupled to the user, devices implanted in the user and devices coupled to the processor. Such control signals may control administration of at least one medicament composition or combinations thereof.

In a still further embodiment of the invention, the invention provides a system for providing metabolic syndrome component management, comprising: a sensor unit measuring concentrations of analytes; an interface unit; calculations by one or more processors coupled to the interface unit; a memory for storing data and instructions which, when executed by the one or more processors, causes the one or more processors to receive data associated with monitored analyte concentrations for a predetermined time period substantially in real time, retrieve one or more therapy profiles associated with the monitored analyte concentrations, and generate one or more modifications to the retrieved one or more therapy profiles based on the data associated with the monitored analyte concentrations.

In a still further embodiment of the invention, the invention provides a providing preferred embodiments of metabolic syndrome treatment, comprising: an analyte monitoring system configured to monitor analyte related levels of a patient substantially in real time; a medication delivery unit operatively for wirelessly receiving data associated with the monitored analyte level of the patient substantially in real time from the analyte monitoring system; and a data processing unit operatively coupled to the one or more of the analyte monitoring system or the medication delivery unit, the data processing unit configured to retrieve one or more therapy profiles associated with the monitored analyte related levels, and generate one or more modifications to the retrieved one or more therapy profiles based on the personalized treatment processes associated with the monitored analyte measurements.

In an embodiment of a system of the invention, the “Highest Risk” for cardiovascular injury and complications from diabetes corresponds to a composite glucose supply and insulin demand SD score generally less than about 1.0. Medicaments such as excessive insulin (SD 0.62-0.79) and secretagogues (SD 0.69-0.81) have the lowest scores and confer the highest CV risk profile and offer the lowest potential benefits. Medicaments such as alpha-glucosidase inhibitors (SD 1.25), TZD's (SD 1.27-1.35), metformin (SD 2.20) Brake™ (SD 3.5) and RYGB surgery (SD 4.0) are associated with the SD scores above 1.0 and teach the greatest potential benefits in the Glucose Supply Side computerized algorithm.

In an embodiment of a system of the invention, the Glucose Supply Side system gauge is segmented into at least one category including “Low Risk”, and “High Risk” For assessing and establishment of treatment modalities.

In an embodiment of a system of the invention, a Cardiovascular risk score is incorporated that is composed of other medicaments that affect the rate of disease progression; such risks are accelerated in a quantitative manner by some of these medicaments. Acceleration can be measured by biomarkers according to the teachings of the Supply Side System.

In another embodiment of a system of the invention, a Cardiovascular risk score is incorporated that is composed of other medicaments that affect the rate of disease progression; such risks are attenuated in a quantitative manner by some of these medicaments. Attenuation can be measured by means of biomarkers according to the teachings of the Supply Side System. A Cardiovascular risk score may be based on the FS index, in this embodiment composed of other medical events that quantify the rate of cardiovascular injury progression in metabolic syndrome using an algorithm and one or more biomarkers of cardiovascular progression in a model and system, wherein such risks are attenuated or accelerated in a quantitative manner by some of the disclosed treatments. Acceleration and attenuation can be measured by means of biomarkers and used to adjust dosages or personalize treatment to individual patients.

### Example 1

### Formulations for Pancreatic Regeneration to Improve T2D

The subject invention for treatment of T2D concerns a pharmaceutical formulation or dosage form comprising a first active drug comprising an ileal brake hormone releasing substance over-coated with an immediate or delayed release layer of a second active drug comprising, preferably, the antihyperglycemic drug metformin or a pharmaceutically acceptable salt thereof, alternatively sitagliptin or an alternative from the listing of available DPP-IV inhibitors as defined herein. The ileal brake hormone releasing substance is delivered in a controlled release manner from a tablet core, preferably an osmotic tablet core without a gelling or swelling polymer.

The composition of the tablet core should include the ileal brake hormone releasing substance and at least one pharmaceutically acceptable excipient. In one embodiment of the present invention the tablet core includes the ileal brake hormone releasing substance, a binding agent and an absorption enhancer, and the tablet core is preferably coated with a polymeric coating to form a membrane around the tablet. The tested formulations to be described herein have the following core composition:

| Core Component | Amount, mg | Allowed Range, mg |
|---|---|---|
| Alfalfa Leaf | 3.00 | 1-10 |
| Chlorella Algae | 3.00 | 1-10 |
| Chlorophyllin | 3.00 | 1-10 |
| Barley Grass Juice Concentrate | 3.00 | 1-10 |
| D-glucose (Dextrose) | 1429.00 | 500-3000 |
| Corn Starch NF | 80.00 | 25-160 |
| Stearic Acid NF | 19.50 | 6.5-35 |
| Magnesium Stearate NF | 7.00 | 2.5-15 |
| Silicon Dioxide FCC | 2.50 | 0.75-5.0 |

All inner core compositions were prepared identically for the single dose study, Briefly, the actives were mixed with corn starch, stearic acid, magnesium stearate and silicon dioxide and pressed into a tablet.

Seven different coatings were prepared and applied to the tablets. These are disclosed in the table below:

| | |
|---|---|
| Formulation 1 | 10% Shellac |
| Formulation 2 | 8% Shellac |
| Formulation 3 | 10% Eudragit S |
| Formulation 4 | 10% Nutrateric - Colorcon |
| Formulation 5 | 10% food glaze |
| Formulation 6 | 8% food glaze |
| Formulation 7 | 6% food glaze |

In this experiment, which was conducted in 45 volunteer subjects to define the specific formulation of Brake™ for use in patients as a mimetic of RYGB, each patient received a single dose of the test formulations prepared (coded 1-7 here) and the subsequent 10 hours was used to monitor blood concentrations of GLP-1, PYY, GLP-2, HOMA-IR, Proinsulin, C-peptide, Glucose, Leptin, IGF 1 and IGF-2.

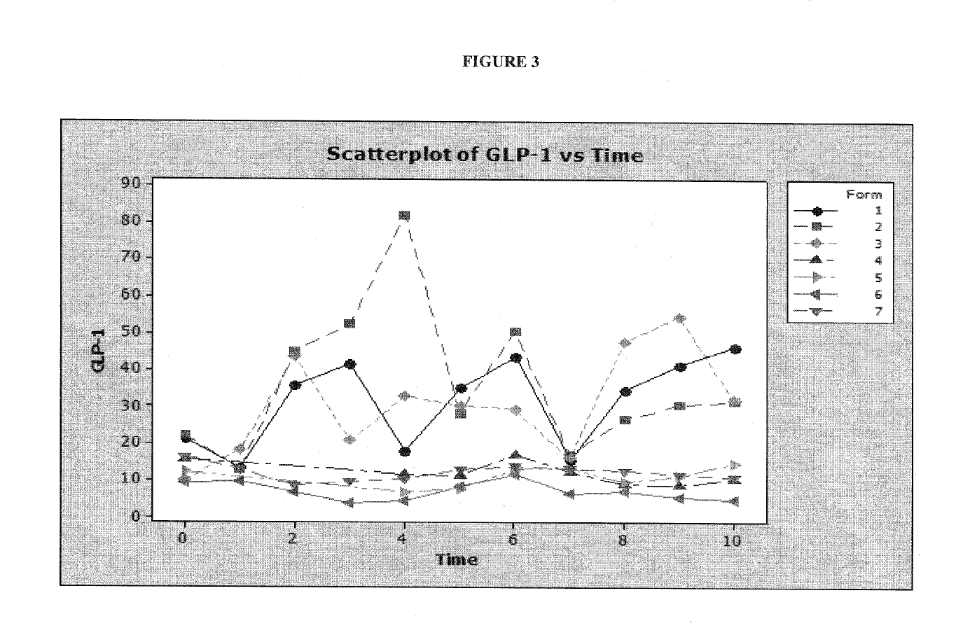

FIG. 3 presents the mean group concentration-time course of GLP-1 values for 10 hours for GLP-1 hormones released from the intestine. These hormones are released from the L-cells after groups of subjects were given each of the 7 formulations of Brake™ tablets, a total of 45 subjects were employed to generate these data, some of whom had metabolic syndrome and/or T2D. A poor GLP-1 response (AUC ˜100) was noted for 4 formulations, and 3 had a good response (AUC ˜250). Thus an efficacious product should produce an AUC above 200. It was notable that a good response was associated with a 3.5 hr GLP-1 concentration above 60, in contrast with values of patients who did not respond to the administration of Brake, where the GLP-1 concentration was usually below 20 for the entire monitoring period.

The purpose of this pharmacology study was to define the impact of 7 different coating formulations on release of GLP-1 from human subjects, each tested for optimal coating to reach the ileal brake and release PYY and GLP-1. From these data, the formulation that provided the best pattern of GLP-1 and PYY was to be chosen for the subsequent clinical use study in patients to compare the oral use of Brake™ oral with RYGB surgery patients.

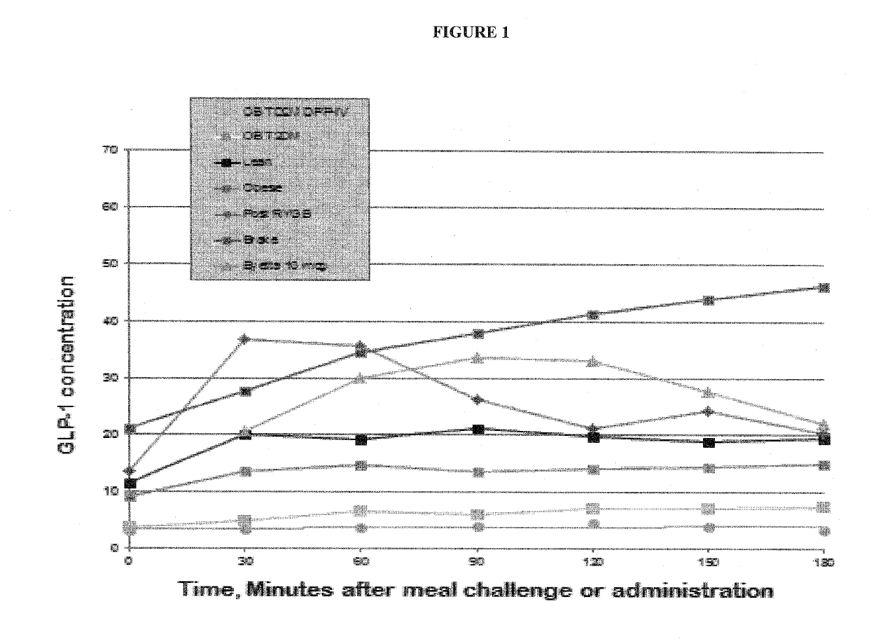

Under the stated calibration conditions, the selected formulation ideally would produce the same 0-10 hr AUC of GLP-1 as observed in a patient having RYGB surgery and illustrated as part of FIG. 1. In this manner, the purpose of the ileal brake hormone releasing formulation is to mimic the action of the RYGB surgery procedure on the distal intestinal Metasensor, including resolution of metabolic syndrome and regeneration of GI, pancreas and Liver.

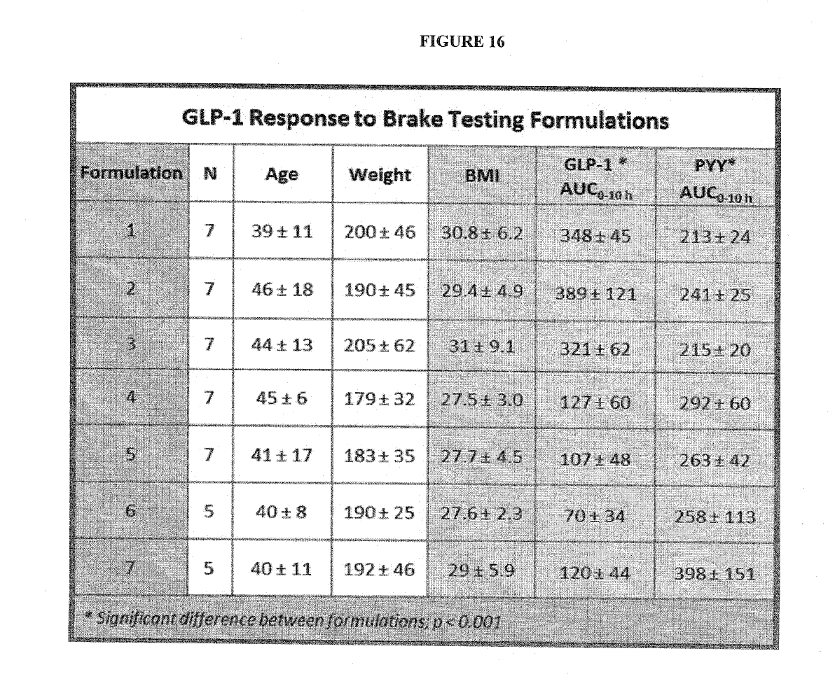

The mean group AUC values for PYY and GLP-1 are provided in FIG. 16. From these testing procedures, formulation #2 was chosen for treatment of metabolic syndrome in patients, on the overall best performance in both GLP-1 release and PYY release from the ileum of the test subjects.

After selecting formulation #2 for use in clinical studies, supplies were manufactured and clinical trials were organized and managed by the inventors. All clinical data presented herein were generated using formulation #2 from this experiment.

### First Clinical Trials of Oral Brake™ Administration

Study Design: Prospective use of Brake™ in patients, and retrospective comparison with RYGB patients
16 subjects with obesity and/or liver enzyme elevation treated with Brake™
Baseline and 6-month observations, with continued follow-up to date
Control patients with serial measurements of FS index and taking pharmaceutical compositions suitable for combination with Brake™.

### Methods:

In separate protocols, RYGB and Brake™ treated subjects were identified and followed for a period of 6-months to identify changes in excess body weight (EBW), systolic blood pressure (SBP), diastolic blood pressure (DBP), low density lipoprotein (LDL), high density lipoprotein (HDL), triglycerides (TG), insulin, fasting plasma glucose (FPG), insulin resistance (HOMA-IR), hemoglobin A₁C (HBA1c), liver function (AST, ALT) and renal function (SCr). Subjects with a baseline elevation in ≧1 metabolic biomarker and 6-month follow-up sampling data were included in the comparative analyses assessing metabolic restoration, medication discontinuation, and safety.

### 6-Month Comparative Outcomes for RYGB and Brake™

Inclusion: Baseline elevation in ≧1 biomarker with pre- and post-sampling
   (a) Excess body weight (>0 lbs)
   (b) Systolic blood pressure (>130 mmHg)
   (c) Diastolic blood pressure (>80 mmHg)
   (d) LDL cholesterol (>100 mg/dl)
   (e) HDL cholesterol (<50 mg./dl)
   (f) Triglycerides (>150 mg/dl)
   (g) Insulin (>10 uU/ml)
   (h) Fasting plasma glucose (>100 mg/dl)
   (i) Hemoglobin A₁C (>6.5%)
   (j) HOMA-IR (>2)
   (k) AST/ALT (>25 U/1)
   (l) Clinical Outcomes:
      (1) Improvement in weight and other metabolic biomarkers
      (2) % Restoration to metabolic targets
      (3) Medication requirements
Statistical Analysis: Data are presented as Mean±SD. Change from baseline was calculated and statistical analysis was performed by paired t-test.

### Results:

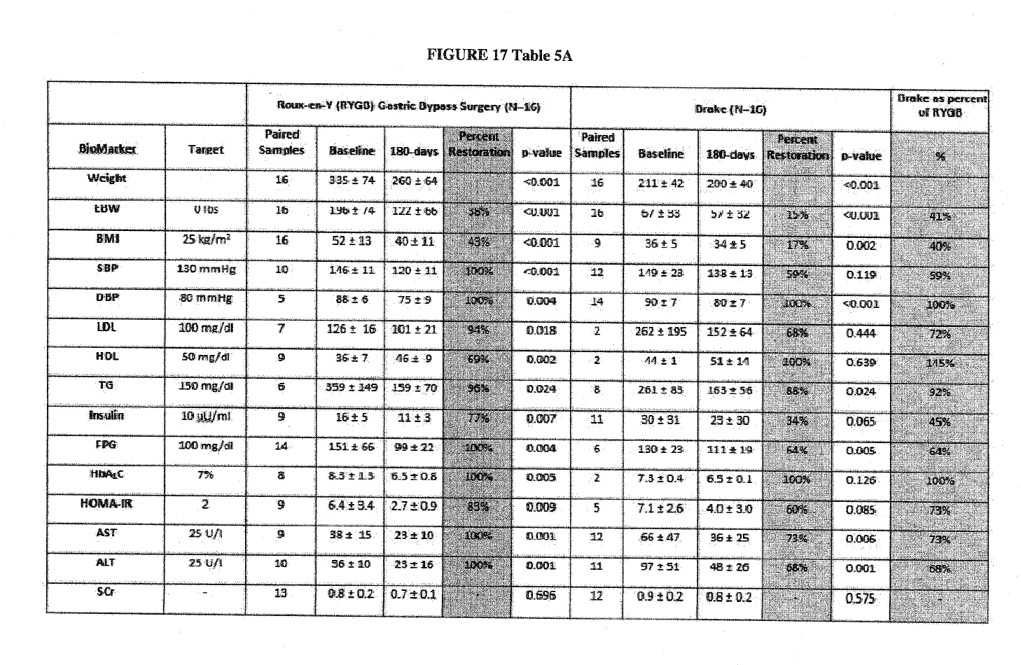

Expectedly, subjects undergoing RYGB had a profound restoration in all metabolic parameters: EBW (38%), SBP (100%), DBP (100%), LDL (94%), HDL (69%), TG (96%), insulin (77%), FPG (100%), HOMA-IR (83%), HBA1c, (100%), and liver enzymes AST (100%) and ALT (100%). Notably, these effects occur with a reduction of antihypertensive, antihyperlipidemic, and antidiabetic medication use. On the other hand, Brake™ treated patients did not experience major weight loss. Accordingly, it was unexpected that Brake™ treated subjects demonstrated effects on FS index parameters that were almost the same as the effect of RYGB: EBW (40%), SBP (59%), DBP (100%), LDL (72%), HDL (140%), TG (92%), insulin (68%), FPG (64%), HOMA-IR (46%), AST (73%), and ALT (68%). These data are found in FIG. 17.

Concomitant medications were not discontinued in Brake™ treated subjects, although few were on medications for T2D. No change in serum creatinine was detected, either in RYGB or Brake™ treated subjects.

### Conclusions:

(1) RYGB induced statistically significant reduction in excess body weight and a profound restorative effect on all evaluated metabolic biomarkers over the 180-day monitoring period of this study. Normalizing all of the FS index parameters leads to the surprising conclusion of regeneration of pancreas, liver and GI tract in these patients.
(2) Brake™ in a daily dosage of 7 pills of formulation 2, induced statistically significant reduction in excess body weight, blood pressure, hypertriglyceridemia, fasting plasma glucose, and liver enzymes over the 180-day monitoring period of this study.
(3) In comparison to RYGB, the daily dose of 7 pills, about 10 gm of the dextrose formulation of Brake™ induced unexpected similar metabolic effects on blood pressure, lipids, and liver enzymes, since body weight did not decrease to the same extent as with RYGB surgery. Comparative effect on excess body weight (41%), insulin resistance (45%), and blood glucose (64%) were a lesser percentage of RYGB. Medications were only discontinued in RYGB. Neither RYGB nor Brake™ increased serum creatinine.
   While not as profound as RYGB, Brake™ induces statistically significant weight loss and improvements in blood pressure, lipids, glucose, and insulin resistance. Liver enzymes indicative of NAFLD are improved significantly in both groups. These relative changes establish the SD ratio of RYGB as 4.0 and the SD ratio of Brake™ at 3.5.

Overall, while not as profoundly associated with weight loss as RYGB, Brake™ is clearly responsible for statistically significant improvements in hypertension, hyperlipidemia, hyperglycemia, hepatic inflammation, and insulin resistance. In each of these cases where a component of metabolic syndrome establishes elevated CV risk, Brake™ was similarly effective to RYGB surgery, meaning that these outcomes were not dependent on weight loss to be beneficial to the patient with elevated CV risk due to metabolic syndrome. Previous investigators in this field have been generally unwilling to recognize that CV risk is not due to obesity directly, even in the face of this strong evidence.

Study of ileal brake hormone derived biomarkers such as GLP-1, allow a demonstration of the ileal brake differences associated with obesity, T2D, and of course the effect of RYGB. In brief, weight gain proceeds as the ileal brake is put to sleep, it becomes hypo-responsive as the patient develops greater central adiposity and the syndrome progresses to T2D, NAFLD, hypertension and ASCVD in patients afflicted by metabolic syndrome. The pancreas response to this progressive loss of ileal brake control is a decline in output of insulin, eventually failing to keep up with the glucose supply coming in from the diet (Monte, US 2011/0097807, now U.S. Pat. No. 8,367,418). It is this process in the precise anatomical location of the ileal brake, that RYGB, and our Brake™ product wakes up and restores, which is the initial event in regeneration of organs and tissues.

We have summarized GLP-1 responses in the various conditions in FIG. 1, where we show that there is a cumulative lack of ileal brake response in patients who are gaining weight and developing T2D as their insulin secretion capacity is unable to keep up with the demands of the dietary glucose load. Furthermore, the use of a DPP-IV inhibitor does not increase the GLP-1 concentration to a significant degree, further indicating that the ileal Brake is hypo-responsive and in the obese patient, unable to provide sufficient GLP-1 output to maintain pancreatic function. RYGB surgery in one of these patients clearly restores the GLP-1 output of the ileal Brake, by specifically stimulating this area and its highly reactive L-cells with carbohydrates and lipids from the dietary ingestion.

Until some work recently performed by us using the SmartPill, there was no explanation for why the obese and obese T2D patient had low output of the ileal brake, as caused by its state of capable of normal response to stimulation (as shown by RYGB) but choosing not to respond.

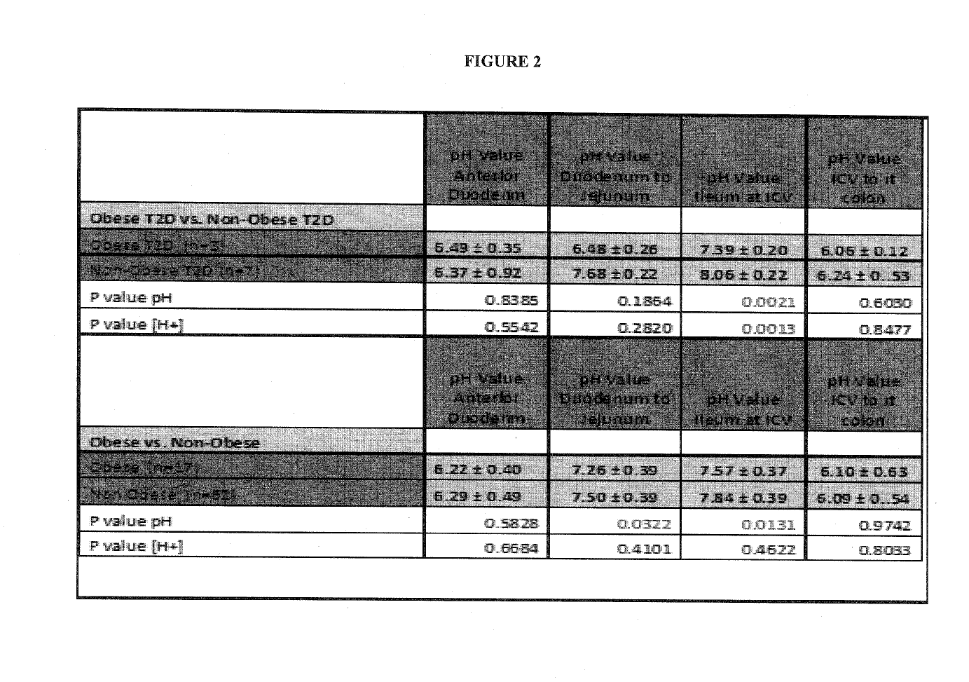

We used SmartPill (SmartPill Corporation, USA) to analyze segmental differences between the ileal brake sites in normals, obese subjects and obese patients with T2D, the differences between the pH values in the ileum of these different populations were profound and unexpected. Basically, as shown in FIG. 2, the ileal segment was more acidic than normal in the obese cases, and progressively more acidic as these patients develop T2D.

These intestinal segment changes in pH seen in different patient populations are consistent with shifts in the probiotic bacterial populations among diabetics and obese patients, as shown by recent work using stool samples as starting material (19, 20)

There were novel and important discoveries in these studies that led to key refinements during development of the Brake™ product. Specifically, we learned that the target pH for release of the formulation contents must be optimized to the values of the sleeping ileal Brake in a T2D patient, those between about 7.2 and 7.5. Had we targeted 7.7 to 8.0 which is a normal pH value in non-obese persons, the product would not release in the ileum of these patients and thus not have an effect on the precise patient population we were treating, and indeed would likely not be released at all. Secondly, we learned that the major defect of the ileal Brake in T2D patients is not atrophy of the L-cells themselves, rather the problem is a lack of signaling. The absence of signal has three novel causes. First, the dietary ingestion of refined sugars leads to a huge bolus of glucose from the duodenum, but surprisingly it is all absorbed by the duodenum and as a result, none of this sugar load reaches the ileum to trigger satiety and/or any of the other beneficial responses to activation of the ileal brake, such as repair and regeneration of pancreas, liver and GI tract. This hyperstimulation of insulin output from a diet of highly refined and immediately available sugar is a primary reason for pancreatic exhaustion in evolving metabolic syndrome and the eventual collapse of pancreatic beta cell insulin production. The absence of an ileal brake signal to regenerate beta cell mass is a consequence of the rapidly absorbed high duodenal load of sugar. This refined sugar-fast forward pathway to central adiposity and eventual T2D may be termed the glucose supply pathway to T2D, which now appears to progress unopposed by the ileal brake. The ileal brake is quiescent if there is no glucose reaching the ileum to signal the brake, and the consequences are rapid weight gain and pancreatic exhaustion.

Secondly, with regard to signaling itself, clearly the intestinal flora change and perhaps become active themselves in signaling the ileal brake to be quiet.(21-26) This is of course in their own self-interest, as a quiet ileal brake means that ingestion to excess available calories continues, increasing the chances of the increased flora for receiving more downstream nutrition, further accelerating their growth. More bacteria, lower ileal pH and thus more signal to the ileal brake to become quiet. The result is more hunger signal, more ingestion of sugar and fats, and consequently central adiposity with high insulin output, termed of course, insulin resistance. Thus we have learned for the first time how the sensor called the ileal brake is integral to the pathogenesis of metabolic syndrome and T2D, via bacterial flora and the type of diet that has been called the “western diet” high in refined sugars and fats, all optimized for rapid absorption and high insulin release.

Hyperglycemia and hyperlipidemia are almost unavoidable in this fast forward nutrition driven cycle, and the only aspect that can be recovered is the ileal brake, which has RYGB as a primary means of awakening via L-cell stimulation, and now our discovery of an oral mimetic, the product herein called Brake™.

All of these findings are also evident with Brake™ treatment, and we did make the observation in one of our patients who discontinued Brake™ treatment, that her T2D did not return for a prolonged period of time after stopping Brake™, and only after she began once again to gain weight. So it is possible to conclude that Brake™ is also producing an improvement in insulin secretory capability and that is the reason why Brake™, like RYGB, can restore previously lost pancreatic functionality, an unexpected result. This is a novel finding, completely unexpected in particular because these Brake™ treated patients lost much less weight than RYGB patients, and most workers in this field of T2D posit that weight loss is the mechanism of improvement in T2D. It is clear from our findings that pancreatic regeneration or renewal is an important and previously undiscovered attribute of precise stimulation of the L-cells of the ileal Brake.

In studies conducted by the inventors with the formulation claimed herein, diabetic patients with elevated HBA1c had nearly complete return to normal HBA1c values when treated longer than 6 months. Of most significance, patients could stop their treatment with the Brake™ formulation, yet their T2D did not return until after considerable weight re-gain and a return of the metabolic syndrome that caused it in the first place. Demonstration of a prolonged and persistent effect is further evidence of pancreatic beta cell regeneration or at least an increase in functional beta cell mass, and we claim this novel pathway as a beneficial attribute of the oral mimetic of RYGB.

A similar profile of hormones are released from the L-cells in the distal intestine regardless of whether the release is caused by RYGB or by the ileal Brake releasing substance disclosed herein as Brake™.

FIG. 3 represents an example of the pattern of GLP-1 and PYY hormones released from 7 formulations of Brake™ tablets when given to a total of 45 subjects, some of whom had metabolic syndrome and/or T2D. The purpose of this pharmacology study was to define the impact of 7 different coating formulations on release of GLP-1 from human subjects, each tested for optimal coating to reach the ileal brake and release of GLP-1. Under the stated calibration conditions, the selected formulation would have the same 0-10 hr AUC of GLP-1 as observed in a patient having RYGB surgery. In this manner, the purpose of the ileal brake hormone releasing formulation is to mimic the action of the RYGB surgery procedure on the distal intestinal Metasensor, including resolution of metabolic syndrome and regeneration of GI, pancreas and Liver.

From these testing procedures, formulation #2 was chosen for treatment of metabolic syndrome in patients, and all clinical data presented herein was generated using formulation #2 from this experiment.

### Example 2

### Pancreatic Beta Cell Regeneration with MetaBrake™

Metformin is the mainstay treatment for T2D worldwide, and all biguanides show a dose related lowering of hyperglycemia. Some studies with metformin in T2D patients have shown a reduction in cardiovascular risk profile. This may be achieved by glucose lowering or it may be result of modest weight reduction, or both. Metformin alone is not known to regenerate the pancreas or liver in patients with T2D nor does it directly impact the cardiovascular system or the vascular endothelium. When we examined our control patients treated with metformin, we confirmed that there was no significant change in any of the parameters that would indicate regeneration, even at dosages of 2.0 grams daily. Specifically, FS index rises on metformin alone, and there is a slow loss of control of their T2D in all parameters. See FIGS. 4, 5, 18 and 19 for illustrations of the rise in FS index and loss of T2D control on metformin, all of which suggest that metformin alone does not have regeneration properties in either pancreas or liver.

RYGB surgery on the other hand, has a major effect on pancreatic regeneration, a modest lowering of cholesterol, and a dramatic evidence of organ and tissue regeneration, providing sufficient amounts of new beta cell formation so that RYGB patients can be removed from insulin therapy within days of the surgical procedure. One aspect of the greater effect of RYGB surgery is its impact across the dietary supply side pathways of sugar and fat, T2D and hyperlipidemia. Evidence favoring the combination approach of an orally active RYGB mimetic with metformin is provided in FIGS. 4, 6, 17 and 19 in the present application. Subsequently, the inventors disclose their own findings demonstrating the synergistic effects of the combination product of controlled release Brake™ over-coated with 500 mg of metformin in immediate release form. Clearly there is no need to put the patient at risk of metformin side effects with the use of 2.0 gram dosages. Thus the synergy of 500 mg metformin with 10 to 20 grams of Brake creates pancreatic beta cell regeneration without risk of metformin side effects.

Metformin is an example of an optimal medicament to use in combination with Brake™. Metformin, which decreases hepatic gluconeogenesis, acts on the glucose supply side of the nutritional pathways of the ileal brake. Metformin is ideally given in combination with Brake™ in a dosage lower than metformin when given alone. In the combination product, Brake™ acts the distally in the same way as RYGB surgery. There is the same sensation of a “malabsorptive emergency” the same activation of L-cells, the output of which produces regeneration and makes hunger for sugar and fats quickly disappear. In this case the additional benefit of metformin is some additional activation of the L-cell pathway and a decrease in the amount of glucose synthesized by the liver. Otherwise the coordinates of the response model are the same as RYGB surgery or Brake™ alone.

By way of example, when apportioning the daily dose of metformin onto the daily dose of ileal brake hormone releasing substance in the enteric coated tablet form, the 1.0 gram tablets are over-coated with the immediate release metformin in a weight ratio of approximately 0.025 to 0.10 parts metformin to each 1.0 part refined sugar, optimally 0.05 parts metformin to each 1.0 part refined sugar; and/or the enteric coated core of the pharmaceutical composition may also comprise approximately 60-80% dextrose and 0-40% of a plant-derived lipid

Use of the disclosed treatments and methods for regeneration of pancreatic beta cells are based on the findings which are presented herein.

By way of illustrative example of the regenerative effect of RYGB or its oral mimetic Brake™ on T2D, consider the diagram shown as FIG. 4, which illustrates the impact of known anti-diabetic agents during the progressive T2D associated loss of beta cell mass. In contrast, the figure displays the effect of RYGB surgery and Brake™ on the same biomarkers. FIG. 4 shows the impact of different points of intervention on HBA1c and beta cell mass in patients with T2D. It also shows the HBA1c patterns of conventionally treated T2D patients, where there is a slow loss of effect of either metformin and/or sulfonylureas (Gibenclamide in this example). HBA1c rises steadily, forcing a change in therapy in most patients over 1-3 years. The conventional T2D regimens slowly lose their effects because they fail to preserve or augment pancreatic beta cell functions in the presence of unrelenting immediate release carbohydrate loading. Conventional T2D progression data are plotted from those in the UK Prospective Diabetes Study. Clearly, the application of RYGB surgery at any point in progression of T2D (arrow) causes pancreatic regeneration and lowers HBA1c to normal as a result. Thus far, oral use of Brake™, when added to metformin or when used alone as a mimetic of RYGB surgery (arrow) has also returned HBA1c to normal, indicating a similar effect on pancreatic regeneration as RYGB surgery.

One female subject, LJ-1, was initially controlled on 2.0 grams of metformin daily and 7 Brake™ tablets. She lost 32 lbs on this combination. Subsequently, she stopped losing weight but was otherwise pleased with the response to the combination. She was converted to 500 mg of metformin daily and felt even better taking the lower dose of metformin. Weight loss resumed and it was felt by the inventors that metformin dosage reduction should be part of all patients' combinations, since patients have fewer metformin side effects at the 500 mg daily dose when compared with the usual dose of 2.0 gm.

Logically, any agent which augments the processes involved in increasing pancreatic beta cell mass are logical to combine with Brake™ in order to further augment the impact on the pancreas. Metformin combinations therefore are important, particularly in view of their surprising efficacy at a lower dose of metformin (low dose metformin) than that used conventionally as monotherapy. It is also novel to combine Brake™ with lower that typical doses of DPP-IV inhibitors such as sitagliptin, particularly in view of the GLP-1 data in FIG. 1 that show no impact of a DPP-IV compound on the obese T2D patient. Brake™ would be the ideal combination product for a DPP-IV because it stimulates endogenous GLP-1 production, which would then confer synergistic benefit to sitagliptin, because this compound interrupts its clearance.

When combined into an oral dosage form of 7 Brake™ tablets overcoats with a DPP-IV inhibitor such as sitagliptin, by way of example, each tablet would contain about 1000 mg of ileal brake hormone releasing substances and 10 mg of sitagliptin. In this manner the total dose of sitagliptin per day would be less than 100 mg (low dose sitagliptin), yet the combined product would, in a completely novel way, control glucose, lower body weight, control triglycerides, lower systemic inflammation and regenerate organs and tissues in a similar manner as RYGB surgery. This combination product of Brake™ and sitagliptin, called JanuBrake™ would be given once or twice daily and be suitable for consumer use of sitagliptin with an increased safety profile over that of sitagliptin alone. Similar gains in potency at lower doses, broad array of treatment responses in metabolic syndrome, and safety advantages over the statin alone would be seen with each of the DPP-IV inhibitors reduced to practice, and the disclosure of invention of a synergistic combination encompasses all DPP-IV inhibitor combinations with Brake™ prepared in this manner for these purposes.

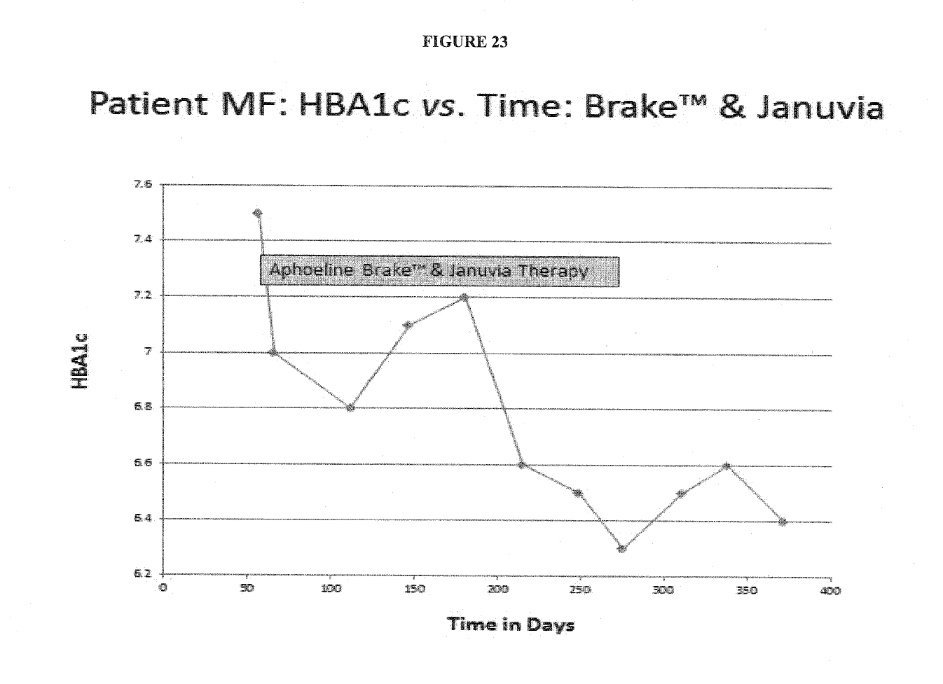

Patient MF was a 49 year old female with a history of chronic hepatitis B, her liver biopsy showed steatosis with stage 1/4 fibrosis. Both her Triglycerides and hepatic enzymes were 2-3× elevated. She had T2D on Metformin and a sulfonylurea with a baseline HBA1c of 7.4. Her diabetic control deteriorated on this regimen, to the point where she was considered a candidate for insulin. As an alternative, patient was started on Januvia (sitagliptin) 100 mg per day and 7 pills of Brake™. After 6 months treatment, her HBA1c became normal at 6.0, indicating pancreatic regeneration from the combination product. She also had nearly complete normalization of her AST, triglycerides and Alpha-fetoprotein in this same timeframe. She lost 35 lbs. Her course is shown in FIG. 23. After 6 months, she stopped the Brake™ therapy but continued sitagliptin. By 6 months later she began to gain weight and her HBA1c rose above 6.0, whereupon she resumed taking Brake™ tablets with return of her HBA1c to normal once again. This case taught the inventors that Brake™ associated organ regeneration is a long lived effect of the combination but not a permanent effect. In fact it is often notable that RYGB patients lose the effects of the surgical procedure after two or more years, and it appears that resumption of dietary indiscretion leads to recrudescence of the metabolic syndrome. Patients are thus advised to be vigilant, particularly if weight gain resumes.

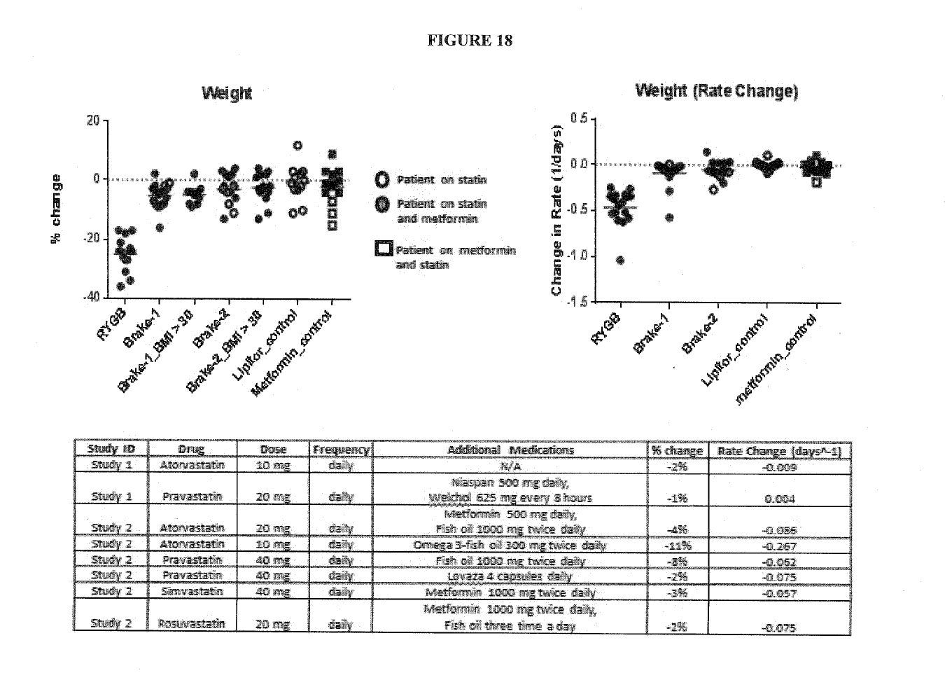

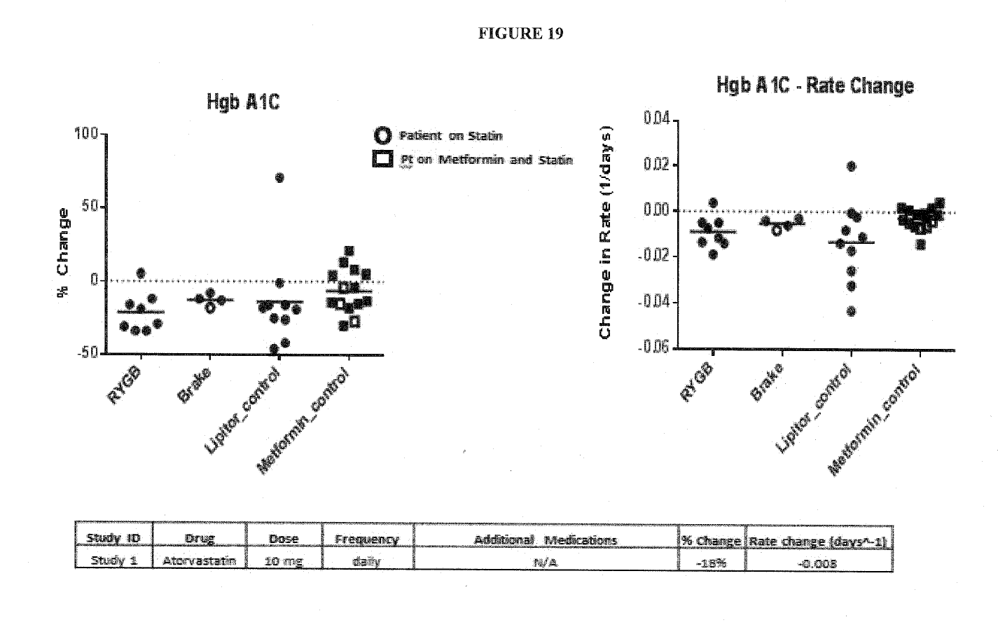

The initial disclosed combination product in this invention is low dose metformin, wherein 500 mg of immediate release metformin is over-coated onto 10 gm of the controlled release ileal brake hormone releasing substance, and the pharmaceutical composition is recommended for 3-6 months of treatment at a minimum to achieve the maximum regeneration of pancreas, liver and GI tract. Some examples of patients treated with metformin and Brake™ together, but as separate pills are presented in FIGS. 18 and 19, along with the respective controls. The figures show metformin alone at a dose of 2.0 gm per day, which has little effect, and Brake™ alone at a dose of 10 gm per day as well as the patients taking both in combination. The figures also show that RYGB patients, by way of reference lose more weight but do not have more effect on metabolic syndrome biomarkers like HBA1c when compared to metformin combined with Brake™.

In particular, the present invention generally proceeds when the steps in practice of the invention include the testing the patient for laboratory biomarker patterns, use of the results of testing to calculate the FS index, determining the risk of organ damaging events from the FS index calculation (when the FS index measures at least about 60, 100, 150, 200, 300, 400 or 500 and above), then the application of personalized treatment to lower the FS index, most preferably by the administration of a pharmaceutical composition targeted to a specific receptor (on the L-cells) in the distal intestine, in a dosage and duration of treatment to lower the FS index of the patient upon repeat measurements.

The effect of said medicament on the measured biomarkers demonstrates beneficial properties of the ileal brake hormone releasing substance on the laboratory tests that comprise the FS index. In the ordinary assessment of the precise sequence of hormonally produced events, the patient experiences cessation of hunger. The patient benefits from the ileal brake hormone release with regeneration of organs and tissues, typically pancreas, liver and gastrointestinal tract.

With respect to the sequence of signaling molecules from the ileum, a response to the medicament entails a wake up stimulation of distal intestinal L-cells that have been quieted by actions of intestinal bacteria or metabolic disease; there is a release of hormones and signals from said L-cells; said released hormones traveling in portal blood to pancreas, liver and GI tract, said organs regenerated from available growth factors and hormone signals, measured biomarkers of the FS index demonstrating the successful regeneration and said regenerated organs then signaling the patient, preferably a human, to resume adequate nutrition seeking behavior as directed by restored signals of hunger.

It is notable that the weight loss is always greater with RYGB surgery, even though surprisingly the organ and tissue regeneration profiles are quite similar between metformin given with either RYGB or with Brake™. Neither metformin alone nor atorvastatin alone as control cases demonstrate resolution of metabolic syndrome manifestations. Atorvastatin effects will be discussed further in example 5.

Other agents are suitable for combination with Brake™ tablets in the regeneration of the pancreas in T2D, and these are disclosed and incorporated into the invention by reference. Some examples follow. It is noted that these other agents are preferably formulated in combination with the ileal hormone stimulating substance (Brake™) at substantially lower doses than when these same agents are administered to a subject alone (in the absence of Brake™), resulting in reduced toxicity with superior therapeutic effect.

The investigators examined the role of flavonoid rich fraction (FRF) of Oreocnide integrifolia leaves using a mouse model of experimental regeneration. BALB/c mice were subjected to ˜70% pancreatectomy (Px) and supplemented with FRF for 7, 14, and 21 days after pancreatectomy. Px animals displayed increased blood glucose levels and decreased insulin titers which were ameliorated by FRF supplementation. FRF-treated mice demonstrated prominent newly formed islets budding off from ducts and depicting increased BrdU incorporation. Additionally, transcripts levels of Ins1/2, Reg-3alpha/gamma, Ngn-3, and Pdx-1 were up-regulated during the initial 1 week. The present study provides evidence of a nutraceutical contributing to islet neogenesis from ductal cells as the mode of beta-cell regeneration and a potential therapeutic for clinical trials in management of diabetic manifestations(27)

The antihyperglycemic function of ginsenoside Rh2 (GS-Rh2) was studied on the regeneration of beta-cells in mice that underwent 70% partial pancreatectomy (PPx). The investigators explored the mechanisms of GS-Rh2-induced beta-cell proliferation. Adult C57BL/6J mice were subjected to PPx or a sham operation. Within 14 days post-PPx, mice that underwent PPx received GS-Rh2 (1 mg/kg body weight) or saline injection. GS-Rh2-treated mice exhibited an improved glycemia and glucose tolerance, an increased serum insulin levels, and beta-cell hyperplasia. Meanwhile, increased beta-cell proliferation percentages and decreased beta-cell apoptosis percentages were also observed in GS-Rh2-treated mice. Further studies on the Akt/Foxo1/PDX-1 signaling pathway revealed that GS-Rh2 probably induced beta-cell proliferation via activation of Akt and PDX-1 and inactivation of Foxo1. Studies on the abundance and activity of cell cycle proteins suggested that GS-Rh2-induced beta-cell proliferation may ultimately be achieved through the regulation of cell cycle proteins. These findings demonstrate that GS-Rh2 administration could inhibit the tendency of apoptosis, and reverse the impaired beta-cell growth potential by modulating Akt/Foxo1/PDX-1 signaling pathway and regulating cell cycle proteins. Induction of islet beta-cell proliferation by GS-Rh2 suggests its therapeutic potential in the treatment of T2D.(28)

Betacellulin (BTC), a member of the epidermal growth factor family, is known to play an important role in regulating growth and differentiation of pancreatic beta cells. Growth-promoting actions of BTC are mediated by epidermal growth factor receptors (ErbBs), namely ErbB-1, ErbB-2, ErbB-3 and ErbB-4; however, the exact mechanism for beta cell proliferation has not been elucidated. Therefore, we investigated which ErbBs are involved and some molecular mechanisms by which BTC regulates beta cell proliferation. The expression of ErbB-1, ErbB-2, ErbB-3, and ErbB-4 mRNA was detected by RT-PCR in both a beta cell line (MIN-6 cells) and C57BL/6 mouse islets. Immunoprecipitation and western blotting analysis showed that BTC treatment of MIN-6 cells induced phosphorylation of only ErbB-1 and ErbB-2 among the four EGF receptors. BTC treatment resulted in DNA synthetic activity, cell cycle progression, and bromodeoxyuridine (BrdU)-positive staining. The proliferative effect was blocked by treatment with AG1478 or AG825, specific tyrosine kinase inhibitors of ErbB-1 and ErbB-2, respectively. BTC treatment increased mRNA and protein levels of insulin receptor substrate-2 (IRS-2), and this was blocked by the ErbB-1 and ErbB-2 inhibitors. Inhibition of IRS-2 by siRNA blocked cell cycle progression induced by BTC treatment. Streptozotocin-induced diabetic mice injected with a recombinant adenovirus expressing BTC and treated with AG1478 or AG825 showed reduced islet size, reduced numbers of BrdU-positive cells in the islets, and did not attain BTC-mediated remission of T2D. These results suggest that BTC exerts proliferative activity on beta cells through the activation of ErbB-1 and ErbB-2 receptors, which may increase IRS-2 expression, contributing to the regeneration of beta cells.(29)

Transgenic expression of gastrin and EGF receptor ligands stimulates islet neogenesis in adult mice, significantly increasing islet mass. The present study aimed to determine whether pharmacological treatment with gastrin and EGF can significantly stimulate beta-cell regeneration in chronic, severe insulin-dependent T1D. In this experiment, T1D was induced by intravenous streptozotocin, resulting in >95% beta cell destruction. Four weeks later, blood glucose levels were restored to normal range by exogenous insulin therapy and rats were treated with EGF/gastrin in combination, gastrin alone, or EGF alone given subcutaneously. After 14 days treatment blood glucose was significantly lower in the EGF/gastrin group compared to the untreated diabetic controls. Along with improved glucose tolerance, EGF/gastrin treatment significantly increased plasma C peptide and pancreatic insulin content compared to diabetic controls. Histological analysis showed that EGF/gastrin treatment significantly increased beta-cell mass as determined by point counting morphometrics. The EGF/gastrin group had a significantly greater number of BrdU labeled beta-cells/section consistent with stimulation of beta-cell replication or neogenesis. An increased number of gastrin receptor positive cells were observed in the EGF/gastrin-treated groups. In contrast to the effectiveness of the EGF/gastrin combination, neither gastrin nor EGF alone improved glucose tolerance in severely streptozotocin-diabetic rats. These studies indicate that physiologically significant improvement in glucose tolerance can be achieved through stimulating beta-cell regeneration with gastrin/EGF administered systemically as conventional pharmacological therapy. (30)

Investigations in NOD mouse models show evidence of pancreatic regeneration in response to GLP-1 agonists alone, and in fact the combination of Gastrin releasing drugs like lansoprazole and the DPP-IV inhibitor sitagliptin (which elevates GLP-1 after ileal brake stimulation) also causes pancreatic beta cell regeneration.

Combination therapy with a dipeptidyl peptidase-IV inhibitor (DPP-IV) and a proton pump inhibitor (PPI) raises endogenous levels of GLP-1 and gastrin, respectively, and restores pancreatic beta-cell mass and normoglycemia in non obese diabetic (NOD) mice with autoimmune diabetes. The aim of this study was to determine whether a DPP-IV and PPI combination could increase beta-cell mass in the adult human (31) pancreas. Pancreatic cells from adult human pancreas donors were implanted in NOD-severe combined immunodeficient (NOD-scid) mice and the mice were treated with a DPP-IV and a PPI for 16 weeks. Human grafts were examined for insulin content and insulin-stained cells. Graft beta-cell function was assessed by intravenous glucose tolerance tests (IVGTT) and by glucose control in human cell-engrafted mice treated with streptozotocin (STZ) to delete mouse pancreatic beta-cells. Plasma GLP-1 and gastrin levels were raised to two- to threefold in DPPIV and PPI-treated mice. Insulin content and insulin-stained cells in human pancreatic cell grafts were increased 9- to 13-fold in DPP-4i and PPI-treated mice and insulin-stained cells were co-localized with pancreatic exocrine duct cells. Plasma human C-peptide responses to IVGTT were significantly higher and STZ-induced hyperglycemia was more completely prevented in DPP-IV- and PPI-treated mice with grafts than in vehicle-treated mice with grafts. In conclusion, DPP-IV and PPI combination therapy raises endogenous levels of GLP-1 and gastrin and greatly expands the functional beta-cell mass in adult human pancreatic cells implanted in immunodeficient mice, largely from pancreatic duct cells. This suggests that a DPP-IV and PPI combination treatment may provide a pharmacologic therapy to correct the beta-cell deficit in T1D.(31)

### IGF-2

Insulin-like growth factor-II (IGF2) is a growth promoting peptide that increases beta cell proliferation and survival. The aim of the study was to determine the effect of IGF2 overexpression on beta cell mass in transplanted islets. Islets infected with adenovirus encoding for IGF2 (Ad-IGF2 group), for luciferase (Ad-Luc control group) or with uninfected islets (control group) were syngeneically transplanted to streptozotocin-diabetic Lewis rats. 800 islets, a minimal mass-model to restore normoglycemia, or 500 islets, a clearly insufficient mass, were transplanted. Rats transplanted with 800 Ad-IGF2 islets showed a better metabolic evolution than control groups. As expected, rats transplanted with 500 Ad-IGF2 or control islets maintained similar hyperglycemia throughout the study ensuring comparable metabolic conditions among both groups. Beta cell replication was higher in Ad-IGF2 group than in control group on days 3 (1.45% (IQR: 0.26) vs. 0.58% (IQR: 0.18), p=0.006), 10 (1.58% (IQR: 1.40) vs. 0.90% (IQR: 0.61), p=0.035) and 28 (1.35% (IQR: 0.35) vs. 0.64% (IQR: 0.28), p=0.004) after transplantation. Beta cell mass was similarly reduced on day 3 after transplantation in Ad-IGF2 and control group [0.36 mg (IQR: 0.26) vs. 0.38 mg (IQR: 0.19)], it increased on day 10, and on day 28 it was higher in Ad-IGF2 than in control group (0.63 mg (IQR: 0.38) vs. 0.42 mg (IQR: 0.31), p=0.008). Apoptosis was similarly increased in Ad-IGF2 and control islets after transplantation. No differences in insulin secretion were found between Ad-IGF2 and uninfected control islets. In summary, IGF2 overexpression in transplanted islets increased beta cell replication, induced the regeneration of the transplanted beta cell mass, and had a beneficial effect on the metabolic outcome reducing the beta cell mass needed to achieve normoglycemia.(32)

Meier et al investigated whether there was evidence of attempted beta cell regeneration in the pancreas obtained from a patient with recent-onset T1D, and if so by what mechanism this occurred. They examined pancreas tissue from a lean 89-year-old patient (BMI 18.0 kg/m(2)) with recent-onset T1D who had had a distal pancreatectomy to remove a low-grade pancreatic intraepithelial neoplasia. In the tumor-free tissue, the fractional beta cell area was 0.54+/−0.2% of pancreas area (about one-third of that in non-diabetic humans). CD3-positive T lymphocytes and macrophages had infiltrated the majority of the islets. Sub-classification of the T cell population revealed a predominance of CD8-positive cells over CD4-positive cells. Beta cell apoptosis (terminal deoxynucleotidyl transferase-mediated dUTP-biotin nick-end labeling [TUNEL] staining) was greatly increased, consistent with ongoing immune-mediated beta cell destruction. There was also a marked increase (more than approximately 100-fold) in the frequency of beta cell replication (0.69+/−0.15% Ki67-positive beta cells) in all blocks examined. The present report provides direct evidence of attempted beta cell regeneration through the mechanism of beta cell replication in a case of newly diagnosed T1D, and affirms that beta cell apoptosis is an important mechanism for beta cell loss in T1D.(33)

There is controversy regarding the roles of bone marrow (BM)-derived cells in pancreatic beta-cell regeneration. To examine these roles in vivo, mice were treated with streptozotocin (STZ), followed by bone marrow transplantation (BMT; lethal irradiation and subsequent BM cell infusion) from green fluorescence protein transgenic mice. BMT improved STZ-induced hyperglycemia, nearly normalizing glucose levels, with partially restored pancreatic islet number and size, whereas simple BM cell infusion without pre-irradiation had no effects. In post-BMT mice, most islets were located near pancreatic ducts and substantial numbers of bromodeoxyuridine-positive cells were detected in islets and ducts. Importantly, green fluorescence protein-positive, i.e. BM-derived, cells were detected around islets and were CD45 positive but not insulin positive. Then to examine whether BM-derived cell mobilization contributes to this process, we used Nos3(−/−) mice as a model of impaired BM-derived cell mobilization. In streptozotocin-treated Nos3(−/−) mice, the effects of BMT on blood glucose, islet number, bromodeoxyuridine-positive cells in islets, and CD45-positive cells around islets were much smaller than those in streptozotocin-treated Nos3(+/+) controls. A series of BMT experiments using Nos3(+/+) and Nos3(−/−) mice showed hyperglycemia-improving effects of BMT to correlate inversely with the severity of myelosuppression and delay of peripheral white blood cell recovery. Thus, mobilization of BM-derived cells is critical for BMT-induced beta-cell regeneration after injury. The present results suggest that homing of donor BM-derived cells in BM and subsequent mobilization into the injured periphery are required for BMT-induced regeneration of recipient pancreatic beta-cells.(34)

Type 1 diabetes (T1D) is an autoimmune disease in which the clinical onset most frequently presents in adolescents who are genetically predisposed. There is accumulating evidence that the endocrine pancreas has regenerative properties, that hematopoietic chimerism can abrogate destruction of beta cells in autoimmune T1D, and that, in this manner, physiologically sufficient endogenous insulin production can be restored in clinically diabetic NOD mice. Recapitulating what also has been seen sporadically in humans, these authors set out to test reliable and clinically translatable alternatives able to achieve these same goals. Recently, Tian and colleagues demonstrated that T1D can be prevented in genetically susceptible mice by substituting a “diabetes-susceptible” class II MHC beta chain with a “diabetes-resistant” allelic transgene on their hematopoietic stem cells through gene supplantation. The expression of the newly formed diabetes-resistant molecule in the re-infused hematopoietic cells was sufficient to prevent T1D onset even in the presence of the native, diabetogenic molecule. If this approach to obtain autoimmunity abrogation could facilitate a possible recovery of autologous insulin production in diabetic patients, safe induction of an autoimmunity-free status might become a new promising therapy for T1D.(35)

T1D is widely held to result from an irreversible loss of insulin-secreting beta cells. However, insulin secretion is detectable in some people with long-standing T1D, indicating either a small population of surviving beta cells or continued renewal of beta cells subject to ongoing autoimmune destruction. The aim of the present study was to evaluate these possibilities. Pancreatic sections from 42 individuals with T1D and 14 non-diabetic individuals were evaluated for the presence of beta cells, beta cell apoptosis and replication, T lymphocytes and macrophages. The presence and extent of periductal fibrosis was also quantified. Beta cells were identified in 88% of individuals with T1D. The number of beta cells was unrelated to duration of disease (range 4-67 years) or age at death (range 14-77 years), but was higher (p<0.05) in individuals with lower mean blood glucose. Beta cell apoptosis was twice as frequent in T1D as in control subjects (p<0.001), but beta cell replication was rare in both groups. The increased beta cell apoptosis in T1D was accompanied by both increased macrophages and T lymphocytes and a marked increase in periductal fibrosis (p<0.001), implying chronic inflammation over many years, consistent with an ongoing supply of beta cells. Most people with long-standing T1D have beta cells that continue to be destroyed. The mechanisms underlying increased beta cell death may involve both ongoing autoimmunity and glucose toxicity. The presence of beta cells despite ongoing apoptosis implies, by definition, that concomitant new beta cell formation must be occurring, even after long-standing T1D. These authors concluded that T1D may be reversed by targeted inhibition of beta cell destruction(36). Both RYGB and Brake™ therapy are expected to accomplish this task to at least a limited degree in T1D, and these benefits will be observed by decline in FS index values when they are treated with these modalities.

Programmed cell death (PCD) is a key phenomenon in regulating cell-numbers. Apoptosis is essentially required for a balanced homeostasis between cell proliferation and cell death in multicellular organisms. Apoptosis is especially relevant in the gastrointestinal tract, as the mammalian intestinal mucosa undergoes continual epithelia cell turnover.(37)

### Amyloid

T2D is a multifactorial disease in which pancreatic islet amyloid is a characteristic histopathological finding. Islet amyloid fibrils consist of the beta-cell protein “islet amyloid polypeptide” (IAPP)/“amylin”. Unlike human IAPP (hIAPP), mouse IAPP cannot form amyloid. In previously generated transgenic mice, high expression of hIAPP as such did not induce islet amyloid formation. To further explore the potential diabetogenic role of amyloidogenic IAPP, these authors introduced a diabetogenic trait (“ob” mutation) in hIAPP transgenic mice. METHODS: Plasma concentrations of IAPP, insulin and glucose were determined at 3.5 (t1), 6 (t2), and 16-19 months of age (t3). At t3, the mice were killed and the pancreas was analyzed immunohistochemically. RESULTS: In non-transgenic ob/ob mice, insulin resistance caused a compensatory increase in insulin production, normalizing the initial hyperglycemia. In transgenic ob/ob mice, concurrent increase in hIAPP production resulted in extensive islet amyloid formation (more often and more extensive than in transgenic non-ob/ob mice), insulin insufficiency and persistent hyperglycemia: At t3, plasma insulin levels in transgenic ob/ob mice with amyloid were fourfold lower than in non-transgenic ob/ob mice (p<0.05), and plasma glucose concentrations in transgenic ob/ob mice were almost twofold higher (p<0.05). In addition, the degree of islet amyloid formation in ob/ob mice was positively correlated to the glucose: insulin ratio (r(s)=0.53, p <0.05). Islet amyloid is a secondary diabetogenic factor which can be both a consequence of insulin resistance and a cause of insulin insufficiency(38).

It is quite clear from the extensive prior studies in animal models of diabetes, summarized earlier in this example, that a biomarker approach can be relied upon to demonstrate favorable effects of RYGB surgery and/or the compositions according to the present invention (Brake™) on pancreatic regeneration. Based on the unexpected but highly beneficial improvement in biomarkers and improved beta cell functioning after RYGB, it is a therapeutic approach to treat early diabetes with a novel combination oral therapy of a diabetes drug, nominally for the first demonstration, metformin and Brake™. In this therapeutic approach, every patient would receive Brake™ treatment that would be demonstrated to be active on the basis of lowered biomarkers of diabetes in a pattern of elevation similar to that observed in our RYGB patients. In combination with oral Brake™ treatment as disclosed herein, the patient would also receive an approved front line treatment for diabetes such as metformin, sitagliptin, or pioglitazone, any of these therapeutic substances could be given in the usual dose or in some novel regimens, given at less than half the usual dose. There are two tested reasons that Brake™ would improve both the efficacy and safety of metformin or sitagliptin in the treatment of diabetes. First, both agents have side effects which are dose related, and in both cases using a lower dosage would still improve the efficacy and yet side effects would decrease. Secondly, the control of underlying metabolic syndrome promises true reversal of the diabetes pathophysiology, which is tied to Brake™ associated reversal of insulin resistance, hyperlipidemia, hyperglycemia, hypertension and central adiposity, all of which will be improved or resolved by including Brake™ in the combination therapy of diabetes patients with metabolic syndromes.

Combination therapy between metformin or sitagliptin (or both) and Brake™ for the surprising reversal of diabetes pathophysiology is hereby incorporated by reference to data disclosed herein, with daily dosages of Metformin of 500 mg per daily dose of Brake™ of 10-20 grams daily, both active agents are presented as micro granules for oral administration to patients with diabetes. This combination has the surprising potential, when used in conjunction with biomarkers defining early risk of diabetes to prevent the onset of metabolic syndrome associated damage to the pancreas, or at least delay its onset by many years. The disclosed combination product would be the first disease modifying treatment for this disease, here-to-fore considered to be irreversible.

Clinical proof of the utility of the synergistic combination of these diabetes therapies including Brake™ necessitate the regular measurement of biomarkers of metabolic syndrome progression such as the FS index, which is an overall biomarker profile that can point to regenerative processes that respond to RYGB or Brake™. Added to the metabolic syndrome biomarker profile would be a biomarker profile of diabetes progression to CV injury. This latter progression profile would focus on cardiac injury, include epigenetics, metabolomics and genomics where applicable, and imaging where applicable to loss of cardiac structure and function. To the extent that these biomarkers are improved by metformin, those effects carry forward. To the extent that the observed improvement is tied to effects beyond those of metformin or sitagliptin, the conclusion would be Brake™ associated recovery or regeneration of pancreatic function.

### Example 3

### Obesity and Linkages to Intestinal Flora

Use of the disclosed treatments and methods of modifying human gastrointestinal flora for purposes of triggering regeneration of pancreatic beta cells, hepatic cells and regeneration of GI tract cells to benefit metabolic syndrome treatment are based on the findings incorporated by reference herein. The probiotic organisms chosen for over-coating in the formulation of the second active ingredient are Faecalibacterium prausnitzii, Bacteroides thetaiotaomicron, and Lactobacillus johnsonii. The approximate dose of these strains for release in the ileum according to the formulation is 10̂6 to 10̂8 colony forming units. It is anticipated that these specific organisms would be co-formulated with typical probiotic organisms such as lactobacilli and bifidobacteria.

Clinical proof of the utility of the synergistic combination of these diabetes therapies including Brake™ and probiotic replacement organisms would be provided by continued monitoring of biomarkers of metabolic syndrome progression, and the FS index would readily demonstrate the added benefit of combinations on regeneration. This effect is a newly discovered impact on the overall biomarker profile that can point to regenerative processes that respond to RYGB or Brake™. Added to the metabolic syndrome biomarker profile of the FS index would be a biomarker profile of T2D progression to CV injury. This latter progression profile would focus on cardiac injury, include epigenetics, metabolomics and genomics where applicable, and imaging where applicable to loss of cardiac structure and function. To the extent that these biomarkers are improved by metformin, those effects carry forward. To the extent that the observed improvement is tied to effects beyond those of metformin or sitagliptin, the conclusion would be Brake™ or RYGB associated recovery or regeneration of pancreatic function, and a greater decline in the previously elevated FS index of said patient.

Grunfeld and colleagues connected intestinal flora, lipid absorption into chylomicrons endotoxin uptake and weight gain and insulin resistance in an editorial entitled Endotoxin in the gut and chylomicrons: translocation or transportation. This is a good review of current evidence for the premise of changing the intestinal flora in order to create a demand for a response by the immune system. Suppression of the immune response to absorbed fats and endotoxin leads to arteriosclerotic cardiovascular disease or ASCVD(39)

Recent data suggest that dietary fat promotes intestinal absorption of lipopolysaccharides (LPS) from the gut micro flora, which might contribute to various inflammatory disorders. The mechanism of fat-induced LPS absorption is unclear, however. Intestinal-epithelial cells can internalize LPS from the apical surface and transport LPS to the Golgi. The Golgi complex also contains newly formed chylomicrons, the lipoproteins that transport dietary long-chain fat through mesenteric lymph and blood. Because LPS has affinity for chylomicrons, these investigators hypothesized that chylomicron formation promotes LPS absorption. In agreement with their hypothesis, they found that CaCo-2 cells released more cell-associated LPS after incubation with oleic-acid (OA), a long-chain fatty acid that induces chylomicron formation, than with butyric acid (BA), a short-chain fatty acid that does not induce chylomicron formation. Moreover, the effect of OA was blocked by the inhibitor of chylomicron formation, Pluronic L-81. They also observed that intragastric triolein (TO) gavage was followed by increased plasma LPS, whereas gavage with tributyrin (TB), or TO plus Pluronic L-81, was not. Most intestinally absorbed LPS was present on chylomicron remnants (CM-R) in the blood. Chylomicron formation also promoted transport of LPS through mesenteric lymph nodes (MLN) and the production of TNFalpha mRNA in the MLN. Together, these data suggest that intestinal epithelial cells may release LPS on chylomicrons from cell-associated pools. Chylomicron-associated LPS may contribute to postprandial inflammatory responses or chronic diet-induced inflammation in chylomicron target tissues.(40)

Erridge and colleagues examined bacterial endotoxin, which is a potently inflammatory antigen that is abundant in the human gut(41). Endotoxin circulates at low concentrations in the blood of all healthy individuals, although elevated concentrations are associated with an increased risk of atherosclerosis. Erridge sought to determine whether a high-fat meal or smoking increases plasma endotoxin concentrations and whether such concentrations are of physiologic relevance. Plasma endotoxin and endotoxin neutralization capacity were measured for 4 h in 12 healthy men after no meal, 3 cigarettes, a high-fat meal, or a high-fat meal with 3 cigarettes by using the limulus assay. Baseline endotoxin concentrations were 8.2 pg/mL (interquartile range: 3.4-13.5 pg/mL) but increased significantly (P<0.05) by approximately 50% after a high-fat meal or after a high-fat meal with cigarettes but not after no meal or cigarettes alone. These results were validated by the observations that a high-fat meal with or without cigarettes, but not no meal or smoking, also significantly (P<0.05) reduced plasma endotoxin neutralization capacity, which is an indirect measure of endotoxin exposure. Human monocytes, but not aortic endothelial cells, were responsive to transient (30 s) or low-dose (10 pg/mL) exposure to endotoxin. However, plasma from whole blood treated with as little as 10 pg endotoxin/mL increased the endothelial cell expression of E-selectin, at least partly via tumor necrosis factor-alpha-induced cellular activation. Low-grade endotoxemia may contribute to the postprandial inflammatory state and could represent a novel potential contributor to endothelial activation and the development of atherosclerosis.(41)

T2D is associated with chronic low-grade inflammation, and adipose tissue (AT) may represent an important site of inflammation. 3T3-L1 studies have demonstrated that lipopolysaccharide (LPS) activates toll-like receptors (TLRs) to cause inflammation. For this study, we 1) examined activation of TLRs and adipocytokines by LPS in human abdominal subcutaneous (AbdSc) adipocytes, 2) examined blockade of NF-kB in human AbdSc adipocytes, 3) examined the innate immune pathway in AbdSc AT from lean, obese, and T2D subjects, and 4) examined the association of circulating LPS in T2D subjects. The findings showed that LPS increased TLR-2 protein expression twofold (P<0.05). Treatment of AbdSc adipocytes with LPS caused a significant increase in TNF-alpha and IL-6 secretion (IL-6, Control: 2.7+/−0.5 vs. LPS: 4.8+/−0.3 ng/ml; P<0.001; TNF-alpha, Control: 1.0+/−0.83 vs. LPS: 32.8+/−6.23 pg/ml; P<0.001). NF-kB inhibitor reduced IL-6 in AbdSc adipocytes (Control: 2.7+/−0.5 vs. NF-kB inhibitor: 2.1+/−0.4 ng/ml; P<0.001). AbdSc AT protein expression for TLR-2, MyD88, TRAF6, and NF-kB was increased in T2D patients (P<0.05), and TLR-2, TRAF-6, and NF-kB were increased in LPS-treated adipocytes (P<0.05). Circulating LPS was 76% higher in T2D subjects compared with matched controls. LPS correlated with insulin in controls (r=0.678, P<0.0001). Rosiglitazone (RSG) significantly reduced both fasting serum insulin levels (reduced by 51%, P=0.0395) and serum LPS (reduced by 35%, P=0.0139) in a subgroup of previously untreated T2D patients. In summary, these results suggest that T2D is associated with increased endotoxemia, with AT able to initiate an innate immune response. Thus, increased adiposity may increase pro-inflammatory cytokines and therefore contribute to the pathogenic risk of T2D.(42)

RYGB results in profound weight loss and resolution of T2D. The mechanism of this remarkable transition remains poorly defined. It has been proposed that endotoxin (lipopolysaccharide [LPS]) sets inflammatory tone, triggers weight gain, and initiates T2D. Because RYGB may diminish LPS from endogenous and exogenous sources, we hypothesized that LPS and the associated cascade of oxidative and inflammatory stress would diminish after RYGB. Fifteen adults with morbid obesity and T2D undergoing RYGB were studied. After an overnight fast, a baseline blood sample was collected the morning of surgery and at 180 days to assess changes in glycemia, insulin resistance, LPS, mononuclear cell nuclear factor (NF)-kB binding and mRNA expression of CD14, TLR-2, TLR-4, and markers of inflammatory stress. At 180 days after RYGB, subjects had a significant decrease in body mass index (52.1+/−13.0 to 40.4+/−11.1), plasma glucose (148+/−8 to 101+1-4 mg/dL), insulin (18.5+/−2.2 mmuU/mL to 8.6+/−1.0 mmuU/mL) and HOMA-IR (7.1+/−1.1 to 2.1+/−0.3). Plasma LPS significantly reduced by 20+/−5% (0.567+/−0.033 U/mL to 0.443+/−0.022E U/mL). NF-kB DNA binding decreased significantly by 21+/−8%, whereas TLR-4, TLR-2, and CD-14 expression decreased significantly by 25+/−9%, 42+/−8%, and 27+/−10%, respectively. Inflammatory mediators CRP, MMP-9, and MCP-1 decreased significantly by 47+/−7% (10.7+/−1.6 mg/L to 5.8+/−1.0 mg/L), 15+/−6% (492+/−42 ng/mL to 356+/−26 ng/mL) and 11+/−4% (522+/−35 ng/mL to 466+/−35 ng/mL), respectively. CONCLUSION: LPS, NF-kB DNA binding, TLR-4, TLR-2, and CD14 expression, CRP, MMP-9, and MCP-1 decreased significantly after RYGB. The mechanism underlying resolution of insulin resistance and T2D after RYGB may be attributable, at least in part, to the reduction of endotoxemia and associated pro-inflammatory mediators.(43)

Non-alcoholic fatty liver disease (NAFLD) is the hepatic manifestation of metabolic syndrome and the leading cause of chronic liver disease in the Western world. Twenty percent of NAFLD individuals develop chronic hepatic inflammation (non-alcoholic steatohepatitis, NASH) associated with cirrhosis, portal hypertension and hepatocellular carcinoma, yet the causes of progression from NAFLD to NASH remain obscure. Here, the investigators show that the NLRP6 and NLRP3 inflammasomes and the effector protein IL-18 negatively regulate NAFLD/NASH progression, as well as multiple aspects of metabolic syndrome via modulation of the gut microbiota. Different mouse models reveal that inflammasome-deficiency-associated changes in the configuration of the gut microbiota are associated with exacerbated hepatic steatosis and inflammation through influx of TLR4 and TLR9 agonists into the portal circulation, leading to enhanced hepatic tumor-necrosis factor (TNF)-alpha expression that drives NASH progression. Furthermore, co-housing of inflammasome-deficient mice with wild-type mice results in exacerbation of hepatic steatosis and obesity. Thus, altered interactions between the gut microbiota and the host, produced by defective NLRP3 and NLRP6 inflammasome sensing, may govern the rate of progression of multiple metabolic syndrome-associated abnormalities, highlighting the central role of the microbiota in the pathogenesis of heretofore seemingly unrelated systemic auto-inflammatory and metabolic disorders.(44)

As noted by Strowig, inflammasomes are a group of protein complexes built around several proteins, including NLRP3, NLRC4, AIM2 and NLRP6. Recognition of a diverse range of microbial, stress and damage signals by inflammasomes results in direct activation of caspase-1, which subsequently induces secretion of potent pro-inflammatory cytokines and a form of cell death called pyroptosis. Inflammasome-mediated processes are important during microbial infections and also in regulating both metabolic processes and mucosal immune responses. In the instant discussion, Strowig and colleagues review the functions of the different inflammasome complexes and discuss how aberrations in them are implicated in the pathogenesis of human diseases.(45)

Nearly a decade ago, the concept of inflammasomes was introduced. Since then, the biochemical characterization of the inflammasomes has led to a richer understanding of innate immune responses in the context of infection and sterile inflammation. This has provided the rationale for successful clinical therapies for a spectrum of hereditary periodic fever syndromes and potentially for some metabolic pathologies.(46)

Central adiposity is associated with metabolic alterations related to glucose homeostasis and cardiovascular risk factors. These metabolic alterations are associated with low-grade inflammation that contributes to the onset of these diseases. The authors provide evidence that gut microbiota participate in whole-body metabolism by affecting energy balance, glucose metabolism, and low-grade inflammation associated with central adiposity and related metabolic disorders. Recently, gut microbiota-derived lipopolysaccharide (LPS) (and metabolic endotoxemia) has been defined as a factor involved in the onset and progression of inflammation and metabolic diseases. In the review, the authors discuss mechanisms involved in the development of metabolic endotoxemia such as the gut permeability. The investigators also discuss these latest discoveries demonstrate a link between the gut microbiota, endocannabinoid system tone, leptin resistance, gut peptides (glucagon-like peptide-1 and -2), and metabolic features. The authors also introduce the role of the gut microbiota in specific dietary treatments (prebiotics and probiotics) and surgical interventions (gastric bypass)(23)

The bridge between food intake and weight is not fully understood. Recently, the role of gut microbiota and bacterial lipopolysacharides (LPS) in weight has been postulated. The objective for the study by Amar was to evaluate the relation between plasma LPS concentration and food intake. A dietary survey was conducted in 1015 subjects randomly recruited in France. The participants were given oral and written instructions on how to keep a consecutive 3 day food record. Plasma LPS was measured in a subsample of 201 men. In humans, no significant relation was observed between cardiovascular disease risk factors, carbohydrate and protein intakes, and plasma LPS concentration. Conversely, positive correlations were observed with fat and energy intakes. In a multivariate analysis, endotoxemia was independently associated with energy intake. In this large sample of healthy men from a population-based sample, Amar and colleagues found a link between food intake and plasma LPS. Experimental data suggest that fat was more efficient in transporting bacterial LPS from the gut lumen into the bloodstream. The results of this study add to the knowledge of mechanisms responsible for relations between food intake and metabolic diseases(47).

T2D and NAFLD are two metabolic diseases characterized by insulin resistance and a low-grade inflammation. Seeking an inflammatory factor causative of the onset of insulin resistance, hepatic steatosis, and T2D, we have identified bacterial lipopolysaccharide (LPS) as a triggering factor. The investigators found that normal endotoxemia increased or decreased during the fed or fasted state, respectively, on a nutritional basis and that a 4-week high-fat diet chronically increased plasma LPS concentration two to three times, a threshold that we have defined as metabolic endotoxemia. Importantly, a high-fat diet increased the proportion of an LPS-containing microbiota in the gut. When metabolic endotoxemia was induced for 4 weeks in mice through continuous subcutaneous infusion of LPS, fasted glycemia and insulinemia and whole-body, liver, and adipose tissue weight gain were increased to a similar extent as in high-fat-fed mice. In addition, adipose tissue F4/80-positive cells and markers of inflammation, and liver triglyceride content, were increased. Furthermore, liver, but not whole-body, insulin resistance was detected in LPS-infused mice. CD14 mutant mice resisted most of the LPS and high-fat diet-induced features of metabolic diseases. This new finding demonstrates that metabolic endotoxemia dysregulates the inflammatory tone and triggers body weight gain and T2D. The authors concluded that the LPS/CD14 system sets the tone of insulin sensitivity and the onset of T2D and NAFLD, and that lowering plasma LPS concentration could be a potent strategy for the control of metabolic diseases.(25)

T2D and obesity are characterized by a low-grade inflammation whose molecular origin is unknown. Cani and colleagues previously determined, first, that metabolic endotoxemia controls the inflammatory tone, body weight gain, and T2D, and second, that high-fat feeding modulates gut microbiota and the plasma concentration of lipopolysaccharide (LPS), i.e., metabolic endotoxemia. Therefore, it remained to demonstrate whether changes in gut microbiota control the occurrence of metabolic diseases. These researchers changed gut microbiota by means of antibiotic treatment to demonstrate, first, that changes in gut microbiota could be responsible for the control of metabolic endotoxemia, the low-grade inflammation, and T2D and, second, to provide some mechanisms responsible for such effect. They found that changes of gut microbiota induced by an antibiotic treatment reduced metabolic endotoxemia and the cecal content of LPS in both high-fat-fed and ob/ob mice. This effect was correlated with reduced glucose intolerance, body weight gain, fat mass development, lower inflammation, oxidative stress, and macrophage infiltration marker mRNA expression in visceral adipose tissue. Importantly, high-fat feeding strongly increased intestinal permeability and reduced the expression of genes coding for proteins of the tight junctions. Furthermore, the absence of CD14 in ob/ob CD14(−)(/)(−) mutant mice mimicked the metabolic and inflammatory effects of antibiotics. This new finding demonstrates that changes in gut microbiota controls metabolic endotoxemia, inflammation, and associated disorders by a mechanism that could increase intestinal permeability. It would thus be useful to develop strategies for changing gut microbiota to control, intestinal permeability, metabolic endotoxemia, and associated disorders.(21)

Central adiposity is now classically characterized by a cluster of several metabolic disorders, and by a low grade inflammation. The evidence that the gut microbiota composition can be different between healthy and or obese and type 2 diabetic patients has led to the study of this environmental factor as a key link between the pathophysiology of metabolic diseases and the gut microbiota. Several mechanisms are proposed linking events occurring in the colon and the regulation of energy metabolism, such as i.e. the energy harvest from the diet, the synthesis of gut peptides involved in energy homeostasis (GLP-1, PYY . . . ), and the regulation of fat storage. Moreover, the development of central adiposity and metabolic disorders following a high-fat diet may be associated to the innate immune system. Indeed, high-sugar, high-fat dietary feeding triggers the development of obesity, inflammation, insulin resistance, T2D and atherosclerosis by mechanisms dependent of the LPS and/or the fatty acids activation of the CD14/TLR4 receptor complex. Importantly, fat feeding is also associated with the development of metabolic endotoxemia in human subjects and participates in the low-grade inflammation, a mechanism associated with the development of atherogenic markers. Finally, data obtained in experimental models and human subjects are in favor of the fact that changing the gut microbiota (with prebiotics and/or probiotics) may participate in the control of the development of metabolic diseases. The investigators opined that it would be useful to find specific strategies for modifying gut microbiota to impact metabolic diseases.(22)

Nowadays, the literature provides evidence that central adiposity, T2D and insulin resistance are characterized by a low grade inflammation. Among the environmental factors involved in such diseases, the gut microbiota has been proposed as a key player. This neglected “organ” has been found to be different between healthy and or obese and type 2 diabetic patients. For example, recent data have proposed that dysbiosis of gut microbiota (at phyla, genus, or species level) affects host metabolism and energy storage. Among the mechanisms, metabolic endotoxemia (higher plasma LPS levels), gut permeability and the modulation of gut peptides (GLP-1 and GLP-2) have been proposed as putative targets. The authors postulate how gut microbiota can be involved in the development or in the control of central adiposity and associated low-grade inflammation.(26)

Obese and diabetic mice display enhanced intestinal permeability and metabolic endotoxemia that participate in the occurrence of metabolic disorders. Recent data support the idea that a selective increase of Bifidobacterium spp. reduces the impact of high-fat diet-induced metabolic endotoxemia and inflammatory disorders. Here, we hypothesized that prebiotic modulation of gut microbiota lowers intestinal permeability, by a mechanism involving glucagon-like peptide-2 (GLP-2) thereby improving inflammation and metabolic disorders during NAFLD and T2D. In the first study, ob/ob mice (Ob-CT) were treated with either prebiotic (Ob-Pre) or non-prebiotic carbohydrates as control (Ob-Cell). In order to assess the impact of GLP-2, Ob-CT and Ob-Pre mice were treated with GLP-2 antagonist or saline. Changes in the gut microbiota, intestinal permeability, gut peptides, intestinal epithelial tight-junction proteins ZO-1 and occludin (qPCR and immunohistochemistry), hepatic and systemic inflammation were all measured. Prebiotic-treated mice exhibited a lower plasma lipopolysaccharide (LPS) and cytokines, and a decreased hepatic expression of inflammatory and oxidative stress markers. This decreased inflammatory tone was associated with a lower intestinal permeability and improved tight-junction integrity compared to controls. Prebiotic increased the endogenous intestinotrophic proglucagon-derived peptide (GLP-2) production whereas the GLP-2 antagonist abolished most of the prebiotic effects. Finally, pharmacological GLP-2 treatment decreased gut permeability, systemic and hepatic inflammatory phenotype associated with metabolic syndrome to a similar extent as that observed following prebiotic-induced changes in gut microbiota. The authors found that a selective gut microbiota change controls and increases endogenous GLP-2 production, and consequently improves gut barrier functions by a GLP-2-dependent mechanism, contributing to the improvement of gut barrier functions during obesity and T2D.(24). This paper provides some background evidence why the ileal brake remodeling of the GI tract uses the GLP-2 pathway, and why the instant invention of Brake™ also increases the production of GLP-2 when used as directed.

Growing evidence supports the role of gut microbiota in the development of hepatic steatosis, T2D, and low-grade inflammation. The endocrine activity of adipose tissue has been found to contribute to the regulation of glucose homeostasis and low-grade inflammation. Among the key hormones produced by this tissue, apelin has been shown to regulate glucose homeostasis. Recently, it has been proposed that gut microbiota participate in adipose tissue metabolism via the endocannabinoid system (eCB) and gut microbiota-derived compounds, namely lipopolysaccharide (LPS). The authors have investigated gut microbiota composition in obese and diabetic leptin-resistant mice (db/db) by combining pyrosequencing and phylogenetic microarray analysis of 16S ribosomal RNA gene sequences. They observed a significant higher abundance of Firmicutes, Proteobacteria, and Fibrobacteres phyla in db/db mice compared to lean mice. The abundance of 10 genera was significantly affected by the genotype. They identified the roles of the eCB and LPS in the regulation of apelinergic system tone (apelin and APJ mRNA expression) in genetic obese and diabetic mice. By using in vivo and in vitro models, it was demonstrated that both the eCB and low-grade inflammation differentially regulate apelin and APJ mRNA expression in adipose tissue. Finally, deep-gut microbiota profiling revealed that the gut microbial community of type 2 diabetic mice is significantly different from that of their lean counterparts. This indicates specific relationships between the gut microbiota and the regulation of the apelinergic system. However, the exact roles of specific bacteria in shaping the phenotype of db/db mice remain to be determined.(48). The scientific linkage needed to complete these experiments was the ileal brake pathway, either by RYGB experiments or the use of Brake™ treatment.

Central adiposity is associated with accumulation of macrophages in white adipose tissue (WAT), which contribute to the development of insulin resistance. Germ-free (GF) mice have reduced adiposity and are protected against diet-induced central adiposity, To investigate whether the gut microbiota and, specifically, gut-derived lipopolysaccharide (LPS) promote WAT inflammation and contribute to impaired glucose metabolism. Macrophage composition and expression of pro-inflammatory and anti-inflammatory markers were compared in WAT of GF, conventionally raised and Escherichia coli-monocolonized mice. Additionally, glucose and insulin tolerance in these mice was determined. The presence of a gut microbiota resulted in impaired glucose metabolism and increased macrophage accumulation and polarization towards the pro-inflammatory M1 phenotype in WAT. Monocolonization of GF mice for 4 weeks with E. coli W3110 or the isogenic strain MLK1067 (which expresses LPS with reduced immunogenicity) resulted in impaired glucose and insulin tolerance and promoted M1 polarization of CD11 b cells in WAT. However, colonization with E. coli W3110 but not MLK1067 promoted macrophage accumulation and up regulation of pro-inflammatory and anti-inflammatory gene expression as well as JNK phosphorylation. Conclusion Gut microbiota induced LPS-dependent macrophage accumulation in WAT, whereas impairment of systemic glucose metabolism was not dependent on LPS. These results indicate that macrophage accumulation in WAT does not always correlate with impaired glucose metabolism. (49)

It seems clear from the extensive studies of interactions between the human microbiome in the gut and the inflammation that follows dysbiotic changes in these flora, that a biomarker approach like the FS index can be relied upon to demonstrate favorable effects of RYGB surgery and/or Brake™ on L-cell signaling, provided that both the bacteria and the L-cells themselves are closely studied in health and disease. Based on the unexpected but highly beneficial improvement in biomarkers and improved beta cell functioning after RYGB, it is an aspect of the invention to treat early diabetes with a novel combination oral therapy of a diabetes drug, nominally for the first demonstration, metformin and Brake™, and add to this a strategy to replace abnormal probiotic species with beneficial ones. Every patient would receive a Brake™ treatment combination regimen that would be demonstrated to be active on the basis of lowered biomarkers of diabetes in a pattern of elevation similar to that observed in our RYGB patients. In combination with oral Brake™ treatment as disclosed herein, the patient would also receive an approved front line treatment for T2D such as metformin, sitagliptin, or pioglitazone, any which could be given in the usual dose or, in many instances, at substantially lower dose than the typical dose given to patients. In fact, in some novel regimens, any of these could be given at half the usual dose or even less. The GI flora alterations and derangements that occur would be treated with replacement strains of the normal GI tract formulation. There are two tested reasons why Brake™ would improve both the efficacy and safety of metformin or sitagliptin in the treatment of T2D, and why pancreatic beta cell recovery would be expected in this patient. First, both agents have side effects which are dose related, and in both cases using a lower dosage would still improve the efficacy and yet side effects would decrease. Secondly, the control of underlying metabolic syndrome promises true reversal of the diabetes pathophysiology, which is tied to revised flora in the GI tract as well as Brake™ associated reversal of insulin resistance, hyperlipidemia, hyperglycemia, hypertension and hepatic steatosis, all of which will be improved or resolved by including Brake™ in the combination therapy of T2D patients with metabolic syndromes.

Combination therapy between metformin or sitagliptin (or both) and Brake™ for the surprising reversal of T2D pathophysiology is hereby incorporated by reference, with dosages of Metformin of, for example, 250-500 mg per doses of Brake™ of 10-20 grams daily or less, both active agents are presented as micro granules for oral administration to patients with T2D. This combination has the surprising potential, when used in conjunction with biomarkers defining early risk of diabetes to prevent the onset of metabolic syndrome associated damage to the pancreas, or at least delay or inhibit its onset by many years. The disclosed combination product would be the first disease modifying treatment for this disease, here-to-fore considered to be irreversible.

### Example 4

### FS Index as a Measure of Regeneration in Response to Brake™ Treatment of Metabolic Syndrome

Use of the disclosed treatments and methods of modifying outcomes of metabolic syndromes by use of FS index are largely the work of the inventors,

Previously we disclosed the Supply/Demand index of cardiovascular risk for T2D treatments (see Monte US patent publication 2011/0097807, issued U.S. Pat. No. 8,367,418 and publications (2, 3)), said application being incorporated by reference herein, wherein we presented a T2D disease progression model that characterizes the effect of conventional antidiabetic therapies on the glucose supply and insulin demand dynamic that defines metabolic syndrome associated T2D, and links this SD index to cardiovascular risk specific to the treatment of T2D patients.

We have recently extended this concept from the T2D-centric HBA1c-SD parameter (2, 3) to create a global index of metabolic syndrome, termed here the FS (Fayad-Schentag) index, a quantitative means of describing progression of Metabolic Syndrome in Patients. The FS index is meant to track the beneficial changes in metabolic syndrome as it is managed by RYGB or by Brake™, in turn a link to measurement of regeneration in the systems affected by the underlying common metabolic syndrome of these patients.

As underlying Metabolic Syndrome has many different manifestations in addition to those considered reflective of T2D, the FS index included hyperlipidemia, weight as BMI, triglycerides, liver enzymes specifically AST, hepatic steatosis and resulting NAFLD, in order to facilitate tracking progression of Metabolic Syndrome in patient populations that may have any or all of these conditions to varying degree. We now use tests for each component of Metabolic Syndrome. As a brief example why FS index is meaningful, it is known that antidiabetic drugs lower glucose but raise lipids or BP, and thus the net effect is to worsen the Metabolic Syndrome and increase CV risk. It was our hypothesis that improved risk scoring could be accomplished via an index that considered a composite of Metabolic Syndrome system components.

The FS index of Metabolic Syndrome is displayed in FIG. 15. The FS index was applied to well-studied patient populations already in our databases, using a neural net model. The database included previously published 45 patients with T2D having AMIs, 45 precisely matched T2D controls without AMIs, 41 patients with RYGB surgery and reversal of MS, 300 patients with COPD and T2D, and 18 patients given Brake™ therapy for Hepatitis C, NAFLD, or prediabetes. FS index values were calculated from serial laboratory and clinical data over timeframes ranging 2-10 years. In these patient populations, a normal FS index value is 20-50. Patients with two or more manifestations of Metabolic Syndrome are above 200 and the highest values are above 500, values seen only when nearly every Metabolic Syndrome component is abnormal, as might be observed in an extremely overweight T2D patient prior to RYGB surgery.

In particular, the present invention generally proceeds when the steps in practice of the invention include testing the patient for laboratory biomarker patterns, use of the results of testing to calculate the FS index, determining the risk of organ damaging events from the FS index calculation (when the FS index measures at least about 60, 100, 150, 200, 300, 400 or 500 and above), the application of personalized treatment to lower the FS index, most preferably by the administration of a pharmaceutical composition targeted to a specific receptor (on the L-cells) in the distal intestine, in a dose and duration of treatment to lower the FS index of the patient upon repeat measurements. Ideally, the present invention can reduce a patient's FS index to the normal range (20-50).

The effect of the medicament on the measured biomarkers demonstrates beneficial properties of the ileal brake hormone releasing substance on the laboratory tests that comprise the FS index. In the ordinary assessment of the precise sequence of hormonally produced events, the patient experiences cessation of hunger. The patient benefits from the ileal brake hormone release with regeneration of organs and tissues, typically pancreas, liver and gastrointestinal tract and in certain instances, heart and vascular tissue.

With respect to the sequence of signaling molecules from the ileum, a response to the medicament entails a wake up stimulation of distal intestinal L-cells that have been quieted by actions of intestinal bacteria or metabolic disease; there is a release of hormones and signals from said L-cells; said released hormones traveling in portal blood to pancreas, liver and GI tract, said organs regenerated from available growth factors and hormone signals, measured biomarkers of the FS index demonstrating the successful regeneration and said regenerated organs then signaling the patient, preferably a human, to resume adequate nutrition seeking behavior as directed by restored signals of hunger.

High FS index values predicted CV risk in this patient population of patients, regardless of the specific components of Metabolic Syndrome that were abnormal. Abnormal and rising FS index values predicted AMI although did not predict the time of the event. A rapid rise in the FS index over 3-6 months was a good predictor of impending CV events. When Metabolic Syndrome is studied as the equal weight of its components using the FS index, it is apparent why clinical strategies treating only one component of Metabolic Syndrome do not remove all risk of CV events. The index also at least and partially explains why drug therapies that improve one aspect of Metabolic Syndrome, but worsen others, may not mitigate CV risk or remove CV events in complex Metabolic Syndrome patients. Abnormal FS index values subsequently normalized, indicated resolution of each component of Metabolic Syndrome, raising the possibility that specific treatments of Metabolic Syndrome might halt progression or reverse Metabolic Syndrome entirely. For example, changes in FS index in patients with RYGB surgery were dramatic, taking scores of these patients from above 250 to values below 20 in most cases. Responses to oral Brake™ were similar to RYGB, even though Brake™ treated patients did not lose as much weight. These data were provided earlier in this application.

FIG. 5 illustrates our use of the neural net model applied to a T2D population of 61 patients treated with metformin alone, and a calculation of parameters such as FS index, HBA1c/SD ratio, and a calculated cumulative CV risk. Clearly, CV risk is relatively low with metformin, but the T2D slowly progresses.

As shown in FIG. 5, the usual pattern of FS index is flat or slowly rising in patients given metformin alone. This indicates that metformin is not a treatment alone for metabolic syndrome. On the other hand, RYGB surgery greatly improves FS index, which does rapidly improve metabolic syndrome.

FIG. 6 shows this improvement in 36 patients, with almost a complete lowering of CV risk to normal.

In FIG. 7, the improvement in FS index and other parameters of metabolic syndrome is shown for 18 patients treated with Brake™. This graph shows that there is about the same lowering of FS index from Brake as RYGB, an observation that is predictive of both of these interventions lowering CV risk in patients. In this manner an expanded benefit can be defined for either RYGB or the oral mimetic of RYGB called Brake™

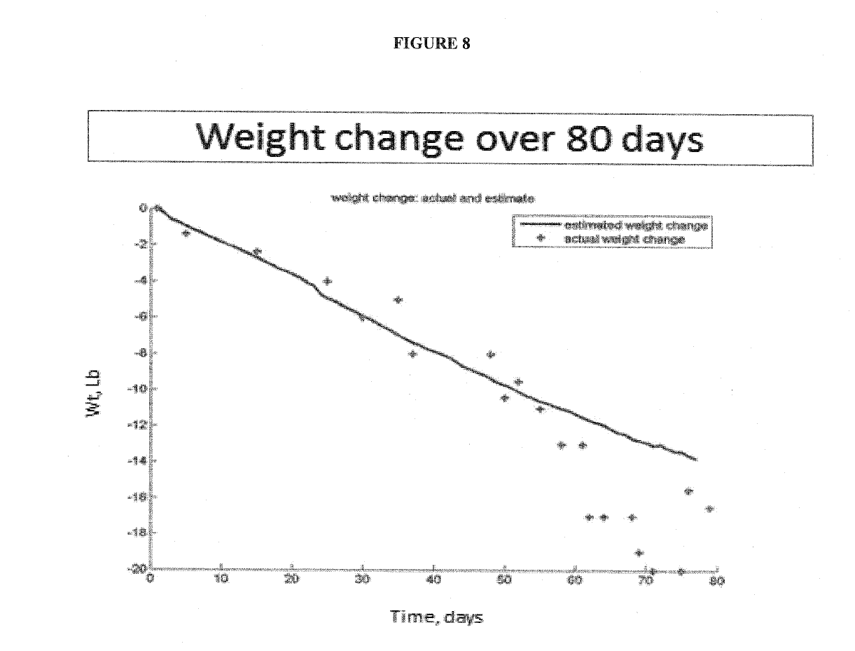

The final model for implementing this metabolic syndrome CV progression model is an application for individual patients on a computer such as a web-enabled cellphone, an I-pad or a Windows 8 tablet. The application will record weight, food intake, calories from specific type of food, and exercise. From these, each patient's insulin output and CV risk is calculated daily and the metabolic syndrome progression is linked to food and lifestyle. Once the links are established for each patient, the application puts the patient onto an optimization plan that should minimize disease and maximize life expectancy. An example of a weight reduction tracked on said application for one patient is FIG. 8.

Weight is plotted in as shown in FIG. 8 as pounds decreased from baseline over a time of 80 days when monitored using said I-pad application. This subject, a 55 year old female, was on a weight reduction program only and did not have abnormalities beyond a mild form of dietary associated metabolic syndrome.

Overall, the FS (Fayad/Schentag) index, which is composed of mostly readily available laboratory and clinical measures, appears to be a promising means of describing progression or amelioration of the end organ manifestations of metabolic syndromes in routine practice, including the changes that occur as a result of organ or system regeneration after RYGB surgery or treatment with Brake™. Its use in aggregate or use of its principle components separately are hereby designated as a primary means of demonstrating direction of metabolic syndrome manifestations (improved or worsening) and the impact of therapeutic interventions designed to improve metabolic syndrome via stop and repair mechanisms of action. To avoid doubt, said therapeutic interventions include both RYGB and combinations of pharmaceuticals wherein the composition of said pharmaceuticals includes Brake™ or its specific components in a dosage range between 2500 mg and 20000 mg, often about 5000 to 12,500 mg, more often about 7500-10,000 mg.

### Example 5

### Reversal of Atherosclerosis and Cardiac Disease

Statins are the mainstay treatment for atherosclerosis and all statins show a dose related lowering of hyperlipidemia. Some statins have shown a reduction in cardiovascular risk profile. This may be achieved by lipid lowering or it may be result of reduced inflammation, or both. Statins alone are not known to regenerate the cardiovascular system or the vascular endothelium. RYGB surgery on the other hand, has a modest lowering of cholesterol, but a dramatic evidence of organ and tissue regeneration, including in the heart and blood vessels. One aspect of the greater effect of RYGB surgery is its impact across the dietary supply side pathways of sugar and fat, T2D and hyperlipidemia. Evidence favoring the combination approach of an orally active RYGB mimetic with a statin is provided below. Subsequently, the inventors disclose our own findings demonstrating the synergistic effects of the combination product of controlled release Brake™ over-coated with 10 mg of a statin. An alternative pharmaceutical agent for over-coating is 10 mg of lisinopril or a suitable ACE inhibitor or AII inhibitor.

The pharmaceutical composition orally active on the ileal brake as disclosed herein may be over-coated with one or more statins in a weight ratio of approximately 0.001 parts atorvastatin or its equivalent potency to each 1.0 part refined sugar or approximately 0.005 part statin:1.0 part refined sugar (e.g. statins selected from the group consisting of atorvastatin, simvastatin, pravastatin, rosuvastatin, lovastatin, fluvastatin and pitavastatin); the enteric coated core of the pharmaceutical composition may also comprise approximately 60-80% refined sugar, 0-40% of a plant-derived lipid and 0-40% of a plant-derived lipid; and/or when apportioning the daily dose of lisinopril onto the daily dose of ileal brake hormone releasing substance in the enteric coated tablet form, the 1.0 gram tablets are over-coated with the immediate release lisinopril in a weight ratio of approximately 0.0005 to 0.002 parts lisinopril to each 1.0 part refined sugar (e.g. ACE inhibitors selected from the group consisting of lisinopril, enalapril, ramipril, perindopril, quinapril, and e.g., any of the AII inhibitors selected from the group consisting of losartan, olmesartan, valsartan, all at dosage equivalents to lisinopril);

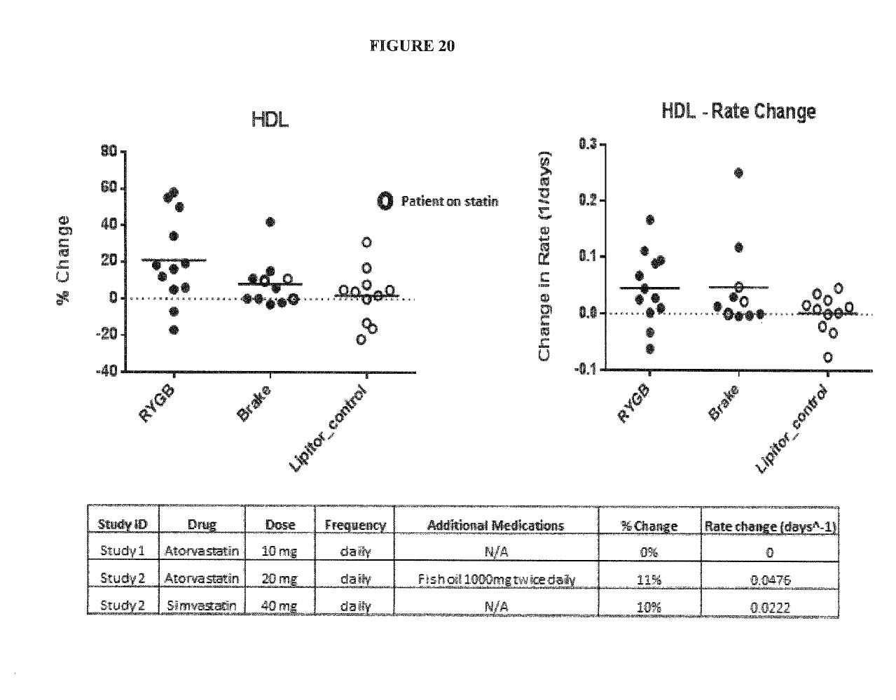

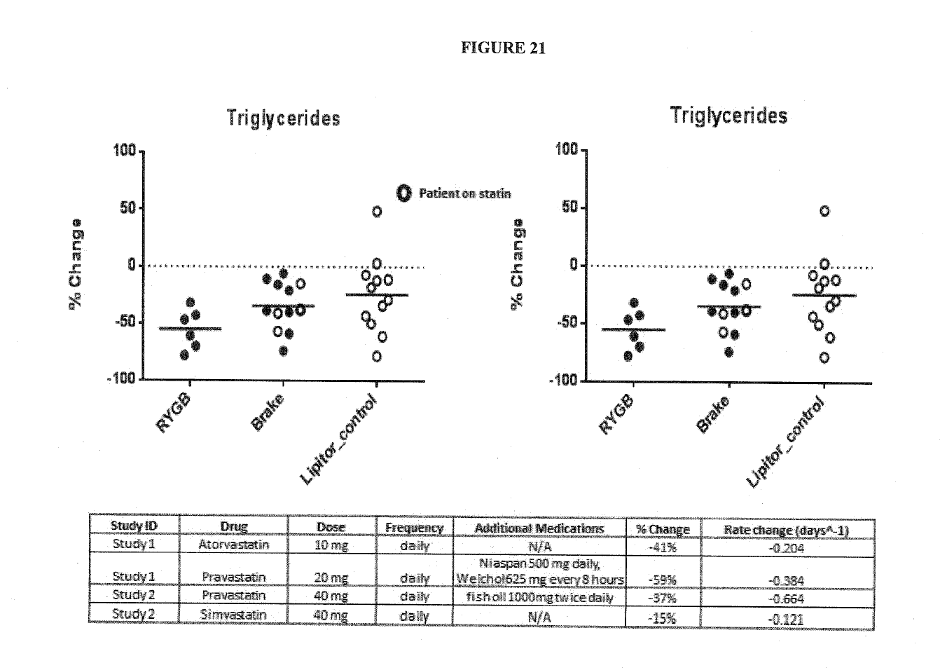

Some examples of patients treated with atorvastatin and Brake™ together, but as separate pills are presented in FIGS. 20 and 21, along with the respective controls. The figures show atorvastatin alone, which has little effect, and Brake™ alone at a dose of 10 gm per day as well as the patients taking both in combination. The figures also show that RYGB patients, by way of reference lose more weight but do not have more effect on metabolic syndrome biomarkers like HDL or TGs when compared to atorvastatin combined with Brake™.

Yu and colleagues examined the effects of early treatment with pravastatin on the progression of glucose intolerance and cardiovascular remodeling in a model of spontaneously developing T2D, the Otsuka Long-Evans Tokushima Fatty (OLETF) rats. The OLETF rats were treated with pravastatin (100 mg/kg/day) from 5 weeks of age and compared with age-matched untreated OLETF rats and normal Long-Evans Tokushima Otsuka (LETO) rats on serial oral glucose tolerance tests (OGTT) and Doppler echocardiography and on histopathological/biochemical analyses of the heart at 30 weeks. The OGTT revealed that 40% and 89% of untreated OLETF rats were diabetic at 20 and 30 weeks, respectively, but 0% and only 30%, respectively, were diabetic in the treated OLETF. Left ventricular diastolic function was found impaired from 20 weeks in untreated OLETF but remained normal in the treated-OLETF. The wall-to-lumen ratio and perivascular fibrosis of coronary arteries were increased in untreated-OLETF but were limited in the treated-OLETF at 30 weeks. Moreover, cardiac expressions of a fibrogenic growth factor, transforming growth factor-beta1 (TGF-beta1), and a pro-inflammatory chemokine, monocyte chemoattractant protein-1 (MCP-1), were increased in untreated-OLETF. However, in the treated-OLETF, over-expressions of TGF-beta1 and MCP-1 were attenuated, which was associated with overexpression of endothelial nitric oxide synthase (eNOS) (2.5-fold of control LETO). Early pravastatin treatment prevented cardiovascular remodeling in the spontaneous DM model by retarding the progression of glucose intolerance, overexpressing cardiac eNOS, and inhibiting over expressions of fibrogenic/pro-inflammatory cytokines.(50). Clearly any of these effects would be synergistic with Brake™ associated regeneration of pancreas and liver.

Infection and inflammation induce the acute-phase response, leading to multiple alterations in lipid and lipoprotein metabolism. Plasma triglyceride levels increase from increased VLDL secretion as a result of adipose tissue lipolysis, increased de novo hepatic fatty acid synthesis, and suppression of fatty acid oxidation. With more severe infection, VLDL clearance decreases secondary to decreased lipoprotein lipase and apolipoprotein E in VLDL. In rodents, hypercholesterolemia occurs attributable to increased hepatic cholesterol synthesis and decreased LDL clearance, conversion of cholesterol to bile acids, and secretion of cholesterol into the bile. Marked alterations in proteins important in HDL metabolism lead to decreased reverse cholesterol transport and increased cholesterol delivery to immune cells. Oxidation of LDL and VLDL increases, whereas HDL becomes a pro-inflammatory molecule. Lipoproteins become enriched in ceramide, glucosylceramide, and sphingomyelin, enhancing uptake by macrophages. Thus, many of the changes in lipoproteins are pro-atherogenic. The molecular mechanisms underlying the decrease in many of the proteins during the acute phase reaction involve coordinated decreases in several nuclear hormone receptors, including peroxisome proliferator-activated receptor, liver×receptor, farnesoid×receptor, and retinoid×receptor. Acute phase response-induced alterations initially protect the host from the harmful effects of bacteria, viruses, and parasites. However, if prolonged, these changes in the structure and function of lipoproteins will contribute to atherogenesis.(51). These pathways are thought to lead to an increased risk in T2D for Cardiovascular events like Myocardial infarction and stroke, and it has recently been shown that obese children already have these findings, predicting risk for early atherosclerosis even in childhood(52).

By way of specific examples of diet associated ASCVD, Shai and colleagues studied the role of dietary intervention in the reversal of atherosclerosis. In a 2-year Dietary Intervention Randomized Controlled Trial-Carotid (DIRECT-Carotid) study, participants were randomized to low-fat, Mediterranean, or low-carbohydrate diets and were followed for changes in carotid artery intima-media thickness, measured with standard B-mode ultrasound, and carotid vessel wall volume (VWV), measured with carotid 3D ultrasound. They found that 2 year weight loss diets can induce a significant regression of measurable carotid VWV. The effect was similar in low-fat, Mediterranean, or low-carbohydrate strategies and appears to be mediated mainly by the weight loss-induced decline in blood pressure(53). Clearly dietary effects of Brake™ are important to reduce the load of sugar and fat on the atherogenic pathways of the body.

These pathways have been associated with progressive accumulation of sugars and fats. There have been clinical studies strongly suggesting that so-called ‘hyperglycemic memory’ which is actually a persisting cumulative record of diabetic damage, can show evidence of chronic abnormalities in diabetic blood vessels that are not easily reversed, even by subsequent, relatively good control of blood glucose. Among various biochemical pathways implicated in diabetic vascular complications, the process of formation and accumulation of advanced glycation end products (AGEs) and their mode of action are most compatible with the theory ‘hyperglycemic memory’. The review by Yamagishi and colleagues discusses the role of AGEs in thrombogenic abnormalities in T2D, especially focusing on the deleterious effects of these macroproteins on endothelial cell function, platelet activation and aggregation, coagulation and fibrinolytic systems(54).

The core areas for regeneration in atherosclerosis are the endovascular walls, and here the damage is accelerated by the combined adverse forces of inflammation, lipid accumulation, tear and repair from hypertension, and micro coagulopathy. Accordingly, vascular improvements may be logically made with lowering of each of these processes, but it does not necessarily follow that lowering any one of them is going to reverse the damage and regenerate endovascular lining. It does appear certain that all of these processes are improved simultaneously by RYGB surgery, as detailed by several authors and summarized below.

It also follows that drugs used in combination with Brake™ for regeneration of endovascular walls would come from the following 4 classes of agents, each of which can be combined with Brake™ for a comprehensive endovascular remodeling and regeneration program for the patients in need. These concomitant drugs, termed second active agents and over-coated onto the Brake™ tablets are as follows:

HMG-CoA reductase inhibitors, also called statins, of which the preferred embodiment is Atorvastatin (Lipitor) in a low dose of 10 mg, or any statin in an equivalent amount, chosen from the alternative listing: Fluvastatin (Lescol), Lovastatin (Mevacor), Pitavastatin (Livalo), Pravastatin (Pravachol), Rosuvastatin (Crestor), Simvastatin (Zocor), among other possible statins.

Angiotensin Converting Enzyme (ACE) inhibitors with preferred example a 10 mg daily dose of Lisinopril (Prinivil, Zestril) or a suitable alternative in an equivalent amount chosen from those marketed ACE inhibitors: benazepril (Lotensin), captopril (Capoten), enalapril (Vasotec), fosinopril (Monopril), moexipril (Univasc), perindopril (Aceon), quinapril (Accupril), ramipril (Altace), trandolapril (Mavik). among other possible alternative ACE inhibitors.

Angiotensin II inhibitors with preferred example an 80 mg dose of Losartan or an equivalent amount of alternative Angiotensin II inhibitor including but not limited to candesartan, irbesartan, valsartan, olmesartan, Telmisartan, among other possible Angiotensin II inhibitors.

Beta Blockers with preferred example propranolol (Inderal) in a dose of 20 mg or a suitable alternative in an equivalent amount chosen from the list of beta blockers: acebutolol (Sectral); atenolol (Tenormin); betaxolol (Kerlone); bisoprolol (Zebeta); carteolol (Cartrol); esmolol (Brevibloc); metoprolol (Lopressor); penbutolol (Levatol); nadolol (Corgard); nebivolol (Bystolic); pindolol (Visken); timolol (Blocadren); sotalol (Betapace); carvedilol (Coreg); labetalol (Trandate), among other possible beta blockers.

Having described these combination products including Brake™, what can be expected when they are used together to regenerate the endovascular cells and the heart itself? The most crucial point of information is the reversal caused by RYGB surgery, as described below:

Several studies point to the reversal of Atherosclerosis by RYGB. On a mechanistic note, illan-Gomez evaluated the relationships between inflammation and atherosclerosis by examining patients for changes in the pro-inflammatory profile of morbidly obese patients after weight loss following bariatric surgery (55). They measured levels of adiponectin, high-sensitivity C-reactive protein (hsCRP), tumor necrosis factor-alpha (TNF-alpha) and interleukin-6 (IL-6) and their relation to insulin resistance and lipid parameters in 60 morbidly obese women at baseline and 3, 6 and 12 months after gastric bypass. Twelve months after RYGB surgery, there was a significant increase in plasma levels of adiponectin (p<0.001) and high-density lipoprotein cholesterol (p<0.01) and a significant decrease in levels of IL-6 (p<0.001), hsCRP (p<0.001), cholesterol (p<0.001), triglycerides (p<0.001), low-density lipoprotein cholesterol (p<0.001), glucose (p<0.001), insulin (p<0.001) and homeostasis model assessment (HOMA; p<0.001). At 12 months, correlations were seen between IL-6 levels and the following: body mass index (BMI) (r=0.53, p<0.001), insulin (r=0.51, p<0.001) and HOMA (r=0.55, p<0.001). Also, hsCRP levels correlated with BMI (r=0.40, p=0.004), triglycerides (r=0.34, p=0.017), insulin (r=0.50, p=0.001) and HOMA (r=0.46, p=0.002). In patients with morbid obesity, significant weight loss is followed by a significant improvement in the inflammatory state, insulin sensitivity and lipid profile. A relationship exists between improved inflammatory profile and lowered insulin resistance(55).

The long term outcomes of the RYGB patients with similar changes in inflammation and lipid parameters have been studied by several groups and will be summarized below.

The objective of Owan and colleagues was to test the hypothesis that RYGB would favorably impact cardiac remodeling and function, which would demonstrate beneficial actions beyond the reversal of atherosclerosis alone. Owan and Colleagues prospectively studied 423 severely obese patients undergoing RYGB and a reference group of severely obese subjects that did not have surgery (n=733). At a 2-year follow up, RYGB subjects had a large reduction in BMI compared with the reference group, and significant reductions in waist circumference, systolic blood pressure, heart rate, triglycerides, low-density lipoprotein cholesterol, and insulin resistance. High-density lipoprotein cholesterol increased. The RYGB group had reductions in left ventricular (LV) mass index and right ventricular (RV) cavity area. Left atrial volume did not change in RYGB but increased in reference subjects. In conjunction with reduced chamber sizes, RYGB subjects also had increased LV midwall fractional shortening and RV fractional area change. In multivariable analysis, age, change in body mass index, severity of nocturnal hypoxemia, E/E′, and sex were independently associated with LV mass index, whereas surgical status, change in waist circumference, and change in insulin resistance were not. They concluded that the RYGB patients had evidence of cardiac remodeling and improved LV and RV function. These data supported the use of RYGB to prevent cardiovascular complications in severe obesity.(56). The data also predict similar outcomes as these patients are treated with Brake™.

The diagnosis of the metabolic syndrome (MS) appears to identify substantial additional cardiovascular risk above and beyond the individual risk factors, even though the pathophysiology underlying this evidence is still incompletely understood. The inflammatory response related to fat accumulation may influence cardiovascular risk through its involvement not only in body weight homeostasis, but also in coagulation, fibrinolysis, endothelial dysfunction, insulin resistance and atherosclerosis. Moreover, there is evidence that oxidative stress may be a mechanistic link between several components of MS and CVD, through its role in inflammation and its ability to disrupt insulin-signaling. The cross-talk between impaired insulin-signaling and inflammatory pathways enhances both metabolic IR and endothelial dysfunction, which synergize to predispose to CVD. Persistent platelet hyper-reactivity/activation emerges as the final pathway driven by intertwined interactions among insulin resistance, adipokine release, inflammation, dyslipidemia and oxidative stress and provides a pathophysiological explanation for the excess risk of atherothrombosis in this setting. Despite the availability of multiple interventions to counteract these metabolic changes, including appropriate diet, regular exercise, anti-obesity drugs and bariatric surgery, relative failure to control the incidence of metabolic syndrome and its complications reflects both the multifactorial nature of these diseases as well as the scarce compliance of patients to established strategies. Evaluation of the impact of these therapeutic strategies on the pathobiology of atherothrombosis, as discussed in this review, will translate into an optimized approach for cardiovascular prevention.(57). These authors are clearly validating the instant invention, since the use of Brake™ in combination with the available front line therapy will manage all of the protean manifestations of metabolic syndrome via activation of the ileal brake pathway.

Metabolic syndrome is commonly associated with multiple conditions imparting adverse cardiovascular risk, including hypertension, dyslipidemia, insulin resistance and T2D. In addition, sleep disordered breathing, inflammation, left ventricular hypertrophy, left atrial enlargement, and subclinical left ventricular systolic and diastolic dysfunction may collectively contribute to increased cardiovascular morbidity and mortality. This review describes improvements in cardiovascular risk factors after bariatric surgery. All of the cardiovascular risk factors listed above are improved or even resolved after RYGB surgery. Cardiac structure and function also have shown consistent improvement after surgically induced weight loss. The amount of improvement in cardiac risk factors is generally proportional to the amount of weight lost. The degree of weight loss varies with different bariatric procedures. On the basis of the improvement in risk profiles, it has been predicted that progression of atherosclerosis could be slowed and the 10-year risk of cardiac events would decline by ˜50% in patients undergoing weight loss surgery. In keeping with these predictions, two studies have demonstrated reductions in 10-year total and cardiovascular mortality of approximately 50% in patients who had bariatric surgery. These encouraging data support the continued, and perhaps expanded, use of surgical procedures to induce weight loss in severely obese patients.(58). Clearly the reversal of the CV and metabolic syndrome biomarkers shown in RYGB patients is only possible with regeneration pathways activated by the ileal brake hormones that are released in these patients (FIG. 1 shows the GLP-1 profiles).

Best and Colleagues considered the CV risk profile of conventional T2D drug therapy in relation to incretin therapies like exenatide. A retrospective database analysis was performed of the Life Link database of medical and pharmaceutical insurance claims for June 2005 through March 2009. Patient outcomes were adjusted for differences in clinical and demographic characteristics and compared using propensity score-weighted discrete time survival analysis with time-varying exposure to exenatide. A total of 39,275 patients with T2D treated with exenatide twice daily, and 381,218 patients were treated with other glucose-lowering therapies. Patients who initiated exenatide were more likely to have prior ischemic heart disease, hyperlipidemia, hypertension, and/or other comorbidities at baseline. Exenatide-treated patients were less likely to have a CVD event than non-exenatide-treated patients (hazard ratio 0.81; 95% CI 0.68-0.95; P=0.01) and lower rates of CVD-related hospitalization (0.88; 0.79-0.98; P=0.02) and all-cause hospitalization (0.94; 0.91-0.97; P<0.001). Exenatide twice-daily treatment was associated with a lower risk of CVD events and hospitalizations than treatment with other glucose-lowering therapies, supporting a lower risk profile associated with the beneficial hormones of the ileal brake (59). The better decline in FS index shown herein supports this conclusion, and favors the use of Brake™ treatment for patients with metabolic syndrome in its various manifestations.

Clearly, RYGB effects as mediated by the ileal brake are beneficial on both atherosclerosis and cardiac functional markers. As with other affected organs that are targets of metabolic syndrome, there is plenty of evidence that RYGB reverses at least a proportion of the injury to the end organ, presumably mediated by the hormones elicited from the L cells of the distal intestine and ileal brake. It would be expected that treatment with Brake™, as an oral mimetic of the RYGB effects on the ileal Brake™, would also be able to demonstrate reversal of atherosclerosis and myocardial injury to a similar degree over a similar time frame, so long as the hormonal responses are similar between RYGB patients and Brake™ treated patients. Data presented herein say that they are.

The notable reversal of CV disease risk following RYGB surgery has been associated with resolution of elevated triglycerides, elevation of HDL, lowering of LDL, and lowering of hepatic inflammation, as was seen using the FS index to monitor the course of these parameters in our patients with RYGB (43) and shown in FIG. 17 for RYGB vs. Brake™ treated cases. Brake™ therapy is most likely to be synergistic on ASCVD regression when its regenerative properties are combined with atorvastatin or a suitable statin. It is clear from the extensive prior studies in animal models of hyperlipidemia and atherosclerosis, summarized earlier in this example, that a biomarker approach can be relied upon to demonstrate favorable effects of Brake™ on reversal of atherosclerosis and associated cardiovascular diseases. Based on the unexpected but highly beneficial improvement in biomarkers and improved beta cell functioning after RYGB, it is an aspect of the invention to treat hyperlipidemia with a novel combination oral therapy of a statin or other hyperlipidemia drug, nominally for the first demonstration, atorvastatin or simvastatin and Brake™.

Combination therapy between a statin and Brake™ for the surprising reversal of atherosclerosis is hereby incorporated by reference, with dosages of Simvastatin, Atorvastatin of 10-20 mg per dose of Brake™ of 10-20 grams daily, both active agents are presented as micro granules for oral administration to patients with atherosclerosis, or alternatively as the immediate release form of atorvastatin over-coated onto the tablets of Brake™. This combination has the surprising potential, when used in conjunction with biomarkers defining early risk of hyperlipidemia to prevent the onset of metabolic syndrome associated damage to the heart and CV system, or at least delay its onset by many years. The disclosed combination product would be the first disease modifying treatment for this disease, here-to-fore considered to be progressive and irreversible.

Clinical proof of the utility of the synergistic combination of these atherosclerosis reversal therapies including Brake™ would necessitate the adoption of biomarkers of metabolic syndrome progression such as the FS index, which is an overall biomarker profile that can point to regenerative processes that respond to RYGB or Brake™. Added to the metabolic syndrome biomarker profile of the FS index would be a biomarker profile of T2D progression to CV injury. This latter progression profile would focus on cardiac injury, include epigenetics, metabolomics and genomics where applicable, and imaging where applicable to loss of cardiac structure and function. To the extent that these biomarkers are improved by statins, those effects carry forward as supportive evidence in the cholesterol pathway itself. To the extent that the observed improvement is tied to effects beyond those of atorvastatin or simvastatin, the conclusion would be Brake™ associated recovery or regeneration of cardiac function.

Every patient would receive Brake™ treatment that would be demonstrated to be active on the basis of lowered biomarkers of ASCVD in a pattern of elevation similar to that observed in our RYGB patients. In combination with oral Brake™ treatment as disclosed herein, the patient would also receive an approved front line treatment for hyperlipidemia such as simvastatin or atorvastatin or other statin, for example the immediate release forms of either of these therapeutics over-coated onto the ileal brake hormone releasing composition Brake™. Atorvastatin when given in this manner is so active in the composition that the atorvastatin dose is a low dose 10 mg per 24 hours, which is nearly the lowest dosage used and is clearly free of the risk of statin side effects such as myopathy. There are two tested reasons that Brake™ would improve both the efficacy and safety of statins in the treatment of T2D. First, both agents have side effects which are dose related, and in both cases using a lower dosage would still improve the efficacy and yet side effects would decrease. Secondly, the control of underlying metabolic syndrome offers the previously unexpected reversal of the atherosclerosis pathophysiology, which is tied to Brake™ associated reversal of insulin resistance, hyperlipidemia, hyperglycemia, hypertension and hepatic steatosis, all of which will be improved or resolved by including Brake™ in the combination therapy of atherosclerosis patients with metabolic syndromes.

Brake™ therapy is likely to be synergistic with acyl-coenzyme A: cholesterol O-acyltransferase (ACAT) inhibitors, which are important in the generation of lipid-filled monocytes-macrophages. In a test of this hypothesis, the ACAT inhibitor CI-976 (2,2-dimethyl-N-(2,4,6-trimethoxyphenyl) dodecanamide) was evaluated relative to selected lipid-lowering agents for their effect on atherosclerotic lesion regression and progression. Atherosclerotic lesions comparable in composition to human fatty streaks were induced by chronic endothelial denudation in the iliac-femoral artery of hypercholesterolemic New Zealand White rabbits before intervention, while naturally occurring fatty streaks developed in the thoracic aorta. CI-976 administered in a hypercholesterolemic diet at a dose that did not lower plasma cholesterol prevented the accumulation of monocytes-macrophages within the pre-established iliac-femoral lesion and reduced the foam cell area by 27-29% relative to the initiation of intervention. CI-976 also blunted the development of thoracic aortic fatty streak-like lesions and decreased the cholesteryl ester enrichment by 46%. CI-976 had no effect on plasma triglycerides and, more importantly, had no effect or decreased liver, iliac-femoral, and thoracic aortic free cholesterol content. Dietary intervention alone increased monocyte-macrophage involvement in the iliac-femoral lesion despite reductions in plasma, liver, and thoracic aortic cholesterol content. Conventional lipid-lowering therapy such as cholestyramine or cholestyramine/niacin required substantial decreases in plasma cholesterol levels to achieve comparable vascular changes. These authors conclude that inhibition of ACAT within the arterial wall by the potent and specific ACAT inhibitor CI-976, even in the absence of plasma cholesterol lowering, can result in the inhibition of atherosclerotic lesion progression and can enhance regression(60)

We believe these data support the thesis of Shai (53) and demonstrate evidence of reversal of atherosclerosis associated damage.

There are some additional compounds that would be useful in combination with oral mimetics of RYGB that release ileal brake hormones as their primary mechanism of action. The following agents would synergistically combine with Brake™ to regenerate endovascular surfaces and thereby mitigate atherosclerosis and lessen the numbers of patients who progress to ASCVD.

One example is ETC-216. Recently, regression of atherosclerosis was achieved in coronary patients by repeated infusions of ETC-216. Thirty-six rabbits underwent perivascular injury at both carotid arteries, followed by a 1.5% cholesterol diet. After 90 days, rabbits were randomly divided into 6 groups and treated 5 times with vehicle or ETC-216 at 5, 10, 20, 40, or 150 mg/kg dose every 4 days. Carotid plaque changes were evaluated in vivo by intravascular ultrasound (IVUS) and magnetic resonance imaging (MRI), performed before and at the end of treatments. Magnetic resonance imaging scans were also recorded after administration of the second dose for rabbits infused with vehicle 40 or 150 mg/kg. Atheroma volume in vehicle-treated rabbits increased dramatically between the first and the second IVUS analyses (+26.53%), whereas in ETC-216-treated animals, a reduced progression at the lower doses and a significant regression at the higher doses, up to −6.83%, was detected. Results obtained by MRI analysis correlated significantly with those at IVUS (r=0.706; p<0.0001). The MRI evaluations after the second infusion established that a significant regression was achieved with only 2 administrations of the highest dose. These results confirm the efficacy of ETC-216 for atherosclerosis treatment and provide guidance for dose selection and frequency to obtain a significant reduction of plaque volume.(61)

RVX-208 is a first-in-class small molecule that inhibits BET bromodomains. RVX-208 functions by removing atherosclerotic plaque via reverse cholesterol transport (RCT), the natural process through which atherosclerotic plaque is transported out of the arteries and removed from the body by the liver. RVX-208 increases production of Apolipoprotein A-I (ApoA-I), the key building block of functional high-density lipoprotein (HDL) particles and the type required for RCT. These newly produced, functional HDL particles are flat and empty and can efficiently remove plaque and stabilize or reverse atherosclerotic disease. Results from the ongoing analysis of its Phase 2b ASSURE clinical trial using intravascular ultrasound (IVUS) to study high-risk cardiovascular disease (CVD) patients for assessing benefits of RVX-208 show statistically significant improvements in coronary IVUS atheroma measurements and Major Adverse Cardiac Events (MACE) in patients with a high (>2.0 mg/dL) serum high sensitivity C-Reactive Protein (hsCRP). Serum levels of this biomarker when >2.0 mg/dL reflect a heightened state of inflammation that is a well-known and major component of CVD risk. Patients with hsCRP>2.0 mg/dL at time of entry into ASSURE totaled n=184 of which n=54 were given placebo while n=130 received RVX-208. In the RVX-208 treated patients, the incidence of MACE was lower by 63% (p=0.023) vs. placebo. The preceding observation is of value in that hsCRP of >2.0 mg/dL is well known to be clinically important in predicting CVD risk.

The VH-IVUS data was analyzed to provide insight into vulnerability of an atherosclerotic plaque to rupture and its relationship to future cardiovascular risk. In ASSURE, while all (n=323) patients were studied using IVUS, 87 of these were examined using the Volcano Revolution catheter to gather VH-IVUS information. This information was used to reflect plaque vulnerability by calculating the ratio of necrotic core to dense calcium (NC/DC) as established by Missel et al. (Am J Cardiol 2008; NC/DC ratio). The NC/DC ratio in RVX-208 treated patients (n=61) was significantly lower by −7.5%. The treatment of ASSURE patients with low HDL-C given rosuvastatin further defined a large high risk population where RVX-208 illustrates profound effects to reduce atheroma volume and plaque vulnerability. Together these findings help explain the observed reduction in MACE events.

RVX-208 is given in doses of 100 mg once daily, and could be readily over-coated onto 7 Brake™ pills for a complete lipid control regimen, which would reverse atherosclerosis and be cardio protective of MACE events in patients receiving statins.

In another aspect of the present invention, there are compounds suitable to combine with the ileal brake hormone releasing substance of the invention, wherein the patient with congestive heart failure (CHF) could greatly benefit from cardiovascular system regeneration. One example is the alpha-beta blocker agent carvedilol, which itself has been beneficial to patients with CHF. In the preferred embodiment of the combination product, the desired dose of carvedilol could be lowered to 12.5 mg every 24 hrs and still be effective in CHF.

### Example 6

### Hepatic Regeneration

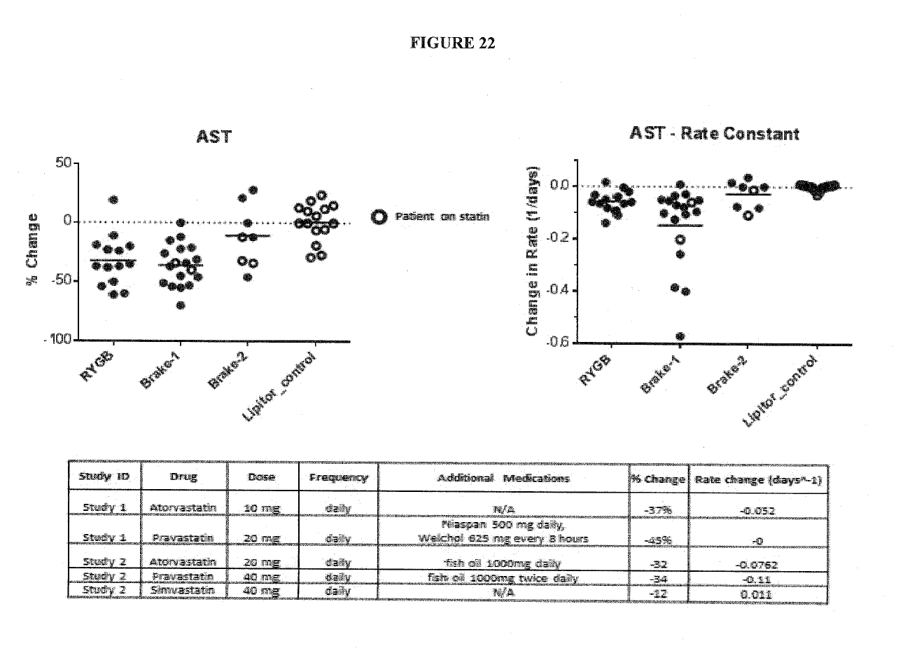

Some examples of patients treated with atorvastatin and Brake™ together, but as separate pills are presented in FIGS. 22, along with the respective controls. The figures show hepatic enzyme (AST, a major component of FS index) decline with atorvastatin alone, which has little effect on hepatic inflammation, and Brake™ alone at a dose of 10 gm per day as well as the patients taking both in combination. The figures also show that RYGB patients, by way of reference lose more weight but do not have more effect on metabolic syndrome biomarkers of hepatic steatosis like AST when compared to atorvastatin combined with Brake™. Hence the novel observation is synergy between a lipid pathway drug and Brake™ in hepatic regeneration. The use of the lowest clinical dose of 10 mg atorvastatin, or its equivalent statin, allows the treatment of hepatic steatosis without risk of statin side effects.

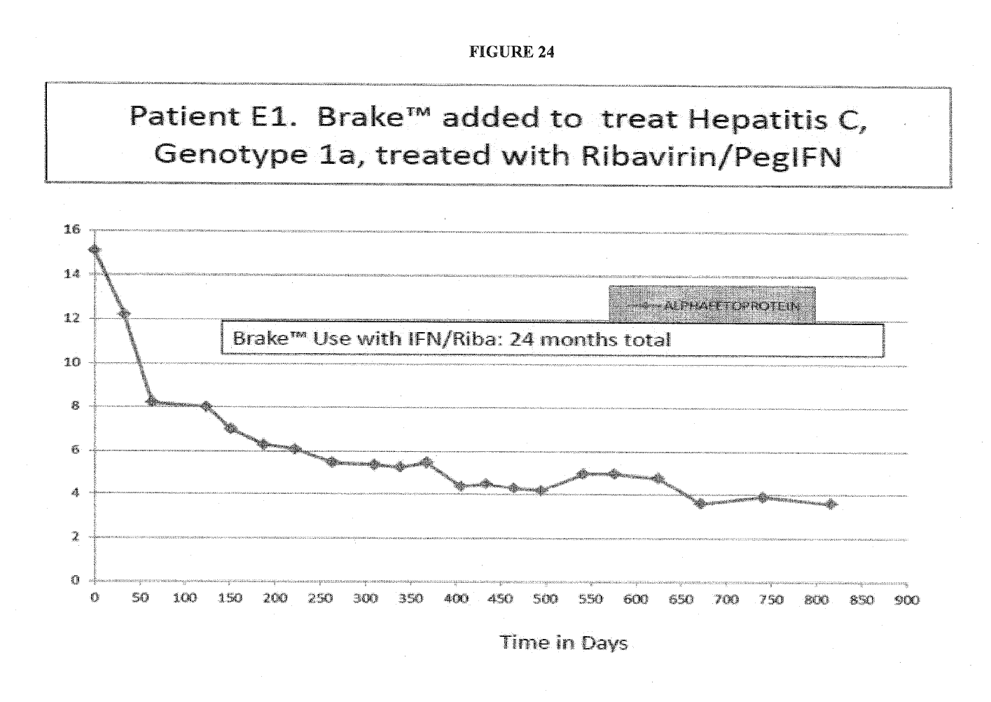

In another preferred embodiment, the use of Brake™ in combination with antiviral compounds is disclosed for the treatment of Hepatic steatosis associated with hepatitis B and C. In this application, the disclosed medicaments in combination regenerate the damaged liver itself, which resolves inflammation and conveys a lowering of hepatic enzymes. In general, as AST falls to normal, the damaged liver has been at least partially regenerated. One additional benefit is the lowering of alpha-fetoprotein, which is a marker of risk for hepatocellular carcinoma. An example of hepatic steatosis resolution in a 36 year old male with hepatitis C, our patient, E2. Initially his weight was 1851b, with a calculated BMI of 29. His Hepatitis C was genotype 1a TC, and when he presented his liver biopsy showed hepatic steatosis and ¼ fibrosis. He was started on interferon and ribavirin, but these agents did not control the viral load. Accordingly, Brake™ was added to his regimen and continued for 24 months. Viral load became undetectable, hepatic enzymes and triglycerides normalized. In FIG. 24, his alpha-fetoprotein normalized, indicating the regeneration of his liver and the removal of the risk for hepatocellular carcinoma. Further embodiments of the use of Brake™ in Hepatic Steatosis are disclosed in US2013/0337055 A1, and are hereby incorporated in their entirety. The Brake™ tablets are over-coated with 600-1200 mg of ribavirin, and this product is called RibaBrake™

In a further practice of the invention, berberine in available forms in a daily amount of 500-1000 mg may be substituted for a statin and be likewise over-coated in the combination formulation.

Berberine is an alkaloid that has been isolated from various anti-diabetic plants used in Traditional Chinese Medicine. Berberine has various mechanisms of action, but tends to be known as an AMPK activator; alongside AMPK activation, berberine also exerts anti-inflammatory effects, benefits to intestinal health and integrity, possible synergism with anti-depressant medications, lipid and cholesterol lowering effects, and strong anti-diabetic effects. The anti-diabetic effects of berberine are the most well-researched, and are partly due to AMPK activation; PTP1B inhibition, which reduces glucose production in the liver, may also contribute, as well as berberine's anti-inflammatory effects. Comparative research in both animals and humans (as well as one meta-analysis on humans) demonstrate that the anti-diabetic effects of 1500 mg of Berberine taken in three doses of 500 mg appear to be equal to those of 1500 mg Metformin or 4 mg Glibenclamide in terms of reducing biomarkers of type 2 diabetes.

Additionally, due to the mechanism of action being AMPK activation, berberine exhibits fairly potent lipid-lowering effects, and via other unrelated mechanisms also reduces circulating cholesterol levels; these side effects make berberine desirable for reducing the risk of cardiac complications associated with diabetes. There are also some less-proven but promising effects associated with berberine supplementation that may protect against diabetic cardiomyopathy and diabetic nephropathy.

Release of ileal brake hormones increases hepatic cell mass and decreases the number of inflamed hepatic cells in the Hepatic Steatosis patient and typically and uniquely normalizes triglycerides, hepatic enzymes, alpha-fetoprotein and cholesterol. As further demonstration of heretofore unexpected hepatocellular regeneration, the effects of daily use of the dosage form for 6 months persist for prolonged periods even if the medication is not taken.

Use of the disclosed treatments and methods of modifying L-cell output of regulatory hormones for purposes of triggering regeneration of pancreatic beta cells, hepatic cells and regeneration of GI tract cells, is advised in order to benefit patients having metabolic syndrome and in need of improved organ function, the changes from which treatment are long lasting and universally beneficial.

Insulin resistance is a key component of the metabolic syndrome (MS) and is strongly associated with liver steatosis. None of the current treatments for metabolic syndrome resolve Insulin resistance, yet resolution of insulin resistance is necessary to effect a regeneration of peripheral systems such as neural tissue or indeed the heart and brain. One clear benefit is to start regeneration early, for example in childhood obesity.

The aim of D'Adamo and colleagues was to evaluate whether metabolic syndrome should be diagnosed already in obese pre-pubertal children and whether its prevalence is influenced by the inclusion of hepatic steatosis as a diagnostic criterion. Eighty-nine obese children (43 boys; age median [range], 8.5 [6-10] years) were enrolled. Metabolic syndrome was diagnosed according to a classic definition: presence of 3 or more of the following criteria-body mass index greater than 2 standard deviation score, triglycerides greater than the 95th percentile, high-density lipoprotein cholesterol less than the fifth percentile, blood pressure greater than the 95th percentile, and impaired glucose tolerance. Afterward, liver steatosis was included as an additional criterion to this definition. Metabolic syndrome was diagnosed in 12 children (13.5%) according to the first definition and in 18 children (20.2%) when liver steatosis was included. The prevalence of metabolic syndrome increased across homeostasis model assessment of insulin resistance tertiles (P for trend=0.01). The prevalence of the single components of the metabolic syndrome was as follows: central adiposity, 100%; hypertriglyceridemia, 27%; low high-density lipoprotein cholesterol, 2.2%; hypertension, 34.8%; impaired glucose tolerance, 4.5%; and nonalcoholic fatty liver disease, 21.3%. In conclusion, metabolic syndrome is common already among pre-pubertal obese children, particularly when hepatic steatosis is included among the diagnostic criteria. Therefore, screening for the metabolic syndrome should be performed in this age group; and hepatic steatosis should be considered as an additional diagnostic criterion.(62)

It seems clear from the extensive prior biomarker studies in animal models of Hepatitis C and NAFLD, summarized earlier in this example, that a biomarker approach can be relied upon to demonstrate favorable effects of Brake™ on reversal of NAFLD and associated cardiovascular diseases. Based on the unexpected but highly beneficial improvement in biomarkers and improved hepatic functioning after RYGB, it is a method of the present invention to treat NAFLD with a novel orally administered combination therapy of a statin, nominally for the first demonstration, atorvastatin or simvastatin and Brake™ Every patient would receive Brake™ treatment that would be demonstrated to be active on the basis of lowered biomarkers of NAFLD in a pattern of elevation similar to that observed in our RYGB patients. In combination with oral Brake™ treatment as disclosed herein, the patient would also receive an approved front line treatment for hyperlipidemia such as simvastatin or atorvastatin, either of the statins given in the usual dose or in some novel regimens, given at less than half the usual dose. There are two tested reasons that Brake™ would improve both the efficacy and safety of statins in the treatment of NAFLD. First, both agents have side effects which are dose related, and in both cases using a lower dosage would still improve the efficacy and yet side effects would decrease. Secondly, the control of underlying metabolic syndrome promises true reversal of the NAFLD pathophysiology, which is tied to Brake™ associated reversal of insulin resistance, hyperlipidemia, hyperglycemia, hypertension and hepatic steatosis, all of which will be improved or resolved by including Brake™ in the combination therapy of NAFLD patients with metabolic syndromes.

Combination therapy between a statin and Brake™ for the surprising reversal of NAFLD is hereby incorporated by reference, with daily dosages of Simvastatin, Atorvastatin of 5-10 mg over-coated onto Brake™ of 10-20 grams daily, both active agents are presented as micro granules for oral administration to patients with NAFLD. This combination has the surprising potential, when used in conjunction with biomarkers defining early risk of hyperlipidemia to prevent the onset of metabolic syndrome associated damage to the heart and CV system, or at least delay its onset by many years. The disclosed combination product would be the first disease modifying treatment for this disease, here-to-fore considered to be irreversible.

Clinical proof of the utility of the synergistic combination of these NAFLD and associated hepatic regeneration therapies including Brake™ would necessitate the adoption of biomarkers of metabolic syndrome progression such as the FS index, which is an overall biomarker profile that can point to regenerative processes that respond to RYGB or Brake™ Added to the metabolic syndrome biomarker profile of the FS index would be a biomarker profile of NAFLD progression to hepatocellular carcinoma or cirrhosis. This latter progression profile would focus on cardiac injury, include epigenetics, metabolomics and genomics where applicable, and imaging where applicable to loss of cardiac structure and function. To the extent that these biomarkers are improved by statins, those effects carry forward. To the extent that the observed improvement is tied to effects beyond those of atorvastatin or simvastatin, the conclusion would be Brake™ associated recovery or regeneration of hepatic function.

### Example 7

### Regeneration of the GI Tract

Use of the disclosed treatments and methods of modifying human gastrointestinal flora and the interaction between bacteria and L-cells of the ileum, for purposes of triggering regeneration of GI tract cells to benefit metabolic syndrome treatment are based on the findings incorporated by reference herein.

According to the teachings of Koehler, GLP-2 exerts pro-absorptive, regenerative, and cytoprotective actions in the normal and injured gut epithelium. Hence, sustained GLP-2 receptor (GLP-2R) activation represents a strategy under investigation for the prevention and treatment of chemotherapy-induced mucositis. It was found that GLP-2R activation engages signaling pathways promoting cell proliferation and cyto-protection in the normal gut epithelium, but also found that sustained direct or indirect modulation of GLP-2R signaling does not modify intestinal tumor cell growth or survival.(63)

Drucker noted that GLP-2 acts proximally to control energy intake by enhancing nutrient absorption and attenuating mucosal injury and is currently marketed as Teduglutide by Takeda for the treatment of short bowel syndrome.(64) Moreover, GLP-2 receptor agonists appear to be promising therapies for the treatment of intestinal disorders. (65) GLP-2 also promotes intestinal cell proliferation and confers resistance to cellular injury in a variety of cell types. Administration of GLP-2 to animals with experimental intestinal injury promotes regeneration of the gastrointestinal epithelial mucosa and confers resistance to apoptosis in an indirect manner via yet-to-be identified. GLP-2 receptor-dependent regulators of mucosal growth and cell survival. These proliferative and anti-apoptotic actions of GLP-2 may contribute to protective and regenerative actions of these peptides in human subjects with T2D and intestinal disorders. (66)

Peptide hormones regulate cell viability and tissue integrity, directly or indirectly, through activation of G-protein-coupled receptors via diverse mechanisms including stimulation of cell proliferation and inhibition of cell death. Glucagon-like peptide-2 (GLP-2) is a 33 amino acid peptide hormone released from intestinal endocrine cells following nutrient ingestion. GLP-2 stimulates intestinal crypt cell proliferation leading to expansion of the gastrointestinal mucosal epithelium. Exogenous GLP-2 administration attenuates intestinal injury in experimental models of gastrointestinal disease and improves intestinal absorption and nutritional status in human patients with intestinal failure secondary to short bowel syndrome. GLP-2 also promotes mucosal integrity via reduction of injury-associated apoptosis in the intestinal mucosa and directly reduces apoptosis in cells expressing the GLP-2 receptor in vitro. Hence, the regenerative and cytoprotective properties of GLP-2 contribute to its therapeutic potential for the treatment of patients with intestinal disease.(67)

Endogenous GLP-2 regulates the intestinotropic response in re-fed mice through modulation of crypt-cell proliferation and villus apoptosis. GLP-2 is therefore a physiologic regulator of the dynamic adaptation of the gut mucosal epithelium in response to luminal nutrients.(68)

Perhaps the most profound example of GLP-2 action, including a near complete regeneration of GI endothelial lining cells themselves, follows RYGB surgery, where one aspect of the surgery is to connect the esophagus to mid jejunum and completely bypass the duodenum. The result is major malabsorption of nutrients, which over the ensuing months after surgery is mitigated by GLP-2 remodeling of the jejunum into a section nearly as efficient as a section of duodenum. This is the very best definition of ileal brake associated GI remodeling and it happens in direct concert with regeneration of pancreatic beta cells and complete resolution of hepatic steatosis, all aspects mediated by the hormones of the ileal brake.

Le Roux studied the mechanistic linkages between changes in crypt cell proliferation and GLP-2 in rodents and man after RYGB. GLP-2 released from intestinal L-cells after nutrient intake stimulates intestinal crypt cell proliferation and mitigates the effects of gut injury. Wistar rats underwent either RYGB (n=6) or sham procedure (n=6) and plasma GLP-2, GLP-1, and PYY were measured after 23 days. In order to study the signaling and time course of these changes, biopsies from the terminal ileum were stained using the antibody to Ki67, which detects cyclins and hence demonstrates cells in the S-phase of the cell cycle. The total number of cells, number of mitosis, and number of labeled cells per crypt were counted. Obese patients (n=6) undergoing RYGB were evaluated following a 420 kcal meal preoperatively, and 1, 3, 6, 12, and 24 months later for responses in 1-cell products such as GLP-2, GLP-1, total PYY, and PYY3-36. Rat GLP-2 levels after RYGB were elevated 91% above sham animals (P=0.02). At necropsy, mitotic rate (P<0.001) and cells positive for the antibody Ki67 (P<0.001) were increased, indicating crypt cell proliferation. Human GLP-2 after RYGB reached a peak at 6 months of 168% (P<0.01) above preoperative values. Area under the curve for GLP-1 (P<0.0001), total PYY (P<0.01), and PYY3-36 (P <0.05) responses increased progressively over 24 months. In both rodents and patients, RYGB leads to increased GLP-2 and mucosal crypt cell proliferation. Other gut hormones from L-cells remain elevated for at least 2 years in humans. These findings may account for the restoration of the absorptive surface area of the gut, which limits malabsorption, regulates the interaction between nutrient intake and fat storage and contributes to the long-term weight loss after RYGB.(69)

One potential combination product with Brake, where the end result is restoration of the integrity of the small bowel, would be use of Brake with a small amount of a locally acting corticosteroid such as budesonide in a daily amount of 3.0 mg, where the goal of the steroid in the combination product is to lower the luminal inflammation in diseases like Crohn's and Ulcerative colitis. As these products are targeted for release also in the ileum or ascending colon, the practical aspect of co-formulation would be to combine the corticosteroid within the ileal brake hormone releasing substance core. In this case, all components of the formulation need to be released at the same site in the intestine, so the coating used for release of the ileal brake hormone releasing substance is sufficient for the entire components, that is to incorporate the second active drug as well. There are other short acting local steroids available as alternatives to budesonide, for example mometasone, ciclesonide, beclomethasone, fluticasone, flunisolide and other similar compounds that are topically active and metabolized locally, generally lacking systemic steroid activity and side effects. Said steroids are typically used by inhalation to treat conditions such as asthma, which also takes full advantage of their local action.

In another preferred embodiment of a Brake combination treatment for inflammatory bowel diseases, the combination may optionally include a probiotic bacterial organism or composition of probiotic organisms, in this case also formulated for release in the ileum or ascending colon, in a dosage of 10̂6 to about 10̂8 colony forming units. The purpose of this additional preferred active ingredient is to repair the intestinal dysbiosis which often accompanies the various forms of inflammatory bowel disease.

Weir and colleagues opined that this should also be an ideal approach to treatment of T1D, shutting off apoptosis and stimulating beta cell regeneration(70)

Bastien-Dione and colleagues have studied epigenetic signaling pathways and have previously shown that the forkhead transcription factor FoxO1 is a prominent transcriptional effector of GLP-1 signaling in the beta-cell. FoxO1 activity is subject to a complex regulation by Akt-dependent phosphorylation and SirT1-mediated deacetylation. In this study, they aimed at investigating the potential role of SirT1 in GLP-1 action. FoxO1 acetylation levels and binding to SirT1 were studied by Western immunoblot analysis in INS832/13 cells. SirT1 activity was evaluated using an in vitro deacetylation assay and correlated with the NAD(+)-to-NADH ratio. The implication of SirT1 in GLP-1-induced proliferation was investigated by BrdU incorporation assay. They determined beta-cell replication and mass in wild-type and transgenic mice with SirT1 gain of function after daily administration of exendin-4 for 1 week. Study data showed that GLP-1 increases FoxO1 acetylation, decreases the binding of SirT1 to FoxO1, and stunts SirT1 activity in beta-INS832/13 cells. GLP-1 decreases both the NAD(+)-to-NADH ratio and SirT1 expression in INS cells and isolated islets, thereby providing possible mechanisms by which GLP-1 could modulate SirT1 activity. Finally, the action of GLP-1 on beta-cell mass expansion is abolished in both transgenic mice and cultured beta-cells with increased dosage of SirT1. This study shows for the first time that GLP-1 modulates SirT1 activity and FoxO1 acetylation in beta-cells. They also identify SirT1 as a negative regulator of beta-cell proliferation.(71)

Paneth cells are locations for intestinal stem cells. Yilmaz et al studied caloric restriction and found that it promotes self-renewal of intestinal stem cells through the inhibition of mammalian target of rapamycin complex 1 (mTORC1) in Paneth cells. Paneth cell are packed together with LGR5 (leu-rich repeat-containing G protein coupled receptor 5)-positive intestinal stem cells at the base of intestinal crypts. Calorie restriction was found to increase the numbers of Paneth cells and ISCs in mice. This observation, coupled with the fact that the number of differentiated enterocytes was reduced following calorie restriction, indicated that reduced calorie intake promotes self-renewal but not differentiation of ISCs. In addition, ISCs from calorie restricted mice displayed increased regenerative capacity as assayed by the ability of isolated crypts to form organoid bodies in vitro.

In summary, GI tract regenerative processes follow the activation of the ileal brake, and the fact that GLP-2 is somewhat specific for regeneration of luminal enterocytes is an advantage. If one can treat local conditions and add an overall stimulant of the ileal brake hormones to that local treatment, then a new and highly synergistic combination regimen can be offered for the treatment of local diseases of the GI tract such as inflammatory bowel diseases. Specific combinations of these components are offered for use in the treatment of inflammatory bowel disease, but it is recognized that there are many other localized GI diseases that may benefit from this approach.

### Example 8

### Kidney Regeneration and Joint Regeneration in RA Patients

Weight-loss surgery may reduce the risk of kidney disease progression in obese people with T2D, according to a small study. The study included 52 patients, mostly female, who were obese and had T2D. Nearly 40 percent of the patients had diabetic nephropathy, a form of kidney damage that can require dialysis and lead to kidney failure. All of the patients underwent RYGB surgery Five years after surgery, nearly 60 percent of the patients who'd had diabetic nephropathy no longer had the condition. They also found that only 25 percent of those who did not have diabetic nephropathy at the time of surgery eventually developed the condition. That's about 50 percent less than the occurrence rate in people with T2D who don't have bariatric surgery. The five-year T2D remission and improvement rates for patients in the study were 44 percent and 33 percent, respectively. Over half the patients who had diabetic nephropathy prior to undergoing bariatric surgery experienced remission. This is a remarkable finding that warrants greater consideration of bariatric surgery in this patient population. About 90 percent of people with T2D worldwide are overweight or obese, according to the World Health Organization. In the study, patients' average body-mass index—a measure of body fat based on height and weight—was 49 at the time of the surgery. A body-mass index of 30 or higher is considered obese. Because this study was presented at a medical meeting, the data and conclusions should be viewed as preliminary until published in a peer-reviewed journal. Experts also note that although the study found an association between weight-loss surgery and less kidney damage, researchers did not prove that the surgery was responsible for the decreased kidney disease.

Angiotensin II inhibitors are the mainstay treatment for diabetic kidney diseases and all AII inhibitors show a dose related lowering of proteinuria. Some AII inhibitors have shown a reduction in cardiovascular risk profile and in the risk of progression to dialysis. This may be achieved by proteinuria lowering or it may be result of reduced inflammation, or both. RYGB surgery on the other hand, has a modest lowering of serum creatinine in our patients, but a dramatic evidence of organ and tissue regeneration, including in the heart and blood vessels. One aspect of the greater effect of RYGB surgery is its impact across the dietary supply side pathways of sugar and fat, T2D and hyperglycemia, all significant risk factors for diabetic nephropathy. Evidence favoring the combination approach of an orally active RYGB mimetic with a statin is provided below. Subsequently, the inventors disclose our own findings demonstrating the synergistic effects of the combination product of controlled release Brake™ over-coated with 10 mg of a lisinopril or a suitable ACE inhibitor or suitable Angiotensin II inhibitors such as Losartan, candesartan, irbesartan, olmesartan, valsartan or any other suitable AII inhibitor.

The pharmaceutical composition used to treat diabetic nephropathy and orally active on the ileal brake as disclosed herein, may be over-coated with any AII inhibitor such that the daily dose is the same as usually given in conjunction with 7 Brake™ pills, in a weight ratio of approximately 0.008 parts AII inhibitor to each 1.0 part refined sugar or approximately 0.005 part AII inhibitor:1.0 part refined sugar (e.g. AII inhibitors selected from the group consisting of olmesartan, losartan, valsartan and any other suitable sartan compound); the enteric coated core of the pharmaceutical composition may also comprise approximately 60-80% refined sugar, 0-40% of a plant-derived lipid; and/or when apportioning the daily dose of losartan onto the daily dose of ileal brake hormone releasing substance in the enteric coated tablet form, the 1.0 gram tablets are over-coated with the immediate release losartan.

An additional embodiment of the Brake™ controlled release of ileal brake hormones formulation is the use of the product for the treatment of rheumatoid arthritis, typically in combination with methotrexate. Methotrexate is effective in relieving joint inflammation and pain, slowing disease progression, and preventing disability by delaying joint destruction. Patients with rheumatoid arthritis may be more likely to continue treatment with methotrexate than with other DMARDs because of favorable results and tolerable side effects. Studies indicate that more than 50% of people who take methotrexate for rheumatoid arthritis continue taking the medicine for more than 3 years, which is longer than any other DMARD.

Methotrexate is often the first DMARD prescribed for rheumatoid arthritis and usually provides relatively fast relief of at least some symptoms. Patients who can tolerate methotrexate, but it is not sufficiently effective, will be given a second DMARD along with methotrexate (combination therapy). Several recent studies report that treatment results are improved when methotrexate is given with another DMARD. For example, one study found that methotrexate used in combination with etanercept, a new DMARD, is more effective at reducing disease activity than methotrexate alone. Studies with infliximab and adalimumab have shown similar results

Combination therapy may allow for lower doses of an individual drug to be used, which may reduce the risk of adverse effects that can occur with higher doses. In one large review of studies, various combinations of DMARDs plus methotrexate were more effective than either methotrexate or another DMARD alone.

Accordingly, a methotrexate daily dose of 1.0 mg will be over-coated onto the 7 Brake™ pills that constitute a single daily dose. The name of this new treatment for Rheumatoid Arthritis is TrexaBrake™

As noted by Westlake and colleagues, patients with RA have an increased prevalence of cardiovascular disease (CVD). This is due to traditional risk factors and the effects of chronic inflammation. Methotrexate (MTX) is the first-choice DMARD in RA. They performed a systematic literature review to determine whether MTX affects the risk of CVD in patients with RA. They searched Medline, Embase, Cochrane database, database of abstracts of reviews of effects, health technology assessment and Science Citation Index from 1980 to 2008. Conference proceedings (British Society of Rheumatology, ACR and EULAR) were searched from 2005 to 2008. Papers were included if they assessed the relationship between MTX use and CVD in patients with RA. Two reviewers independently assessed each title and abstract for relevance and quality. A total of 2420 abstracts were identified, of which 18 fulfilled the inclusion criteria. Two studies assessed the relationship between MTX use and CVD mortality, one demonstrated a significant reduction in CVD mortality and the second a trend towards reduction. Five studies considered all-cause CVD morbidity. Four demonstrated a significant reduction in CVD morbidity and the fifth a trend towards reduction. MTX use in the year prior to the development of RA decreased the risk of CVD for 3-4 years. Four studies considered myocardial infarction, one demonstrated a decreased risk and three a trend towards decreased risk with MTX use. According to Westlake, MTX use is associated with a reduced risk of CVD events in patients with RA. This suggests that reducing the inflammation in RA using MTX not only improves disease-specific outcomes but may also reduce collateral damage such as atherosclerosis.(72). TrexaBrake is anticipated to be highly protective of the CV system when used in the treatment of Rheumatoid Arthritis.

### Example 9

### Treatment of COPD and Regeneration of Pulmonary Function and Lung Integrity

The pathophysiology of COPD involves a complex series of chronic inflammatory processes that progressively destroy the pulmonary vasculature and lung parenchyma. Two main pathophysiological processes occur in COPD: inflammation and unopposed oxidation.

The inflammatory process is believed to be mediated by chemical factors, such as tumor necrosis factor-alpha (TNF-α), interleukin-8 (IL-8), and leukotriene B₄. When noxious gases or particles have been introduced into the lungs and irritation has occurred, the chemical “messengers” propagate the inflammatory process and recruit neutrophils, macrophages, and lymphocytes to the site of injury.

The second pathophysiological process involves a shift in the balance of normal defense mechanisms, resulting in unopposed oxidation. Guidelines from the Global Initiative for Chronic Obstructive Lung Disease (GOLD) identify disruption of the oxidant/antioxidant or trypsin/antitrypsin balance as a major determinant of damage to the lung parenchyma. Tobacco has been implicated in the disruption of both processes by (1) increasing oxidation, thereby overwhelming antioxidant protective factors, and (2) inducing proteases from macrophages and neutrophils. Tobacco smoke has thus been identified as the single greatest risk factor for COPD because of the processes of cellular damage and because of the high incidence of tobacco use worldwide.

The impact of central adiposity on pulmonary function remains unclear, particularly beyond the obvious mechanical challenges to breathing conferred by central adiposity. Reductions in chest wall compliance and respiratory muscle strength due to a high percent body fat and localized fat distribution contributes to impaired pulmonary function and the occurrence of adverse respiratory symptoms. Effective weight loss after bariatric surgery may improve cardiovascular disease risk factors, including T2D, hypertension, dyslipidemia, atherosclerosis, inflammation, chronic kidney disease, obstructive sleep apnea, and hypoventilation syndrome. Bariatric surgery has also been associated with significantly improved respiratory symptoms and pulmonary function, and the authors present a review of principal studies that correspond to the reversal of respiratory symptoms and impaired pulmonary function after bariatric surgery.(73) Clearly, there is an element of improvement that is notably linked to weight reduction, which is expected when there is restriction of the chest wall. However, the data do favor some improvements in pulmonary function itself. There is some debate whether lungs of adult humans regenerate(74), but on a logical basis it would be more surprising if they did not, rather than they did. Perhaps the best evidence for pulmonary regeneration comes from patients that have undergone pneumonectomy, usually for a resectable carcinoma of the lung. By way of example, a recent paper by Butler and colleagues reported on a 33-year-old woman who underwent a right-sided pneumonectomy in 1995 for treatment of a lung adenocarcinoma. As expected, there was an abrupt decrease in her vital capacity to approximately half of normal, but unexpectedly, it increased during the subsequent 15 years to reach values similar to normal for age. Serial computed tomographic (CT) scans on this patient showed progressive enlargement of the remaining left lung and an increase in tissue density. Magnetic resonance imaging (MRI) with the use of hyperpolarized helium-3 gas showed overall acinar-airway dimensions that were consistent with an increase in the alveolar number rather than the enlargement of existing alveoli, but the alveoli in the growing lung were shallower than in normal lungs. This study provides evidence that new lung growth can occur in an adult human.(75) On the basis of this demonstrable growth and the improvements after RYGB surgery, It is judged that Brake™ treatment would produce similar regeneration evidence but not necessarily as much weight loss related improvement as RYGB, since Brake™ treated patients do not lose as much weight as RYGB treated patients.

Apart from smoking cessation, there are no other treatments that slow the decline in lung function. Roflumilast and cilomilast are oral phosphodiesterase 4 (PDE-IV) inhibitors proposed to reduce the airway inflammation and bronchoconstriction seen in COPD.

On a cellular level, PDE₄ converts cAMP to adenosine monophosphate (AMP), terminating the cellular messaging initiated by cAMP. Roflumilast blocks the effect of PDE-IV, leading to an accumulation of cAMP within target cells and a corresponding increase in cAMP messaging. The clinical relevance of blocking PDE-IV is unknown. However, it is thought that the accumulation of cAMP within localized immune cells and lung tissue is important in preventing the pathogenesis of COPD, particularly inflammation.

Recently, Chong and colleagues reviewed the efficacy and safety of PDE-IV inhibitors in the management of people with stable COPD. Outcomes included lung function, quality of life, symptoms, exacerbations and adverse effects, in all cases where PDE-IV inhibitors were compared to placebo. Twenty-three separate RCTs studying roflumilast (nine trials, 9211 patients) or cilomilast (fourteen trials, 6457 patients) met the inclusion criteria. None of the trials exceeded a year in duration. Treatment with a PDE-IV inhibitor was associated with a significant improvement in FEV(1) over the trial period compared with placebo (MD 45.59 mL; 95% confidence interval (CI) 39.15 to 52.03), regardless of COPD severity or concomitant COPD treatment. There were some small improvements in quality of life (St George's Respiratory Questionnaire MD −1.04; 95% CI −1.66 to −0.41) and COPD-related symptoms, but no change in exercise tolerance. Treatment with a PDE-IV inhibitor was associated with a reduced likelihood of COPD exacerbation (OR 0.78; 95% CI 0.72 to 0.85). More participants in the treatment groups experienced non-serious adverse events compared with controls, particularly gastrointestinal symptoms and headache. Roflumilast was associated with weight loss during the trial period. In the conclusion of the authors, PDE-IV inhibitors offered benefit over placebo in improving lung function and reducing likelihood of exacerbations, however, they had little impact on quality of life or symptoms. Gastrointestinal adverse effects and weight loss were common. Longer-term trials are needed to determine whether or not PDE-IV inhibitors modify FEV(1) decline, healthcare utilization or mortality in COPD.(76)

Clearly, adding further control of metabolic syndrome to the PDE-IV pathway, such as combining Brake™ with an over-coating of Roflumilast, would be the most promising means of regeneration of pulmonary function. The daily dose of Roflumilast in this case would be the same as typically used, 500 mcg per day,

However, the study of Butler and colleagues show that the combination should be given for longer time periods in order to clearly improve pulmonary function, since the rate of regeneration appears to be slower than short term studies which have been conducted thus far. Although most of the Brake™ combination products have effected maximal regeneration at 6 months, it is not clear at this time if 6 months of the combination product would be sufficient to demonstrate pulmonary regeneration.

### Example 10

### Alzheimer's Disease Biomarkers and Treatments

In recent years a rapidly increasing number of studies have examined the relationship between dementia and metabolic disorders such as T2D, central adiposity, hypertension, and dyslipidemia. Etiological heterogeneity and comorbidity pose challenges for determining relationships among metabolic disorders. The independent and interactive effects of brain vascular injury and classic pathological agents such as beta-amyloid have also proved difficult to distinguish in human patients, blurring the lines between Alzheimer's disease and vascular dementia. Craft and colleagues highlight recent work aimed at identifying convergent mechanisms such as insulin resistance that may underlie comorbid metabolic disorders and thereby increase dementia risk.(77)

Recent studies demonstrate that metabolic syndrome manifestations including central adiposity are independently associated with poor neurocognitive outcomes, including cognitive impairment, increased risk for dementia, and regional alterations in brain structure.(78-89) RYGB surgery is an effective treatment for obesity and initial findings by Stanek and others suggest that it may result in cognitive improvements.(90). Our own findings (1) show marked improvement in biomarkers of Alzheimer's disease following RYGB surgery, effectively positioning the instant invention Brake™ for use concomitantly with known treatments for this condition, such as memantine.

Our recent study of RYGB patients using Alzheimer's biomarkers support the clinical case for improvement in cognition as metabolic syndrome relents. RYGB demonstrates a novel pathway for mitigation of Alzheimer's and we propose that RYGB is impacting cognition by virtue of its impact on underlying metabolic syndrome. RYGB may have other beneficial effects, such as reduction in beta amyloid accumulation in neural tissues. Accordingly we present the evidence linking progression of Alzheimer's disease to progression of metabolic syndrome. Ghanim and Colleagues reported on Alzheimer's biomarkers in patients who had RYGB surgery.(1). Obesity and T2D are known to be associated with an increase in the incidence and prevalence of Alzheimer's disease (AD) and an impaired cognitive function. Because peripheral blood mononuclear cells (MNC) express amyloid precursor protein (APP), the precursor of beta-amyloid, which forms the pathognomonic plaques in the brain, they hypothesized that APP expression diminishes after the marked caloric restriction and reduction in systemic inflammation associated with RYGB surgery. Fifteen T2D patients with morbid obesity (BMI, 52.1+/−13) underwent RYGB, and the expression of inflammatory and AD-related genes was examined before and after 6 months in plasma and in MNC. BMI fell to 40.4+/−11.1 at 6 months after RYGB. There was a significant fall in plasma concentrations of glucose and insulin and in HOMA-IR. The expression of APP mRNA fell by 31+/−9%, and that of protein fell by 36+/−14%. In addition, there was a reduction in the expression of other AD-related genes including presinilin-2, ADAM-9, GSK-3beta, PICALM, SORL-1, and clusterin (P<0.05 for all). Additionally, the expression of c-Fos, a subunit of the pro-inflammatory transcription factor AP-1, was also suppressed after RYGB. These changes occurred in parallel with reductions in other pro-inflammatory mediators including C-reactive protein and monocyte chemoattractant protein-1. Thus, the reversal of the pro-inflammatory state of metabolic syndrome is associated with a concomitant reduction in the expression of APP and other AD-related genes in MNC. If indeed, this effect also occurs in the brain, there are major implications for the pathogenesis and the treatment of AD. It is relevant that cognitive function has been shown to improve with weight loss following RYGB surgery(90).

Based on the unexpected but highly beneficial improvement in biomarkers and cognition after RYGB, it is an additional aspect of the present invention to treat early Alzheimer's disease with a novel combination oral therapy of an Alzheimer's drug and Brake™. Every patient would receive Brake™ treatment that would be demonstrated to be active on the basis of lowered biomarkers of Alzheimer's in a pattern of elevation similar to that observed in our RYGB patients. In combination with oral Brake™ treatment as disclosed herein, the patient would also receive an approved front line treatment for Alzheimer's such as donepezil or memantine, either of these therapeutics given in the usual dose over-coated onto the 7 Brake™ tablets, or in some novel regimens, given at half the usual dose.

There are two tested reasons that Brake™ would improve both the efficacy and safety of donepezil or memantine in Alzheimer's disease. First, both agents have side effects which are dose related, and in both cases using a lower dosage would still improve the efficacy and yet side effects would decrease. Secondly, the control of underlying metabolic syndrome promises true reversal of the Alzheimer's pathophysiology, which is tied to Brake™ associated reversal of insulin resistance, hyperlipidemia, hyperglycemia, hypertension and hepatic steatosis, all of which will be improved or resolved by including Brake™ in the combination therapy of Alzheimer's patients with metabolic syndromes.

Combination therapy between donepezil and Brake™ for the surprising reversal of Alzheimer's disease pathophysiology is hereby incorporated by reference, with dosages of Donepezil of 5-10 mg daily and doses of Brake™ of 10-20 grams daily, both active agents are presented as micro granules for oral administration to patients with Alzheimer's disease. This combination has the surprising potential, when used in conjunction with biomarkers defining early risk of Alzheimer's to prevent the onset of metabolic syndrome associated damage leading to Alzheimer's, or at least inhibit or delay its onset by many years. The disclosed combination product would be the first disease modifying treatment for this disease, here-to-fore considered to be irreversible.

Clinical proof of the utility of the synergistic combination of these Alzheimer's disease therapies including Brake™ would necessitate the adoption of biomarkers of metabolic syndrome progression such as the FS index, which is an overall biomarker profile that can point to regenerative processes that respond to RYGB or Brake™. Added to the metabolic syndrome biomarker profile of the FS index would be a biomarker profile of Alzheimer's disease progression. This latter progression profile would focus on cognition, genomics where applicable, and imaging where applicable to loss of brain tissue and neuronal mass. To the extent that these biomarkers are improved by donepezil, those effects carry forward. To the extent that the observed improvement is tied to effects beyond those of donepezil, the conclusion would be Brake™ associated recovery or regeneration of functioning neurons.

Okereke and colleagues have studied the relationships between dietary factors and cognitive decline. Their study examined dietary fat types in relation to cognitive change in healthy community-based elders. Among 6,183 older participants in the Women's Health Study, they related intake of major fatty acids (saturated [SFA], monounsaturated [MUFA], total polyunsaturated [PUFA], trans-unsaturated) to late-life cognitive trajectory. Serial cognitive testing, conducted over 4 years, began 5 years after initial dietary assessment. Primary outcomes were global cognition and verbal memory. They used analyses of response profiles and logistic regression to estimate multivariate-adjusted differences in cognitive trajectory and risk of worst cognitive change (worst 10%) by fat intake. Higher SFA intake was associated with worse global cognitive (p for linear trend=0.008) and verbal memory (p for linear trend=0.01) trajectories. There was a higher risk of worst cognitive change, comparing highest versus lowest SFA quintiles; the multivariate-adjusted odds ratio (OR) with 95% confidence interval (CI) was 1.64 (1.04-2.58) for global cognition and 1.65 (1.04-2.61) for verbal memory. By contrast, higher MUFA intake was related to better global cognitive (p for linear trend <0.001) and verbal memory (p for linear trend=0.009) trajectories, and lower OR (95% CI) of worst cognitive change in global cognition (0.52 [0.31-0.88]) and verbal memory (0.56 [0.34-0.94]). Total fat, PUFA, and trans-fat intakes were not associated with cognitive trajectory. Thus, higher SFA intake was associated with worse global cognitive and verbal memory trajectories, whereas higher MUFA intake was related to better trajectories (91)

Bayer-Carter and colleagues also examined dietary links to Alzheimer's using similar methods. They compared the effects of a 4-week high-saturated fat/high-glycemic index (HIGH) diet with a low-saturated fat/low-glycemic index (LOW) diet on insulin and lipid metabolism, cerebrospinal fluid (CSF) markers of Alzheimer disease, and cognition for healthy adults and adults with amnestic mild cognitive impairment (aMCI). The study was performed in a clinical research unit. Forty-nine older adults (20 healthy adults with a mean [SD] age of 69.3 [7.4] years and 29 adults with aMCI with a mean [SD] age of 67.6 [6.8] years) received the HIGH diet (fat, 45% [saturated fat, >25%]; carbohydrates, 35%-40% [glycemic index, >70]; and protein, 15%-20%) or the LOW diet (fat, 25%; [saturated fat, <7%]; carbohydrates, 55%-60% [glycemic index, <55]; and protein, 15%-20%) for 4 weeks. Cognitive tests, an oral glucose tolerance test, and lumbar puncture were conducted at baseline and during the fourth week of the diet. CSF concentrations of beta-amyloid (Abeta42 and Abeta40), tau protein, insulin, F2-isoprostanes, and apolipoprotein E, and plasma lipids and insulin, and measures of cognition were all performed. For the aMCI group, the LOW diet increased CSF Abeta42 concentrations, contrary to the pathologic pattern of lowered CSF Abeta42 typically observed in Alzheimer's disease. The LOW diet had the opposite effect for healthy adults, i.e., decreasing CSF Abeta42, whereas the HIGH diet increased CSF Abeta42. The CSF apolipoprotein E concentration was increased by the LOW diet and decreased by the HIGH diet for both groups. For the aMCI group, the CSF insulin concentration increased with the LOW diet, but the HIGH diet lowered the CSF insulin concentration for healthy adults. The HIGH diet increased and the LOW diet decreased plasma lipids, insulin, and CSF F2-isoprostane concentrations. Delayed visual memory improved for both groups after completion of 4 weeks of the LOW diet. These results suggested that diet may be a powerful environmental factor that modulates Alzheimer disease risk through its effects on central nervous system concentrations of Abeta42, lipoproteins, oxidative stress, and insulin.(92)

Patients with Alzheimer's disease (AD) have elevations of fasting plasma insulin that are hypothesized to be associated with disrupted brain insulin metabolism. Craft and colleagues examined paired fasted plasma and CSF insulin levels in 25 patients with AD and 14 healthy age-matched adults and determined whether insulin levels were related to severity of dementia and apolipoprotein E-epsilon-4 homozygosity, a known genetic risk factor for AD. The AD patients had lower CSF insulin, higher plasma insulin, and a reduced CSF-to-plasma insulin ratio when compared with healthy adults. The differences were greater for patients with more advanced AD. Patients who were not apolipoprotein E-epsilon4 homozygotes had higher plasma insulin levels and reduced CSF-to-plasma ratios, whereas epsilon4 homozygotes with AD had normal values. Both plasma and CSF insulin levels are abnormal in AD, and there are metabolic differences among apolipoprotein E genotypes.(93)

Because little is known regarding factors such as insulin and soluble amyloid beta peptide (Abeta) concentrations in humans at late midlife, Townsend et al measured plasma Abeta42, Abeta40, fasting insulin, and c-peptide in 468 women without T2D, aged 59 to 69 years (median 63 y). Before blood draw, participants reported BMI, waist circumference, physical activity, alcohol intake, hypertension, and T2D family history. Linear regression was used to calculate age-adjusted mean differences in Abeta42 to Abeta40 ratio, and Abeta42 levels, by insulin and insulin-related factors. The ratio of Abeta42 to Abeta40 was statistically significantly lower in women with family history of T2D, and Abeta42 was significantly lower with less physical activity, greater waist circumference, hypertension, and family history of T2D (P<0.05 for all). Abeta42 to Abeta40 ratio, and Abeta42 levels, appeared lower with higher c-peptide levels (P trend=0.07 and 0.06, respectively), although these were not statistically significant. In summary, insulin-related factors appear associated with lower plasma Abeta42 to Abeta40 ratio, and Abeta42, at late midlife, consistent with increased brain sequestration of Abeta42 (relative to Abeta40), suggesting insulin merits focus in strategies to prevent dementia. (94)

Scanning is a recognized technology for evaluating progression of Alzheimer's disease. Novak and colleagues examined the effects of inflammation on perfusion regulation and brain volumes in T2D. A total of 147 subjects (71 diabetic and 76 non-diabetic, aged 65.2+/−8 years) were studied using 3T anatomical and continuous arterial spin labeling MRI. Analysis focused on the relationship between serum soluble vascular and intercellular adhesion molecules (sVCAM and sICAM, respectively, both markers of endothelial integrity), regional vasoreactivity, and tissue volumes. T2D subjects had greater vasoconstriction reactivity, more atrophy, depression, and slower walking. Adhesion molecules were specifically related to gray matter atrophy (P=0.04) and altered vasoreactivity (P=0.03) in the diabetic and control groups. Regionally, sVCAM and sICAM were linked to exaggerated vasoconstriction, blunted vasodilatation, and increased cortical atrophy in the frontal, temporal, and parietal lobes (P=0.04-0.003). sICAM correlated with worse functionality. Via MRI, T2D was associated with cortical atrophy, vasoconstriction, and worse performance. Adhesion molecules, as markers of vascular health, contributed to altered vasoregulation and atrophy.(95)

The review article by Sellbom integrates the recent literature regarding patterns of obesity-related cognitive dysfunction and brain alterations and also indicates potential mechanisms for these neuropathological changes. The Sellbom review culminates in a preliminary model of obesity-related cognitive dysfunction and suggestions for future research, including the potential reversibility of these changes with weight-loss. (80) Increasing incidence of metabolic syndrome manifestations including obesity and T2D are already firmly linked to progression of Alzheimer's and there are a large series of biomarkers and mediators summarized below from available literature, and in addition to the summary provided below, they are hereby incorporated into this application by reference.

A third approach to Alzheimer's disease is then enabled by the results with Brake™ in combination with these older front line drugs, and that is the potential for combination between Brake™ and newer molecules that act to reverse Alzheimer's action in the brain itself. One example of this is Bapineuzumab (96-106), which is thought to remove amyloid from brain tissues by blocking the action of the gene coding ApoE4. Combination therapy between Bapineuzumab and Brake™ for the surprising reversal of Alzheimer's disease pathophysiology is hereby incorporated by reference, with effective injected dosages of Bapineuzumab and daily oral doses of Brake™ of 10-20 grams daily, to patients with Alzheimer's disease. This combination has the surprising potential, when used in conjunction with biomarkers defining early risk of Alzheimer's to prevent the brain progression of Alzheimer's disease as well as prevent the onset or progression of metabolic syndrome associated Alzheimer's, or at least delay it by many years. The disclosed combination product would be a new disease modifying treatment for this disease here-to-fore considered to be irreversible.

It is apparent that one skilled in the art, who also had discovered another disease modifying molecule for treatment of Alzheimer's disease, could combine said agent with Brake™ and produce a highly effect and very broad spectrum treatment for Alzheimer's disease, and these combinations are hereby incorporated by reference.

In recent years a rapidly increasing number of studies have examined the relationship between dementia and metabolic disorders such as T2D, hepatic steatosis, hypertension, and dyslipidemia. Etiological heterogeneity and comorbidity pose challenges for determining relationships among metabolic disorders. The independent and interactive effects of brain vascular injury and classic pathological agents such as beta-amyloid have also proved difficult to distinguish in human patients, blurring the lines between Alzheimer's disease and vascular dementia. Craft and colleagues highlight recent work aimed at identifying convergent mechanisms such as insulin resistance that may underlie comorbid metabolic disorders and thereby increase dementia risk.(77)

Bayer-Carter and colleagues also examined dietary links to Alzheimer's using similar methods. They compared the effects of a 4-week high-saturated fat/high-glycemic index (HIGH) diet with a low-saturated fat/low-glycemic index (LOW) diet on insulin and lipid metabolism, cerebrospinal fluid (CSF) markers of Alzheimer disease, and cognition for healthy adults and adults with amnestic mild cognitive impairment (aMCI). The study was performed in a clinical research unit. Forty-nine older adults (20 healthy adults with a mean [SD] age of 69.3 [7.4] years and 29 adults with aMCI with a mean [SD] age of 67.6 [6.8] years) received the HIGH diet (fat, 45% [saturated fat, >25%]; carbohydrates, 35%-40% [glycemic index, >70]; and protein, 15%-20%) or the LOW diet (fat, 25%; [saturated fat, <7%]; carbohydrates, 55%-60% [glycemic index, <55]; and protein, 15%-20%) for 4 weeks. Cognitive tests, an oral glucose tolerance test, and lumbar puncture were conducted at baseline and during the fourth week of the diet. CSF concentrations of beta-amyloid (Abeta42 and Abeta40), tau protein, insulin, F2-isoprostanes, and apolipoprotein E, and plasma lipids and insulin, and measures of cognition were all performed. For the aMCI group, the LOW diet increased CSF Abeta42 concentrations, contrary to the pathologic pattern of lowered CSF Abeta42 typically observed in Alzheimer's disease. The LOW diet had the opposite effect for healthy adults, i.e., decreasing CSF Abeta42, whereas the HIGH diet increased CSF Abeta42. The CSF apolipoprotein E concentration was increased by the LOW diet and decreased by the HIGH diet for both groups. For the aMCI group, the CSF insulin concentration increased with the LOW diet, but the HIGH diet lowered the CSF insulin concentration for healthy adults. The HIGH diet increased and the LOW diet decreased plasma lipids, insulin, and CSF F2-isoprostane concentrations. Delayed visual memory improved for both groups after completion of 4 weeks of the LOW diet. These results suggested that diet may be a powerful environmental factor that modulates Alzheimer disease risk through its effects on central nervous system concentrations of Abeta42, lipoproteins, oxidative stress, and insulin.(92)

Patients with Alzheimer's disease (AD) have elevations of fasting plasma insulin that are hypothesized to be associated with disrupted brain insulin metabolism. Craft and colleagues examined paired fasted plasma and CSF insulin levels in 25 patients with AD and 14 healthy age-matched adults and determined whether insulin levels were related to severity of dementia and apolipoprotein E-epsilon4 homozygosity, a known genetic risk factor for AD. The AD patients had lower CSF insulin, higher plasma insulin, and a reduced CSF-to-plasma insulin ratio when compared with healthy adults. The differences were greater for patients with more advanced AD. Patients who were not apolipoprotein E-epsilon4 homozygotes had higher plasma insulin levels and reduced CSF-to-plasma ratios, whereas epsilon4 homozygotes with AD had normal values. Both plasma and CSF insulin levels are abnormal in AD, and there are metabolic differences among apolipoprotein E genotypes.(93)

Because little is known regarding factors such as insulin and soluble amyloid beta peptide (Abeta) concentrations in humans at late midlife, Townsend et al measured plasma Abeta42, Abeta40, fasting insulin, and c-peptide in 468 women without T2D, aged 59 to 69 years (median 63 y). Before blood draw, participants reported BMI, waist circumference, physical activity, alcohol intake, hypertension, and T2D family history. Linear regression was used to calculate age-adjusted mean differences in Abeta42 to Abeta40 ratio, and Abeta42 levels, by insulin and insulin-related factors. The ratio of Abeta42 to Abeta40 was statistically significantly lower in women with family history of T2D, and Abeta42 was significantly lower with less physical activity, greater waist circumference, hypertension, and family history of T2D (P<0.05 for all). Abeta42 to Abeta40 ratio, and Abeta42 levels, appeared lower with higher c-peptide levels (P trend=0.07 and 0.06, respectively), although these were not statistically significant. In summary, insulin-related factors appear associated with lower plasma Abeta42 to Abeta40 ratio, and Abeta42, at late midlife, consistent with increased brain sequestration of Abeta42 (relative to Abeta40), suggesting insulin merits focus in strategies to prevent dementia.(94)

Scanning is a recognized technology for evaluating progression of Alzheimer's disease. Novak and colleagues examined the effects of inflammation on perfusion regulation and brain volumes in T2D. A total of 147 subjects (71 diabetic and 76 non diabetic, aged 65.2 +/−8 years) were studied using 3T anatomical and continuous arterial spin labeling MRI. Analysis focused on the relationship between serum soluble vascular and intercellular adhesion molecules (sVCAM and sICAM, respectively, both markers of endothelial integrity), regional vasoreactivity, and tissue volumes. T2D subjects had greater vasoconstriction reactivity, more atrophy, depression, and slower walking. Adhesion molecules were specifically related to gray matter atrophy (P=0.04) and altered vasoreactivity (P=0.03) in the diabetic and control groups. Regionally, sVCAM and sICAM were linked to exaggerated vasoconstriction, blunted vasodilatation, and increased cortical atrophy in the frontal, temporal, and parietal lobes (P=0.04-0.003). sICAM correlated with worse functionality. Via MRI, T2D was associated with cortical atrophy, vasoconstriction, and worse performance. Adhesion molecules, as markers of vascular health, contributed to altered vasoregulation and atrophy.(95)

The Sellbom review culminates in a preliminary model of obesity-related cognitive dysfunction and suggestions for future research, including the potential reversibility of these changes with weight-loss. (80) Increasing obesity and T2D are already firmly linked to progression of Alzheimer's and there are a large series of biomarkers and mediators summarized below from available literature, and in addition to the summary provided below, they are hereby incorporated into this application by reference.

Recent studies demonstrate that metabolic syndrome is independently associated with poor neurocognitive outcomes, including cognitive impairment, increased risk for dementia, and regional alterations in brain structure.(78-89) RYGB surgery is an effective treatment for metabolic syndrome and initial findings by Stanek and others suggest that it may result in cognitive improvements.(90).

Based on the unexpected but highly beneficial improvement in biomarkers and cognition after RYGB, it is a further aspect of the invention to treat early Alzheimer's disease with a novel combination oral therapy of an Alzheimer's drug and Brake™. Every patient would receive Brake™ treatment that would be demonstrated to be active on the basis of lowered biomarkers of Alzheimer's in a pattern of elevation similar to that observed in our RYGB patients. In combination with oral Brake™ treatment as disclosed herein, the patient would also receive an approved front line treatment for Alzheimer's such as donepezil or memantine, either of these therapeutics given in the usual dose or in some novel regimens, given at 50% to 80% or even less (e.g. 20% to 35%) of the usual dose. There are two tested reasons that Brake™ would improve both the efficacy and safety of donepezil or memantine in Alzheimer's disease. First, both agents have side effects which are dose related, and in both cases using a lower dosage would still improve the efficacy and yet side effects would decrease. Secondly, the control of underlying metabolic syndrome promises true reversal of the Alzheimer's pathophysiology, which is tied to Brake™ associated reversal of insulin resistance, hyperlipidemia, hyperglycemia, hypertension and hepatic steatosis, all of which will be improved or resolved by including Brake™ in the combination therapy of Alzheimer's patients with metabolic syndromes.

Combination therapy between donepezil and Brake™ for the surprising reversal of Alzheimer's disease pathophysiology is hereby incorporated by reference, with dosages of Donepezil of 5-10 mg daily and doses of Brake™ of 10-20 grams daily, both active agents are presented as micro granules for oral administration to patients with Alzheimer's disease. This combination has the surprising potential, when used in conjunction with biomarkers defining early risk of Alzheimer's to prevent the onset of metabolic syndrome associated damage leading to Alzheimer's, or at least delay its onset by many years. The disclosed combination product would be the first disease modifying treatment for this disease, here-to-fore considered to be irreversible.

Clinical proof of the utility of the synergistic combination of these Alzheimer's disease therapies including Brake™ would necessitate the adoption of biomarkers of metabolic syndrome progression such as the FS index, which is an overall biomarker profile that can point to regenerative processes that respond to RYGB or Brake™. Added to the metabolic syndrome biomarker profile of the FS index would be a biomarker profile of Alzheimer's disease progression. This latter progression profile would focus on cognition, genomics where applicable, and imaging where applicable to loss of brain tissue and neuronal mass (apoptosis). To the extent that these biomarkers are improved by donepezil, those effects carry forward. To the extent that the observed improvement is tied to effects beyond those of donepezil, the conclusion would be Brake™ associated recovery or regeneration of functioning neurons.

It is apparent that one skilled in the art who also had discovered another disease modifying molecule for treatment of Alzheimer's (an anti-Alzheimer's agent) could combine said agent with Brake™ and produce a highly effect and very broad spectrum treatment for Alzheimer's disease, and these combinations are another aspect of the present invention.

### REFERENCES AND LITERATURE CITED

1. Ghanim H, Monte S V, Sia C L, Abuaysheh S, Green K, Caruana J A, et al. Reduction in Inflammation and the Expression of Amyloid Precursor Protein and Other Proteins Related to Alzheimer's Disease following Gastric Bypass Surgery. J Clin Endocrinol Metab. 2012; 97 (7):E1197-201.
2. Monte S V, Schentag J J, Adelman M H, Paladino J A. Characterization of cardiovascular outcomes in a type 2 diabetes glucose supply and insulin demand model. J Diabetes Sci Technol. 2010; 4(2):382-90.
3. Monte S V, Schentag J J, Adelman M H, Paladino J A. Glucose supply and insulin demand dynamics of antidiabetic agents. J Diabetes Sci Technol. 2010; 4(2):365-81.
4. Adams R J, Appleton S, Wilson D H, Taylor A W, Dal Grande E, Chittleborough C, et al. Population comparison of two clinical approaches to the metabolic syndrome: implications of the new International Diabetes Federation consensus definition. Diabetes Care. 2005; 28(11):2777-9.
5. Aguilar-Salinas Calif., Rojas R, Gomez-Perez F J, Valles V, Rios-Torres J M, Franco A, et al. Analysis of the agreement between the World Health Organization criteria and the National Cholesterol Education Program-III definition of the metabolic syndrome: results from a population-based survey. Diabetes Care. 2003; 26(5):1635.
6. Alberti K G, Zimmet P, Shaw J. The metabolic syndrome—a new worldwide definition. Lancet. 2005; 366(9491):1059-62.
7. Assmann G, Guerra R, Fox G, Cullen P, Schulte H, Willett D, et al. Harmonizing the definition of the metabolic syndrome: comparison of the criteria of the Adult Treatment Panel III and the International Diabetes Federation in United States American and European populations. Am J Cardiol. 2007; 99(4):541-8.
8. Chen H J, Pan W H. Probable blind spot in the International Diabetes Federation definition of metabolic syndrome. Obesity (Silver Spring). 2007; 15(5):1096-100.
9. de Simone G, Devereux R B, Chinali M, Best L G, Lee E T, Galloway J M, et al. Prognostic impact of metabolic syndrome by different definitions in a population with high prevalence of obesity and diabetes: the Strong Heart Study. Diabetes Care. 2007; 30(7):1851-6.
10. Demacker P N. The metabolic syndrome: definition, pathogenesis and therapy. Eur J Clin Invest. 2007; 37(2):85-9.
11. Grundy S M, Brewer H B, Jr., Cleeman J I, Smith S C, Jr., Lenfant C. Definition of metabolic syndrome: Report of the National Heart, Lung, and Blood Institute/American Heart Association conference on scientific issues related to definition. Circulation. 2004; 109(3):433-8.
12. Heng D, Ma S, Lee J J, Tai B C, Mak K H, Hughes K, et al. Modification of the N CEP ATP III definitions of the metabolic syndrome for use in Asians identifies individuals at risk of ischemic heart disease. Atherosclerosis. 2006; 186(2):367-73.
13. Jorgensen M E, Borch-Johnsen K. The metabolic syndrome—is one global definition possible? Diabet Med. 2004; 21(10):1064-5.
14. Lorenzo C, Williams K, Hunt K J, Haffner S M. The National Cholesterol Education Program-Adult Treatment Panel III, International Diabetes Federation, and World Health Organization definitions of the metabolic syndrome as predictors of incident cardiovascular disease and diabetes. Diabetes Care. 2007; 30(1):8-13.
15. Onat A, Uyarel H, Hergenc G, Karabulut A, Albayrak S, Can G. Determinants and definition of abdominal obesity as related to risk of diabetes, metabolic syndrome and coronary disease in Turkish men: a prospective cohort study. Atherosclerosis. 2007; 191(1):182-90.
16. Sandhofer A, Iglseder B, Paulweber B, Ebenbichler C F, Patsch J R. Comparison of different definitions of the metabolic syndrome. Eur J Clin Invest. 2007; 37(2):109-16.
17. Zimmet P, Magliano D, Matsuzawa Y, Alberti G, Shaw J. The metabolic syndrome: a global public health problem and a new definition. J Atheroscler Thromb. 2005; 12(6):295-300.
18. Rissanen A, Heliovaara M, Knekt P, Reunanen A, Aromaa A, Maatela J. Risk of disability and mortality due to overweight in a Finnish population. Bmj. 1990; 301(6756):835-7.
19. Furet J P, Kong L C, Tap J, Poitou C, Basdevant A, Bouillot J L, et al. Differential adaptation of human gut microbiota to bariatric surgery-induced weight loss: links with metabolic and low-grade inflammation markers. Diabetes. 2010; 59(12):3049-57.
20. Zhang H, DiBaise J K, Zuccolo A, Kudrna D, Braidotti M, Yu Y, et al. Human gut microbiota in obesity and after gastric bypass. Proc Natl Acad Sci USA. 2009; 106(7):2365-70.
21. Cani P D, Bibiloni R, Knauf C, Waget A, Neyrinck A M, Delzenne N M, et al. Changes in gut microbiota control metabolic endotoxemia-induced inflammation in high-fat diet-induced obesity and diabetes in mice. Diabetes. 2008; 57(6):1470-81.
22. Cani P D, Delzenne N M. The role of the gut microbiota in energy metabolism and metabolic disease. Curr Pharm Des. 2009; 15(13):1546-58.
23. Cani P D, Osto M, Geurts L, Everard A. Involvement of gut microbiota in the development of low-grade inflammation and type 2 diabetes associated with obesity. Gut Microbes. 2012; 3 (4).
24. Cani P D, Possemiers S, Van de Wiele T, Guiot Y, Everard A, Rottier 0, et al. Changes in gut microbiota control inflammation in obese mice through a mechanism involving GLP-2-driven improvement of gut permeability. Gut. 2009; 58(8):1091-103.
25. Cani P D, Amar J, Iglesias M A, Poggi M, Knauf C, Bastelica D, et al. Metabolic endotoxemia initiates obesity and insulin resistance. Diabetes. 2007; 56(7):1761-72.
26. Cani P D, Delzenne N M. Involvement of the gut microbiota in the development of low grade inflammation associated with obesity: focus on this neglected partner. Acta Gastroenterol Belg. 2010; 73(2):267-9.
27. Ansarullah, Bharucha B, Umarani M, Dwivedi M, Laddha N C, Begum R, et al. Oreocnide integrifolia Flavonoids Augment Reprogramming for Islet Neogenesis and beta-Cell Regeneration in Pancreatectomized BALB/c Mice. Evid Based Complement Alternat Med. 2012; 2012:260467.
28. Wang Y, Wang H, Liu Y, Li C, Qi P, Bao J. Antihyperglycemic effect of ginsenoside Rh2 by inducing islet beta-cell regeneration in mice. Horm Metab Res. 2012; 44(1):33-40.
29. Oh Y S, Shin S, Lee Y J, Kim E H, Jun H S. Betacellulin-induced beta cell proliferation and regeneration is mediated by activation of ErbB-1 and ErbB-2 receptors. PLoS One. 2011; 6 (8):e23894.
30. Brand S J, Tagerud S, Lambert P, Magil S G, Tatarkiewicz K, Doiron K, et al. Pharmacological treatment of chronic diabetes by stimulating pancreatic beta-cell regeneration with systemic co-administration of EGF and gastrin. Pharmacol Toxicol. 2002; 91(6):414-20.
31. Suarez-Pinzon W L, Rabinovitch A. Combination therapy with a dipeptidyl peptidase-4 inhibitor and a proton pump inhibitor induces beta-cell neogenesis from adult human pancreatic duct cells implanted in immunodeficient mice. Cell Transplant. 2011; 20(9):1343-9.
32. Estil Les E, Tellez N, Escoriza J, Montanya E. Increased beta cell replication, and beta cell mass regeneration in syngeneically transplanted rat islets overexpressing insulin-like growth factor-II. Cell Transplant. 2012.
33. Meier J J, Lin J C, Butler A E, Galasso R, Martinez D S, Butler P C. Direct evidence of attempted beta cell regeneration in an 89-year-old patient with recent-onset type 1 diabetes. Diabetologia. 2006; 49(8):1838-44.
34. Hasegawa Y, Ogihara T, Yamada T, Ishigaki Y, Imai J, Uno K, et al. Bone marrow (BM) transplantation promotes beta-cell regeneration after acute injury through BM cell mobilization. Endocrinology. 2007; 148(5):2006-15.
35. Trucco M. Is facilitating pancreatic beta cell regeneration a valid option for clinical therapy? Cell Transplant. 2006; 15 Suppl 1:S75-84.
36. Meier J J, Bhushan A, Butler A E, Rizza R A, Butler P C. Sustained beta cell apoptosis in patients with long-standing type 1 diabetes: indirect evidence for islet regeneration? Diabetologia. 2005; 48(11):2221-8.
37. Dunker N. Transforming growth factor beta mediated programmed cell death in tissue regeneration of the murine intestinal mucosa. Ann Anat. 2003; 185(4):299-300.
38. Hoppener J W, Oosterwijk C, Nieuwenhuis M G, Posthuma G, Thijssen J H, Vroom T M, et al. Extensive islet amyloid formation is induced by development of Type II diabetes mellitus and contributes to its progression: pathogenesis of diabetes in a mouse model. Diabetologia. 1999; 42(4):427-34.
39. Grunfeld C, Feingold K R. Endotoxin in the gut and chylomicrons: translocation or transportation? J Lipid Res. 2009; 50(1):1-2.
40. Ghoshal S, Witta J, Zhong J, de Villiers W, Eckhardt E. Chylomicrons promote intestinal absorption of lipopolysaccharides. J Lipid Res. 2009; 50(1):90-7.
41. Erridge C, Attina T, Spickett C M, Webb D J. A high-fat meal induces low-grade endotoxemia: evidence of a novel mechanism of postprandial inflammation. Am J Clin Nutr. 2007; 86(5):1286-92.
42. Creely S J, McTernan P G, Kusminski C M, Fisher M, Da Silva N F, Khanolkar M, et al. Lipopolysaccharide activates an innate immune system response in human adipose tissue in obesity and type 2 diabetes. Am J Physiol Endocrinol Metab. 2007; 292 (3):E740-7.
43. Monte S V, Caruana J A, Ghanim H, Sia C L, Korzeniewski K, Schentag J J, et al. Reduction in endotoxemia, oxidative and inflammatory stress, and insulin resistance after Roux-en-Y gastric bypass surgery in patients with morbid obesity and type 2 diabetes mellitus. Surgery. 2011.
44. Henao-Mejia J, Elinav E, Jin C, Hao L, Mehal W Z, Strowig T, et al. Inflammasome-mediated dysbiosis regulates progression of NAFLD and obesity. Nature. 2012; 482(7384):179-85.
45. Strowig T, Henao-Mejia J, Elinav E, Flavell R. Inflammasomes in health and disease. Nature. 2012; 481(7381):278-86.
46. Henao-Mejia J, Elinav E, Strowig T, Flavell R A. Inflammasomes: far beyond inflammation. Nat Immunol. 2012; 13(4):321-4.
47. Amar J, Burcelin R, Ruidavets J B, Cani P D, Fauvel J, Alessi M C, et al. Energy intake is associated with endotoxemia in apparently healthy men. Am J Clin Nutr. 2008; 87(5):1219-23.
48. Geurts L, Lazarevic V, Derrien M, Everard A, Van Roye M, Knauf C, et al. Altered gut microbiota and endocannabinoid system tone in obese and diabetic leptin-resistant mice: impact on apelin regulation in adipose tissue. Front Microbiol. 2011; 2:149.
49. Caesar R, Reigstad C S, Backhed H K, Reinhardt C, Ketonen M, Ostergren Lunden G, et al. Gut-derived lipopolysaccharide augments adipose macrophage accumulation but is not essential for impaired glucose or insulin tolerance in mice. Gut. 2012.
50. Yu Y, Ohmori K, Chen Y, Sato C, Kiyomoto H, Shinomiya K, et al. Effects of pravastatin on progression of glucose intolerance and cardiovascular remodeling in a type II diabetes model. J Am Coll Cardiol. 2004; 44(4):904-13.
51. Khovidhunkit W, Kim M S, Memon R A, Shigenaga J K, Moser A H, Feingold K R, et al. Effects of infection and inflammation on lipid and lipoprotein metabolism: mechanisms and consequences to the host. J Lipid Res. 2004; 45(7):1169-96.
52. Giannini C, de Giorgis T, Scarinci A, Cataldo I, Marcovecchio M L, Chiarelli F, et al. Increased carotid intima-media thickness in pre-pubertal children with constitutional leanness and severe obesity: the speculative role of insulin sensitivity, oxidant status, and chronic inflammation. Eur J Endocrinol. 2009; 161(1):73-80.
53. Shai I, Spence J D, Schwarzfuchs D, Henkin Y, Parraga G, Rudich A, et al. Dietary intervention to reverse carotid atherosclerosis. Circulation. 2010; 121(10):1200-8.
54. Yamagishi S, Matsui T, Ueda S, Nakamura K, Imaizumi T. Advanced glycation end products (AGEs) and cardiovascular disease (CVD) in diabetes. Cardiovasc Hematol Agents Med Chem. 2007; 5(3):236-40.
55. Illan-Gomez F, Gonzalvez-Ortega M, Orea-Soler I, Alcaraz-Tafalla M S, Aragon-Alonso A, Pascual-Diaz M, et al. Obesity and inflammation: change in adiponectin, C-reactive protein, tumour necrosis factor-alpha and interleukin-6 after bariatric surgery. Obes Surg. 2012; 22(6):950-5.
56. Owan T, Avelar E, Morley K, Jiji R, Hall N, Krezowski J, et al. Favorable changes in cardiac geometry and function following gastric bypass surgery: 2-year follow-up in the Utah obesity study. J Am Coll Cardiol. 2011; 57(6):732-9.
57. Vazzana N, Santilli F, Sestili S, Cuccurullo C, Davi G. Determinants of increased cardiovascular disease in obesity and metabolic syndrome. Curr Med Chem. 2011; 18(34):5267-80.
58. Benraouane F, Litwin S E. Reductions in cardiovascular risk after bariatric surgery. Curr Opin Cardiol. 2011; 26(6):555-61.
59. Best J H, Hoogwerf B J, Herman W H, Pelletier E M, Smith D B, Wenten M, et al. Risk of cardiovascular disease events in patients with type 2 diabetes prescribed the glucagon-like peptide 1 (GLP-1) receptor agonist exenatide twice daily or other glucose-lowering therapies: a retrospective analysis of the LifeLink database. Diabetes. Care. 2011; 34(1):90-5.
60. Bocan T M, Mueller S B, Uhlendorf P D, Newton R S, Krause B R. Comparison of CI-976, an A CAT inhibitor, and selected lipid-lowering agents for antiatherosclerotic activity in iliac-femoral and thoracic aortic lesions. A biochemical, morphological, and morphometric evaluation. Arterioscler Thromb. 1991; 11(6):1830-43.
61. Parolini C, Marchesi M, Lorenzon P, Castano M, Balconi E, Miragoli L, et al. Dose-related effects of repeated ETC-216 (recombinant apolipoprotein A-I Milano/1-palmitoyl-2-oleoyl phosphatidylcholine complexes) administrations on rabbit lipid-rich soft plaques: in vivo assessment by intravascular ultrasound and magnetic resonance imaging. J Am Coll Cardiol. 2008; 51(11):1098-103.
62. D'Adamo E, Marcovecchio M L, Giannini C, Capanna R, Impicciatore M, Chiarelli F, et al. The possible role of liver steatosis in defining metabolic syndrome in prepubertal children. Metabolism. 2010; 59(5):671-6.
63. Koehler J A, Harper W, Barnard M, Yusta B, Drucker D J. Glucagon-like peptide-2 does not modify the growth or survival of murine or human intestinal tumor cells. Cancer Res. 2008; 68(19):7897-904.
64. Drucker D J. Biologic actions and therapeutic potential of the proglucagon-derived peptides. Nat Clin Pract Endocrinol Metab. 2005; 1(1):22-31.
65. Sinclair E M, Drucker D J. Proglucagon-derived peptides: mechanisms of action and therapeutic potential. Physiology (Bethesda). 2005; 20:357-65.
66. Brubaker P L, Drucker D J. Minireview: Glucagon-like peptides regulate cell proliferation and apoptosis in the pancreas, gut, and central nervous system. Endocrinology. 2004; 145(6):2653-9.
67. Estall J L, Drucker D J. Dual regulation of cell proliferation and survival via activation of glucagon-like peptide-2 receptor signaling. J Nutr. 2003; 133(11):3708-11.
68. Shin E D, Estall J L, Izzo A, Drucker D J, Brubaker P L. Mucosal adaptation to enteral nutrients is dependent on the physiologic actions of glucagon-like peptide-2 in mice. Gastroenterology. 2005; 128(5):1340-53.
69. le Roux C W, Borg C, Wallis K, Vincent R P, Bueter M, Goodlad R, et al. Gut hypertrophy after gastric bypass is associated with increased glucagon-like peptide 2 and intestinal crypt cell proliferation. Ann Surg. 2010; 252(1):50-6.
70. Weir G C, Bonner-Weir S. Dreams for type 1 diabetes: shutting off autoimmunity and stimulating beta-cell regeneration. Endocrinology. 2010; 151(7):2971-3.
71. Bastien-Dionne P O, Valenti L, Kon N, Gu W, Buteau J. Glucagon-like peptide 1 inhibits the sirtuin deacetylase SirT1 to stimulate pancreatic beta-cell mass expansion. Diabetes. 2011; 60(12):3217-22.
72. Westlake S L, Colebatch A N, Baird J, Kiely P, Quinn M, Choy E, et al. The effect of methotrexate on cardiovascular disease in patients with rheumatoid arthritis: a systematic literature review. Rheumatology (Oxford). 2010; 49(2):295-307.
73. Wei Y F, Wu H D. Candidates for bariatric surgery: morbidly obese patients with pulmonary dysfunction. J Obes. 2012; 2012:878371.
74. Mechanisms and limits of induced postnatal lung growth. Am J Respir Crit Care Med. 2004; 170(3):319-43.
75. Butler J P, Loring S H, Patz S, Tsuda A, Yablonskiy D A, Mentzer S J. Evidence for adult lung growth in humans. N Engl J Med. 2012; 367(3):244-7.
76. Chong J, Poole P, Leung B, Black P N. Phosphodiesterase 4 inhibitors for chronic obstructive pulmonary disease. Cochrane Database Syst Rev. 2011 (5):CD002309.
77. Craft S. The role of metabolic disorders in Alzheimer disease and vascular dementia: two roads converged. Arch Neurol. 2009; 66(3):300-5.
78. Alosco M L, Spitznagel M B, van Dulmen M, Raz N, Cohen R, Sweet L H, et al. The additive effects of type-2 diabetes on cognitive function in older adults with heart failure. Cardiol Res Pract. 2012; 2012:348054.
79. Miller L A, Spitznagel M B, Alosco M L, Cohen R A, Raz N, Sweet L H, et al. Cognitive profiles in heart failure: a cluster analytic approach. J Clin Exp Neuropsychol. 2012; 34(5):509-20.
80. Sellbom K S, Gunstad J. Cognitive function and decline in obesity. J Alzheimers Dis. 2012; 30 (0):S89-95.
81. Spitznagel M B, Garcia S, Miller L A, Strain G, Devlin M, Wing R, et al. Cognitive function predicts weight loss after bariatric surgery. Surg Obes Relat Dis. 2011.
82. Galioto R, Spitznagel M B, Strain G, Devlin M, Cohen R, Paul R, et al. Cognitive function in morbidly obese individuals with and without binge eating disorder. Compr Psychiatry. 2012; 53(5):490-5.
83. Bellar D, Glickman E L, Juvancic-Heltzel J, Gunstad J. Serum insulin like growth factor-1 is associated with working memory, executive function and selective attention in a sample of healthy, fit older adults. Neuroscience. 2011; 178:133-7.
84. Miller L A, Spitznagel M B, Busko S, Potter V, Juvancic-Heltzel J, Istenes N, et al. Structured exercise does not stabilize cognitive function in individuals with mild cognitive impairment residing in a structured living facility. Int J Neurosci. 2011; 121(4):218-23.
85. Stanek K M, Grieve S M, Brickman A M, Korgaonkar M S, Paul R H, Cohen R A, et al. Obesity is associated with reduced white matter integrity in otherwise healthy adults. Obesity (Silver Spring). 2011; 19(3):500-4.
86. Gunstad J, Lhotsky A, Wendell C R, Ferrucci L, Zonderman A B. Longitudinal examination of obesity and cognitive function: results from the Baltimore longitudinal study of aging. Neuroepidemiology. 2010; 34(4):222-9.
87. Gunstad J, Keary T A, Spitznagel M B, Poppas A, Paul R H, Sweet L H, et al. Blood pressure and cognitive function in older adults with cardiovascular disease. Int J Neurosci. 2009; 119(12):2228-42.
88. Lokken K L, Boeka A G, Austin H M, Gunstad J, Harmon C M. Evidence of executive dysfunction in extremely obese adolescents: a pilot study. Surg Obes Relat Dis. 2009; 5(5):547-52.
89. Luyster F S, Hughes J W, Gunstad J. Depression and anxiety symptoms are associated with reduced dietary adherence in heart failure patients treated with an implantable cardioverter defibrillator. J Cardiovasc Nurs. 2009; 24(1):10-7.
90. Stanek K M, Gunstad J. Can bariatric surgery reduce risk of Alzheimer's disease? Prog Neuropsychopharmacol Biol Psychiatry. 2012.
91. Okereke O I, Rosner B A, Kim D H, Kang J H, Cook N R, Manson J E, et al. Dietary fat types and 4-year cognitive change in community-dwelling older women. Ann Neurol. 2012; 72(1):124-34.
92. Bayer-Carter J L, Green P S, Montine T J, VanFossen B, Baker L D, Watson G S, et al. Diet intervention and cerebrospinal fluid biomarkers in amnestic mild cognitive impairment. Arch Neurol. 2011; 68(6):743-52.
93. Craft S, Peskind E, Schwartz M W, Schellenberg G D, Raskind M, Porte D, Jr. Cerebrospinal fluid and plasma insulin levels in Alzheimer's disease: relationship to severity of dementia and apolipoprotein E genotype. Neurology. 1998; 50(1):164-8.
94. Townsend M K, Okereke O I, Xia W, Yang T, Selkoe D J, Grodstein F. Relation between insulin, insulin-related factors, and plasma amyloid beta peptide levels at midlife in a population-based study. Alzheimer Dis Assoc Disord. 2012; 26(1):50-4.
95. Novak V, Zhao P, Manor B, Sejdic E, Alsop D, Abduljalil A, et al. Adhesion molecules, altered vasoreactivity, and brain atrophy in type 2 diabetes. Diabetes Care. 2011; 34(11):2438-41.
96. Blennow K, Zetterberg H, Rinne J O, Salloway S, Wei J, Black R, et al. Effect of Immunotherapy With Bapineuzumab on Cerebrospinal Fluid Biomarker Levels in Patients With Mild to Moderate Alzheimer Disease. Arch Neurol. 2012.
97. Sperling R, Salloway S, Brooks D J, Tampieri D, Barakos J, Fox N C, et al. Amyloid-related imaging abnormalities in patients with Alzheimer's disease treated with bapineuzumab: a retrospective analysis. Lancet Neurol. 2012; 11(3):241-9.
98. Panza F, Frisardi V, Imbimbo B P, Seripa D, Paris F, Santamato A, et al. Anti-beta-amyloid immunotherapy for Alzheimer's disease: focus on bapineuzumab. Curr Alzheimer Res. 2011; 8(8):808-17.
99. Roher A E, Maarouf C L, Daugs I D, Kokjohn T A, Hunter J M, Sabbagh M N, et al. Neuropathology and amyloid-beta spectrum in a bapineuzumab immunotherapy recipient. J Alzheimers Dis. 2011; 24(2):315-25.
100. Panza F, Frisardi V, Imbimbo B P, D'Onofrio G, Pietrarossa G, Seripa D, et al. Bapineuzumab: anti-beta-amyloid monoclonal antibodies for the treatment of Alzheimer's disease. Immunotherapy. 2010; 2(6):767-82.
101. Laskowitz D T, Kolls B J. A phase 2 multiple ascending dose trial of bapineuzumab in mild to moderate Alzheimer disease. Neurology. 2010; 74(24):2026; author reply-7.
102. Black R S, Sperling R A, Safirstein B, Motter R N, Pallay A, Nichols A, et al. A single ascending dose study of bapineuzumab in patients with Alzheimer disease. Alzheimer Dis Assoc Disord. 2010; 24(2):198-203.
103. Kerchner G A, Boxer A L. Bapineuzumab. Expert Opin Biol Ther. 2010; 10(7):1121-30.
104. Wilcock G K. Bapineuzumab in Alzheimer's disease: where now? Lancet Neurol. 2010; 9(2):134-6.
105. Salloway S, Sperling R, Gilman S, Fox N C, Blennow K, Raskind M, et al. A phase 2 multiple ascending dose trial of bapineuzumab in mild to moderate Alzheimer disease. Neurology. 2009; 73(24):2061-70.
106. Kaufer D, Gandy S. APOE {epsilon}4 and bapineuzumab: Infusing pharmacogenomics into Alzheimer disease therapeutics. Neurology. 2009; 73(24):2052-3.

1.-126. (canceled)

127. A method of inhibiting damage to organs and tissues or regenerating and/or remodeling organs and tissues in a subject suffering from one or more organ or tissue manifestations caused by glucose supply side associated metabolic syndrome, the method comprising:
(a) confirming that the subject suffers from or is at risk for suffering from organ and/or tissue damage associated with a glucose supply side associated metabolic syndrome; and
(b) co-administering to the subject an effective amount of a pharmaceutical composition in oral dosage form comprising a first and optionally a second active composition, said first active composition comprising an ileal brake hormone releasing substance at least 50% by weight of which is released within said subject's ileum and ascending colon causing release of ileal brake hormones from L-cells of said subject after administration, said optional second active composition being formulated in immediate and/or early release form in an over coating onto said enteric coating, wherein said second composition is beneficial to at least one aspect of said subject's metabolic syndrome manifestations.

128. The method according to claim 127, wherein said organ or tissue manifestations of said metabolic syndrome associated disease may include one or more of pancreatic beta cell damage, cardiovascular diseases such as myocardial infarction, stroke, angina, congestive heart failure, hypertension, ASCVD, diabetic nephropathy leading to kidney failure, atherosclerosis, obesity, hepatic steatosis, NASH, NAFLD, hyperlipidemia, elevated triglycerides, abdominal adiposity, reduced lung capacity (COPD), Rheumatoid arthritis, gastrointestinal tract damage, gastrointestinal dysbiosis, inflammatory bowel disease, neurodegenerative disorders, diabetic neuropathy, Alzheimer's disease, cognitive impairment associated with obesity and early Alzheimer's disease.

129. The method according to claim 127, wherein the first active composition comprises dextrose in an effective amount, and optionally a plant-derived lipid.

130. The method according to claim 127, wherein the second active composition is absent from the ileal brake hormone releasing composition.

131. The method according to claim 127 wherein said subject has non-alcoholic fatty liver disease (NAFLD) and/or non-alcoholic steatohepatitis (NASH) and/or liver fibrosis alone or in combination with a Hepatitis C (HCV) infection and said method regenerates, remodels and/or inhibits further damage to the subject's liver, said composition releasing at least 50% by weight of said ileal brake hormone releasing substance in the ileum of said subject.

132. The treatment method of claim 131 wherein said second active composition comprises at least one compound selected from the group consisting of ribavirin, boceprevir, daclatasvir, asunaprevir, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, sofosbuvir, PSI-7851, SCY-635, TLR9 Agonist, PHX1766, SP-30 and mixtures thereof, wherein said second composition in an effective amount is over coated onto said ileal brake hormone releasing component in said pharmaceutical composition.

133. The method of claim 127 wherein said subject in need of said treatment has insulin resistance and Type 2 Diabetes Mellitus, and said subject's pancreas is effectively regenerated, remodeled or further damage is inhibited by the administration of said pharmaceutical composition.

134. The method according to claim 133 wherein said pharmaceutical composition comprises an effective amount of at least one second active composition selected from the group consisting of metformin, a DPP-IV inhibitor, an insulin sensitizer, a thiazolidinedione, a PPAR modulator, a PPAR-sparing medicament, an alpha glucosidase inhibitor, a colesevelam mimetic agent, a HMG-CoA reductase inhibitor, an angiotensin II inhibitor, a PDE-5 inhibitor, said second active composition(s) being formulated in an over coating of said ileal brake hormone releasing composition.

135. The method of claim 127 wherein said subject in need has Alzheimer's disease, and wherein said method regenerates, remodels or inhibits further damage of said subject's brain neuronal tissue.

136. The method of claim 135 wherein said second active composition is at least one composition selected from the group consisting of memantine, donepezil, a tau inhibiting substance, an amyloid inhibiting substance, a reversible acetylcholinesterase inhibitor, an NMDA regulator, an inhibitor of beta amyloid protein formation, an Angiotensin II inhibitor, a GLP-1 pathway mimetic, a short acting corticosteroid and mixtures thereof, said second active composition(s) being formulated in an overcoating of said ileal brake hormone releasing composition.

137. The method of claim 127, wherein said confirming step is evidenced in said subject by the existence of metabolic syndrome and insulin resistance as determined by an elevated HOMA-IR measurement and optionally, a diagnosis of prediabetes, type 2 diabetes, NAFLD, NASH or elevated triglycerides.

138. The method according to claim 127, wherein said pharmaceutical composition has an enteric coating comprising at least one component selected from the group consisting of cellulose acetate trimellitiate (CAT), hydroxypropylmethyl cellulose phthalate (HPMCP), hydroxypropylmethyl cellulose, ethyl cellulose and mixtures of hydroxypropylmethyl cellulose and ethyl cellulose, polyvinyl acetate phthalate (PVAP), cellulose acetate phthalate (CAP), shellac, copolymers of methacrylic acid and ethyl acrylate, copolymers of methacrylic acid and ethyl acrylate to which a monomer of methylacrylate has been added during polymerization, and mixtures thereof.

139. The method according to claim 138, wherein said enteric coating comprises one or more compositions selected from the group consisting of shellac, Eudragit® L, Eudragit® S, Eudragit® RL, Eudragit® RS and mixtures thereof.

140. A pharmaceutical composition in unit oral dosage form comprising a first composition and a second composition, said first composition comprising a daily dose of between about 5 grams to about 20 grams of an ileal brake hormone releasing agent which is encapsulated within an enteric coating which dissolves in vivo at a pH of around 7.2 to around 7.5 and releases said first composition within said subject's ileum and ascending colon causing release of at least one ileal brake hormone from L-cells of said subject, said second active composition being formulated in said pharmaceutical composition in immediate and/or early release form in an over coating on said enteric coating, wherein said second composition works in concert with said first composition to treat a subject's metabolic syndrome manifestations.

141. The composition according to claim 140 wherein said released ileal brake hormone is at least one hormone selected from the group consisting of GLP-1, glicentin, oxyntomodulin or a peptide fragment thereof, C-terminally glycine-extended GLP-1 (7 37) intervening peptide-2, GLP-2, GRPP, PYY 1-36, PYY 3-36, enteroglucagon and neurotensin.

142. The method of claim 127 wherein said confirming step evidences one or more of a FS index of at least about 60 in said patient, a GLP-1 concentration below 20 in said patient, a HOMA-IR of about 4.0 in said patient or an ileum pH of around 7.2 to around 7.5 in said patient.

143. The method of claim 127, wherein subsequent to administration of said pharmaceutical composition to the subject results in the subject's FS index to fall to below 50 and/or the subject's AUC of GLP-1 expression is increased by between 50% and 90% compared to pre-treatment levels.

144. The method of claim 127, whereby said patient treated with said ileal brake hormone releasing substance achieves said threshold AUC of ileal brake hormone outputs of GLP-1 of approximately 250 and said threshold AUC of ileal brake hormone outputs of PYY of approximately 350, wherein said second composition is beneficial to at least one aspect of said subject's metabolic syndrome manifestations.

145. The method of claim 127 wherein said second active composition comprises metformin, sitagliptin, saxagliptin, methotrexate, olanzapine, donepezil, memantine, risperidone, ziprasidone, colesevelam or a mixture thereof.

146. The method of claim 127 wherein said second active composition comprises methotrexate, lorcaserin, topiramate, olanzapine, risperidone, ziprasidone or a mixture thereof.

147. The method of claim 127 wherein in each tablet said first active composition is over coated with metformin in an amount ranging from about 70 to about 150 mg.

148. A medicament for use in the regeneration of organs and tissues in a subject suffering from one or more organ or tissue manifestations of glucose supply side associated metabolic syndrome, said medicament comprising an oral pharmaceutical dosage form comprising an inner controlled release component and an optional outer immediate or early release component, said inner controlled release component comprising an ileal brake hormone releasing substance comprising about 10 grams to about 20 grams of a refined sugar and optionally, a plant lipid, said ileal brake hormone releasing substance being encapsulated within an enteric coating which releases at least about 50% by weight of said ileal brake hormone releasing substance in the ileum and ascending colon of said subject after administration, said optional outer release component being formulated in an immediate or early release over coating of said inner controlled release component, said second active medicament acting synergistically with the inner core ileal brake hormone releasing substance upon one or more manifestations of said patient's metabolic syndrome.

149. A method of regenerating pancreatic beta cells in a subject suffering from type I diabetes, the method comprising:
(a) confirming that the subject with type I diabetes suffers from pancreatic beta cell damage;
(b) administering to the subject an effective amount of a pharmaceutical composition comprising between about 10 grams to about 20 grams of a refined sugar which is microencapsulated within an enteric coating which dissolves in vivo at a pH of around 7.2 to around 7.5, and optionally an effective amount of an additional bioactive agent selected from the group consisting of a proton pump inhibitor, a DPP-IV inhibitor, and mixtures thereof, said additional bioactive agent being formulated in an overcoating on said enteric coating, said overcoating being in immediate release or early release form; and.
(c) thereafter, confirming pancreatic beta cell regeneration by determining an increase in expression levels of one or more markers selected from the group consisting of insulin, proinsulin, c-peptide, Ki67, MCM-7 and PCNA and/or confirming pancreatic beta cell regeneration by determining an increase over time in an elevation in C-peptide concentrations, an increase in insulin output and a reduction in required dose of insulin needed to control hyperglycemia.

150. A method of regenerating organs and tissues in a subject suffering from one or more organ or tissue manifestations of glucose supply side associated metabolic syndrome, the method comprising:
(a) confirming that the subject suffers from or is at risk for suffering from organ and/or tissue damage associated with metabolic syndrome; and
(b) administering to the subject an effective amount of a pharmaceutical composition comprising between about 10 grams to about 20 grams of a refined sugar which is microencapsulated within an enteric coating which dissolves in vivo in the ileum of said subject at a pH of around 7.2 to around 7.5, wherein said organ to be regenerated is the subject's liver, GI tract, cardiovascular system, kidney, lungs and brain.

151. The method according to claim 150 wherein said organ to be regenerated is the subject's liver.

152. The method according to claim 150 wherein said organ to be regenerated is the subject's brain and said regeneration improves the patient's cognition.

153. the method according to claim 151 wherein said subject suffers from Alzheimer's disease.
